(19) United States
(12) Patent Application Publication
(10) Pub. No.: US 2021/0277148 A1
(43) Pub. Date: Sep. 9, 2021

(54) CHIMERIC RECEPTORS TO STEAP1 AND METHODS OF USE THEREOF

(71) Applicant: AMGEN INC., Thousand Oaks, CA (US)

(72) Inventor: Olivier NOLAN-STEVAUX, Millbrae, CA (US)

(73) Assignee: AMGEN INC., Thousand Oaks, CA (US)

(21) Appl. No.: 17/260,977

(22) PCT Filed: Jul. 17, 2019
(86) PCT No.: PCT/US2019/042245

§ 371 Date: Jan. 15, 2021

Related U.S. Application Data
(60) Provisional application No. 62/700,178, filed on Jul. 18, 2018.

(51) Int. Cl. C07K 16/40; C07K 14/705; C07K 14/725; A61K 35/17; C07K 14/715; C07K 14/73

(52) U.S. Cl. CPC C07K 16/40; A61K 38/00; C07K 14/70521; C07K 14/7051; A61K 35/17; C07K 14/70507; C07K 14/70578; C07K 14/7151; C07K 14/70503; C07K 14/7155; C07K 14/70514; C07K 14/70546; C07K 14/70596; C07K 2317/565; C07K 2317/515; C07K 2317/51; C07K 2317/622; C07K 14/70517

(57) ABSTRACT

Antigen binding molecules, chimeric receptors, and engineered immune cells to STEAP1 are disclosed in accordance with the invention. The invention further relates to vectors, compositions, and methods of treatment and/or detection using the STEAP1 antigen binding molecules and engineered immune cells.

### BACKGROUND OF THE INVENTION

Prostate cancer is the most frequently diagnosed cancer in men aside from skin cancer. With an estimated 28,170 deaths in 2012, prostate cancer is the second-leading cause of cancer death in men. Hormonal therapy, chemotherapy, radiation, or a combination of these treatments is used to treat more advanced disease. Despite the above identified advances in prostate cancer therapy, there is a great need for additional therapeutic agents capable of effectively inhibiting prostate cancer progression including in androgen receptor inhibitor naive prostate cancer.

Engineered immune cells have been shown to possess desired qualities in therapeutic treatments, particularly in oncology. Two main types of engineered immune cells are those that contain chimeric antigen receptors (termed “CARs” or “CAR-Ts”) and T-cell receptors (“TCRs”). These engineered cells are engineered to endow them with antigen specificity while retaining or enhancing their ability to recognize and kill a target cell. Chimeric antigen receptors may comprise, for example, (i) an antigen-specific component (“antigen binding molecule”), (ii) one or more costimulatory domains, and (iii) one or more activating domains. Each domain may be heterogeneous, that is, comprised of sequences derived from different protein chains. Chimeric antigen receptor-expressing immune cells (such as T cells) may be used in various therapies, including cancer therapies. It will be appreciated that costimulating polypeptides as defined herein may be used to enhance the activation of CAR-expressing cells against target antigens, and therefore increase the potency of adoptive immunotherapy.

T cells can be engineered to possess specificity to one or more desired targets. For example, T cells can be transduced with DNA or other genetic material encoding an antigen binding molecule, such as one or more single chain variable fragment (“scFv”) of an antibody, in conjunction with one or more signaling molecules, and/or one or more activating domains, such as CD3 zeta.

In addition to the CAR-T cells' ability to recognize and destroy the targeted cells, successful T cell therapy benefits from the CAR-T cells' ability to persist and maintain the ability to proliferate in response to antigen.

A need exists to identify novel and improved therapies for treating STEAP1 related diseases and disorders.

### SUMMARY OF THE INVENTION

The invention relates to engineered immune cells (such as CARs or TCRs), antigen binding molecules (including but not limited to, antibodies, scFvs, heavy and/or light chains, and CDRs of these antigen binding molecules) with specificity to STEAP1.

Chimeric antigen receptors of the invention typically comprise: (i) a STEAP1 specific antigen binding molecule, (ii) one or more costimulatory domain, and (iii) one or more activating domain. It will be appreciated that each domain may be heterogeneous, thus comprised of sequences derived from different protein chains.

In some embodiments, the invention relates to a chimeric antigen receptor comprising an antigen binding molecule that specifically binds to STEAP1, wherein the antigen binding molecule comprises at least one of: (a) a variable heavy chain CDR1 comprising an amino acid sequence differing from that of SEQ ID NOs:89, 99, 109, 119, 129, or 139 by not more than 3, 2, 1, or 0 amino acid residues; (b) a variable heavy chain CDR2 comprising an amino acid sequence differing from that of SEQ ID NOs:90, 100, 110, 120, 130, or 140 by not more than 3, 2, 1, or 0 amino acid residues; (c) a variable heavy chain CDR3 comprising an amino acid sequence differing from that of SEQ ID NOs:91, 101, 111, 121, 131, or 141 by not more than 3, 2, 1, or 0 amino acid residues; (d) a variable light chain CDR1 comprising an amino acid sequence differing from that of SEQ ID NOs:94, 104, 114, 124, 134, or 14 by not more than 3, 2, 1, or 0 amino acid residues; (e) a variable light chain CDR2 comprising an amino acid sequence differing from that of SEQ ID NOs:95, 105, 115, 125, 135, or 145 by not more than 3, 2, 1, or 0 amino acid residues; (0 a variable light chain CDR3 comprising an amino acid sequence differing from that of SEQ IDs:96, 106, 116, 126, 136, or 146 by not more than 3, 2, 1, or 0 amino acid residues.

In other embodiments, the chimeric antigen receptor further comprises at least one costimulatory domain. In further embodiments, the chimeric antigen receptor further comprises at least one activating domain.

In certain embodiments the costimulatory domain is a signaling region of CD28, CD28T, OX-40, 4-1BB/CD137, CD2, CD7, CD27, CD30, CD40, Programmed Death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD1 1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

In some embodiments, the costimulatory domain is derived from 4-1BB. In other embodiments, the costimulatory domain is derived from OX40. See also Hombach et al., Oncoimmunology. 2012 Jul. 1; 1(4): 458-466. In still other embodiments, the costimulatory domain comprises ICOS as described in Guedan et al., Aug. 14, 2014; Blood: 124 (7) and Shen et al., Journal of Hematology & Oncology (2013) 6:33. In still other embodiments, the costimulatory domain comprises CD27 as described in Song et al., Oncoimmunology. 2012 Jul. 1; 1(4): 547-549.

In certain embodiments, the CD28 costimulatory domain comprises SEQ ID NO:2, SEQ ID NO:4, SEQ ID NO:6, or SEQ ID NO:8. In additional embodiments, the CD8 costimulatory domain comprises SEQ ID NO:14. In further embodiments, the activating domain comprises CD3, CD3 zeta, or CD3 zeta having the sequence set forth in SEQ ID NO:10.

In other embodiments, the invention relates to a chimeric antigen receptor wherein the costimulatory domain comprises SEQ ID NO:2 and the activating domain comprises SEQ ID NO:10.

The invention further relates to polynucleotides encoding the chimeric antigen receptors, and vectors comprising the polynucleotides. The vector can be, for example, a retroviral vector, a DNA vector, a plasmid, a RNA vector, an adenoviral vector, an adenovirus associated vector, a lentiviral vector, or any combination thereof. The invention further relates to immune cells comprising the vectors. In some embodiments, the lentiviral vector is a pGAR vector.

Exemplary immune cells include, but are not limited to T cells, tumor infiltrating lymphocytes (TILs), NK cells, TCR-expressing cells, dendritic cells, or NK-T cells. The T cells can be autologous, allogeneic, or heterologous. In other embodiments, the invention relates to pharmaceutical compositions comprising the immune cells of described herein.

In certain embodiments, the invention relates to antigen binding molecules (and chimeric antigen receptors comprising these molecules) comprising at least one of:
(a) a VH region differing from the amino acid sequence of the VH region of 2F3 by no more than 10, 9, 8, 7, 6, 5, 4, 3, 2, 1, or 0 amino acid residues and a VL region differing from the amino acid sequence of the VL region of 2F3 by no more than 10, 9, 8, 7, 6, 5, 4, 3, 2, 1, or 0 amino acid residues;
(b) a VH region differing from the amino acid sequence of the VH region of 11C2 by no more than 10, 9, 8, 7, 6, 5, 4, 3, 2, 1, or 0 amino acid residues and a VL region differing from the amino acid sequence of the VL region of 11C2 by no more than 10, 9, 8, 7, 6, 5, 4, 3, 2, 1, or 0 amino acid residues;
(c) a VH region differing from the amino acid sequence of the VH region of 1A1 by no more than 10, 9, 8, 7, 6, 5, 4, 3, 2, 1, or 0 amino acid residues and a VL region differing from the amino acid sequence of the VL region of 1A1 by no more than 10, 9, 8, 7, 6, 5, 4, 3, 2, 1, or 0 amino acid residues;
(d) a VH region differing from the amino acid sequence of the VH region of 7A4 by no more than 10, 9, 8, 7, 6, 5, 4, 3, 2, 1, or 0 amino acid residues and a VL region differing from the amino acid sequence of the VL region of 7A4 by no more than 10, 9, 8, 7, 6, 5, 4, 3, 2, 1, or 0 amino acid residues; and
(e) a VH region differing from the amino acid sequence of the VH region of 7A5 by no more than 10, 9, 8, 7, 6, 5, 4, 3, 2, 1, or 0 amino acid residues and a VL region differing from the amino acid sequence of the VL region of 7A5 by no more than 10, 9, 8, 7, 6, 5, 4, 3, 2, 1, or 0 amino acid residues;
(f) a VH region differing from the amino acid sequence of the VH region of 14C1 by no more than 10, 9, 8, 7, 6, 5, 4, 3, 2, 1, or 0 amino acid residues and a VL region differing from the amino acid sequence of the VL region of 14C1 by no more than 10, 9, 8, 7, 6, 5, 4, 3, 2, 1, or 0 amino acid residues;
and wherein the VH and VL region or regions are linked by at least one linker.

In other embodiments, the invention relates to antigen binding molecules (and chimeric antigen receptors comprising these molecules) wherein the linker comprises at least one of the scFv G4S linker and the scFv Whitlow linker.

In other embodiments, the invention relates to vectors encoding the polypeptides of the invention and to immune cells comprising these polypeptides. Preferred immune cells include T cells, tumor infiltrating lymphocytes (TILs), NK cells, TCR-expressing cells, dendritic cells, or NK-T cells. The T cells may be autologous, allogeneic, or heterologous.

In other embodiments, the invention relates to isolated polynucleotides encoding a chimeric antigen receptor (CAR) or T cell receptor (TCR) comprising an antigen binding molecule that specifically binds to STEAP1, wherein the antigen binding molecule comprises a variable heavy (VII) chain CDR3 comprising an amino acid sequence of SEQ ID NO:19 or SEQ ID NO:27. The polynucleotides may further comprise an activating domain. In preferred embodiments, the activating domain is CD3, more preferably CD3 zeta, more preferably the amino acid sequence set forth in SEQ ID NO:9.

In other embodiments, the invention includes a costimulatory domain, such as CD28, CD28T, OX40, 4-1BB/CD137, CD2, CD3 (alpha, beta, delta, epsilon, gamma, zeta), CD4, CD5, CD7, CD9, CD16, CD22, CD27, CD30, CD 33, CD37, CD40, CD 45, CD64, CD80, CD86, CD134, CD137, CD154, PD-1, ICOS, lymphocyte function-associated antigen-1 (LFA-1 (CD1 1a/CD18), CD247, CD276 (B7-H3), LIGHT (tumor necrosis factor superfamily member 14; TNFSF14), NKG2C, Ig alpha (CD79a), DAP-10, Fc gamma receptor, MHC class I molecule, TNF, TNFr, integrin, signaling lymphocytic activation molecule, BTLA, Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1-1d, ITGAE, CD103, ITGAL, CD1-1a, LFA-1, ITGAM, CD1-1b, ITGAX, CD1-1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, or fragments or combinations thereof. Preferred costimulatory domains are recited hereinbelow.

The invention further relates to methods of treating a disease or disorder in a subject in need thereof comprising administering to the subject the antigen binding molecules, the CARs, TCRs, polynucleotides, vectors, cells, or compositions according to the invention. Suitable diseases for treatment include, but are not limited to, prostate cancer, including metastatic castration resistant prostate cancer.

### BRIEF DESCRIPTION OF THE FIGURES

FIG. 1, depicts flow cytometric analysis of STEAP1 cell surface expression on human cell lines.

FIG. 2, depicts CAR expression in primary human T cells electroporated with mRNA encoding for various CARs.

FIG. 3, depicts cytolytic activity of electroporated CART cells against multiple cell lines.

FIG. 4, comprising of FIGS. 3A, and 3B, depicts IFNγ, IL-2, and TNFα production by electroporated CAR T cells.

FIG. 5, depicts CAR expression in lentivirus transduced primary human T cells from two healthy donors.

FIG. 6, depicts the pGAR vector map.

### DETAILED DESCRIPTION OF THE INVENTION

It will be appreciated that chimeric antigen receptors (CARs or CAR-Ts) and T cell receptors (TCRs) are genetically engineered receptors. These engineered receptors can be readily inserted into and expressed by immune cells, including T cells in accordance with techniques known in the art. With a CAR, a single receptor can be programmed to both recognize a specific antigen and, when bound to that antigen, activate the immune cell to attack and destroy the cell bearing that antigen. When these antigens exist on tumor cells, an immune cell that expresses the CAR can target and kill the tumor cell.

CARs can be engineered to bind to an antigen (such as a cell-surface antigen) by incorporating an antigen binding molecule that interacts with that targeted antigen. Preferably, the antigen binding molecule is an antibody fragment thereof, and more preferably one or more single chain antibody fragment (“scFv”). An scFv is a single chain antibody fragment having the variable regions of the heavy and light chains of an antibody linked together. See U.S. Pat. Nos. 7,741,465, and 6,319,494 as well as Eshhar et al., Cancer Immunol Immunotherapy (1997) 45: 131-136. An scFv retains the parent antibody's ability to specifically interact with target antigen. scFvs are preferred for use in chimeric antigen receptors because they can be engineered to be expressed as part of a single chain along with the other CAR components. Id. See also Krause et al., J. Exp. Med., Volume 188, No. 4, 1998 (619-626); Finney et al., Journal of Immunology, 1998, 161: 2791-2797. It will be appreciated that the antigen binding molecule is typically contained within the extracellular portion of the CAR such that it is capable of recognizing and binding to the antigen of interest. Bispecific and multispecific CARs are contemplated within the scope of the invention, with specificity to more than one target of interest.

Costimulatory Domains. Chimeric antigen receptors may incorporate costimulatory (signaling) domains to increase their potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Annu. Rev. Pharmacol. Toxicol. 56:59-83 (2016). For example, CD28 is a costimulatory protein found naturally on T-cells. The complete native amino acid sequence of CD28 is described in NCBI Reference Sequence: NP_006130.1. The complete native CD28 nucleic acid sequence is described in NCBI Reference Sequence: NM_006139.1.

Certain CD28 domains have been used in chimeric antigen receptors. In one embodiment, a novel CD28 extracellular domain, termed “CD28T”, can be used, and has been unexpectedly found to provide certain benefits when utilized in a CAR construct.

The nucleotide sequence of the CD28T molecule, including the extracellular CD28T domain, and the CD28 transmembrane and intracellular domains is set forth in SEQ ID NO:1:

CTTGATAATGAAAAGTCAAACGGAACAATCATTCACGTGAAGGGCAAGCA
CCTCTGTCCGTCACCCTTGTTCCCTGGTCCATCCAAGCCATTCTGGGTGT
TGGTCGTAGTGGGTGGAGTCCTCGCTTGTTACTCTCTGCTCGTCACCGTG
GCTTTTATAATCTTCTGGGTTAGATCCAAAAGAAGCCGCCTGCTCCATAG
CGATTACATGAATATGACTCCACGCCGCCCTGGCCCCACAAGGAAACACT
ACCAGCCTTACGCACCACCTAGAGATTTCGCTGCCTATCGGAGC

The corresponding amino acid sequence is set forth in SEQ ID NO:2:

LDNEKSNGTIIHVKGKHLCPSPLFPGPSKPFWVLVVVGGVLACYSLLVTV
AFIIFWVRSK RSRLLHSDYM NMTPRRPGPT RKHYQPYAPP RDFAAY
RS

The nucleotide sequence of the extracellular portion of CD28T is set forth in SEQ ID NO:3:

CTTGATAATGAAAAGTCAAACGGAACAATCATTCACGTGAAGGGCAAGCA
CCTCTGTCCGTCACCCTTGTTCCCTGGTCCATCCAAGCCA

The corresponding amino acid sequence of the CD28T extracellular domain is set forth in SEQ ID NO:4: LDNEKSNGTI IHVKGKHLCP SPLFPGPSKP

The nucleotide sequence of the CD28 transmembrane domain is set forth in SEQ ID NO:5):

TTCTGGGTGTTGGTCGTAGTGGGTGGAGTCCTCGCTTGTTACTCTCTGCT
CGTCACCGTGGCTTTTATAATCTTCTGGGTT

The amino acid sequence of the CD28 transmembrane domain is set forth in SEQ ID NO:6:

FWVLVVVGGV LACYSLLVTV AFIIFWV

The nucleotide sequence of the CD28 intracellular signaling domain is set forth in SEQ ID NO:7:

AGATCCAAAAGAAGCCGCCTGCTCCATAGCGATTACATGAATATGACTCC
ACGCCGCCCTGGCCCCACAAGGAAACACTACCAGCCTTACGCACCACCTA
GAGATTTCGCTGCCTATCGGAGC

The amino acid sequence of the CD28 intracellular signaling domain is set forth in SEQ ID NO:8: RSKRSRLLHSDYMNMTPRRPGPTRKHYQPYAPPRDFAAYRS

Additional CD28 sequences suitable for use in the invention include the CD28 nucleotide sequence set forth in SEQ ID NO:11:

ATTGAGGTGATGTATCCACCGCCTTACCTGGATAACGAAAAGAGTAACGG
TACCATCATTCACGTGAAAGGTAAACACCTGTGTCCTTCTCCCCTCTTCC
CCGGGCCATCAAAGCCC

The corresponding amino acid sequence is set forth in SEQ ID NO:12: IEVMYPPPYLDNEKSNGTIIHVKGKHLCPSPLFPGPSKP

Other suitable extracellular or transmembrane sequences can be derived from CD8. The nucleotide sequence of a suitable CD8 extracellular and transmembrane domain is set forth in SEQ ID NO:13:

GCTGCAGCATTGAGCAACTCAATAATGTATTTTAGTCACTTTGTACCAGT
GTTCTTGCCGGCTAAGCCTACTACCACACCCGCTCCACGGCCACCTACCC
CAGCTCCTACCATCGCTTCACAGCCTCTGTCCCTGCGCCCAGAGGCTTGC
CGACCGGCCGCAGGGGGCGCTGTTCATACCAGAGGACTGGATTTCGCCTG
CGATATCTATATCTGGGCACCCCTGGCCGGAACCTGCGGCGTACTCCTGC
TGTCCCTGGTCATCACGCTCTATTGTAATCACAGGAAC

The corresponding amino acid sequence is set forth in SEQ ID NO:14:

AAALSNSIMYFSHFVPVFLPAKPTTTPAPRPPTPAPTIASQPLSLRPEAC
RPAAGGAVHTRGLDFACDIYIWAPLAGTCGVLLLSLVITLYCNHRN

Other suitable intracellular signaling sequences can be derived from 41-BB. The nucleotide sequence of a suitable 41-BB intracellular signaling domain is set forth in SEQ ID NO:15:

CGCTTTTCCGTCGTTAAGCGGGGGAGAAAAAAGCTGCTGTACATTTTCAA
ACAGCCGTTTATGAGGCCGGTCCAAACGACTCAGGAAGAGGACGGCTGCT
CCTGCCGCTTTCCTGAGGAGGAGGAGGGCGGGTGCGAACTG

The corresponding amino acid sequence is set forth in SEQ ID NO:16:

RFSVVKRGRKKLLYIFKQPFMRPVQTTQEEDGCSCRFPEEEEGGCEL

Suitable costimulatory domains within the scope of the invention can be derived from, among other sources, CD28, CD28T, OX40, 4-1BB/CD137, CD2, CD3 (alpha, beta, delta, epsilon, gamma, zeta), CD4, CD5, CD7, CD9, CD16, CD22, CD27, CD30, CD 33, CD37, CD40, CD 45, CD64, CD80, CD86, CD134, CD137, CD154, PD-1, ICOS, lymphocyte function-associated antigen-1 (LFA-1 (CD11a/CD18), CD247, CD276 (B7-H3), LIGHT (tumor necrosis factor superfamily member 14; TNFSF14), NKG2C, Ig alpha (CD79a), DAP-10, Fc gamma receptor, MHC class I molecule, TNF, TNFr, integrin, signaling lymphocytic activation molecule, BTLA, Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1-1d, ITGAE, CD103, ITGAL, CD1-1a, LFA-1, ITGAM, CD1-1b, ITGAX, CD1-1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, or fragments or combinations thereof

Activating Domains.

CD3 is an element of the T cell receptor on native T cells, and has been shown to be an important intracellular activating element in CARs. In a preferred embodiment, the CD3 is CD3 zeta, the nucleotide sequence of which is set forth in SEQ ID NO:9:

AGGGTGAAGTTTTCCAGATCTGCAGATGCACCAGCGTATCAGCAGGGCCA
GAACCAACTGTATAACGAGCTCAACCTGGGACGCAGGGAAGAGTATGACG
TTTTGGACAAGCGCAGAGGACGGGACCCTGAGATGGGTGGCAAACCAAGA
CGAAAAAACCCCCAGGAGGGTCTCTATAATGAGCTGCAGAAGGATAAGAT
GGCTGAAGCCTATTCTGAAATAGGCATGAAAGGAGAGCGGAGAAGGGGAA
AAGGGCACGACGGTTTGTACCAGGGACTCAGCACTGCTACGAAGGATACT
TATGACGCTCTCCACATGCAAGCCCTGCCACCTAGG

The corresponding amino acid of intracellular CD3 zeta is set forth in SEQ ID NO:10:

RVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYD
ALHMQALPPR

### Domain Orientation

Structurally, it will appreciated that these domains correspond to locations relative to the immune cell. Thus, these domains can be part of the (i) “hinge” or extracellular (EC) domain (EC), (ii) the transmembrane (TM) domain, and/or (iii) the intracellular (cytoplasmic) domain (IC). The intracellular component frequently comprises in part a member of the CD3 family, preferably CD3 zeta, which is capable of activating the T cell upon binding of the antigen binding molecule to its target. In one embodiment, the hinge domain is typically comprised of at least one costimulatory domain as defined herein.

It will also be appreciated that the hinge region may also contain some or all of a member of the immunoglobulin family such as IgG1, IgG2, IgG3, IgG4, IgA, IgD, IgE, IgM, or fragment thereof.

Exemplary CAR constructs in accordance with the invention are set forth in Table 1.

TABLE 1

| | | Costimulatory | Activating |
|---|---|---|---|
| Construct Name | scFv | Domain | Domain |
| 2F3-CD28T-CD28-41BB | 2F3 | CD28T, 4-1BB | CD3 zeta |
| 2F3-CD28T-CD28 | 2F3 | CD28T | CD3 zeta |
| 2F3-CD28T-41BB | 2F3 | 4-1BB | CD3 zeta |
| 2F3-C8K-CD28 | 2F3 | CD28T | CD3 zeta |
| 2F3-C8K-41BB | 2F3 | 4-1BB | CD3 zeta |
| 11C2-CD28T-CD28-41BB | 11C2 | CD28T, 4-1BB | CD3 zeta |
| 11C2-CD28T-CD28 | 11C2 | CD28T | CD3 zeta |
| 11C2-CD28T-41BB | 11C2 | 4-1BB | CD3 zeta |
| 11C2-C8K-CD28 | 11C2 | CD28T | CD3 zeta |
| 11C2-C8K-41BB | 11C2 | 4-1BB | CD3 zeta |
| 1A1-CD28T-CD28-41BB | 1A1 | CD28T, 4-1BB | CD3 zeta |
| 1A1-CD28T-CD28 | 1A1 | CD28T | CD3 zeta |
| 1A1-CD28T-41BB | 1A1 | 4-1BB | CD3 zeta |
| 1A1-C8K-CD28 | 1A1 | CD28T | CD3 zeta |
| 1A1-C8K-41BB | 1A1 | 4-1BB | CD3 zeta |
| 7A4-CD28T-CD28-41BB | 7A4 | CD28T, 4-1BB | CD3 zeta |
| 7A4-CD28T-CD28 | 7A4 | CD28T | CD3 zeta |
| 7A4-CD28T-41BB | 7A4 | 4-1BB | CD3 zeta |
| 7A4-C8K-CD28 | 7A4 | CD28T | CD3 zeta |
| 7A4-C8K-41 BB | 7A4 | 4-1BB | CD3 zeta |
| 7A5-CD28T-CD28-41BB | 7A5 | CD28T, 4-1BB | CD3 zeta |
| 7A5-CD28T-CD28 | 7A5 | CD28T | CD3 zeta |
| 7A5-CD28T-41BB | 7A5 | 4-1BB | CD3 zeta |
| 7A5-C8K-CD28 | 7A5 | CD28T | CD3 zeta |
| 7A5-C8K-41BB | 7A5 | 4-1BB | CD3 zeta |
| 14C1-CD28T-CD28-41BB | 14C1 | CD28T, 4-1BB | CD3 zeta |
| 14C1-CD28T-CD28 | 14C1 | CD28T | CD3 zeta |
| 14C1-CD28T-41BB | 14C1 | 4-1BB | CD3 zeta |
| 14C1-C8K-CD28 | 14C1 | CD28T | CD3 zeta |
| 14C1-C8K-41BB | 14C1 | 4-1BB | CD3 zeta |

### Domains Relative to the Cell

It will be appreciated that relative to the cell bearing the receptor, the engineered T cells of the invention comprise an antigen binding molecule (such as an scFv), an extracellular domain (which may comprise a “hinge” domain), a transmembrane domain, and an intracellular domain. The intracellular domain comprises at least in part an activating domain, preferably comprised of a CD3 family member such as CD3 zeta, CD3 epsilon, CD3 gamma, or portions thereof. It will further be appreciated that the antigen binding molecule (e.g., one or more scFvs) is engineered such that it is located in the extracellular portion of the molecule/construct, such that it is capable of recognizing and binding to its target or targets.

Extracellular Domain. The extracellular domain is beneficial for signaling and for an efficient response of lymphocytes to an antigen. Extracellular domains of particular use in this invention may be derived from (i.e., comprise) CD28, CD28T, CD8, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof. The extracellular domain may be derived either from a natural or from a synthetic source.

As described herein, extracellular domains often comprise a hinge portion. This is a portion of the extracellular domain, sometimes referred to as a “spacer” region. A variety of hinges can be employed in accordance with the invention, including costimulatory molecules as discussed above, as well as immunoglobulin (Ig) sequences or other suitable molecules to achieve the desired special distance from the target cell. In some embodiments, the entire extracellular region comprises a hinge region. In some embodiments, the hinge region comprises CD28T, or the EC domain of CD28.

Transmembrane Domain. The CAR can be designed to comprise a transmembrane domain that is fused to the extracellular domain of the CAR. It can similarly be fused to the intracellular domain of the CAR. In one embodiment, the transmembrane domain that naturally is associated with one of the domains in a CAR is used. In some instances, the transmembrane domain can be selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this invention may be derived from (i.e. comprise) CD28, CD28T, CD8, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

Optionally, short linkers may form linkages between any or some of the extracellular, transmembrane, and intracellular domains of the CAR.

In one embodiment, the transmembrane domain in the CAR of the invention is a CD8 transmembrane domain. In one embodiment, the CD8 transmembrane domain comprises the transmembrane portion of the nucleic acid sequence of SEQ ID NO:13. In another embodiment, the CD8 transmembrane domain comprises the nucleic acid sequence that encodes the transmembrane amino acid sequence contained within SEQ ID NO:14.

In certain embodiments, the transmembrane domain in the CAR of the invention is the CD28 transmembrane domain. In one embodiment, the CD28 transmembrane domain comprises the nucleic acid sequence of SEQ ID NO:5. In one embodiment, the CD28 transmembrane domain comprises the nucleic acid sequence that encodes the amino acid sequence of SEQ ID NO:6. In another embodiment, the CD28 transmembrane domain comprises the amino acid sequence of SEQ ID NO:6.

Intracellular (Cytoplasmic) Domain. The intracellular (cytoplasmic) domain of the engineered T cells of the invention can provide activation of at least one of the normal effector functions of the immune cell. Effector function of a T cell, for example, may be cytolytic activity or helper activity including the secretion of cytokines.

It will be appreciated that suitable intracellular molecules include (i.e., comprise), but are not limited to CD28, CD28T, CD8, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

In a preferred embodiment, the cytoplasmic domain of the CAR can be designed to comprise the CD3 zeta signaling domain by itself or combined with any other desired cytoplasmic domain(s) useful in the context of the CAR of the invention. For example, the cytoplasmic domain of the CAR can comprise a CD3 zeta chain portion and a costimulatory signaling region.

The cytoplasmic signaling sequences within the cytoplasmic signaling portion of the CAR of the invention may be linked to each other in a random or specified order.

In one preferred embodiment, the cytoplasmic domain is designed to comprise the signaling domain of CD3 zeta and the signaling domain of CD28. In another embodiment, the cytoplasmic domain is designed to comprise the signaling domain of CD3 zeta and the signaling domain of 4-1BB, wherein the cytoplasmic CD28 comprises the nucleic acid sequence set forth in SEQ ID NO:15 and the amino acid sequence set forth in SEQ ID NO:16. In another embodiment, the cytoplasmic domain in the CAR of the invention is designed to comprise a portion of CD28 and CD3 zeta, wherein the cytoplasmic CD28 comprises the nucleic acid sequence set forth in SEQ ID NO:7 and the amino acid sequence set forth in SEQ ID NO:8. The CD3 zeta nucleic acid sequence is set forth in SEQ ID NO:9, and the amino acid sequence is set forth in SEQ ID NO:8.

It will be appreciated that one preferred orientation of the CARs in accordance with the invention comprises an antigen binding domain (such as scFv) in tandem with a costimulatory domain and an activating domain. The costimulatory domain can comprise one or more of an extracellular portion, a transmembrane portion, and an intracellular portion. It will be further appreciated that multiple costimulatory domains can be utilized in tandem.

In some embodiments, nucleic acids are provided comprising a promoter operably linked to a first polynucleotide encoding an antigen binding molecule, at least one costimulatory molecule, and an activating domain.

In some embodiments, the nucleic acid construct is contained within a viral vector. In some embodiments, the viral vector is selected from the group consisting of retroviral vectors, murine leukemia virus vectors, SFG vectors, adenoviral vectors, lentiviral vectors, adeno-associated virus (AAV) vectors, Herpes virus vectors, and vaccinia virus vectors. In some embodiments, the nucleic acid is contained within a plasmid.

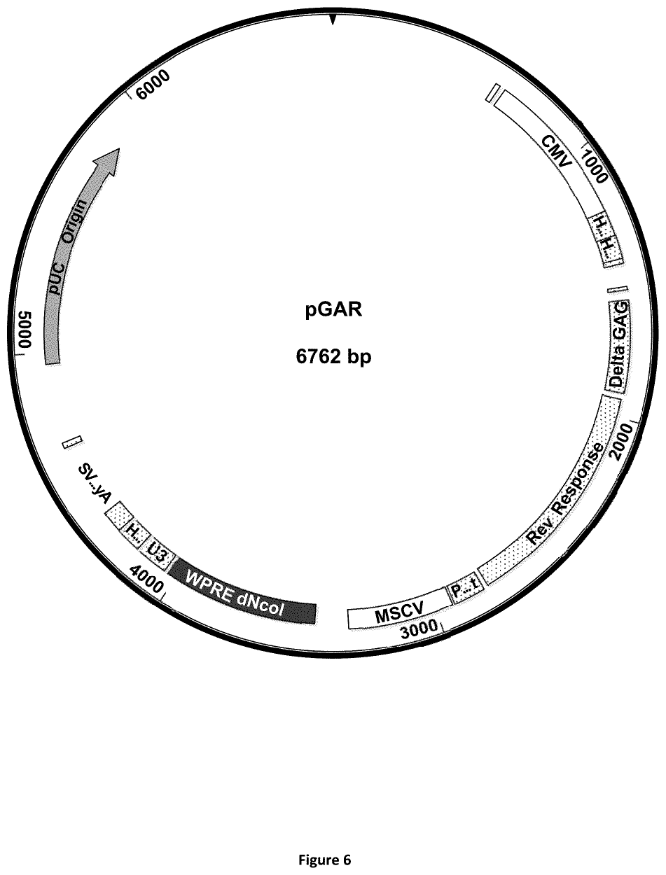

The invention further relates to isolated polynucleotides encoding the chimeric antigen receptors, and vectors comprising the polynucleotides. Any vector known in the art can be suitable for the present invention. In some embodiments, the vector is a viral vector. In some embodiments, the vector is a retroviral vector (such as pMSVG1), a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector (such as pGAR), or any combination thereof. The pGAR vector map is shown in FIG. 6. The pGAR sequence is as follows:

(SEQ ID NO: 147)
CTGACGCGCCCTGTAGCGGCGCATTAAGCGCGGCGGGTGTGGTGGTTACGC
GCAGCGTGACCGCTACACTTGCCAGCGCCCTAGCGCCCGCTCCTTTCGCTT
TCTTCCCTTCCTTTCTCGCCACGTTCGCCGGCTTTCCCCGTCAAGCTCTAA
ATCGGGGGCTCCCTTTAGGGTTCCGATTTAGTGCTTTACGGCACCTCGACC
CCAAAAAACTTGATTAGGGTGATGGTTCACGTAGTGGGCCATCGCCCTGAT
AGACGGTTTTTCGCCCTTTGACGTTGGAGTCCACGTTCTTTAATAGTGGAC
TCTTGTTCCAAACTGGAACAACACTCAACCCTATCTCGGTCTATTCTTTTG
ATTTATAAGGGATTTTGCCGATTTCGGCCTATTGGTTAAAAAATGAGCTGA
TTTAACAAAAATTTAACGCGAATTTTAACAAAATATTAACGCTTACAATTT
GCCATTCGCCATTCAGGCTGCGCAACTGTTGGGAAGGGCGATCGGTGCGGG
CCTCTTCGCTATTACGCCAGCTGGCGAAAGGGGGATGTGCTGCAAGGCGAT
TAAGTTGGGTAACGCCAGGGTTTTCCCAGTCACGACGTTGTAAAACGACGG
CCAGTGAATTGTAATACGACTCACTATAGGGCGACCCGGGGATGGCGCGCC
AGTAATCAATTACGGGGTCATTAGTTCATAGCCCATATATGGAGTTCCGCG
TTACATAACTTACGGTAAATGGCCCGCCTGGCTGACCGCCCAACGACCCCC
GCCCATTGACGTCAATAATGACGTATGTTCCCATAGTAACGCCAATAGGGA
CTTTCCATTGACGTCAATGGGTGGAGTATTTACGGTAAACTGCCCACTTGG
CAGTACATCAAGTGTATCATATGCCAAGTACGCCCCCTATTGACGTCAATG
ACGGTAAATGGCCCGCCTGGCATTATGCCCAGTACATGACCTTATGGGACT
TTCCTACTTGGCAGTACATCTACGTATTAGTCATCGCTATTACCATGCTGA
TGCGGTTTTGGCAGTACATCAATGGGCGTGGATAGCGGTTTGACTCACGGG
GATTTCCAAGTCTCCACCCCATTGACGTCAATGGGAGTTTGTTTTGGCACC
AAAATCAACGGGACTTTCCAAAATGTCGTAACAACTCCGCCCCATTGACGC
AAATGGGCGGTAGGCGTGTACGGTGGGAGGTCTATATAAGCAGAGCTGGTT
TAGTGAACCGGGGTCTCTCTGGTTAGACCAGATCTGAGCCTGGGAGCTCTC
TGGCTAACTAGGGAACCCACTGCTTAAGCCTCAATAAAGCTTGCCTTGAGT
GCTTCAAGTAGTGTGTGCCCGTCTGTTGTGTGACTCTGGTAACTAGAGATC
CCTCAGACCCTTTTAGTCAGTGTGGAAAATCTCTAGCAGTGGCGCCCGAAC
AGGGACTTGAAAGCGAAAGGGAAACCAGAGGAGCTCTCTCGACGCAGGACT
CGGCTTGCTGAAGCGCGCACGGCAAGAGGCGAGGGGCGGCGACTGGTGAGT
ACGCCAAAAATTTTGACTAGCGGAGGCTAGAAGGAGAGAGATGGGTGCGAG
AGCGTCAGTATTAAGCGGGGGAGAATTAGATCGCGATGGGAAAAAATTCGG
TTAAGGCCAGGGGGAAAGAAAAAATATAAATTAAAACATATAGTATGGGCA
AGCAGGGAGCTAGAACGATTCGCAGTTAATCCTGGCCTGTTAGAAACATCA
GAAGGCTGTAGACAAATACTGGGACAGCTACAACCATCCCTTCAGACAGGA
TCAGAAGAACTTAGATCATTATATAATACAGTAGCAACCCTCTATTGTGTG
CATCAAAGGATAGAGATAAAAGACACCAAGGAAGCTTTAGACAAGATAGAG
GAAGAGCAAAACAAAAGTAAGACCACCGCACAGCAAGCCGCCGCTGATCTT
CAGACCTGGAGGAGGAGATATGAGGGACAATTGGAGAAGTGAATTATATAA
ATATAAAGTAGTAAAAATTGAACCATTAGGAGTAGCACCCACCAAGGCAAA
GAGAAGAGTGGTGCAGAGAGAAAAAAGAGCAGTGGGAATAGGAGCTTTGTT
CCTTGGGTTCTTGGGAGCAGCAGGAAGCACTATGGGCGCAGCGTCAATGAC
GCTGACGGTACAGGCCAGACAATTATTGTCTGGTATAGTGCAGCAGCAGAA
CAATTTGCTGAGGGCTATTGAGGCGCAACAGCATCTGTTGCAACTCACAGT
CTGGGGCATCAAGCAGCTCCAGGCAAGAATCCTGGCTGTGGAAAGATACCT
AAAGGATCAACAGCTCCTGGGGATTTGGGGTTGCTCTGGAAAACTCATTTG
CACCACTGCTGTGCCTTGGAATGCTAGTTGGAGTAATAAATCTCTGGAACA
GATTTGGAATCACACGACCTGGATGGAGTGGGACAGAGAAATTAACAATTA
CACAAGCTTAATACACTCCTTAATTGAAGAATCGCAAAACCAGCAAGAAAA
GAATGAACAAGAATTATTGGAATTAGATAAATGGGCAAGTTTGTGGAATTG
GTTTAACATAACAAATTGGCTGTGGTATATAAAATTATTCATAATGATAGT
AGGAGGCTTGGTAGGTTTAAGAATAGTTTTTGCTGTACTTTCTATAGTGAA
TAGAGTTAGGCAGGGATATTCACCATTATCGTTTCAGACCCACCTCCCAAC
CCCGAGGGGACCCGACAGGCCCGAAGGAATAGAAGAAGAAGGTGGAGAGAG
AGACAGAGACAGATCCATTCGATTAGTGAACGGATCTCGACGGTATCGGTT
AACTTTTAAAAGAAAAGGGGGGATTGGGGGGTACAGTGCAGGGGAAAGAAT
AGTAGACATAATAGCAACAGACATACAAACTAAAGAATTACAAAAACAAAT
TACAAAATTCAAAATTTTATCGCGATCGCGGAATGAAAGACCCCACCTGTA
GGTTTGGCAAGCTAGCTTAAGTAACGCCATTTTGCAAGGCATGGAAAATAC
ATAACTGAGAATAGAGAAGTTCAGATCAAGGTTAGGAACAGAGAGACAGCA
GAATATGGGCCAAACAGGATATCTGTGGTAAGCAGTTCCTGCCCCGGCTCA
GGGCCAAGAACAGATGGTCCCCAGATGCGGTCCCGCCCTCAGCAGTTTCTA
GAGAACCATCAGATGTTTCCAGGGTGCCCCAAGGACCTGAAAATGACCCTG
TGCCTTATTTGAACTAACCAATCAGTTCGCTTCTCGCTTCTGTTCGCGCGC
TTCTGCTCCCCGAGCTCAATAAAAGAGCCCACAACCCCTCACTCGGCGCGC
CAGTCCTTCGAAGTAGATCTTTGTCGATCCTACCATCCACTCGACACACCC
GCCAGCGGCCGCTGCCAAGCTTCCGAGCTCTCGAATTAATTCACGGTACCC
ACCATGGCCTAGGGAGACTAGTCGAATCGATATCAACCTCTGGATTACAAA
ATTTGTGAAAGATTGACTGGTATTCTTAACTATGTTGCTCCTTTTACGCTA
TGTGGATACGCTGCTTTAATGCCTTTGTATCATGCTATTGCTTCCCGTATG
GCTTTCATTTTCTCCTCCTTGTATAAATCCTGGTTGCTGTCTCTTTATGAG
GAGTTGTGGCCCGTTGTCAGGCAACGTGGCGTGGTGTGCACTGTGTTTGCT
GACGCAACCCCCACTGGTTGGGGCATTGCCACCACCTGTCAGCTCCTTTCC
GGGACTTTCGCTTTCCCCCTCCCTATTGCCACGGCGGAACTCATCGCCGCC
TGCCTTGCCCGCTGCTGGACAGGGGCTCGGCTGTTGGGCACTGACAATTCC
GTGGTGTTGTCGGGGAAGCTGACGTCCTTTTCATGGCTGCTCGCCTGTGTT
GCCACCTGGATTCTGCGCGGGACGTCCTTCTGCTACGTCCCTTCGGCCCTC
AATCCAGCGGACCTTCCTTCCCGCGGCCTGCTGCCGGCTCTGCGGCCTCTT
CCGCGTCTTCGCCTTCGCCCTCAGACGAGTCGGATCTCCCTTTGGGCCGCC
TCCCCGCCTGGTTAATTAAAGTACCTTTAAGACCAATGACTTACAAGGCAG
CTGTAGATCTTAGCCACTTTTTAAAAGAAAAGGGGGGACTGGAAGGGCGAA
TTCACTCCCAACGAAGACAAGATCTGCTTTTTGCTTGTACTGGGTCTCTCT
GGTTAGACCAGATCTGAGCCTGGGAGCTCTCTGGCTAACTAGGGAACCCAC
TGCTTAAGCCTCAATAAAGCTTGCCTTGAGTGCTTCAAGTAGTGTGTGCCC
GTCTGTTGTGTGACTCTGGTAACTAGAGATCCCTCAGACCCTTTTAGTCAG
TGTGGAAAATCTCTAGCAGGCATGCCAGACATGATAAGATACATTGATGAG
TTTGGACAAACCACAACTAGAATGCAGTGAAAAAAATGCTTTATTTGTGAA
ATTTGTGATGCTATTGCTTTATTTGTAACCATTATAAGCTGCAATAAACAA
GTTAACAACAACAATTGCATTCATTTTATGTTTCAGGTTCAGGGGGAGGTG
TGGGAGGTTTTTTGGCGCGCCATCGTCGAGGTTCCCTTTAGTGAGGGTTAA
TTGCGAGCTTGGCGTAATCATGGTCATAGCTGTTTCCTGTGTGAAATTGTT
ATCCGCTCACAATTCCACACAACATACGAGCCGGAAGCATAAAGTGTAAAG
CCTGGGGTGCCTAATGAGTGAGCTAACTCACATTAATTGCGTTGCGCTCAC
TGCCCGCTTTCCAGTCGGGAAACCTGTCGTGCCAGCTGCATTAATGAATCG
GCCAACGCGCGGGGAGAGGCGGTTTGCGTATTGGGCGCTCTTCCGCTTCCT
CGCTCACTGACTCGCTGCGCTCGGTCGTTCGGCTGCGGCGAGCGGTATCAG
CTCACTCAAAGGCGGTAATACGGTTATCCACAGAATCAGGGGATAACGCAG
GAAAGAACATGTGAGCAAAAGGCCAGCAAAAGGCCAGGAACCGTAAAAAGG
CCGCGTTGCTGGCGTTTTTCCATAGGCTCCGCCCCCCTGACGAGCATCACA
AAAATCGACGCTCAAGTCAGAGGTGGCGAAACCCGACAGGACTATAAAGAT
ACCAGGCGTTTCCCCCTGGAAGCTCCCTCGTGCGCTCTCCTGTTCCGACCC
TGCCGCTTACCGGATACCTGTCCGCCTTTCTCCCTTCGGGAAGCGTGGCGC
TTTCTCATAGCTCACGCTGTAGGTATCTCAGTTCGGTGTAGGTCGTTCGCT
ACCAAGCTGGGCTGTGTGCACGAACCCCCCGTTCGCCCGACCGCTGCGCCT
TATCCGGTAACTATCGTCTTGAGTCCAACCCGGTAAGACACGACTTATCGC
CACTGGCAGCAGCCACTGGTAACAGGATTAGCAGAGCGAGGTATGTAGGCG
GTGCTACAGAGTTCTTGAAGTGGTGGCCTAACTACGGCTACACTAGAAGAA
CAGTATTTGGTATCTGCGCTCTGCTGAAGCCAGTTACCTTCGGAAAAAGAG
TTGGTAGCTCTTGATCCGGCAAACAAACCACCGCTGGTAGCGGTGGTTTTT
TTGTTTGCAAGCAGCAGATTACGCGCAGAAAAAAAGGATCTCAAGAAGATC
CTTTGATCTTTTCTACGGGGTCTGACGCTCAGTGGAACGAAAACTCACGTT
AAGGGATTTTGGTCATGAGATTATCAAAAAGGATCTTCACCTAGATCCTTT
CTAAATTAAAAATGAAGTTTTAAATCAATTAAAGTATATATGAGTAAACTT
GGTCTGACAGTTACCAATGCTTAATCAGTGAGGCACCTATCTCAGCGATCT
GTCTATTTCGTTCATCCATAGTTGCCTGACTCCCCGTCGTGTAGATAACTA
CGATACGGGAGGGCTTACCATCTGGCCCCAGTGCTGCAATGATACCGCGAG
ACCCACGCTCACCGGCTCCAGATTTATCAGCAATAAACCAGCCAGCCGGAA
GGGCCGAGCGCAGAAGTGGTCCTGCAACTTTATCCGCCTCCATCCAGTCTA
TTAATTGTTGCCGGGAAGCTAGAGTAAGTAGTTCGCCAGTTAATAGTTTGC
GCAACGTTGTTGCCATTGCTACAGGCATCGTGGTGTCACGCTCGTCGTTTG
GTATGGCTTCATTCAGCTCCGGTTCCCAACGATCAAGGCGAGTTACATGAT
CCCCCATGTTGTGCAAAAAAGCGGTTAGCTCCTTCGGTCCTCCGATCGTTG
TCAGAAGTAAGTTGGCCGCAGTGTTATCACTCATGGTTATGGCAGCACTGC
ATAATTCTCTTACTGTCATGCCATCCGTAAGATGCTTTTCTGTGACTGGTG
AGTACTCAACCAAGTCATTCTGAGAATAGTGTATGCGGCGACCGAGTTGCT
CTTGCCCGGCGTCAATACGGGATAATACCGCGCCACATAGCAGAACTTTAA
AAGTGCTCATCATTGGAAAACGTTCTTCGGGGCGAAAACTCTCAAGGATCT
TACCGCTGTTGAGATCCAGTTCGATGTAACCCACTCGTGCACCCAACTGAT
CTTCAGCATCTTTTACTTTCACCAGCGTTTCTGGGTGAGCAAAAACAGGAA
GGCAAAATGCCGCAAAAAAGGGAATAAGGGCGACACGGAAATGTTGAATAC
TCATACTCTTCCTTTTTCAATATTATTGAAGCATTTATCAGGGTTATTGTC
TCATGAGCGGATACATATTTGAATGTATTTAGAAAAATAAACAAATAGGGG
TTCCGCGCACATTTCCCCGAAAAGTGCCAC

Suitable additional exemplary vectors include e.g., pBABE-puro, pBABE-neo largeTcDNA, pBABE-hygro-hTERT, pMKO.1 GFP, MSCV-IRES-GFP, pMSCV PIG (Puro IRES GFP empty plasmid), pMSCV-loxp-dsRed-loxp-eGFP-Puro-WPRE, MSCV IRES Luciferase, pMIG, MDH1-PGK-GFP_2.0, TtRMPVIR, pMSCV-IRES-mCherry FP, pRetroX GFP T2A Cre, pRXTN, pLncEXP, and pLXIN-Luc.

In some embodiments, the engineered immune cell is a T cell, tumor infiltrating lymphocyte (TIL), NK cell, TCR-expressing cell, dendritic cell, or NK-T cell. In some embodiments, the cell is obtained or prepared from peripheral blood. In some embodiments, the cell is obtained or prepared from peripheral blood mononuclear cells (PBMCs). In some embodiments, the cell is obtained or prepared from bone marrow. In some embodiments, the cell is obtained or prepared from umbilical cord blood. In some embodiments, the cell is a human cell. In some embodiments, the cell is transfected or transduced by the nucleic acid vector using a method selected from the group consisting of electroporation, sonoporation, biolistics (e.g., Gene Gun), lipid transfection, polymer transfection, nanoparticles, or polyplexes.

In some embodiments, chimeric antigen receptors are expressed in the engineered immune cells that comprise the nucleic acids of the present application. These chimeric antigen receptors of the present application may comprise, in some embodiments, (i) an antigen binding molecule (such as an scFv), (ii) a transmembrane region, and (iii) a T cell activation molecule or region.

### Antigen Binding Molecules

Antigen binding molecules are within the scope of the invention.

An “antigen binding molecule” as used herein means any protein that binds a specified target antigen. In the instant application, the specified target antigen is the STEAP1 protein or fragment thereof. Antigen binding molecules include, but are not limited to antibodies and binding parts thereof, such as immunologically functional fragments. Peptibodies (i.e., Fc fusion molecules comprising peptide binding domains) are another example of suitable antigen binding molecules.

In some embodiments, the antigen binding molecule binds to an antigen on a tumor cell. In some embodiments, the antigen binding molecule binds to an antigen on a cell involved in a hyperproliferative disease or to a viral or bacterial antigen. In certain embodiments, the antigen binding molecule binds to STEAP1. In further embodiments, the antigen binding molecule is an antibody of fragment thereof, including one or more of the complementarity determining regions (CDRs) thereof. In further embodiments, the antigen binding molecule is a single chain variable fragment (scFv).

The term “immunologically functional fragment” (or “fragment”) of an antigen binding molecule is a species of antigen binding molecule comprising a portion (regardless of how that portion is obtained or synthesized) of an antibody that lacks at least some of the amino acids present in a full-length chain but which is still capable of specifically binding to an antigen. Such fragments are biologically active in that they bind to the target antigen and can compete with other antigen binding molecules, including intact antibodies, for binding to a given epitope. In some embodiments, the fragments are neutralizing fragments. In some embodiments, the fragments can block or reduce the activity of STEAP1. In one aspect, such a fragment will retain at least one CDR present in the full-length light or heavy chain, and in some embodiments will comprise a single heavy chain and/or light chain or portion thereof. These fragments can be produced by recombinant DNA techniques, or can be produced by enzymatic or chemical cleavage of antigen binding molecules, including intact antibodies.

Immunologically functional immunoglobulin fragments include, but are not limited to, scFv fragments, Fab fragments (Fab′, F(ab′)₂, and the like), one or more CDR, a diabody (heavy chain variable domain on the same polypeptide as a light chain variable domain, connected via a short peptide linker that is too short to permit pairing between the two domains on the same chain), domain antibodies, and single-chain antibodies. These fragments can be derived from any mammalian source, including but not limited to human, mouse, rat, camelid or rabbit. As will be appreciated by one of skill in the art, an antigen binding molecule can include non-protein components.

Variants of the antigen binding molecules are also within the scope of the invention, e.g., variable light and/or variable heavy chains that each have at least 70-80%, 80-85%, 85-90%, 90-95%, 95-97%, 97-99%, or above 99% identity to the amino acid sequences of the sequences described herein. In some instances, such molecules include at least one heavy chain and one light chain, whereas in other instances the variant forms contain two identical light chains and two identical heavy chains (or subparts thereof). A skilled artisan will be able to determine suitable variants of the antigen binding molecules as set forth herein using well-known techniques. In certain embodiments, one skilled in the art can identify suitable areas of the molecule that may be changed without destroying activity by targeting regions not believed to be important for activity.

In certain embodiments, the polypeptide structure of the antigen binding molecules is based on antibodies, including, but not limited to, monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the antigen binding molecule comprises or consists of avimers.

In some embodiments, an antigen binding molecule to STEAP1 is administered alone. In other embodiments, the antigen binding molecule to STEAP1 is administered as part of a CAR, TCR, or other immune cell. In such immune cells, the antigen binding molecule to STEAP1 can be under the control of the same promoter region, or a separate promoter. In certain embodiments, the genes encoding protein agents and/or an antigen binding molecule to STEAP1 can be in separate vectors.

The invention further provides for pharmaceutical compositions comprising an antigen binding molecule to STEAP1 together with a pharmaceutically acceptable diluent, carrier, solubilizer, emulsifier, preservative and/or adjuvant. In certain embodiments, pharmaceutical compositions will include more than one different antigen binding molecule to STEAP1. In certain embodiments, pharmaceutical compositions will include more than one antigen binding molecule to STEAP1 wherein the antigen binding molecules to STEAP1 bind more than one epitope. In some embodiments, the various antigen binding molecules will not compete with one another for binding to STEAP1.

In other embodiments, the pharmaceutical composition can be selected for parenteral delivery, for inhalation, or for delivery through the digestive tract, such as orally. The preparation of such pharmaceutically acceptable compositions is within the ability of one skilled in the art. In certain embodiments, buffers are used to maintain the composition at physiological pH or at a slightly lower pH, typically within a pH range of from about 5 to about 8. In certain embodiments, when parenteral administration is contemplated, a therapeutic composition can be in the form of a pyrogen-free, parenterally acceptable aqueous solution comprising a desired antigen binding molecule to STEAP1, with or without additional therapeutic agents, in a pharmaceutically acceptable vehicle. In certain embodiments, a vehicle for parenteral injection is sterile distilled water in which an antigen binding molecule to STEAP1, with or without at least one additional therapeutic agent, is formulated as a sterile, isotonic solution, properly preserved. In certain embodiments, the preparation can involve the formulation of the desired molecule with polymeric compounds (such as polylactic acid or polyglycolic acid), beads or liposomes that can provide for the controlled or sustained release of the product which can then be delivered via a depot injection. In certain embodiments, implantable drug delivery devices can be used to introduce the desired molecule.

In some embodiments, the antigen binding molecule is used as a diagnostic or validation tool. The antigen binding molecule can be used to assay the amount of STEAP1 present in a sample and/or subject. In some embodiments, the diagnostic antigen binding molecule is not neutralizing. In some embodiments, the antigen binding molecules disclosed herein are used or provided in an assay kit and/or method for the detection of STEAP1 in mammalian tissues or cells in order to screen/diagnose for a disease or disorder associated with changes in levels of STEAP1. The kit can comprise an antigen binding molecule that binds STEAP1, along with means for indicating the binding of the antigen binding molecule with STEAP1, if present, and optionally STEAP1 protein levels.

The antigen binding molecules will be further understood in view of the definitions and descriptions below.

An “Fc” region comprises two heavy chain fragments comprising the CH1 and CH2 domains of an antibody. The two heavy chain fragments are held together by two or more disulfide bonds and by hydrophobic interactions of the CH3 domains.

A “Fab fragment” comprises one light chain and the CH1 and variable regions of one heavy chain. The heavy chain of a Fab molecule cannot form a disulfide bond with another heavy chain molecule. A “Fab′” fragment” comprises one light chain and a portion of one heavy chain that contains the VH domain and the CH1 domain and also the region between the CH1 and CH2 domains, such that an interchain disulfide bond can be formed between the two heavy chains of two Fab′ fragments to form an F(ab′)₂ molecule. An “F(ab′)₂ fragment” contains two light chains and two heavy chains containing a portion of the constant region between the CH1 and CH2 domains, such that an interchain disulfide bond is formed between the two heavy chains. An F(ab′)₂ fragment thus is composed of two Fab′ fragments that are held together by a disulfide bond between the two heavy chains.

The “Fv region” comprises the variable regions from both the heavy and light chains, but lacks the constant regions.

“Single chain variable fragment” (“scFv”, also termed “single-chain antibody”) refers to Fv molecules in which the heavy and light chain variable regions have been connected by a flexible linker to form a single polypeptide chain, which forms an antigen binding region. See PCT application WO88/01649 and U.S. Pat. Nos. 4,946,778 and 5,260,203, the disclosures of which are incorporated by reference in their entirety.

A “bivalent antigen binding molecule” comprises two antigen binding sites. In some instances, the two binding sites have the same antigen specificities. Bivalent antigen binding molecules can be bispecific. A “multispecific antigen binding molecule” is one that targets more than one antigen or epitope. A “bispecific,” “dual-specific” or “bifunctional” antigen binding molecule is a hybrid antigen binding molecule or antibody, respectively, having two different antigen binding sites. The two binding sites of a bispecific antigen binding molecule will bind to two different epitopes, which can reside on the same or different protein targets.

An antigen binding molecule is said to “specifically bind” its target antigen when the dissociation constant (K_{d}) is ˜1×10⁻⁷ M. The antigen binding molecule specifically binds antigen with “high affinity” when the K_{d} is 1-5×10⁻⁹ M, and with “very high affinity” when the K_{d} is 1-5×10⁻¹⁰ M. In one embodiment, the antigen binding molecule has a K_{d} of 10⁻⁹ M. In one embodiment, the off-rate is <1×10⁻⁵. In other embodiments, the antigen binding molecules will bind to human STEAP1 with a K_{d} of between about 10⁻⁷ M and 10⁻¹³ M, and in yet another embodiment the antigen binding molecules will bind with a K_{d} 1.0-5×10⁻¹⁰.

An antigen binding molecule is said to be “selective” when it binds to one target more tightly than it binds to a second target.

The term “antibody” refers to an intact immunoglobulin of any isotype, or a fragment thereof that can compete with the intact antibody for specific binding to the target antigen, and includes, for instance, chimeric, humanized, fully human, and bispecific antibodies. An “antibody” is a species of an antigen binding molecule as defined herein. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be chimeric, that is, different portions of the antibody can be derived from two different antibodies as described further below. The antigen binding molecules, antibodies, or binding fragments can be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”) and fragments thereof, respectively.

The variable regions typically exhibit the same general structure of relatively conserved framework regions (FR) joined by the 3 hypervariable regions (i.e., “CDRs”). The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. By convention, CDR regions in the heavy chain are typically referred to as HC CDR1, CDR2, and CDR3. The CDR regions in the light chain are typically referred to as LC CDR1, CDR2, and CDR3. The assignment of amino acids to each domain is typically in accordance with the definitions of Kabat (Segs of Proteins of Immunological Interest (NIH, Bethesda, Md. (1987 and 1991)), or Chothia (J. Mol. Biol., 196:901-917 (1987); Chothia et al., Nature, 342:878-883 (1989)). Various methods of analysis can be employed to identify or approximate the CDR regions, including not only Kabat or Chothia, but also the AbM definition.

The term “light chain” includes a full-length light chain and fragments thereof having sufficient variable region sequence to confer binding specificity. A full-length light chain includes a variable region domain, V_{L}, and a constant region domain, C_{L}. The variable region domain of the light chain is at the amino-terminus of the polypeptide. Light chains include kappa chains and lambda chains.

The term “heavy chain” includes a full-length heavy chain and fragments thereof having sufficient variable region sequence to confer binding specificity. A full-length heavy chain includes a variable region domain, V_{H}, and three constant region domains, CH1, CH2, and CH3. The V_{H} domain is at the amino-terminus of the polypeptide, and the CH domains are at the carboxyl-terminus, with the CH3 being closest to the carboxy-terminus of the polypeptide. Heavy chains can be of any isotype, including IgG (including IgG1, IgG2, IgG3 and IgG4 subtypes), IgA (including IgA1 and IgA2 subtypes), IgM and IgE.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. The variable region of an antibody typically determines specificity of a particular antibody for its target.

Variability is not evenly distributed throughout the variable domains of antibodies; it is concentrated in sub-domains of each of the heavy and light chain variable regions. These subdomains are called “hypervariable regions” or “complementarity determining regions” (CDRs). The more conserved (i.e., non-hypervariable) portions of the variable domains are called the “framework” regions (FRM or FR) and provide a scaffold for the six CDRs in three dimensional space to form an antigen-binding surface. The variable domains of naturally occurring heavy and light chains each comprise four FRM regions (FR1, FR2, FR3, and FR4), largely adopting a (3-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding site (see Kabat et al., loc. cit.).

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDRH1, CDR-H2 and CDR-H3). CDRs contain most of the residues responsible for specific interactions of the antibody with the antigen and hence contribute to the functional activity of an antibody molecule: they are the main determinants of antigen specificity.

The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit.; Chothia et al., J. MoI. Biol, 1987, 196: 901-917; and MacCallum et al., J. Mol. Biol, 1996, 262: 732). Still another standard for characterizing the antigen binding site is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia and Lesk, J. Mol. Biol., 1987, 196: 901; Chothia et al., Nature, 1989, 342: 877; Martin and Thornton, J. Mol. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “canonical structure” may also include considerations as to the linear sequence of the antibody, for example, as catalogued by Kabat (Kabat et al., loc. cit.). The Kabat numbering scheme (system) is a widely adopted standard for numbering the amino acid residues of an antibody variable domain in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al. and/or revealed by other techniques, for example, crystallography and two- or three-dimensional computational modeling. Accordingly, a given antibody sequence may be placed into a canonical class which allows for, among other things, identifying appropriate chassis sequences (e.g., based on a desire to include a variety of canonical structures in a library). Kabat numbering of antibody amino acid sequences and structural considerations as described by Chothia et al., loc. cit. and their implications for construing canonical aspects of antibody structure, are described in the literature. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988.

The CDR3 of the light chain and, particularly, the CDR3 of the heavy chain may constitute the most important determinants in antigen binding within the light and heavy chain variable regions. In some antibody constructs, the heavy chain CDR3 appears to constitute the major area of contact between the antigen and the antibody. In vitro selection schemes in which CDR3 alone is varied can be used to vary the binding properties of an antibody or determine which residues contribute to the binding of an antigen. Hence, CDR3 is typically the greatest source of molecular diversity within the antibody-binding site. H3, for example, can be as short as two amino acid residues or greater than 26 amino acids.

The term “neutralizing” refers to an antigen binding molecule, scFv, or antibody, respectively, that binds to a ligand and prevents or reduces the biological effect of that ligand. This can be done, for example, by directly blocking a binding site on the ligand or by binding to the ligand and altering the ligand's ability to bind through indirect means (such as structural or energetic alterations in the ligand). In some embodiments, the term can also denote an antigen binding molecule that prevents the protein to which it is bound from performing a biological function.

The term “target” or “antigen” refers to a molecule or a portion of a molecule capable of being bound by an antigen binding molecule. In certain embodiments, a target can have one or more epitopes.

The term “compete” when used in the context of antigen binding molecules that compete for the same epitope means competition between antigen binding molecules as determined by an assay in which the antigen binding molecule (e.g., antibody or immunologically functional fragment thereof) being tested prevents or inhibits (e.g., reduces) specific binding of a reference antigen binding molecule to an antigen. Numerous types of competitive binding assays can be used to determine if one antigen binding molecule competes with another, for example: solid phase direct or indirect radioimmunoassay (RIA), solid phase direct or indirect enzyme immunoassay (EIA), sandwich competition assay (Stahli et al., 1983, Methods in Enzymology 9:242-253); solid phase direct biotin-avidin EIA (Kirkland et al., 1986, J. Immunol. 137:3614-3619), solid phase direct labeled assay, solid phase direct labeled sandwich assay (Harlow and Lane, 1988, Antibodies, A Laboratory Manual, Cold Spring Harbor Press); solid phase direct label RIA using 1-125 label (Morel et al., 1988, Molec. Immunol. 25:7-15); solid phase direct biotin-avidin EIA (Cheung, et al., 1990, Virology 176:546-552); and direct labeled RIA (Moldenhauer et al., 1990, Scand. J. Immunol. 32:77-82). The term “epitope” includes any determinant capable of being bound by an antigen binding molecule, such as an scFv, antibody, or immune cell of the invention. An epitope is a region of an antigen that is bound by an antigen binding molecule that targets that antigen, and when the antigen is a protein, includes specific amino acids that directly contact the antigen binding molecule.

As used herein, the terms “label” or “labeled” refers to incorporation of a detectable marker, e.g., by incorporation of a radiolabeled amino acid or attachment to a polypeptide of biotin moieties that can be detected by marked avidin (e.g., streptavidin containing a fluorescent marker or enzymatic activity that can be detected by optical or colorimetric methods). In certain embodiments, the label or marker can also be therapeutic. Various methods of labeling polypeptides and glycoproteins are known in the art and can be used.

In accordance with the invention, on-off or other types of control switch techniques may be incorporated herein. These techniques may employ the use of dimerization domains and optional activators of such domain dimerization. These techniques include, e.g., those described by Wu et al., Science 2014 350 (6258) utilizing FKBP/Rapalog dimerization systems in certain cells, the contents of which are incorporated by reference herein in their entirety. Additional dimerization technology is described in, e.g., Fegan et al. Chem. Rev. 2010, 110, 3315-3336 as well as U.S. Pat. Nos. 5,830,462; 5,834,266; 5,869,337; and 6,165,787, the contents of which are also incorporated by reference herein in their entirety. Additional dimerization pairs may include cyclosporine-A/cyclophilin, receptor, estrogen/estrogen receptor (optionally using tamoxifen), glucocorticoids/glucocorticoid receptor, tetracycline/tetracycline receptor, vitamin D/vitamin D receptor. Further examples of dimerization technology can be found in e.g., WO 2014/127261, WO 2015/090229, US 2014/0286987, US 2015/0266973, US 2016/0046700, U.S. Pat. No. 8,486,693, US 2014/0171649, and US 2012/0130076, the contents of which are further incorporated by reference herein in their entirety.

### Methods of Treatment

Using adoptive immunotherapy, native T cells can be (i) removed from a patient, (ii) genetically engineered to express a chimeric antigen receptor (CAR) that binds to at least one tumor antigen (iii) expanded ex vivo into a larger population of engineered T cells, and (iv) reintroduced into the patient. See e.g., U.S. Pat. Nos. 7,741,465, and 6,319,494, Eshhar et al. (Cancer Immunol, supra); Krause et al. (supra); Finney et al. (supra). After the engineered T cells are reintroduced into the patient, they mediate an immune response against cells expressing the tumor antigen. See e.g., Krause et al., J. Exp. Med., Volume 188, No. 4, 1998 (619-626). This immune response includes secretion of IL-2 and other cytokines by T cells, the clonal expansion of T cells recognizing the tumor antigen, and T cell-mediated specific killing of target-positive cells. See Hombach et al., Journal of Immun. 167: 6123-6131 (2001).

In some aspects, the invention therefore comprises a method for treating or preventing a condition associated with undesired and/or elevated STEAP1 levels in a patient, comprising administering to a patient in need thereof an effective amount of at least one isolated antigen binding molecule, CAR, or TCR disclosed herein.

Methods are provided for treating diseases or disorders, including cancer. In some embodiments, the invention relates to creating a T cell-mediated immune response in a subject, comprising administering an effective amount of the engineered immune cells of the present application to the subject. In some embodiments, the T cell-mediated immune response is directed against a target cell or cells. In some embodiments, the engineered immune cell comprises a chimeric antigen receptor (CAR), or a T cell receptor (TCR). In some embodiments, the target cell is a tumor cell. In some aspects, the invention comprises a method for treating or preventing a malignancy, said method comprising administering to a subject in need thereof an effective amount of at least one isolated antigen binding molecule described herein. In some aspects, the invention comprises a method for treating or preventing a malignancy, said method comprising administering to a subject in need thereof an effective amount of at least one immune cell, wherein the immune cell comprises at least one chimeric antigen receptor, T cell receptor, and/or isolated antigen binding molecule as described herein.

In some aspects, the invention comprises a pharmaceutical composition comprising at least one antigen binding molecule as described herein and a pharmaceutically acceptable excipient. In some embodiments, the pharmaceutical composition further comprises an additional active agent.

The antigen binding molecules, CARs, TCRs, immune cells, and the like of the invention can be used to treat STEAP1 expressing diseases including but not limited to prostate cancer, and in one preferred embodiment, in metastatic castration resistant prostate cancer.

It will be appreciated that target doses for CAR⁺/CAR-T⁺/TCR⁺ cells can range from 1×10⁶-2×10¹⁰ cells/kg, preferably 2×10⁶ cells/kg, more preferably. It will be appreciated that doses above and below this range may be appropriate for certain subjects, and appropriate dose levels can be determined by the healthcare provider as needed. Additionally, multiple doses of cells can be provided in accordance with the invention.

Also provided are methods for reducing the size of a tumor in a subject, comprising administering to the subject an engineered cell of the present invention to the subject, wherein the cell comprises a chimeric antigen receptor, a T cell receptor, or a T cell receptor based chimeric antigen receptor comprising an antigen binding molecule binds to an antigen on the tumor. In some embodiments, the subject has a solid tumor, or a blood malignancy such as lymphoma or leukemia. In some embodiments, the engineered cell is delivered to a tumor bed. In some embodiments, the cancer is present in the bone marrow of the subject.

In some embodiments, the engineered cells are autologous T cells. In some embodiments, the engineered cells are allogeneic T cells. In some embodiments, the engineered cells are heterologous T cells. In some embodiments, the engineered cells of the present application are transfected or transduced in vivo. In other embodiments, the engineered cells are transfected or transduced ex vivo.

The methods can further comprise administering one or more chemotherapeutic agent. In certain embodiments, the chemotherapeutic agent is a lymphodepleting (preconditioning) chemotherapeutic. Beneficial preconditioning treatment regimens, along with correlative beneficial biomarkers are described in U.S. Provisional Patent Applications 62/262,143 and 62/167,750 which are hereby incorporated by reference in their entirety herein. These describe, e.g., methods of conditioning a patient in need of a T cell therapy comprising administering to the patient specified beneficial doses of cyclophosphamide (between 200 mg/m²/day and 2000 mg/m²/day) and specified doses of fludarabine (between 20 mg/m²/day and 900 mg/m²/day). A preferred dose regimen involves treating a patient comprising administering daily to the patient about 500 mg/m²/day of cyclophosphamide and about 60 mg/m²/day of fludarabine for three days prior to administration of a therapeutically effective amount of engineered T cells to the patient.

In other embodiments, the antigen binding molecule, transduced (or otherwise engineered) cells (such as CARs or TCRs), and the chemotherapeutic agent are administered each in an amount effective to treat the disease or condition in the subject.

In certain embodiments, compositions comprising CAR-expressing immune effector cells disclosed herein may be administered in conjunction with any number of chemotherapeutic agents. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL™, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS2000; difluoromethylomithine (DMFO); retinoic acid derivatives such as Targretin™ (bexarotene), Panretin™, (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan®), Doxorubicin (hydroxydoxorubicin), Vincristine (Oncovin®), and Prednisone.

In some embodiments, the chemotherapeutic agent is administered at the same time or within one week after the administration of the engineered cell or nucleic acid. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell or nucleic acid. In other embodiments, the chemotherapeutic agent is administered at least 1 month before administering the cell or nucleic acid. In some embodiments, the methods further comprise administering two or more chemotherapeutic agents.

A variety of additional therapeutic agents may be used in conjunction with the compositions described herein. For example, potentially useful additional therapeutic agents include PD-1 inhibitors such as nivolumab (Opdivo®), pembrolizumab (Keytruda®), pembrolizumab, pidilizumab, and atezolizumab, and CTLA-4 inhibitors, such as ipilimumab (Yervoy®).

Additional therapeutic agents suitable for use in combination with the invention include, but are not limited to, abiraterone acetate, apalutamide, bicalutamide, cabazitaxel, casodex (bicalutamide), degarelix, docetaxel, enzalutamide, Erleada® (apalutamide), flutamide, goserelin acetate, Jevtana® (cabazitaxel), leuprolide acetate, Lupron® (leuprolide acetate), Lupron Depot (leuprolide acetate), Lupron Depot-Ped (leuprolide acetate), mitoxantrone hydrochloride, Nilandron® (nilutamide), nilutamide, Provenge® (Sipuleucel-T), radium 223 dichloride, sipuleucel-T, taxotere (docetaxel), Viadur (leuprolide acetate), Xofigo (radium 223 dichloride), Xtandi (enzalutamide), Zoladex (goserelin acetate), or Zytiga (abiraterone acetate).

In additional embodiments, the composition comprising CAR-containing immune can be administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

In certain embodiments, the compositions described herein are administered in conjunction with a cytokine. “Cytokine” as used herein is meant to refer to proteins released by one cell population that act on another cell as intercellular mediators. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and —II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In some aspects, the invention comprises an antigen binding molecule that binds to STEAP1 with a K_{d} that is smaller than 100 pM. In some embodiments, the antigen binding molecule binds with a K_{d} that is smaller than 10 pM. In other embodiments, the antigen binding molecule binds with a K_{d} that is less than 5 pM.

### Methods of Making

A variety of known techniques can be utilized in making the polynucleotides, polypeptides, vectors, antigen binding molecules, immune cells, compositions, and the like according to the invention.

Prior to the in vitro manipulation or genetic modification of the immune cells described herein, the cells may be obtained from a subject. In some embodiments, the immune cells comprise T cells. T cells can be obtained from a number of sources, including peripheral blood mononuclear cells (PBMCs), bone marrow, lymph nodes tissue, cord blood, thymus tissue, tissue from a site of infection, ascites, pleural effusion, spleen tissue, and tumors. In certain embodiments, T cells can be obtained from a unit of blood collected from the subject using any number of techniques known to the skilled person, such as FICOLL™ separation. Cells may preferably be obtained from the circulating blood of an individual by apheresis. The apheresis product typically contains lymphocytes, including T cells, monocytes, granulocytes, B cells, other nucleated white blood cells, red blood cells, and platelets. In certain embodiments, the cells collected by apheresis may be washed to remove the plasma fraction, and placed in an appropriate buffer or media for subsequent processing. The cells may be washed with PBS. As will be appreciated, a washing step may be used, such as by using a semiautomated flowthrough centrifuge—for example, the Cobe™ 2991 cell processor, the Baxter CytoMate™, or the like. After washing, the cells may be resuspended in a variety of biocompatible buffers, or other saline solution with or without buffer. In certain embodiments, the undesired components of the apheresis sample may be removed.

In certain embodiments, T cells are isolated from PBMCs by lysing the red blood cells and depleting the monocytes, for example, using centrifugation through a PERCOLL™ gradient. A specific subpopulation of T cells, such as CD28⁺, CD4⁺, CD8⁺, CD45RA⁺, and CD45RO⁺ T cells can be further isolated by positive or negative selection techniques known in the art. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. Flow cytometry and cell sorting may also be used to isolate cell populations of interest for use in the present invention.

PBMCs may be used directly for genetic modification with the immune cells (such as CARs or TCRs) using methods as described herein. In certain embodiments, after isolating the PBMCs, T lymphocytes can be further isolated and both cytotoxic and helper T lymphocytes can be sorted into naive, memory, and effector T cell subpopulations either before or after genetic modification and/or expansion.

In some embodiments, CD8⁺ cells are further sorted into naive, central memory, and effector cells by identifying cell surface antigens that are associated with each of these types of CD8⁺ cells. In some embodiments, the expression of phenotypic markers of central memory T cells include CD45RO, CD62L, CCR7, CD28, CD3, and CD127 and are negative for granzyme B. In some embodiments, central memory T cells are CD45RO⁺, CD62L⁺, CD8⁺ T cells. In some embodiments, effector T cells are negative for CD62L, CCR7, CD28, and CD127, and positive for granzyme B and perforin. In certain embodiments, CD4⁺ T cells are further sorted into subpopulations. For example, CD4⁺ T helper cells can be sorted into naive, central memory, and effector cells by identifying cell populations that have cell surface antigens.

The immune cells, such as T cells, can be genetically modified following isolation using known methods, or the immune cells can be activated and expanded (or differentiated in the case of progenitors) in vitro prior to being genetically modified. In another embodiment, the immune cells, such as T cells, are genetically modified with the chimeric antigen receptors described herein (e.g., transduced with a viral vector comprising one or more nucleotide sequences encoding a CAR) and then are activated and/or expanded in vitro. Methods for activating and expanding T cells are known in the art and are described, for example, in U.S. Pat. Nos. 6,905,874; 6,867,041; 6,797,514; and PCT WO2012/079000, the contents of which are hereby incorporated by reference in their entirety. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is The Dynabeads® system, a CD3/CD28 activator/stimulator system for physiological activation of human T cells.

In other embodiments, the T cells may be activated and stimulated to proliferate with feeder cells and appropriate antibodies and cytokines using methods such as those described in U.S. Pat. Nos. 6,040,177; 5,827,642; and WO2012129514, the contents of which are hereby incorporated by reference in their entirety.

Certain methods for making the constructs and engineered immune cells of the invention are described in PCT application PCT/US15/14520, the contents of which are hereby incorporated by reference in their entirety. Additional methods of making the constructs and cells can be found in U.S. provisional patent application No. 62/244,036 the contents of which are hereby incorporated by reference in their entirety.

It will be appreciated that PBMCs can further include other cytotoxic lymphocytes such as NK cells or NKT cells. An expression vector carrying the coding sequence of a chimeric receptor as disclosed herein can be introduced into a population of human donor T cells, NK cells or NKT cells. Successfully transduced T cells that carry the expression vector can be sorted using flow cytometry to isolate CD3 positive T cells and then further propagated to increase the number of these CAR expressing T cells in addition to cell activation using anti-CD3 antibodies and IL-2 or other methods known in the art as described elsewhere herein. Standard procedures are used for cryopreservation of T cells expressing the CAR for storage and/or preparation for use in a human subject. In one embodiment, the in vitro transduction, culture and/or expansion of T cells are performed in the absence of non-human animal derived products such as fetal calf serum and fetal bovine serum.

For cloning of polynucleotides, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells containing the vector provided herein. The host cells containing the vector may be useful in expression or cloning of the polynucleotide contained in the vector. Suitable host cells can include, without limitation, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobactehaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The vector can be introduced to the host cell using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art. In a further embodiment, a mixture of different expression vectors can be used in genetically modifying a donor population of immune effector cells wherein each vector encodes a different CAR as disclosed herein. The resulting transduced immune effector cells form a mixed population of engineered cells, with a proportion of the engineered cells expressing more than one different CARs.

In one embodiment, the invention provides a method of storing genetically engineered cells expressing CARs or TCRs which target a STEAP1 protein. This involves cryopreserving the immune cells such that the cells remain viable upon thawing. A fraction of the immune cells expressing the CARs can be cryopreserved by methods known in the art to provide a permanent source of such cells for the future treatment of patients afflicted with a malignancy. When needed, the cryopreserved transformed immune cells can be thawed, grown and expanded for more such cells.

As used herein, “cryopreserve” refers to the preservation of cells by cooling to sub-zero temperatures, such as (typically) 77 Kelvin or −196° C. (the boiling point of liquid nitrogen). Cryoprotective agents are often used at sub-zero temperatures to prevent the cells being preserved from damage due to freezing at low temperatures or warming to room temperature. Cryopreservative agents and optimal cooling rates can protect against cell injury. Cryoprotective agents which can be used in accordance with the invention include but are not limited to: dimethyl sulfoxide (DMSO) (Lovelock & Bishop, Nature (1959); 183: 1394-1395; Ashwood-Smith, Nature (1961); 190: 1204-1205), glycerol, polyvinylpyrrolidine (Rinfret, Ann. N.Y. Acad. Sci. (1960); 85: 576), and polyethylene glycol (Sloviter & Ravdin, Nature (1962); 196: 48). The preferred cooling rate is 1°-3° C./minute.

The term, “substantially pure,” is used to indicate that a given component is present at a high level. The component is desirably the predominant component present in a composition. Preferably it is present at a level of more than 30%, of more than 50%, of more than 75%, of more than 90%, or even of more than 95%, said level being determined on a dry weight/dry weight basis with respect to the total composition under consideration. At very high levels (e.g. at levels of more than 90%, of more than 95% or of more than 99%) the component can be regarded as being in “pure form.” Biologically active substances of the present invention (including polypeptides, nucleic acid molecules, antigen binding molecules, moieties) can be provided in a form that is substantially free of one or more contaminants with which the substance might otherwise be associated. When a composition is substantially free of a given contaminant, the contaminant will be at a low level (e.g., at a level of less than 10%, less than 5%, or less than 1% on the dry weight/dry weight basis set out above).

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion media can be any isotonic medium formulation, typically normal saline, Normosol™ R (Abbott) or Plasma-Lyte™ A (Baxter), but also 5% dextrose in water or Ringer's lactate can be utilized. The infusion medium can be supplemented with human serum albumin.

Desired treatment amounts of cells in the composition is generally at least 2 cells (for example, at least 1 CD8⁺ central memory T cell and at least 1 CD4⁺ helper T cell subset) or is more typically greater than 10² cells, and up to 10⁶, up to and including 10⁸ or 10⁹ cells and can be more than 10¹⁰ cells. The number of cells will depend upon the desired use for which the composition is intended, and the type of cells included therein. The density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present invention, particularly since all the infused cells will be redirected to a particular target antigen (STEAP1), lower numbers of cells, in the range of 10⁶/kilogram (10⁶-10¹¹ per patient) may be administered. CAR treatments may be administered multiple times at dosages within these ranges. The cells may be autologous, allogeneic, or heterologous to the patient undergoing therapy.

The CAR expressing cell populations of the present invention may be administered either alone, or as a pharmaceutical composition in combination with diluents and/or with other components such as IL-2 or other cytokines or cell populations. Pharmaceutical compositions of the present invention may comprise a CAR or TCR expressing cell population, such as T cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

The pharmaceutical compositions (solutions, suspensions or the like), may include one or more of the following: sterile diluents such as water for injection, saline solution, preferably physiological saline, Ringer's solution, isotonic sodium chloride, fixed oils such as synthetic mono- or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

It will be appreciated that adverse events may be minimized by transducing the immune cells (containing one or more CARs or TCRs) with a suicide gene. It may also be desired to incorporate an inducible “on” or “accelerator” switch into the immune cells. Suitable techniques include use of inducible caspase-9 (U.S. Appl. 2011/0286980) or a thymidine kinase, before, after or at the same time, as the cells are transduced with the CAR construct of the present invention. Additional methods for introducing suicide genes and/or “on” switches include TALENS, zinc fingers, RNAi, siRNA, shRNA, antisense technology, and other techniques known in the art.

It will be understood that descriptions herein are exemplary and explanatory only and are not restrictive of the invention as claimed. In this application, the use of the singular includes the plural unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. As utilized in accordance with the present disclosure, the following terms, unless otherwise indicated, shall be understood to have the following meanings:

In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

The term “STEAP1 activity” includes any biological effect of STEAP1. In certain embodiments, STEAP1 activity includes the ability of STEAP1 to interact or bind to a substrate or receptor.

The term “polynucleotide”, “nucleotide”, or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphoro-diselenoate, phosphoro-anilothioate, phoshoraniladate and phosphoroamidate.

The term “oligonucleotide” refers to a polynucleotide comprising 200 or fewer nucleotides. Oligonucleotides can be single stranded or double stranded, e.g., for use in the construction of a mutant gene. Oligonucleotides can be sense or antisense oligonucleotides. An oligonucleotide can include a label, including a radiolabel, a fluorescent label, a hapten or an antigenic label, for detection assays. Oligonucleotides can be used, for example, as PCR primers, cloning primers or hybridization probes.

The term “control sequence” refers to a polynucleotide sequence that can affect the expression and processing of coding sequences to which it is ligated. The nature of such control sequences can depend upon the host organism. In particular embodiments, control sequences for prokaryotes can include a promoter, a ribosomal binding site, and a transcription termination sequence. For example, control sequences for eukaryotes can include promoters comprising one or a plurality of recognition sites for transcription factors, transcription enhancer sequences, and transcription termination sequence. “Control sequences” can include leader sequences (signal peptides) and/or fusion partner sequences.

As used herein, “operably linked” means that the components to which the term is applied are in a relationship that allows them to carry out their inherent functions under suitable conditions.

The term “vector” means any molecule or entity (e.g., nucleic acid, plasmid, bacteriophage or virus) used to transfer protein coding information into a host cell. The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct can include, but is not limited to, sequences that affect or control transcription, translation, and, if introns are present, affect RNA splicing of a coding region operably linked thereto.

The term “host cell” refers to a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA can recombine with that of the cell by physically integrating into a chromosome of the cell, or can be maintained transiently as an episomal element without being replicated, or can replicate independently as a plasmid. A cell is considered to have been “stably transformed” when the transforming DNA is replicated with the division of the cell.

The term “transfection” refers to the uptake of foreign or exogenous DNA by a cell. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197.

The term “transduction” refers to the process whereby foreign DNA is introduced into a cell via viral vector. See Jones et al., (1998). Genetics: principles and analysis. Boston: Jones & Bartlett Publ.

The terms “polypeptide” or “protein” refer to a macromolecule having the amino acid sequence of a protein, including deletions from, additions to, and/or substitutions of one or more amino acids of the native sequence. The terms “polypeptide” and “protein” specifically encompass STEAP1 antigen binding molecules, antibodies, or sequences that have deletions from, additions to, and/or substitutions of one or more amino acid of antigen-binding protein. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. Useful polypeptide fragments include immunologically functional fragments of antigen binding molecules. Useful fragments include but are not limited to one or more CDR regions, variable domains of a heavy and/or light chain, a portion of other portions of an antibody chain, and the like.

The term “isolated” means (i) free of at least some other proteins with which it would normally be found, (ii) is essentially free of other proteins from the same source, e.g., from the same species, (iii) separated from at least about 50 percent of polynucleotides, lipids, carbohydrates, or other materials with which it is associated in nature, (iv) operably associated (by covalent or noncovalent interaction) with a polypeptide with which it is not associated in nature, or (v) does not occur in nature.

A “variant” of a polypeptide (e.g., an antigen binding molecule, or an antibody) comprises an amino acid sequence wherein one or more amino acid residues are inserted into, deleted from and/or substituted into the amino acid sequence relative to another polypeptide sequence. Variants include fusion proteins.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”).

To calculate percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

As used herein, the twenty conventional (e.g., naturally occurring) amino acids and their abbreviations follow conventional usage. See Immunology—A Synthesis (2nd Edition, Golub and Gren, Eds., Sinauer Assoc., Sunderland, Mass. (1991)), which is incorporated herein by reference for any purpose. Stereoisomers (e.g., D-amino acids) of the twenty conventional amino acids, unnatural amino acids such as alpha-, alpha-disubstituted amino acids, N-alkyl amino acids, lactic acid, and other unconventional amino acids can also be suitable components for polypeptides of the present invention. Examples of unconventional amino acids include: 4-hydroxyproline, .gamma.-carboxyglutamate, epsilon-N,N,N-trimethyllysine, e-N-acetyllysine, O-phosphoserine, N-acetylserine, N-formylmethionine, 3-methylhistidine, 5-hydroxylysine, .sigma-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Conservative amino acid substitutions can encompass non-naturally occurring amino acid residues, which are typically incorporated by chemical peptide synthesis rather than by synthesis in biological systems. These include peptidomimetics and other reversed or inverted forms of amino acid moieties. Naturally occurring residues can be divided into classes based on common side chain properties:

a) hydrophobic: norleucine, Met, Ala, Val, Leu, Ile;

b) neutral hydrophilic: Cys, Ser, Thr, Asn, Gln;

c) acidic: Asp, Glu;

d) basic: His, Lys, Arg;

e) residues that influence chain orientation: Gly, Pro; and

f) aromatic: Trp, Tyr, Phe.

For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the antigen binding molecule, the costimulatory or activating domains of the engineered T cell, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). See Kyte et al., J. Mol. Biol., 157:105-131 (1982). It is known that certain amino acids can be substituted for other amino acids having a similar hydropathic index or score and still retain a similar biological activity. It is also understood in the art that the substitution of like amino acids can be made effectively on the basis of hydrophilicity, particularly where the biologically functional protein or peptide thereby created is intended for use in immunological embodiments, as in the present case. Exemplary amino acid substitutions are set forth in Table 2.

TABLE 2

| Original | | Preferred |
|---|---|---|
| Residues | Exemplary Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, 1,4 Diamino-butyric Acid, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified antigen binding molecule can have a greater circulating half-life than an antigen binding molecule that is not chemically modified. In some embodiments, a derivative antigen binding molecule is covalently modified to include one or more water soluble polymer attachments, including, but not limited to, polyethylene glycol, polyoxyethylene glycol, or polypropylene glycol.

Peptide analogs are commonly used in the pharmaceutical industry as non-peptide drugs with properties analogous to those of the template peptide. These types of non-peptide compound are termed “peptide mimetics” or “peptidomimetics.” Fauchere, J., Adv. Drug Res., 15:29 (1986); Veber & Freidinger, TINS, p. 392 (1985); and Evans et al., J. Med. Chem., 30:1229 (1987), which are incorporated herein by reference for any purpose.

The term “therapeutically effective amount” refers to the amount of a STEAP1 antigen binding molecule determined to produce a therapeutic response in a mammal. Such therapeutically effective amounts are readily ascertained by one of ordinary skill in the art.

The terms “patient” and “subject” are used interchangeably and include human and non-human animal subjects as well as those with formally diagnosed disorders, those without formally recognized disorders, those receiving medical attention, those at risk of developing the disorders, etc.

The term “treat” and “treatment” includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. The term “prevent” does not require the 100% elimination of the possibility of an event. Rather, it denotes that the likelihood of the occurrence of the event has been reduced in the presence of the compound or method.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

The following sequences will further exemplify the invention.

CD28T DNA Extracellular, Transmembrane, Intracellular

(SEQ ID NO: 1)
CTTGATAATGAAAAGTCAAACGGAACAATCATTCACGTGAAGGGCAAGCAC
CTCTGTCCGTCACCCTTGTTCCCTGGTCCATCCAAGCCATTCTGGGTGTTG
GTCGTAGTGGGTGGAGTCCTCGCTTGTTACTCTCTGCTCGTCACCGTGGCT
TTTATAATCTTCTGGGTTAGATCCAAAAGAAGCCGCCTGCTCCATAGCGAT
TACATGAATATGACTCCACGCCGCCCTGGCCCCACAAGGAAACACTACCAG
CCTTACGCACCACCTAGAGATTTCGCTGCCTATCGGAGC

CD28T Extracellular, Transmembrane, Intracellular AA:

(SEQ ID NO: 2)
LDNEKSNGTI IHVKGKHLCP SPLFPGPSKP FWVLVVVGGV
LACYSLLVTV AFIIFWVRSK RSRLLHSDYM NMTPRRPGPT
RKHYQPYAPP RDFAAYRS

CD28T DNA—Extracellular

(SEQ ID NO: 3)
CTTGATAATGAAAAGTCAAACGGAACAATCATTCACGTGAAGGGCAAGCAC
CTCTGTCCGTCACCCTTGTTCCCTGGTCCATCCAAGCCA

CD28T AA—Extracellular

(SEQ ID NO: 4)
LDNEKSNGTI IHVKGKHLCP SPLFPGPSKP

CD28 DNA Transmembrane Domain

(SEQ ID NO: 5)
TTCTGGGTGTTGGTCGTAGTGGGTGGAGTCCTCGCTTGTTACTCTCTGCTC
GTCACCGTGGCTTTTATAATCTTCTGGGTT

CD28 AA Transmembrane Domain:

(SEQ ID NO: 6)
FWVLVVVGGV LACYSLLVTV AFIIFWV

CD28 DNA Intracellular Domain:

(SEQ ID NO: 7)
AGATCCAAAAGAAGCCGCCTGCTCCATAGCGATTACATGAATATGACTCCA
CGCCGCCCTGGCCCCACAAGGAAACACTACCAGCCTTACGCACCACCTAGA
GATTTCGCTGCCTATCGGAGC

CD28 AA Intracellular Domain

(SEQ ID NO: 8)
RSKRSRLLHSDYMNMTPRRPGPTRKHYQPYAPPRDFAAYRS

CD3 zeta DNA

(SEQ ID NO: 9)
AGGGTGAAGTTTTCCAGATCTGCAGATGCACCAGCGTATCAGCAGGGCCAG
AACCAACTGTATAACGAGCTCAACCTGGGACGCAGGGAAGAGTATGACGTT
TTGGACAAGCGCAGAGGACGGGACCCTGAGATGGGTGGCAAACCAAGACGA
AAAAACCCCCAGGAGGGTCTCTATAATGAGCTGCAGAAGGATAAGATGGCT
GAAGCCTATTCTGAAATAGGCATGAAAGGAGAGCGGAGAAGGGGAAAAGGG
CACGACGGTTTGTACCAGGGACTCAGCACTGCTACGAAGGATACTTATGAC
GCTCTCCACATGCAAGCCCTGCCACCTAGG

CD3 zeta AA

(SEQ ID NO: 10)
RVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYD
ALHMQALPPR

CD28 DNA

(SEQ ID NO: 11)
ATTGAGGTGATGTATCCACCGCCTTACCTGGATAACGAAAAGAGTAACGGT
ACCATCATTCACGTGAAAGGTAAACACCTGTGTCCTTCTCCCCTCTTCCCC
GGGCCATCAAAGCCC

CD28 AA

(SEQ ID NO: 12)
IEVMYPPPYL DNEKSNGTII HVKGKHLCPS PLFPGPSKP

CD8 DNA Extracellular & Transmembrane Domain

(SEQ ID NO: 13)
GCTGCAGCATTGAGCAACTCAATAATGTATTTTAGTCACTTTGTACCAGTG
TTCTTGCCGGCTAAGCCTACTACCACACCCGCTCCACGGCCACCTACCCCA
GCTCCTACCATCGCTTCACAGCCTCTGTCCCTGCGCCCAGAGGCTTGCCGA
CCGGCCGCAGGGGGCGCTGTTCATACCAGAGGACTGGATTTCGCCTGCGAT
ATCTATATCTGGGCACCCCTGGCCGGAACCTGCGGCGTACTCCTGCTGTCC
CTGGTCATCACGCTCTATTGTAATCACAGGAAC

CD8 AA Extracellular & Transmembrane Domain

(SEQ ID NO: 14)
AAALSNSIMYFSHFVPVFLPAKPTTTPAPRPPTPAPTIASQPLSLRPEACR
PAAGGAVHTRGLDFACDIYIWAPLAGTCGVLLLSLVITLYCNHRN

4-1BB DNA Intracellular Domain

(SEQ ID NO: 15)
CGCTTTTCCGTCGTTAAGCGGGGGAGAAAAAAGCTGCTGTACATTTTCAAA
CAGCCGTTTATGAGGCCGGTCCAAACGACTCAGGAAGAGGACGGCTGCTCC
TGCCGCTTTCCTGAGGAGGAGGAGGGCGGGTGCGAACTG

4-1BB AA Intracellular Domain

(SEQ ID NO: 16)
RFSVVKRGRKKLLYIFKQPFMRPVQTTQEEDGCSCRFPEEEEGGCEL

Clone 2F3 HC DNA

(SEQ ID NO: 87)
CAGGTTCAGCTGCAGCAGTCTGGAGCTGAGATGATGAAGCCTGGGGCCTCA
GTGAAGATATCCTGCAAGGCTACTGGCTACACATTCAGTACCTACTGGATA
GAGTGGGTAAAGCAGAGGCCTGGACATGGCCTTGAGTGGATTGGAGAGATT
TTACCTGGAAGTGGTAATACTGACTTCAATGAGAAGTTCAAGGGCAAGGCC
ACATTCACTGCAGATACATCCTCCGACACAGCCTACATGCATCTCAGCAGC
CTGACATCTGAGGACTCTGCCGTCTATTACTGTACAAGATGGGGGTACTAC
GGTACTAGGGGGTACTTCAATGTCTGGGGCGCAGGGTCCACGGTCACCGTC
TCCTCA

Clone 2F3 HC AA—CDRs Underlined

(SEQ ID NO: 88)
QVQLVQSGAEVKKPGASVKVSCKASGYTFSTYWIEWVRQAPGQRLEWMGEI
LPGSGNTDFNEKFQGRVTFTADTSSDTAYMELSSLRSEDTAVYYCTRWGYY
GTRGYFNVWGQGTLVTVSS

Clone 2F3 HC AA CDR1:

(SEQ ID NO: 89)
TYWIE

Clone 2F3 HC AA CDR2:

(SEQ ID NO: 90)
EILPGSGNTDFNEKFQG

Clone 2F3 HC AA CDR3:

(SEQ ID NO: 91)
WGYYGTRGYFNV

Clone 2F3 LC DNA

(SEQ ID NO: 92)
CAAATTGTTCTCACCCAGTCTCCAGCAATCATGTCTGCATCTCCAGGGGAG
AAGGTCACCATAACGTGCAGTGCCAGCTCAAGTGTAAGTTACATGCACTGG
TTCCAGCAGAAGCCAGGCACTTCTCCCAAACTCTGGATTTATAGCACCTCC
AACCTGGCTTCTGGAGTCCCTGCTCGCTTCAGTGGCAGTGGATCTGGGACC
TCTTACTCTCTCACAATCAGCCGAATGGAGGCTGAAGATGCTGCCACTTAT
TACTGCCAGCAAAGGCGTAGTTTCCCGTACACGTTCGGAGGGGGGACCAAG
CTGGAAATTAAA

Clone 2F3 LC AA (CDRs Underlined)

(SEQ ID NO: 93)
EIVLTQSPATLSLSPGERATLSCRASSSVSYMHWFQQKPGQAPRLLIYSTS
NLASGIPARFSGSGSGTDYTLTISSLEPEDFAVYYCQQRRSFPYTFGQGTK
LEIK

Clone 2F3 LC CDR1 AA:

(SEQ ID NO: 94)
RASSSVSYMH

Clone 2F3 LC CDR2 AA:

(SEQ ID NO: 95)
STSNLAS

Clone 2F3 LC CDR3 AA:

(SEQ ID NO: 96)
QQRRSFPYT

Clone 11C2 HC DNA

(SEQ ID NO: 97)
CAGATCCAGTTGGTGCAGTCTGGACCTGAACTGAAGAAGCCTGGAGAGACA
GTCAAGATCTCCTGCAAGGCTTCTGGATATACCTTCACAAACTATGGAATG
AACTGGGTGAAGCAGGCTCCAGGAAAGGGTTTACAGTGGATGGGCTGGATG
AACACTTATACTGGAGAGCCAACATATGCTGATGACTTCAAGGGACGGTTT
GCCTTCTCTTTGGAAACCTCTGCCAGAACTGTCTCTTTGGACATCAACGAC
CTCAAAAATGAGGACACGGCTACATATTTCTGTACAAGAGCAGGGGGACAA
CTCAGGCCCGGGGCTATGGACTACTGGGGTCAAGGAACCTCAGTCACCGTC
TCCTCA

Clone 11C2 HC AA (CDRs Underlined)

(SEQ ID NO: 98)
QLVQSGAEVKKPGATVKISCKASGYTFTNYGMNWVQQAPGQGLEWMGWMNT
YTGEPTYADKFQGRVTFTLDTSARTVYMELSSLRSEDTAVYFCARAGGQLR
PGAMDYWGQGTMVTVSS

Clone 11C2 HC AA CDR1:

(SEQ ID NO: 99)
NYGMN

Clone 11C2 HC AA CDR2:

(SEQ ID NO: 100)
WMNTYTGEPTYADKFQG

Clone 11C2 HC AA CDR3:

(SEQ ID NO: 101)
AGGQLRPGAMDY

Clone 11C2 LC DNA

(SEQ ID NO: 102)
GACATTGTGCTGACCCAATCTCCAGCTTCTTTGGCTGTGTCTCTAGGGCAG
AGGGCCACCATCTCCTGCAAGGCCAGCCAAAGTGTTGATTATGATGGTGAT
AGTTTTATGAACTGGTACCAACAGAAACCAGGACAGCCACCCAAACTCCTC
ATCTATGTTGCATCCAATCTAGAATCTGGGATCCCAGACAGGTTTAGTGGC
AGTGGGTCTGGGACAGACTTCACCCTCAACATCCATCCTGTGGAGGAGGAG
GATGCTGCAACCTATTATTGTCAGCAAAGTAATGAGGAACCTCCGACGTTC
GGTGGAGGCACCAAGCTGGAAATCAAA

Clone 11C2 LC AA (CDRs Underlined)

(SEQ ID NO: 103)
DIVLTQTPLSLSVTPGQPASISCKASQSVDYDGDSFMNWYLQKPGQPPQLL
IYVASNLESGVPDRFSGSGSGTDFTLKISRVEAEDVGVYYCQQSNEEPPTF
GQGTKLEIK

Clone 11C2 LC AA CDR1:

(SEQ ID NO: 104)
KASQSVDYDGDSFMN

Clone 11C2 LC AA CDR2:

(SEQ ID NO: 105)
VASNLES

Clone 11C2 LHC AA CDR3:

(SEQ ID NO: 106)
QQSNEEPPT

Clone 1A1 HC DNA

(SEQ ID NO: 107)
CAGATACAACTGGTGGAGTCTGGGGGAGGCGTGGTCCAGCCTGGGAGGTCC
CTGAGACTCTCCTGTGTAGCGTCTGGATTCACCTTCAAGAACTATGGCATG
CACTGGGTCCGCCAGGCTCCAGGCAAGGGGCTGGAGTGGGTGGCAGTTATT
TGGTATGATGGAAGTAATGAATACTATGGAGACCCCGTGAAGGGCCGATTC
ACCATCTCCAGAGACAACTCCAAGAACATGTTGTATCTGCAAATGAACAGC
CTGAGAGCCGATGACACGGCTGTGTATTACTGTGCGAGGTCGGGAATAGCA
GTGGCTGGGGCCTTTGACTACTGGGGCCAGGGAACCCTGGTCACCGTCTCC
TCA

Clone 1A1 HC AA (CDRs Underlined)

(SEQ ID NO: 108)
QVQLVQSGAEVKKPGASVKVSCKASGYTFTTYWMHWVRQAPGQGLEWMGEI
NPSSGRTNYNEKFKTRVTMTRDTSTSTVYMELSSLRSEDTAVYYCAKLGPG
PQYYAMDYWGQGTMVTVSS

Clone 1A1 HC AA CDR1:

(SEQ ID NO: 109)
TYWMH

Clone 1A1 HC AA CDR2:

(SEQ ID NO: 110)
EINPSSGRTNYNEKFKT

Clone 1A1 HC AA CDR3:

(SEQ ID NO: 111)
LGPGPQYYAMDY

Clone 1A1 LC DNA

(SEQ ID NO: 112)
GAAATTGTGTTGACGCAGTCTCCAGACACCCTGTCTTTGTCTCCAGGGAAA
GAAGCCACCCTCTCCTGCAGGGCCAGTCAGAGTGTTAGCAGCAGCTTCTTG
GCCTGGTACCAGCAGAAACCTGGACAGGCTCCCAGTCTCCTCATCTATGTT
GCATCCAGAAGGGCCGCTGGCATCCCTGACAGGTTCAGTGGCAGTGGGTCT
GGGACAGACTTCACTCTCACCATCAGCAGACTGGAGCCTGAAGATTTTGGA
ATGTTTTACTGTCAACACTATGGTAGGACACCATTCACTTTCGGCCCTGGG
ACCAAAGTGGATATCAAACGA

Clone 1A1 LC AA (CDRs Underlined)

(SEQ ID NO: 113)
DIQMTQSPSSLSASVGDRVTITCHASQNINVWLSWYQQKPGKAPKLLIYKA
SKLHTGVPSRFSGSGSGTDFTLTISSLQPEDFATYYCQQGQSYPWTFGQGT
KLEIK

Clone 1A1 LC AA CDR1:

(SEQ ID NO: 114)
HASQNINVWLS

Clone 1A1 LC AA CDR2:

(SEQ ID NO: 115)
KASKLHT

Clone 1A1 LC AA CDR3:

(SEQ ID NO: 116)
QQGQSYPWT

Clone 7A4 HC DNA

(SEQ ID NO: 117)
CAGGTGCAGCTGGTGCAGTCTGGGGCTGAGGTGAAGAAGCCTGGGGCCTC
AGTGAAGGTCTCCTGCAAGGCTTCTGGATACACCTTCACCGGCTACTATA
TACACTGGGTGCGACAGGCCCCTGAACAAGGGCTTGAGTGGATGGGATGG
ATCAACCCTAACAGTGGTGGCACAAACTATGCACAGAAGTTTCAGGGCAG
GGTCACCATGGCCAGGGACACGTCCATCAGCACAGTTTACATGGACCTGA
GCAGGCTGAGATCTGACGACACGGCCGTGTATTACTGTGCGAGAATACGC
GGTGGTAACTCGGTCTTTGACTACTGGGGCCAGGGAACCCTGGTCACCGT
CTCCTCA

Clone 7A4 HC AA (CDRs Underlined)

(SEQ ID NO: 118)
QVQLVQSGAEVKKPGASVKVSCKASGYSFTSYDINWVRQATGQGLEWMGW
MNPNSGNTGYAQKFQGRVTMTRDTSISTAYMELSSLRSEDTAVYYCGRAG
YYYYFGMDVWGQGTTVTVSS

Clone 7A4 HC AA CDR1:

(SEQ ID NO: 119)
SYDIN

Clone 7A4 HC AA CDR2:

(SEQ ID NO: 120)
WMNPNSGNTGYAQKFQG

Clone 7A4 HC AA CDR3:

(SEQ ID NO: 121)
AGYYYYFGMDV

Clone 7A4 LC DNA

(SEQ ID NO: 122)
GACATCGTGATGACCCAGTCTCCAGACTCCCTGGCTGTGTCTCTGGGCGA
GAGGGCCACCATCAACTGCAAGTCCACCCAGAGTATTTTATACACCTCCA
ACAATAAGAACTTCTTAGCTTGGTACCAGCAGAAACCAGGGCAGCCTCCT
AAACTGCTCATTTCCTGGGCATCTATCCGGGAATCCGGGGTCCCTGACCG
ATTCAGTGGCAGCGGGTCTGGGACAGATTTCGCTCTCACCATCAGCAGCC
TGCAGGCTGAAGATGTGGCAGTTTATTACTGTCAACAATATTTTAGTACT
ATGTTCAGTTTTGGCCAGGGGACCAAGCTGGAGATCAAACGA

Clone 7A4 LC AA (CDRs Underlined)

(SEQ ID NO: 123)
EIVLTQSPGTLSLSPGERATLSCRAGQSVTSSSFAWYQQKPGQAPRLLIY
QTSTRATGIPDRFSGSGSGTDFTLTISRLEPEDFAVYYCQQYGGSRSFGQ
GTKVELKR

Clone 7A4 LC AA CDR1:

(SEQ ID NO: 124)
RAGQSVTSSSFA

Clone 7A4 LC AA CDR2:

(SEQ ID NO: 125)
QTSTRAT

Clone 7A4 LC AA CDR3:

(SEQ ID NO: 126)
QQYGGSRS

Clone 7A5 HC DNA

(SEQ ID NO: 127)
CAGGTGCAGCTGCAGGAGTCGGGCCCAGGACTGGTGAAGCCTTCACAGAC
CCTGTCCCTCACCTGCACTGTCTCTGGTGGCTCCATCAGTAGTGGTGCAT
ACTACTGGACTTGGATCCGCCAGCACCCAGGGAAGGGCCTGGAGTGGATT
GGGTACATCCATTACAGTGGGAGCACCTACTCCAACCCGTCCCTCAAGAG
TCGAATTACCATATCGTTAGACACGTCTAAGAACCAGTTCTCCCTGAAGC
TGAACTCTGTGACTGCCGCGGACACGGCCGTGTATTACTGTGCGAGACAA
GAGGACTACGGTGGTTTGTTTGACTACTGGGGCCAGGGAACCCTGGTCAC
CGTTTCCTCA

Clone 7A5 HC AA (CDRs Underlined)

(SEQ ID NO: 128)
QVQLVQSGAEVKKPGASVKVSCKASGYSFTSYDINWVRQATGQGLEWMGW
MNPNSGNTGYAQKFQGRVTMTRDTSISTAYMELSSLRSEDTAVYYCGRAG
YYYYFGMDVWGQGTTVTVSS

Clone 7A5 HC AA CDR1:

(SEQ ID NO: 129)
SYDIN

Clone 7A5 HC AA CDR2:

(SEQ ID NO: 130)
WMNPNSGNTGYAQKFQG

Clone 7A5 HC AA CDR3:

(SEQ ID NO: 131)
AGYYYYFGMDV

Clone 7A5 LC DNA

(SEQ ID NO: 132)
GAAATAGTGATGACGCAGTCTCCAGCCACCCTGTCTGTGTCTCCAGGGGAA
AGAATCACCCTCTCCTGCAGGGCCAGTCAGAGTGTTACCACCGACTTAGCC
TGGTACCAGCAGATGCCTGGACAGGCTCCCCGGCTCCTCATCTATGATGCT
TCCACCAGGGCCACTGGTTTCCCAGCCAGATTCAGTGGCAGTGGGTCTGGG
ACAGACTTCACGCTCACCATCAGCAGCCTGCAGGCTGAAGATTTTGCAGTT
TATTACTGTCAACATTATAAAACCTGGCCTCTCACTTTCGGCGGAGGGACT
AAGGTGGAGATCAAACGA

Clone 7A5 LC AA (CDRs Underlined)

(SEQ ID NO: 133)
EIVLTQSPGTLSLSPGERATLSCRAGQSVTSSSLAWYQQKPGQAPRLLIYQ
TSTRATGIPDRFSGSGSGTDFTLTISRLEPEDFAVYYCQQYGGSRAFGQGT
KVELKR

Clone 7A5 LC AA CDR1:

(SEQ ID NO: 134)
RAGQSVTSSSLA

Clone 7A5 LC AA CDR2:

(SEQ ID NO: 135)
QTSTRAT

Clone 7A5 LC AA CDR3:

(SEQ ID NO: 136)
QQYGGSRA

Clone 14C11 HC DNA

(SEQ ID NO: 137)
CAGGTGCAGCTGCAGGAGTCGGGCCCAGGACTGGTGAAGCCTTCACAGACC
CTGTCCCTCACCTGCACTGTCTCTGGTGGCTCCATCAGTAGTGGTGCATAC
TACTGGACTTGGATCCGCCAGCACCCAGGGAAGGGCCTGGAGTGGATTGGG
TACATCCATTACAGTGGGAGCACCTACTCCAACCCGTCCCTCAAGAGTCGA
ATTACCATATCGTTAGACACGTCTAAGAACCAGTTCTCCCTGAAGCTGAAC
TCTGTGACTGCCGCGGACACGGCCGTGTATTACTGTGCGAGACAAGAGGAC
TACGGTGGTTTGTTTGACTACTGGGGCCAGGGAACCCTGGTCACCGTTTCC
TCA

Clone 14C11 HC AA (CDRs Underlined)

(SEQ ID NO: 138)
QVQLVQSGAEVKKPGASVKVSCKASGYTFTGYYMHWVRQAPGQGLEWMGWI
NPNSGGTNSAQKFQGRVTMTRDTSISTAYMELNRLRSDDTAVYYCARGWLQ
TYYFDNWGQGTLVTVSS

Clone 14C11 HC AA CDR1:

(SEQ ID NO: 139)
GYYMH

Clone 14C1 HC AA CDR2:

(SEQ ID NO: 140)
WINPNSGGTNSAQKFQG

Clone 14C1 HC AA CDR3:

(SEQ ID NO: 141)
GWLQTYYFDN

Clone 14C11 LC DNA

(SEQ ID NO: 142)
GAAATAGTGATGACGCAGTCTCCAGCCACCCTGTCTGTGTCTCCAGGGGAA
AGAATCACCCTCTCCTGCAGGGCCAGTCAGAGTGTTACCACCGACTTAGCC
TGGTACCAGCAGATGCCTGGACAGGCTCCCCGGCTCCTCATCTATGATGCT
TCCACCAGGGCCACTGGTTTCCCAGCCAGATTCAGTGGCAGTGGGTCTGGG
ACAGACTTCACGCTCACCATCAGCAGCCTGCAGGCTGAAGATTTTGCAGTT
TATTACTGTCAACATTATAAAACCTGGCCTCTCACTTTCGGCGGAGGGACT
AAGGTGGAGATCAAACGA

Clone 14C11 LC AA (CDRs Underlined)

(SEQ ID NO: 143)
DIVMTQSPDSLAVSLGERATIYCKSSQTVLTSSNNKNFLAWYQQKLGQPPK
LLISWASTRESGVPDRFSGSGSGTDFTLTISSLQAEDVAIYYCQHYYTSPL
TFGGGTKVEIKR

Clone 14C11 LC AA CDR1:

(SEQ ID NO: 144)
TVLTSSNNKNFLA

Clone 14C1 LC AA CDR2:

(SEQ ID NO: 145)
WASTRES

Clone 14C1 LC AA CDR3:

(SEQ ID NO: 146)
QHYYTSPLT

Construct S1-2F3-CD28T-CD28-41BB DNA (Signal Sequence in Bold)

(SEQ ID NO: 17)
ATGGCACTCCCCGTAACTGCTCTGCTGCTGCCGTTGGCATTGCTCCTGCA
CGCCGCACGCCCGCAGGTGCAGCTGGTGCAGAGCGGAGCCGAGGTGAAGA
AGCCCGGAGCCAGCGTGAAGGTGAGCTGCAAAGCCAGCGGCTACACCTTC
TCCACCTACTGGATCGAGTGGGTGAGACAGGCCCCGGACAGAGGCTGGAA
TGGATGGGAGAGATCCTGCCCGGCAGCGGCAACACCGACTTCAACGAGAA
GTTCCAGGGCAGAGTGACCTTCACCGCCGATACCAGCAGCGACACCGCCT
ACATGGAACTGAGCAGCCTGAGAAGCGAGGATACCGCCGTCTACTACTGC
ACCAGATGGGGCTACTACGGCACCAGGGGCTATTTCAACGTGTGGGGCCA
GGGAACCCTCGTGACCGTGAGCAGCGGAGGCGGAGGATCTGGTGGCGGTG
GTTCTGGCGGCGGAGGCTCCGAGATTGTGCTGACCCAGAGCCCTGCTACA
CTGAGCCTGAGCCCCGGCGAGAGAGCCACACTGAGCTGCAGAGCCAGCAG
CAGCGTGAGCTACATGCACTGGTTCCAGCAAAAGCCCGGCCAGGCCCCTA
GGCTGCTGATCTACAGCACATCCAACCTGGCCAGCGGCATCCCTGCCAGA
TTCAGCGGTTCTGGCTCCGGCACCGACTACACCCTGACCATCTCCAGCCT
GGAGCCCGAGGACTTTGCCGTGTATTACTGCCAGCAGAGGAGGAGCTTCC
CCTACACATTCGGCCAGGGCACCAAACTGGAGATCAAGGCCGCTGCCCTT
GATAATGAAAAGTCAAACGGAACAATCATTCACGTGAAGGGCAAGCACCT
CTGTCCGTCACCCTTGTTCCCTGGTCCATCCAAGCCATTCTGGGTGTTGG
TCGTAGTGGGTGGAGTCCTCGCTTGTTACTCTCTGCTCGTCACCGTGGCT
TTTATAATCTTCTGGGTTAGATCCAAAAGAAGCCGCCTGCTCCATAGCGA
TTACATGAATATGACTCCACGCCGCCCTGGCCCCACAAGGAAACACTACC
AGCCTTACGCACCACCTAGAGATTTCGCTGCCTATCGGAGCCGCTTTTCC
GTCGTTAAGCGGGGGAGAAAAAAGCTGCTGTACATTTTCAAACAGCCGTT
TATGAGGCCGGTCCAAACGACTCAGGAAGAGGACGGCTGCTCCTGCCGCT
TTCCTGAGGAGGAGGAGGGCGGGTGCGAACTGAGGGTGAAGTTTTCCAGA
TCTGCAGATGCACCAGCGTATCAGCAGGGCCAGAACCAACTGTATAACGA
GCTCAACCTGGGACGCAGGGAAGAGTATGACGTTTTGGACAAGCGCAGAG
GACGGGACCCTGAGATGGGTGGCAAACCAAGACGAAAAAACCCCCAGGAG
GGTCTCTATAATGAGCTGCAGAAGGATAAGATGGCTGAAGCCTATTCTGA
AATAGGCATGAAAGGAGAGCGGAGAAGGGGAAAAGGGCACGACGGTTTGT
ACCAGGGACTCAGCACTGCTACGAAGGATACTTATGACGCTCTCCACATG
CAAGCCCTGCCACCTAGGGCCAAGAGAAGTGGCAGCGGGGAGGGCCGGGG
ATCTCTCCTTACATGTGGGGACGTGGAAGAAAATCCGGGGCCTATGGGTG
CCGGCGCCACGGGAAGGGCTATGGATGGCCCGCGACTGCTTCTCCTGCTG
TTGTTGGGCGTGTCTCTCGGAGGCGCTAAGGAGGCCTGTCCAACGGGCCT
CTACACTCACTCCGGTGAATGTTGCAAAGCCTGTAACCTTGGCGAGGGCG
TCGCACAACCTTGTGGTGCTAACCAGACAGTCTGTGAACCATGCCTGGAT
TCAGTGACATTCAGCGATGTTGTCTCAGCCACCGAGCCTTGCAAGCCTTG
TACCGAATGTGTGGGCCTTCAGTCCATGTCCGCCCCCTGTGTCGAAGCCG
ATGATGCAGTGTGCAGATGTGCCTATGGATATTACCAGGACGAAACTACC
GGGCGGTGTGAGGCCTGCCGGGTGTGTGAAGCCGGCTCTGGCCTCGTGTT
CAGTTGCCAGGATAAGCAAAACACAGTATGTGAGGAGTGTCCAGACGGAA
CCTACAGCGACGAGGCGAACCACGTCGACCCTTGCTTGCCGTGCACCGTC
TGCGAGGATACCGAACGCCAGCTGAGAGAGTGTACGCGCTGGGCAGACGC
TGAGTGCGAGGAGATCCCTGGGAGATGGATCACCCGGAGCACACCTCCTG
AGGGATCAGACAGTACAGCCCCGAGTACCCAAGAACCGGAGGCCCCTCCA
GAGCAGGACCTGATCGCTTCTACAGTTGCTGGCGTGGTGACGACAGTCAT
GGGATCCTCACAACCAGTCGTGACGCGGGGCACAACCGACAATCTGATTC
CTGTCTACTGTAGCATCTTGGCAGCCGTGGTCGTGGGCCTGGTAGCCTAC
ATCGCCTTTAAGAGATGACCTAGGTAA

Construct S1-2F3-CD28T-CD28-41BB AA (Signal Sequence in Bold; CDRs Underlined)

(SEQ ID NO: 18)
MALPVTALLLPLALLLHAARPQVQLVQSGAEVKKPGASVKVSCKASGYTF
STYWIEWVRQAPGQRLEWMGEILPGSGNTDFNEKFQGRVTFTADTSSDTA
YMELSSLRSEDTAVYYCTRWGYYGTRGYFNVWGQGTLVTVSSGGGGSGGG
GSGGGGSEIVLTQSPATLSLSPGERATLSCRASSSVSYMHWFQQKPGQAP
RLLIYSTSNLASGIPARFSGSGSGTDYTLTISSLEPEDFAVYYCQQRRSF
PYTFGQGTKLEIKAAALDNEKSNGTIIHVKGKHLCPSPLFPGPSKPFWVL
VVVGGVLACYSLLVTVAFIIFWVRSKRSRLLHSDYMNMTPRRPGPTRKHY
QPYAPPRDFAAYRSRFSVVKRGRKKLLYIFKQPFMRPVQTTQEEDGCSCR
FPEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDGL
YQGLSTATKDTYDALHMQALPPRAKRSGSGEGRGSLLTCGDVEENPGPMG
AGATGRAMDGPRLLLLLLLGVSLGGAKEACPTGLYTHSGECCKACNLGEG
VAQPCGANQTVCEPCLDSVTFSDVVSATEPCKPCTECVGLQSMSAPCVEA
DDAVCRCAYGYYQDETTGRCEACRVCEAGSGLVFSCQDKQNTVCEECPDG
TYSDEANHVDPCLPCTVCEDTERQLRECTRWADAECEEIPGRWITRSTPP
EGSDSTAPSTQEPEAPPEQDLIASTVAGVVTTVMGSSQPVVTRGTTDNLI
PVYCSILAAVVVGLVAYIAFKR

Construct S1-2F3-CD28T-CD28 DNA (Signal Sequence in Bold)

(SEQ ID NO: 19)
ATGGCACTCCCCGTAACTGCTCTGCTGCTGCCGTTGGCATTGCTCCTGCA
CGCCGCACGCCCGCAGGTGCAGCTGGTGCAGAGCGGAGCCGAGGTGAAGA
AGCCCGGAGCCAGCGTGAAGGTGAGCTGCAAAGCCAGCGGCTACACCTTC
TCCACCTACTGGATCGAGTGGGTGAGACAGGCCCCCGGACAGAGGCTGGA
ATGGATGGGAGAGATCCTGCCCGGCAGCGGCAACACCGACTTCAACGAGA
AGTTCCAGGGCAGAGTGACCTTCACCGCCGATACCAGCAGCGACACCGCC
TACATGGAACTGAGCAGCCTGAGAAGCGAGGATACCGCCGTCTACTACTG
CACCAGATGGGGCTACTACGGCACCAGGGGCTATTTCAACGTGTGGGGCC
AGGGAACCCTCGTGACCGTGAGCAGCGGAGGCGGAGGATCTGGTGGCGGT
GGTTCTGGCGGCGGAGGCTCCGAGATTGTGCTGACCCAGAGCCCTGCTAC
ACTGAGCCTGAGCCCCGGCGAGAGAGCCACACTGAGCTGCAGAGCCAGCA
GCAGCGTGAGCTACATGCACTGGTTCCAGCAAAAGCCCGGCCAGGCCCCT
AGGCTGCTGATCTACAGCACATCCAACCTGGCCAGCGGCATCCCTGCCAG
ATTCAGCGGTTCTGGCTCCGGCACCGACTACACCCTGACCATCTCCAGCC
TGGAGCCCGAGGACTTTGCCGTGTATTACTGCCAGCAGAGGAGGAGCTTC
CCCTACACATTCGGCCAGGGCACCAAACTGGAGATCAAGGCCGCTGCCCT
TGATAATGAAAAGTCAAACGGAACAATCATTCACGTGAAGGGCAAGCACC
TCTGTCCGTCACCCTTGTTCCCTGGTCCATCCAAGCCATTCTGGGTGTTG
GTCGTAGTGGGTGGAGTCCTCGCTTGTTACTCTCTGCTCGTCACCGTGGC
TTTTATAATCTTCTGGGTTAGATCCAAAAGAAGCCGCCTGCTCCATAGCG
ATTACATGAATATGACTCCACGCCGCCCTGGCCCCACAAGGAAACACTAC
CAGCCTTACGCACCACCTAGAGATTTCGCTGCCTATCGGAGCAGGGTGAA
GTTTTCCAGATCTGCAGATGCACCAGCGTATCAGCAGGGCCAGAACCAAC
TGTATAACGAGCTCAACCTGGGACGCAGGGAAGAGTATGACGTTTTGGAC
AAGCGCAGAGGACGGGACCCTGAGATGGGTGGCAAACCAAGACGAAAAAA
CCCCCAGGAGGGTCTCTATAATGAGCTGCAGAAGGATAAGATGGCTGAAG
CCTATTCTGAAATAGGCATGAAAGGAGAGCGGAGAAGGGGAAAAGGGCAC
GACGGTTTGTACCAGGGACTCAGCACTGCTACGAAGGATACTTATGACGC
TCTCCACATGCAAGCCCTGCCACCTAGGGCCAAGAGAAGTGGCAGCGGGG
AGGGCCGGGGATCTCTCCTTACATGTGGGGACGTGGAAGAAAATCCGGGG
CCTATGGGTGCCGGCGCCACGGGAAGGGCTATGGATGGCCCGCGACTGCT
TCTCCTGCTGTTGTTGGGCGTGTCTCTCGGAGGCGCTAAGGAGGCCTGTC
CAACGGGCCTCTACACTCACTCCGGTGAATGTTGCAAAGCCTGTAACCTT
GGCGAGGGCGTCGCACAACCTTGTGGTGCTAACCAGACAGTCTGTGAACC
ATGCCTGGATTCAGTGACATTCAGCGATGTTGTCTCAGCCACCGAGCCTT
GCAAGCCTTGTACCGAATGTGTGGGCCTTCAGTCCATGTCCGCCCCCTGT
GTCGAAGCCGATGATGCAGTGTGCAGATGTGCCTATGGATATTACCAGGA
CGAAACTACCGGGCGGTGTGAGGCCTGCCGGGTGTGTGAAGCCGGCTCTG
GCCTCGTGTTCAGTTGCCAGGATAAGCAAAACACAGTATGTGAGGAGTGT
CCAGACGGAACCTACAGCGACGAGGCGAACCACGTCGACCCTTGCTTGCC
GTGCACCGTCTGCGAGGATACCGAACGCCAGCTGAGAGAGTGTACGCGCT
GGGCAGACGCTGAGTGCGAGGAGATCCCTGGGAGATGGATCACCCGGAGC
ACACCTCCTGAGGGATCAGACAGTACAGCCCCGAGTACCCAAGAACCGGA
GGCCCCTCCAGAGCAGGACCTGATCGCTTCTACAGTTGCTGGCGTGGTGA
CGACAGTCATGGGATCCTCACAACCAGTCGTGACGCGGGGCACAACCGAC
AATCTGATTCCTGTCTACTGTAGCATCTTGGCAGCCGTGGTCGTGGGCCT
GGTAGCCTACATCGCCTTTAAGAGATGACCTAGGTAA

Construct S1-2F3-CD28T-CD28 AA (Signal Sequence in Bold; CDRs Underlined)

(SEQ ID NO: 20)
MALPVTALLLPLALLLHAARPQVQLVQSGAEVKKPGASVKVSCKASGYTF
STYWIEWVRQAPGQRLEWMGEILPGSGNTDFNEKFQGRVTFTADTSSDTA
YMELSSLRSEDTAVYYCTRWGYYGTRGYFNVWGQGTLVTVSSGGGGSGGG
GSGGGGSEIVLTQSPATLSLSPGERATLSCRASSSVSYMHWFQQKPGQAP
RLLIYSTSNLASGIPARFSGSGSGTDYTLTISSLEPEDFAVYYCQQRRSF
PYTFGQGTKLEIKAAALDNEKSNGTIIHVKGKHLCPSPLFPGPSKPFWVL
VVVGGVLACYSLLVTVAFIIFWVRSKRSRLLHSDYMNMTPRRPGPTRKHY
QPYAPPRDFAAYRSRVKFSRSADAPAYQQGQNQLYNELNLGRREEYDVLD
KRRGRDPEMGGKPRRKNPQEGLYNELQKDKMAEAYSEIGMKGERRRGKGH
DGLYQGLSTATKDTYDALHMQALPPRAKRSGSGEGRGSLLTCGDVEENPG
PMGAGATGRAMDGPRLLLLLLLGVSLGGAKEACPTGLYTHSGECCKACNL
GEGVAQPCGANQTVCEPCLDSVTFSDVVSATEPCKPCTECVGLQSMSAPC
VEADDAVCRCAYGYYQDETTGRCEACRVCEAGSGLVFSCQDKQNTVCEEC
PDGTYSDEANHVDPCLPCTVCEDTERQLRECTRWADAECEEIPGRWITRS
TPPEGSDSTAPSTQEPEAPPEQDLIASTVAGVVTTVMGSSQPVVTRGTTD
NLIPVYCSILAAVVVGLVAYIAFKR

Construct S1-2F3-CD28T-41BB DNA (Signal Sequence in Bold)

(SEQ ID NO: 21)
ATGGCACTCCCCGTAACTGCTCTGCTGCTGCCGTTGGCATTGCTCCTGCA
CGCCGCACGCCCGCAGGTGCAGCTGGTGCAGAGCGGAGCCGAGGTGAAGA
AGCCCGGAGCCAGCGTGAAGGTGAGCTGCAAAGCCAGCGGCTACACCTTC
TCCACCTACTGGATCGAGTGGGTGAGACAGGCCCCCGGACAGAGGCTGGA
ATGGATGGGAGAGATCCTGCCCGGCAGCGGCAACACCGACTTCAACGAGA
AGTTCCAGGGCAGAGTGACCTTCACCGCCGATACCAGCAGCGACACCGCC
TACATGGAACTGAGCAGCCTGAGAAGCGAGGATACCGCCGTCTACTACTG
CACCAGATGGGGCTACTACGGCACCAGGGGCTATTTCAACGTGTGGGGCC
AGGGAACCCTCGTGACCGTGAGCAGCGGAGGCGGAGGATCTGGTGGCGGT
GGTTCTGGCGGCGGAGGCTCCGAGATTGTGCTGACCCAGAGCCCTGCTAC
ACTGAGCCTGAGCCCCGGCGAGAGAGCCACACTGAGCTGCAGAGCCAGCA
GCAGCGTGAGCTACATGCACTGGTTCCAGCAAAAGCCCGGCCAGGCCCCT
AGGCTGCTGATCTACAGCACATCCAACCTGGCCAGCGGCATCCCTGCCAG
ATTCAGCGGTTCTGGCTCCGGCACCGACTACACCCTGACCATCTCCAGCC
TGGAGCCCGAGGACTTTGCCGTGTATTACTGCCAGCAGAGGAGGAGCTTC
CCCTACACATTCGGCCAGGGCACCAAACTGGAGATCAAGGCCGCTGCCCT
TGATAATGAAAAGTCAAACGGAACAATCATTCACGTGAAGGGCAAGCACC
TCTGTCCGTCACCCTTGTTCCCTGGTCCATCCAAGCCATTCTGGGTGTTG
GTCGTAGTGGGTGGAGTCCTCGCTTGTTACTCTCTGCTCGTCACCGTGGC
TTTTATAATCTTCTGGGTTCGCTTTTCCGTCGTTAAGCGGGGGAGAAAAA
AGCTGCTGTACATTTTCAAACAGCCGTTTATGAGGCCGGTCCAAACGACT
CAGGAAGAGGACGGCTGCTCCTGCCGCTTTCCTGAGGAGGAGGAGGGCGG
GTGCGAACTGAGGGTGAAGTTTTCCAGATCTGCAGATGCACCAGCGTATC
AGCAGGGCCAGAACCAACTGTATAACGAGCTCAACCTGGGACGCAGGGAA
GAGTATGACGTTTTGGACAAGCGCAGAGGACGGGACCCTGAGATGGGTGG
CAAACCAAGACGAAAAAACCCCCAGGAGGGTCTCTATAATGAGCTGCAGA
AGGATAAGATGGCTGAAGCCTATTCTGAAATAGGCATGAAAGGAGAGCGG
AGAAGGGGAAAAGGGCACGACGGTTTGTACCAGGGACTCAGCACTGCTAC
GAAGGATACTTATGACGCTCTCCACATGCAAGCCCTGCCACCTAGGGCCA
AGAGAAGTGGCAGCGGGGAGGGCCGGGGATCTCTCCTTACATGTGGGGAC
GTGGAAGAAAATCCGGGGCCTATGGGTGCCGGCGCCACGGGAAGGGCTAT
GGATGGCCCGCGACTGCTTCTCCTGCTGTTGTTGGGCGTGTCTCTCGGAG
GCGCTAAGGAGGCCTGTCCAACGGGCCTCTACACTCACTCCGGTGAATGT
TGCAAAGCCTGTAACCTTGGCGAGGGCGTCGCACAACCTTGTGGTGCTAA
CCAGACAGTCTGTGAACCATGCCTGGATTCAGTGACATTCAGCGATGTTG
TCTCAGCCACCGAGCCTTGCAAGCCTTGTACCGAATGTGTGGGCCTTCAG
TCCATGTCCGCCCCCTGTGTCGAAGCCGATGATGCAGTGTGCAGATGTGC
CTATGGATATTACCAGGACGAAACTACCGGGCGGTGTGAGGCCTGCCGGG
TGTGTGAAGCCGGCTCTGGCCTCGTGTTCAGTTGCCAGGATAAGCAAAAC
ACAGTATGTGAGGAGTGTCCAGACGGAACCTACAGCGACGAGGCGAACCA
CGTCGACCCTTGCTTGCCGTGCACCGTCTGCGAGGATACCGAACGCCAGC
TGAGAGAGTGTACGCGCTGGGCAGACGCTGAGTGCGAGGAGATCCCTGGG
AGATGGATCACCCGGAGCACACCTCCTGAGGGATCAGACAGTACAGCCCC
GAGTACCCAAGAACCGGAGGCCCCTCCAGAGCAGGACCTGATCGCTTCTA
CAGTTGCTGGCGTGGTGACGACAGTCATGGGATCCTCACAACCAGTCGTG
ACGCGGGGCACAACCGACAATCTGATTCCTGTCTACTGTAGCATCTTGGC
AGCCGTGGTCGTGGGCCTGGTAGCCTACATCGCCTTTAAGAGATGACCTA
GGTAA

Construct S1-2F3-CD28T-41BB AA (Signal Sequence in Bold; CDRs Underlined)

(SEQ ID NO: 22)
MALPVTALLLPLALLLHAARPQVQLVQSGAEVKKPGASVKVSCKASGYTF
STYWIEWVRQAPGQRLEWMGEILPGSGNTDFNEKFQGRVTFTADTSSDTA
YMELSSLRSEDTAVYYCTRWGYYGTRGYFNVWGQGTLVTVSSGGGGSGGG
GSGGGGSEIVLTQSPATLSLSPGERATLSCRASSSVSYMHWFQQKPGQAP
RLLIYSTSNLASGIPARFSGSGSGTDYTLTISSLEPEDFAVYYCQQRRSF
PYTFGQGTKLEIKAAALDNEKSNGTIIHVKGKHLCPSPLFPGPSKPFWVL
VVVGGVLACYSLLVTVAFIIFWVRFSVVKRGRKKLLYIFKQPFMRPVQTT
QEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGER
RRGKGHDGLYQGLSTATKDTYDALHMQALPPRAKRSGSGEGRGSLLTCGD
VEENPGPMGAGATGRAMDGPRLLLLLLLGVSLGGAKEACPTGLYTHSGEC
CKACNLGEGVAQPCGANQTVCEPCLDSVTFSDVVSATEPCKPCTECVGLQ
SMSAPCVEADDAVCRCAYGYYQDETTGRCEACRVCEAGSGLVFSCQDKQN
TVCEECPDGTYSDEANHVDPCLPCTVCEDTERQLRECTRWADAECEEIPG
RWITRSTPPEGSDSTAPSTQEPEAPPEQDLIASTVAGVVTTVMGSSQPVV
TRGTTDNLIPVYCSILAAVVVGLVAYIAFKR

Construct S1-2F3-C8K-CD28 DNA (Signal Sequence in Bold)

(SEQ ID NO: 23)
ATGGCACTCCCCGTAACTGCTCTGCTGCTGCCGTTGGCATTGCTCCTGCA
CGCCGCACGCCCGCAGGTGCAGCTGGTGCAGAGCGGAGCCGAGGTGAAGA
AGCCCGGAGCCAGCGTGAAGGTGAGCTGCAAAGCCAGCGGCTACACCTTC
TCCACCTACTGGATCGAGTGGGTGAGACAGGCCCCCGGACAGAGGCTGGA
ATGGATGGGAGAGATCCTGCCCGGCAGCGGCAACACCGACTTCAACGAGA
AGTTCCAGGGCAGAGTGACCTTCACCGCCGATACCAGCAGCGACACCGCC
TACATGGAACTGAGCAGCCTGAGAAGCGAGGATACCGCCGTCTACTACTG
CACCAGATGGGGCTACTACGGCACCAGGGGCTATTTCAACGTGTGGGGCC
AGGGAACCCTCGTGACCGTGAGCAGCGGAGGCGGAGGATCTGGTGGCGGT
GGTTCTGGCGGCGGAGGCTCCGAGATTGTGCTGACCCAGAGCCCTGCTAC
ACTGAGCCTGAGCCCCGGCGAGAGAGCCACACTGAGCTGCAGAGCCAGCA
GCAGCGTGAGCTACATGCACTGGTTCCAGCAAAAGCCCGGCCAGGCCCCT
AGGCTGCTGATCTACAGCACATCCAACCTGGCCAGCGGCATCCCTGCCAG
ATTCAGCGGTTCTGGCTCCGGCACCGACTACACCCTGACCATCTCCAGCC
TGGAGCCCGAGGACTTTGCCGTGTATTACTGCCAGCAGAGGAGGAGCTTC
CCCTACACATTCGGCCAGGGCACCAAACTGGAGATCAAGGCCGCTGCCTT
CGTGCCTGTTTTTCTGCCCGCGAAACCCACAACTACCCCCGCCCCTCGGC
CCCCAACTCCTGCACCAACTATCGCTTCCCAACCCCTGTCTCTGAGACCT
GAGGCATGCCGCCCCGCGGCAGGCGGCGCCGTGCACACTAGAGGCCTGGA
CTTCGCCTGCGATATTTATATCTGGGCCCCCCTTGCCGGGACATGCGGGG
TACTGCTGCTGTCTCTGGTGATTACCCTCTACTGCAACCACAGAAACAGA
TCCAAAAGAAGCCGCCTGCTCCATAGCGATTACATGAATATGACTCCACG
CCGCCCTGGCCCCACAAGGAAACACTACCAGCCTTACGCACCACCTAGAG
ATTTCGCTGCCTATCGGAGCAGGGTGAAGTTTTCCAGATCTGCAGATGCA
CCAGCGTATCAGCAGGGCCAGAACCAACTGTATAACGAGCTCAACCTGGG
ACGCAGGGAAGAGTATGACGTTTTGGACAAGCGCAGAGGACGGGACCCTG
AGATGGGTGGCAAACCAAGACGAAAAAACCCCCAGGAGGGTCTCTATAAT
GAGCTGCAGAAGGATAAGATGGCTGAAGCCTATTCTGAAATAGGCATGAA
AGGAGAGCGGAGAAGGGGAAAAGGGCACGACGGTTTGTACCAGGGACTCA
GCACTGCTACGAAGGATACTTATGACGCTCTCCACATGCAAGCCCTGCCA
CCTAGGGCCAAGAGAAGTGGCAGCGGGGAGGGCCGGGGATCTCTCCTTAC
ATGTGGGGACGTGGAAGAAAATCCGGGGCCTATGGGTGCCGGCGCCACGG
GAAGGGCTATGGATGGCCCGCGACTGCTTCTCCTGCTGTTGTTGGGCGTG
TCTCTCGGAGGCGCTAAGGAGGCCTGTCCAACGGGCCTCTACACTCACTC
CGGTGAATGTTGCAAAGCCTGTAACCTTGGCGAGGGCGTCGCACAACCTT
GTGGTGCTAACCAGACAGTCTGTGAACCATGCCTGGATTCAGTGACATTC
AGCGATGTTGTCTCAGCCACCGAGCCTTGCAAGCCTTGTACCGAATGTGT
GGGCCTTCAGTCCATGTCCGCCCCCTGTGTCGAAGCCGATGATGCAGTGT
GCAGATGTGCCTATGGATATTACCAGGACGAAACTACCGGGCGGTGTGAG
GCCTGCCGGGTGTGTGAAGCCGGCTCTGGCCTCGTGTTCAGTTGCCAGGA
TAAGCAAAACACAGTATGTGAGGAGTGTCCAGACGGAACCTACAGCGACG
AGGCGAACCACGTCGACCCTTGCTTGCCGTGCACCGTCTGCGAGGATACC
GAACGCCAGCTGAGAGAGTGTACGCGCTGGGCAGACGCTGAGTGCGAGGA
GATCCCTGGGAGATGGATCACCCGGAGCACACCTCCTGAGGGATCAGACA
GTACAGCCCCGAGTACCCAAGAACCGGAGGCCCCTCCAGAGCAGGACCTG
ATCGCTTCTACAGTTGCTGGCGTGGTGACGACAGTCATGGGATCCTCACA
ACCAGTCGTGACGCGGGGCACAACCGACAATCTGATTCCTGTCTACTGTA
GCATCTTGGCAGCCGTGGTCGTGGGCCTGGTAGCCTACATCGCCTTTAAG
AGATGACCTAGGTAA

Construct S1-2F3-C8K-CD28 AA (Signal Sequence in Bold; CDRs Underlined)

(SEQ ID NO: 24)
MALPVTALLLPLALLLHAARPQVQLVQSGAEVKKPGASVKVSCKASGYTF
STYWIEWVRQAPGQRLEWMGEILPGSGNTDFNEKFQGRVTFTADTSSDTA
YMELSSLRSEDTAVYYCTRWGYYGTRGYFNVWGQGTLVTVSSGGGGSGGG
GSGGGGSEIVLTQSPATLSLSPGERATLSCRASSSVSYMHWFQQKPGQAP
RLLIYSTSNLASGIPARFSGSGSGTDYTLTISSLEPEDFAVYYCQQRRSF
PYTFGQGTKLEIKAAAFVPVFLPAKPTTTPAPRPPTPAPTIASQPLSLRP
EACRPAAGGAVHTRGLDFACDIYIWAPLAGTCGVLLLSLVITLYCNHRNR
SKRSRLLHSDYMNMTPRRPGPTRKHYQPYAPPRDFAAYRSRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PRAKRSGSGEGRGSLLTCGDVEENPGPMGAGATGRAMDGPRLLLLLLLGV
SLGGAKEACPTGLYTHSGECCKACNLGEGVAQPCGANQTVCEPCLDSVTF
SDVVSATEPCKPCTECVGLQSMSAPCVEADDAVCRCAYGYYQDETTGRCE
ACRVCEAGSGLVFSCQDKQNTVCEECPDGTYSDEANHVDPCLPCTVCEDT
ERQLRECTRWADAECEEIPGRWITRSTPPEGSDSTAPSTQEPEAPPEQDL
IASTVAGVVTTVMGSSQPVVTRGTTDNLIPVYCSILAAVVVGLVAYIAFK
R

Construct S1-2F3-C8K-41BB DNA (Signal Sequence in Bold)

(SEQ ID NO: 25)
ATGGCACTCCCCGTAACTGCTCTGCTGCTGCCGTTGGCATTGCTCCTGCA
CGCCGCACGCCCGCAGGTGCAGCTGGTGCAGAGCGGAGCCGAGGTGAAGA
AGCCCGGAGCCAGCGTGAAGGTGAGCTGCAAAGCCAGCGGCTACACCTTC
TCCACCTACTGGATCGAGTGGGTGAGACAGGCCCCCGGACAGAGGCTGGA
ATGGATGGGAGAGATCCTGCCCGGCAGCGGCAACACCGACTTCAACGAGA
AGTTCCAGGGCAGAGTGACCTTCACCGCCGATACCAGCAGCGACACCGCC
TACATGGAACTGAGCAGCCTGAGAAGCGAGGATACCGCCGTCTACTACTG
CACCAGATGGGGCTACTACGGCACCAGGGGCTATTTCAACGTGTGGGGCC
AGGGAACCCTCGTGACCGTGAGCAGCGGAGGCGGAGGATCTGGTGGCGGT
GGTTCTGGCGGCGGAGGCTCCGAGATTGTGCTGACCCAGAGCCCTGCTAC
ACTGAGCCTGAGCCCCGGCGAGAGAGCCACACTGAGCTGCAGAGCCAGCA
GCAGCGTGAGCTACATGCACTGGTTCCAGCAAAAGCCCGGCCAGGCCCCT
AGGCTGCTGATCTACAGCACATCCAACCTGGCCAGCGGCATCCCTGCCAG
ATTCAGCGGTTCTGGCTCCGGCACCGACTACACCCTGACCATCTCCAGCC
TGGAGCCCGAGGACTTTGCCGTGTATTACTGCCAGCAGAGGAGGAGCTTC
CCCTACACATTCGGCCAGGGCACCAAACTGGAGATCAAGGCCGCTGCCTT
CGTGCCTGTTTTTCTGCCCGCGAAACCCACAACTACCCCCGCCCCTCGGC
CCCCAACTCCTGCACCAACTATCGCTTCCCAACCCCTGTCTCTGAGACCT
GAGGCATGCCGCCCCGCGGCAGGCGGCGCCGTGCACACTAGAGGCCTGGA
CTTCGCCTGCGATATTTATATCTGGGCCCCCCTTGCCGGGACATGCGGGG
TACTGCTGCTGTCTCTGGTGATTACCCTCTACTGCAACCACAGAAACCGC
TTTTCCGTCGTTAAGCGGGGGAGAAAAAAGCTGCTGTACATTTTCAAACA
GCCGTTTATGAGGCCGGTCCAAACGACTCAGGAAGAGGACGGCTGCTCCT
GCCGCTTTCCTGAGGAGGAGGAGGGCGGGTGCGAACTGAGGGTGAAGTTT
TCCAGATCTGCAGATGCACCAGCGTATCAGCAGGGCCAGAACCAACTGTA
TAACGAGCTCAACCTGGGACGCAGGGAAGAGTATGACGTTTTGGACAAGC
GCAGAGGACGGGACCCTGAGATGGGTGGCAAACCAAGACGAAAAAACCCC
CAGGAGGGTCTCTATAATGAGCTGCAGAAGGATAAGATGGCTGAAGCCTA
TTCTGAAATAGGCATGAAAGGAGAGCGGAGAAGGGGAAAAGGGCACGACG
GTTTGTACCAGGGACTCAGCACTGCTACGAAGGATACTTATGACGCTCTC
CACATGCAAGCCCTGCCACCTAGGGCCAAGAGAAGTGGCAGCGGGGAGGG
CCGGGGATCTCTCCTTACATGTGGGGACGTGGAAGAAAATCCGGGGCCTA
TGGGTGCCGGCGCCACGGGAAGGGCTATGGATGGCCCGCGACTGCTTCTC
CTGCTGTTGTTGGGCGTGTCTCTCGGAGGCGCTAAGGAGGCCTGTCCAAC
GGGCCTCTACACTCACTCCGGTGAATGTTGCAAAGCCTGTAACCTTGGCG
AGGGCGTCGCACAACCTTGTGGTGCTAACCAGACAGTCTGTGAACCATGC
CTGGATTCAGTGACATTCAGCGATGTTGTCTCAGCCACCGAGCCTTGCAA
GCCTTGTACCGAATGTGTGGGCCTTCAGTCCATGTCCGCCCCCTGTGTCG
AAGCCGATGATGCAGTGTGCAGATGTGCCTATGGATATTACCAGGACGAA
ACTACCGGGCGGTGTGAGGCCTGCCGGGTGTGTGAAGCCGGCTCTGGCCT
CGTGTTCAGTTGCCAGGATAAGCAAAACACAGTATGTGAGGAGTGTCCAG
ACGGAACCTACAGCGACGAGGCGAACCACGTCGACCCTTGCTTGCCGTGC
ACCGTCTGCGAGGATACCGAACGCCAGCTGAGAGAGTGTACGCGCTGGGC
AGACGCTGAGTGCGAGGAGATCCCTGGGAGATGGATCACCCGGAGCACAC
CTCCTGAGGGATCAGACAGTACAGCCCCGAGTACCCAAGAACCGGAGGCC
CCTCCAGAGCAGGACCTGATCGCTTCTACAGTTGCTGGCGTGGTGACGAC
AGTCATGGGATCCTCACAACCAGTCGTGACGCGGGGCACAACCGACAATC
TGATTCCTGTCTACTGTAGCATCTTGGCAGCCGTGGTCGTGGGCCTGGTA
GCCTACATCGCCTTTAAGAGATGACCTAGGTAA

Construct S1-2F3-C8K-41BB AA (Signal Sequence in Bold; CDRs Underlined)

(SEQ ID NO: 26)
MALPVTALLLPLALLLHAARPQVQLVQSGAEVKKPGASVKVSCKASGYTF
STYWIEWVRQAPGQRLEWMGEILPGSGNTDFNEKFQGRVTFTADTSSDTA
YMELSSLRSEDTAVYYCTRWGYYGTRGYFNVWGQGTLVTVSSGGGGSGGG
GSGGGGSEIVLTQSPATLSLSPGERATLSCRASSSVSYMHWFQQKPGQAP
RLLIYSTSNLASGIPARFSGSGSGTDYTLTISSLEPEDFAVYYCQQRRSF
PYTFGQGTKLEIKAAAFVPVFLPAKPTTTPAPRPPTPAPTIASQPLSLRP
EACRPAAGGAVHTRGLDFACDIYIWAPLAGTCGVLLLSLVITLYCNHRNR
FSVVKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDAL
HMQALPPRAKRSGSGEGRGSLLTCGDVEENPGPMGAGATGRAMDGPRLLL
LLLLGVSLGGAKEACPTGLYTHSGECCKACNLGEGVAQPCGANQTVCEPC
LDSVTFSDVVSATEPCKPCTECVGLQSMSAPCVEADDAVCRCAYGYYQDE
TTGRCEACRVCEAGSGLVFSCQDKQNTVCEECPDGTYSDEANHVDPCLPC
TVCEDTERQLRECTRWADAECEEIPGRWITRSTPPEGSDSTAPSTQEPEA
PPEQDLIASTVAGVVTTVMGSSQPVVTRGTTDNLIPVYCSILAAVVVGLV
AYIAFKR

Construct S1-11C2-CD28T-CD28-41BB DNA (Signal Sequence in Bold)

(SEQ ID NO: 27)
ATGGCACTCCCCGTAACTGCTCTGCTGCTGCCGTTGGCATTGCTCCTGCA
CGCCGCACGCCCGGAGATTCAGCTGGTGCAGAGCGGCGCCGAGGTGAAAA
AGCCCGGAGCCACCGTGAAGATCAGCTGCAAGGCCAGCGGCTACACATTC
ACCAACTACGGCATGAACTGGGTGCAACAGGCCCCCGGCCAGGGACTGGA
GTGGATGGGCTGGATGAACACATACACCGGCGAGCCCACCTACGCCGACA
AGTTCCAGGGCAGGGTGACATTCACACTCGATACCAGCGCCAGAACCGTG
TATATGGAACTGAGCAGCCTGAGGAGCGAGGACACCGCTGTGTACTTCTG
CGCAAGGGCTGGAGGCCAGCTGAGACCTGGCGCTATGGACTACTGGGGCC
AGGGCACCATGGTGACCGTGAGCTCCGGAGGCGGAGGATCTGGTGGCGGT
GGTTCTGGCGGCGGAGGCTCCGACATCGTGCTGACCCAGACACCTCTCTC
CCTGTCCGTGACCCCCGGACAGCCTGCCTCCATTTCCTGTAAGGCCTCCC
AGAGCGTGGACTATGACGGCGACAGCTTCATGAACTGGTACCTGCAGAAG
CCCGGACAACCCCCCCAGCTGCTGATCTACGTGGCCAGCAACCTGGAGTC
CGGCGTGCCTGACAGGTTTTCCGGCTCCGGCAGCGGCACCGACTTCACCC
TGAAGATCAGCAGGGTGGAAGCCGAGGACGTGGGCGTGTACTACTGCCAG
CAGAGCAACGAGGAGCCCCCTACCTTCGGACAGGGCACCAAGCTGGAGAT
CAAGGCCGCTGCCCTTGATAATGAAAAGTCAAACGGAACAATCATTCACG
TGAAGGGCAAGCACCTCTGTCCGTCACCCTTGTTCCCTGGTCCATCCAAG
CCATTCTGGGTGTTGGTCGTAGTGGGTGGAGTCCTCGCTTGTTACTCTCT
GCTCGTCACCGTGGCTTTTATAATCTTCTGGGTTAGATCCAAAAGAAGCC
GCCTGCTCCATAGCGATTACATGAATATGACTCCACGCCGCCCTGGCCCC
ACAAGGAAACACTACCAGCCTTACGCACCACCTAGAGATTTCGCTGCCTA
TCGGAGCCGCTTTTCCGTCGTTAAGCGGGGGAGAAAAAAGCTGCTGTACA
TTTTCAAACAGCCGTTTATGAGGCCGGTCCAAACGACTCAGGAAGAGGAC
GGCTGCTCCTGCCGCTTTCCTGAGGAGGAGGAGGGCGGGTGCGAACTGAG
GGTGAAGTTTTCCAGATCTGCAGATGCACCAGCGTATCAGCAGGGCCAGA
ACCAACTGTATAACGAGCTCAACCTGGGACGCAGGGAAGAGTATGACGTT
TTGGACAAGCGCAGAGGACGGGACCCTGAGATGGGTGGCAAACCAAGACG
AAAAAACCCCCAGGAGGGTCTCTATAATGAGCTGCAGAAGGATAAGATGG
CTGAAGCCTATTCTGAAATAGGCATGAAAGGAGAGCGGAGAAGGGGAAAA
GGGCACGACGGTTTGTACCAGGGACTCAGCACTGCTACGAAGGATACTTA
TGACGCTCTCCACATGCAAGCCCTGCCACCTAGGGCCAAGAGAAGTGGCA
GCGGGGAGGGCCGGGGATCTCTCCTTACATGTGGGGACGTGGAAGAAAAT
CCGGGGCCTATGGGTGCCGGCGCCACGGGAAGGGCTATGGATGGCCCGCG
ACTGCTTCTCCTGCTGTTGTTGGGCGTGTCTCTCGGAGGCGCTAAGGAGG
CCTGTCCAACGGGCCTCTACACTCACTCCGGTGAATGTTGCAAAGCCTGT
AACCTTGGCGAGGGCGTCGCACAACCTTGTGGTGCTAACCAGACAGTCTG
TGAACCATGCCTGGATTCAGTGACATTCAGCGATGTTGTCTCAGCCACCG
AGCCTTGCAAGCCTTGTACCGAATGTGTGGGCCTTCAGTCCATGTCCGCC
CCCTGTGTCGAAGCCGATGATGCAGTGTGCAGATGTGCCTATGGATATTA
CCAGGACGAAACTACCGGGCGGTGTGAGGCCTGCCGGGTGTGTGAAGCCG
GCTCTGGCCTCGTGTTCAGTTGCCAGGATAAGCAAAACACAGTATGTGAG
GAGTGTCCAGACGGAACCTACAGCGACGAGGCGAACCACGTCGACCCTTG
CTTGCCGTGCACCGTCTGCGAGGATACCGAACGCCAGCTGAGAGAGTGTA
CGCGCTGGGCAGACGCTGAGTGCGAGGAGATCCCTGGGAGATGGATCACC
CGGAGCACACCTCCTGAGGGATCAGACAGTACAGCCCCGAGTACCCAAGA
ACCGGAGGCCCCTCCAGAGCAGGACCTGATCGCTTCTACAGTTGCTGGCG
TGGTGACGACAGTCATGGGATCCTCACAACCAGTCGTGACGCGGGGCACA
ACCGACAATCTGATTCCTGTCTACTGTAGCATCTTGGCAGCCGTGGTCGT
GGGCCTGGTAGCCTACATCGCCTTTAAGAGATGACCTAGGTAA

Construct S1-11C2-CD28T-CD28-41BB AA (Signal Sequence in Bold; CDRs Underlined)

(SEQ ID NO: 28)
MALPVTALLLPLALLLHAARPEIQLVQSGAEVKKPGATVKISCKASGYTF
TNYGMNWVQQAPGQGLEWMGWMNTYTGEPTYADKFQGRVTFTLDTSARTV
YMELSSLRSEDTAVYFCARAGGQLRPGAMDYWGQGTMVTVSSGGGGSGGG
GSGGGGSDIVLTQTPLSLSVTPGQPASISCKASQSVDYDGDSFMNWYLQK
PGQPPQLLIYVASNLESGVPDRFSGSGSGTDFTLKISRVEAEDVGVYYCQ
QSNEEPPTFGQGTKLEIKAAALDNEKSNGTIIHVKGKHLCPSPLFPGPSK
PFWVLVVVGGVLACYSLLVTVAFIIFWVRSKRSRLLHSDYMNMTPRRPGP
TRKHYQPYAPPRDFAAYRSRFSVVKRGRKKLLYIFKQPFMRPVQTTQEED
GCSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRGK
GHDGLYQGLSTATKDTYDALHMQALPPRAKRSGSGEGRGSLLTCGDVEEN
PGPMGAGATGRAMDGPRLLLLLLLGVSLGGAKEACPTGLYTHSGECCKAC
NLGEGVAQPCGANQTVCEPCLDSVTFSDVVSATEPCKPCTECVGLQSMSA
PCVEADDAVCRCAYGYYQDETTGRCEACRVCEAGSGLVFSCQDKQNTVCE
ECPDGTYSDEANHVDPCLPCTVCEDTERQLRECTRWADAECEEIPGRWIT
RSTPPEGSDSTAPSTQEPEAPPEQDLIASTVAGVVTTVMGSSQPVVTRGT
TDNLIPVYCSILAAVVVGLVAYIAFKR

Construct S1-11C2-CD28T-CD28 DNA (Signal Sequence in Bold)

(SEQ ID NO: 29)
ATGGCACTCCCCGTAACTGCTCTGCTGCTGCCGTTGGCATTGCTCCTGCA
CGCCGCACGCCCGGAGATTCAGCTGGTGCAGAGCGGCGCCGAGGTGAAAA
AGCCCGGAGCCACCGTGAAGATCAGCTGCAAGGCCAGCGGCTACACATTC
ACCAACTACGGCATGAACTGGGTGCAACAGGCCCCCGGCCAGGGACTGGA
GTGGATGGGCTGGATGAACACATACACCGGCGAGCCCACCTACGCCGACA
AGTTCCAGGGCAGGGTGACATTCACACTCGATACCAGCGCCAGAACCGTG
TATATGGAACTGAGCAGCCTGAGGAGCGAGGACACCGCTGTGTACTTCTG
CGCAAGGGCTGGAGGCCAGCTGAGACCTGGCGCTATGGACTACTGGGGCC
AGGGCACCATGGTGACCGTGAGCTCCGGAGGCGGAGGATCTGGTGGCGGT
GGTTCTGGCGGCGGAGGCTCCGACATCGTGCTGACCCAGACACCTCTCTC
CCTGTCCGTGACCCCCGGACAGCCTGCCTCCATTTCCTGTAAGGCCTCCC
AGAGCGTGGACTATGACGGCGACAGCTTCATGAACTGGTACCTGCAGAAG
CCCGGACAACCCCCCCAGCTGCTGATCTACGTGGCCAGCAACCTGGAGTC
CGGCGTGCCTGACAGGTTTTCCGGCTCCGGCAGCGGCACCGACTTCACCC
TGAAGATCAGCAGGGTGGAAGCCGAGGACGTGGGCGTGTACTACTGCCAG
CAGAGCAACGAGGAGCCCCCTACCTTCGGACAGGGCACCAAGCTGGAGAT
CAAGGCCGCTGCCCTTGATAATGAAAAGTCAAACGGAACAATCATTCACG
TGAAGGGCAAGCACCTCTGTCCGTCACCCTTGTTCCCTGGTCCATCCAAG
CCATTCTGGGTGTTGGTCGTAGTGGGTGGAGTCCTCGCTTGTTACTCTCT
GCTCGTCACCGTGGCTTTTATAATCTTCTGGGTTAGATCCAAAAGAAGCC
GCCTGCTCCATAGCGATTACATGAATATGACTCCACGCCGCCCTGGCCCC
ACAAGGAAACACTACCAGCCTTACGCACCACCTAGAGATTTCGCTGCCTA
TCGGAGCAGGGTGAAGTTTTCCAGATCTGCAGATGCACCAGCGTATCAGC
AGGGCCAGAACCAACTGTATAACGAGCTCAACCTGGGACGCAGGGAAGAG
TATGACGTTTTGGACAAGCGCAGAGGACGGGACCCTGAGATGGGTGGCAA
ACCAAGACGAAAAAACCCCCAGGAGGGTCTCTATAATGAGCTGCAGAAGG
ATAAGATGGCTGAAGCCTATTCTGAAATAGGCATGAAAGGAGAGCGGAGA
AGGGGAAAAGGGCACGACGGTTTGTACCAGGGACTCAGCACTGCTACGAA
GGATACTTATGACGCTCTCCACATGCAAGCCCTGCCACCTAGGGCCAAGA
GAAGTGGCAGCGGGGAGGGCCGGGGATCTCTCCTTACATGTGGGGACGTG
GAAGAAAATCCGGGGCCTATGGGTGCCGGCGCCACGGGAAGGGCTATGGA
TGGCCCGCGACTGCTTCTCCTGCTGTTGTTGGGCGTGTCTCTCGGAGGCG
CTAAGGAGGCCTGTCCAACGGGCCTCTACACTCACTCCGGTGAATGTTGC
AAAGCCTGTAACCTTGGCGAGGGCGTCGCACAACCTTGTGGTGCTAACCA
GACAGTCTGTGAACCATGCCTGGATTCAGTGACATTCAGCGATGTTGTCT
CAGCCACCGAGCCTTGCAAGCCTTGTACCGAATGTGTGGGCCTTCAGTCC
ATGTCCGCCCCCTGTGTCGAAGCCGATGATGCAGTGTGCAGATGTGCCTA
TGGATATTACCAGGACGAAACTACCGGGCGGTGTGAGGCCTGCCGGGTGT
GTGAAGCCGGCTCTGGCCTCGTGTTCAGTTGCCAGGATAAGCAAAACACA
GTATGTGAGGAGTGTCCAGACGGAACCTACAGCGACGAGGCGAACCACGT
CGACCCTTGCTTGCCGTGCACCGTCTGCGAGGATACCGAACGCCAGCTGA
GAGAGTGTACGCGCTGGGCAGACGCTGAGTGCGAGGAGATCCCTGGGAGA
TGGATCACCCGGAGCACACCTCCTGAGGGATCAGACAGTACAGCCCCGAG
TACCCAAGAACCGGAGGCCCCTCCAGAGCAGGACCTGATCGCTTCTACAG
TTGCTGGCGTGGTGACGACAGTCATGGGATCCTCACAACCAGTCGTGACG
CGGGGCACAACCGACAATCTGATTCCTGTCTACTGTAGCATCTTGGCAGC
CGTGGTCGTGGGCCTGGTAGCCTACATCGCCTTTAAGAGATGACCTAGGT
AA

Construct S1-11C2-CD28T-CD28 AA (Signal Sequence in Bold; CDRs Underlined)

(SEQ ID NO: 30)
MALPVTALLLPLALLLHAARPEIQLVQSGAEVKKPGATVKISCKASGYTF
TNYGMNWVQQAPGQGLEWMGWMNTYTGEPTYADKFQGRVTFTLDTSARTV
YMELSSLRSEDTAVYFCARAGGQLRPGAMDYWGQGTMVTVSSGGGGSGGG
GSGGGGSDIVLTQTPLSLSVTPGQPASISCKASQSVDYDGDSFMNWYLQK
PGQPPQLLIYVASNLESGVPDRFSGSGSGTDFTLKISRVEAEDVGVYYCQ
QSNEEPPTFGQGTKLEIKAAALDNEKSNGTIIHVKGKHLCPSPLFPGPSK
PFWVLVVVGGVLACYSLLVTVAFIIFWVRSKRSRLLHSDYMNMTPRRPGP
TRKHYQPYAPPRDFAAYRSRVKFSRSADAPAYQQGQNQLYNELNLGRREE
YDVLDKRRGRDPEMGGKPRRKNPQEGLYNELQKDKMAEAYSEIGMKGERR
RGKGHDGLYQGLSTATKDTYDALHMQALPPRAKRSGSGEGRGSLLTCGDV
EENPGPMGAGATGRAMDGPRLLLLLLLGVSLGGAKEACPTGLYTHSGECC
KACNLGEGVAQPCGANQTVCEPCLDSVTFSDVVSATEPCKPCTECVGLQS
MSAPCVEADDAVCRCAYGYYQDETTGRCEACRVCEAGSGLVFSCQDKQNT
VCEECPDGTYSDEANHVDPCLPCTVCEDTERQLRECTRWADAECEEIPGR
WITRSTPPEGSDSTAPSTQEPEAPPEQDLIASTVAGVVTTVMGSSQPVVT
RGTTDNLIPVYCSILAAVVVGLVAYIAFKR

Construct S1-11C2-CD28T-41BB DNA (Signal Sequence in Bold)

(SEQ ID NO: 31)
ATGGCACTCCCCGTAACTGCTCTGCTGCTGCCGTTGGCATTGCTCCTGCA
CGCCGCACGCCCGGAGATTCAGCTGGTGCAGAGCGGCGCCGAGGTGAAAA
AGCCCGGAGCCACCGTGAAGATCAGCTGCAAGGCCAGCGGCTACACATTC
ACCAACTACGGCATGAACTGGGTGCAACAGGCCCCCGGCCAGGGACTGGA
GTGGATGGGCTGGATGAACACATACACCGGCGAGCCCACCTACGCCGACA
AGTTCCAGGGCAGGGTGACATTCACACTCGATACCAGCGCCAGAACCGTG
TATATGGAACTGAGCAGCCTGAGGAGCGAGGACACCGCTGTGTACTTCTG
CGCAAGGGCTGGAGGCCAGCTGAGACCTGGCGCTATGGACTACTGGGGCC
AGGGCACCATGGTGACCGTGAGCTCCGGAGGCGGAGGATCTGGTGGCGGT
GGTTCTGGCGGCGGAGGCTCCGACATCGTGCTGACCCAGACACCTCTCTC
CCTGTCCGTGACCCCCGGACAGCCTGCCTCCATTTCCTGTAAGGCCTCCC
AGAGCGTGGACTATGACGGCGACAGCTTCATGAACTGGTACCTGCAGAAG
CCCGGACAACCCCCCCAGCTGCTGATCTACGTGGCCAGCAACCTGGAGTC
CGGCGTGCCTGACAGGTTTTCCGGCTCCGGCAGCGGCACCGACTTCACCC
TGAAGATCAGCAGGGTGGAAGCCGAGGACGTGGGCGTGTACTACTGCCAG
CAGAGCAACGAGGAGCCCCCTACCTTCGGACAGGGCACCAAGCTGGAGAT
CAAGGCCGCTGCCCTTGATAATGAAAAGTCAAACGGAACAATCATTCACG
TGAAGGGCAAGCACCTCTGTCCGTCACCCTTGTTCCCTGGTCCATCCAAG
CCATTCTGGGTGTTGGTCGTAGTGGGTGGAGTCCTCGCTTGTTACTCTCT
GCTCGTCACCGTGGCTTTTATAATCTTCTGGGTTCGCTTTTCCGTCGTTA
AGCGGGGGAGAAAAAAGCTGCTGTACATTTTCAAACAGCCGTTTATGAGG
CCGGTCCAAACGACTCAGGAAGAGGACGGCTGCTCCTGCCGCTTTCCTGA
GGAGGAGGAGGGCGGGTGCGAACTGAGGGTGAAGTTTTCCAGATCTGCAG
ATGCACCAGCGTATCAGCAGGGCCAGAACCAACTGTATAACGAGCTCAAC
CTGGGACGCAGGGAAGAGTATGACGTTTTGGACAAGCGCAGAGGACGGGA
CCCTGAGATGGGTGGCAAACCAAGACGAAAAAACCCCCAGGAGGGTCTCT
ATAATGAGCTGCAGAAGGATAAGATGGCTGAAGCCTATTCTGAAATAGGC
ATGAAAGGAGAGCGGAGAAGGGGAAAAGGGCACGACGGTTTGTACCAGGG
ACTCAGCACTGCTACGAAGGATACTTATGACGCTCTCCACATGCAAGCCC
TGCCACCTAGGGCCAAGAGAAGTGGCAGCGGGGAGGGCCGGGGATCTCTC
CTTACATGTGGGGACGTGGAAGAAAATCCGGGGCCTATGGGTGCCGGCGC
CACGGGAAGGGCTATGGATGGCCCGCGACTGCTTCTCCTGCTGTTGTTGG
GCGTGTCTCTCGGAGGCGCTAAGGAGGCCTGTCCAACGGGCCTCTACACT
CACTCCGGTGAATGTTGCAAAGCCTGTAACCTTGGCGAGGGCGTCGCACA
ACCTTGTGGTGCTAACCAGACAGTCTGTGAACCATGCCTGGATTCAGTGA
CATTCAGCGATGTTGTCTCAGCCACCGAGCCTTGCAAGCCTTGTACCGAA
TGTGTGGGCCTTCAGTCCATGTCCGCCCCCTGTGTCGAAGCCGATGATGC
AGTGTGCAGATGTGCCTATGGATATTACCAGGACGAAACTACCGGGCGGT
GTGAGGCCTGCCGGGTGTGTGAAGCCGGCTCTGGCCTCGTGTTCAGTTGC
CAGGATAAGCAAAACACAGTATGTGAGGAGTGTCCAGACGGAACCTACAG
CGACGAGGCGAACCACGTCGACCCTTGCTTGCCGTGCACCGTCTGCGAGG
ATACCGAACGCCAGCTGAGAGAGTGTACGCGCTGGGCAGACGCTGAGTGC
GAGGAGATCCCTGGGAGATGGATCACCCGGAGCACACCTCCTGAGGGATC
AGACAGTACAGCCCCGAGTACCCAAGAACCGGAGGCCCCTCCAGAGCAGG
ACCTGATCGCTTCTACAGTTGCTGGCGTGGTGACGACAGTCATGGGATCC
TCACAACCAGTCGTGACGCGGGGCACAACCGACAATCTGATTCCTGTCTA
CTGTAGCATCTTGGCAGCCGTGGTCGTGGGCCTGGTAGCCTACATCGCCT
TTAAGAGATGACCTAGGTAA

Construct S1-11C2-CD28T-41BB AA (Signal Sequence in Bold; CDRs Underlined)

(SEQ ID NO: 32)
MALPVTALLLPLALLLHAARPEIQLVQSGAEVKKPGATVKISCKASGYTF
TNYGMNWVQQAPGQGLEWMGWMNTYTGEPTYADKFQGRVTFTLDTSARTV
YMELSSLRSEDTAVYFCARAGGQLRPGAMDYWGQGTMVTVSSGGGGSGGG
GSGGGGSDIVLTQTPLSLSVTPGQPASISCKASQSVDYDGDSFMNWYLQK
PGQPPQLLIYVASNLESGVPDRFSGSGSGTDFTLKISRVEAEDVGVYYCQ
QSNEEPPTFGQGTKLEIKAAALDNEKSNGTIIHVKGKHLCPSPLFPGPSK
PFWVLVVVGGVLACYSLLVTVAFIIFWVRFSVVKRGRKKLLYIFKQPFMR
PVQTTQEEDGCSCRFPEEEEGGCELRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPRAKRSGSGEGRGSL
LTCGDVEENPGPMGAGATGRAMDGPRLLLLLLLGVSLGGAKEACPTGLYT
HSGECCKACNLGEGVAQPCGANQTVCEPCLDSVTFSDVVSATEPCKPCTE
CVGLQSMSAPCVEADDAVCRCAYGYYQDETTGRCEACRVCEAGSGLVFSC
QDKQNTVCEECPDGTYSDEANHVDPCLPCTVCEDTERQLRECTRWADAEC
EEIPGRWITRSTPPEGSDSTAPSTQEPEAPPEQDLIASTVAGVVTTVMGS
SQPVVTRGTTDNLIPVYCSILAAVVVGLVAYIAFKR

Construct S1-11C2-C8K-CD28 DNA (Signal Sequence in Bold)

(SEQ ID NO: 33)
ATGGCACTCCCCGTAACTGCTCTGCTGCTGCCGTTGGCATTGCTCCTGCA
CGCCGCACGCCCGGAGATTCAGCTGGTGCAGAGCGGCGCCGAGGTGAAAA
AGCCCGGAGCCACCGTGAAGATCAGCTGCAAGGCCAGCGGCTACACATTC
ACCAACTACGGCATGAACTGGGTGCAACAGGCCCCCGGCCAGGGACTGGA
GTGGATGGGCTGGATGAACACATACACCGGCGAGCCCACCTACGCCGACA
AGTTCCAGGGCAGGGTGACATTCACACTCGATACCAGCGCCAGAACCGTG
TATATGGAACTGAGCAGCCTGAGGAGCGAGGACACCGCTGTGTACTTCTG
CGCAAGGGCTGGAGGCCAGCTGAGACCTGGCGCTATGGACTACTGGGGCC
AGGGCACCATGGTGACCGTGAGCTCCGGAGGCGGAGGATCTGGTGGCGGT
GGTTCTGGCGGCGGAGGCTCCGACATCGTGCTGACCCAGACACCTCTCTC
CCTGTCCGTGACCCCCGGACAGCCTGCCTCCATTTCCTGTAAGGCCTCCC
AGAGCGTGGACTATGACGGCGACAGCTTCATGAACTGGTACCTGCAGAAG
CCCGGACAACCCCCCCAGCTGCTGATCTACGTGGCCAGCAACCTGGAGTC
CGGCGTGCCTGACAGGTTTTCCGGCTCCGGCAGCGGCACCGACTTCACCC
TGAAGATCAGCAGGGTGGAAGCCGAGGACGTGGGCGTGTACTACTGCCAG
CAGAGCAACGAGGAGCCCCCTACCTTCGGACAGGGCACCAAGCTGGAGAT
CAAGGCCGCTGCCTTCGTGCCTGTTTTTCTGCCCGCGAAACCCACAACTA
CCCCCGCCCCTCGGCCCCCAACTCCTGCACCAACTATCGCTTCCCAACCC
CTGTCTCTGAGACCTGAGGCATGCCGCCCCGCGGCAGGCGGCGCCGTGCA
CACTAGAGGCCTGGACTTCGCCTGCGATATTTATATCTGGGCCCCCCTTG
CCGGGACATGCGGGGTACTGCTGCTGTCTCTGGTGATTACCCTCTACTGC
AACCACAGAAACAGATCCAAAAGAAGCCGCCTGCTCCATAGCGATTACAT
GAATATGACTCCACGCCGCCCTGGCCCCACAAGGAAACACTACCAGCCTT
ACGCACCACCTAGAGATTTCGCTGCCTATCGGAGCAGGGTGAAGTTTTCC
AGATCTGCAGATGCACCAGCGTATCAGCAGGGCCAGAACCAACTGTATAA
CGAGCTCAACCTGGGACGCAGGGAAGAGTATGACGTTTTGGACAAGCGCA
GAGGACGGGACCCTGAGATGGGTGGCAAACCAAGACGAAAAAACCCCCAG
GAGGGTCTCTATAATGAGCTGCAGAAGGATAAGATGGCTGAAGCCTATTC
TGAAATAGGCATGAAAGGAGAGCGGAGAAGGGGAAAAGGGCACGACGGTT
TGTACCAGGGACTCAGCACTGCTACGAAGGATACTTATGACGCTCTCCAC
ATGCAAGCCCTGCCACCTAGGGCCAAGAGAAGTGGCAGCGGGGAGGGCCG
GGGATCTCTCCTTACATGTGGGGACGTGGAAGAAAATCCGGGGCCTATGG
GTGCCGGCGCCACGGGAAGGGCTATGGATGGCCCGCGACTGCTTCTCCTG
CTGTTGTTGGGCGTGTCTCTCGGAGGCGCTAAGGAGGCCTGTCCAACGGG
CCTCTACACTCACTCCGGTGAATGTTGCAAAGCCTGTAACCTTGGCGAGG
GCGTCGCACAACCTTGTGGTGCTAACCAGACAGTCTGTGAACCATGCCTG
GATTCAGTGACATTCAGCGATGTTGTCTCAGCCACCGAGCCTTGCAAGCC
TTGTACCGAATGTGTGGGCCTTCAGTCCATGTCCGCCCCCTGTGTCGAAG
CCGATGATGCAGTGTGCAGATGTGCCTATGGATATTACCAGGACGAAACT
ACCGGGCGGTGTGAGGCCTGCCGGGTGTGTGAAGCCGGCTCTGGCCTCGT
GTTCAGTTGCCAGGATAAGCAAAACACAGTATGTGAGGAGTGTCCAGACG
GAACCTACAGCGACGAGGCGAACCACGTCGACCCTTGCTTGCCGTGCACC
GTCTGCGAGGATACCGAACGCCAGCTGAGAGAGTGTACGCGCTGGGCAGA
CGCTGAGTGCGAGGAGATCCCTGGGAGATGGATCACCCGGAGCACACCTC
CTGAGGGATCAGACAGTACAGCCCCGAGTACCCAAGAACCGGAGGCCCCT
CCAGAGCAGGACCTGATCGCTTCTACAGTTGCTGGCGTGGTGACGACAGT
CATGGGATCCTCACAACCAGTCGTGACGCGGGGCACAACCGACAATCTGA
TTCCTGTCTACTGTAGCATCTTGGCAGCCGTGGTCGTGGGCCTGGTAGCC
TACATCGCCTTTAAGAGATGACCTAGGTAA

Construct S1-11C2-C8K-CD28 AA (Signal Sequence in Bold; CDRs Underlined)

(SEQ ID NO: 34)
MALPVTALLLPLALLLHAARPEIQLVQSGAEVKKPGATVKISCKASGYTF
TNYGMNWVQQAPGQGLEWMGWMNTYTGEPTYADKFQGRVTFTLDTSARTV
YMELSSLRSEDTAVYFCARAGGQLRPGAMDYWGQGTMVTVSSGGGGSGGG
GSGGGGSDIVLTQTPLSLSVTPGQPASISCKASQSVDYDGDSFMNWYLQK
PGQPPQLLIYVASNLESGVPDRFSGSGSGTDFTLKISRVEAEDVGVYYCQ
QSNEEPPTFGQGTKLEIKAAAFVPVFLPAKPTTTPAPRPPTPAPTIASQP
LSLRPEACRPAAGGAVHTRGLDFACDIYIWAPLAGTCGVLLLSLVITLYC
NHRNRSKRSRLLHSDYMNMTPRRPGPTRKHYQPYAPPRDFAAYRSRVKFS
RSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALH
MQALPPRAKRSGSGEGRGSLLTCGDVEENPGPMGAGATGRAMDGPRLLLL
LLLGVSLGGAKEACPTGLYTHSGECCKACNLGEGVAQPCGANQTVCEPCL
DSVTFSDVVSATEPCKPCTECVGLQSMSAPCVEADDAVCRCAYGYYQDET
TGRCEACRVCEAGSGLVFSCQDKQNTVCEECPDGTYSDEANHVDPCLPCT
VCEDTERQLRECTRWADAECEEIPGRWITRSTPPEGSDSTAPSTQEPEAP
PEQDLIASTVAGVVTTVMGSSQPVVTRGTTDNLIPVYCSILAAVVVGLVA
YIAFKR

Construct S1-11C2-C8K-41BB DNA (Signal Sequence in Bold)

(SEQ ID NO: 35)
ATGGCACTCCCCGTAACTGCTCTGCTGCTGCCGTTGGCATTGCTCCTGCA
CGCCGCACGCCCGGAGATTCAGCTGGTGCAGAGCGGCGCCGAGGTGAAAA
AGCCCGGAGCCACCGTGAAGATCAGCTGCAAGGCCAGCGGCTACACATTC
ACCAACTACGGCATGAACTGGGTGCAACAGGCCCCCGGCCAGGGACTGGA
GTGGATGGGCTGGATGAACACATACACCGGCGAGCCCACCTACGCCGACA
AGTTCCAGGGCAGGGTGACATTCACACTCGATACCAGCGCCAGAACCGTG
TATATGGAACTGAGCAGCCTGAGGAGCGAGGACACCGCTGTGTACTTCTG
CGCAAGGGCTGGAGGCCAGCTGAGACCTGGCGCTATGGACTACTGGGGCC
AGGGCACCATGGTGACCGTGAGCTCCGGAGGCGGAGGATCTGGTGGCGGT
GGTTCTGGCGGCGGAGGCTCCGACATCGTGCTGACCCAGACACCTCTCTC
CCTGTCCGTGACCCCCGGACAGCCTGCCTCCATTTCCTGTAAGGCCTCCC
AGAGCGTGGACTATGACGGCGACAGCTTCATGAACTGGTACCTGCAGAAG
CCCGGACAACCCCCCCAGCTGCTGATCTACGTGGCCAGCAACCTGGAGTC
CGGCGTGCCTGACAGGTTTTCCGGCTCCGGCAGCGGCACCGACTTCACCC
TGAAGATCAGCAGGGTGGAAGCCGAGGACGTGGGCGTGTACTACTGCCAG
CAGAGCAACGAGGAGCCCCCTACCTTCGGACAGGGCACCAAGCTGGAGAT
CAAGGCCGCTGCCTTCGTGCCTGTTTTTCTGCCCGCGAAACCCACAACTA
CCCCCGCCCCTCGGCCCCCAACTCCTGCACCAACTATCGCTTCCCAACCC
CTGTCTCTGAGACCTGAGGCATGCCGCCCCGCGGCAGGCGGCGCCGTGCA
CACTAGAGGCCTGGACTTCGCCTGCGATATTTATATCTGGGCCCCCCTTG
CCGGGACATGCGGGGTACTGCTGCTGTCTCTGGTGATTACCCTCTACTGC
AACCACAGAAACCGCTTTTCCGTCGTTAAGCGGGGGAGAAAAAAGCTGCT
GTACATTTTCAAACAGCCGTTTATGAGGCCGGTCCAAACGACTCAGGAAG
AGGACGGCTGCTCCTGCCGCTTTCCTGAGGAGGAGGAGGGCGGGTGCGAA
CTGAGGGTGAAGTTTTCCAGATCTGCAGATGCACCAGCGTATCAGCAGGG
CCAGAACCAACTGTATAACGAGCTCAACCTGGGACGCAGGGAAGAGTATG
ACGTTTTGGACAAGCGCAGAGGACGGGACCCTGAGATGGGTGGCAAACCA
AGACGAAAAAACCCCCAGGAGGGTCTCTATAATGAGCTGCAGAAGGATAA
GATGGCTGAAGCCTATTCTGAAATAGGCATGAAAGGAGAGCGGAGAAGGG
GAAAAGGGCACGACGGTTTGTACCAGGGACTCAGCACTGCTACGAAGGAT
ACTTATGACGCTCTCCACATGCAAGCCCTGCCACCTAGGGCCAAGAGAAG
TGGCAGCGGGGAGGGCCGGGGATCTCTCCTTACATGTGGGGACGTGGAAG
AAAATCCGGGGCCTATGGGTGCCGGCGCCACGGGAAGGGCTATGGATGGC
CCGCGACTGCTTCTCCTGCTGTTGTTGGGCGTGTCTCTCGGAGGCGCTAA
GGAGGCCTGTCCAACGGGCCTCTACACTCACTCCGGTGAATGTTGCAAAG
CCTGTAACCTTGGCGAGGGCGTCGCACAACCTTGTGGTGCTAACCAGACA
GTCTGTGAACCATGCCTGGATTCAGTGACATTCAGCGATGTTGTCTCAGC
CACCGAGCCTTGCAAGCCTTGTACCGAATGTGTGGGCCTTCAGTCCATGT
CCGCCCCCTGTGTCGAAGCCGATGATGCAGTGTGCAGATGTGCCTATGGA
TATTACCAGGACGAAACTACCGGGCGGTGTGAGGCCTGCCGGGTGTGTGA
AGCCGGCTCTGGCCTCGTGTTCAGTTGCCAGGATAAGCAAAACACAGTAT
GTGAGGAGTGTCCAGACGGAACCTACAGCGACGAGGCGAACCACGTCGAC
CCTTGCTTGCCGTGCACCGTCTGCGAGGATACCGAACGCCAGCTGAGAGA
GTGTACGCGCTGGGCAGACGCTGAGTGCGAGGAGATCCCTGGGAGATGGA
TCACCCGGAGCACACCTCCTGAGGGATCAGACAGTACAGCCCCGAGTACC
CAAGAACCGGAGGCCCCTCCAGAGCAGGACCTGATCGCTTCTACAGTTGC
TGGCGTGGTGACGACAGTCATGGGATCCTCACAACCAGTCGTGACGCGGG
GCACAACCGACAATCTGATTCCTGTCTACTGTAGCATCTTGGCAGCCGTG
GTCGTGGGCCTGGTAGCCTACATCGCCTTTAAGAGATGACCTAGGTAA

Construct S1-11C2-C8K-41BB AA (Signal Sequence in Bold; CDRs Underlined)

(SEQ ID NO: 36)
MALPVTALLLPLALLLHAARPEIQLVQSGAEVKKPGATVKISCKASGYTF
TNYGMNWVQQAPGQGLEWMGWMNTYTGEPTYADKFQGRVTFTLDTSARTV
YMELSSLRSEDTAVYFCARAGGQLRPGAMDYWGQGTMVTVSSGGGGSGGG
GSGGGGSDIVLTQTPLSLSVTPGQPASISCKASQSVDYDGDSFMNWYLQK
PGQPPQLLIYVASNLESGVPDRFSGSGSGTDFTLKISRVEAEDVGVYYCQ
QSNEEPPTFGQGTKLEIKAAAFVPVFLPAKPTTTPAPRPPTPAPTIASQP
LSLRPEACRPAAGGAVHTRGLDFACDIYIWAPLAGTCGVLLLSLVITLYC
NHRNRFSVVKRGRKKLLYIFKQPFMRPVQTTQEEDGCSCRFPEEEEGGCE
LRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPRAKRSGSGEGRGSLLTCGDVEENPGPMGAGATGRAMDG
PRLLLLLLLGVSLGGAKEACPTGLYTHSGECCKACNLGEGVAQPCGANQT
VCEPCLDSVTFSDVVSATEPCKPCTECVGLQSMSAPCVEADDAVCRCAYG
YYQDETTGRCEACRVCEAGSGLVFSCQDKQNTVCEECPDGTYSDEANHVD
PCLPCTVCEDTERQLRECTRWADAECEEIPGRWITRSTPPEGSDSTAPST
QEPEAPPEQDLIASTVAGVVTTVMGSSQPVVTRGTTDNLIPVYCSILAAV
VVGLVAYIAFKR

Construct S2-1A1-CD28T-CD28-41BB DNA (Signal Sequence in Bold)

(SEQ ID NO: 37)
ATGGCACTCCCCGTAACTGCTCTGCTGCTGCCGTTGGCATTGCTCCTGCA
CGCCGCACGCCCGCAGGTGCAGCTGGTGCAGTCCGGCGCCGAAGTGAAGA
AGCCCGGAGCCAGCGTGAAGGTGAGCTGCAAGGCCTCCGGCTACACCTTC
ACCACCTACTGGATGCACTGGGTCAGACAGGCTCCCGGACAGGGCCTGGA
ATGGATGGGCGAAATCAACCCCTCCTCCGGCAGGACCAACTACAACGAGA
AGTTCAAGACCAGGGTGACCATGACCAGGGACACCAGCACCAGCACCGTG
TACATGGAGCTGTCCAGCCTGAGGAGCGAGGACACCGCCGTGTACTACTG
TGCCAAGCTGGGACCCGGCCCCCAGTACTATGCCATGGACTACTGGGGCC
AAGGCACCATGGTGACCGTGAGCAGCGGAGGCGGAGGATCTGGTGGCGGT
GGTTCTGGCGGCGGAGGCTCCGACATCCAGATGACCCAGAGCCCCTCCAG
CCTGTCCGCTAGCGTGGGCGACAGGGTCACCATTACCTGCCACGCCAGCC
AGAACATCAACGTGTGGCTGAGCTGGTATCAGCAGAAACCCGGCAAGGCT
CCCAAGCTGCTGATCTACAAGGCCAGCAAGCTGCACACCGGCGTGCCCAG
CAGGTTTAGCGGTTCTGGCTCCGGCACCGACTTCACCCTCACCATCAGCA
GCCTGCAGCCCGAAGACTTCGCTACCTACTACTGCCAGCAGGGACAAAGC
TACCCCTGGACCTTCGGCCAGGGAACCAAGCTGGAAATCAAGGCCGCTGC
CCTTGATAATGAAAAGTCAAACGGAACAATCATTCACGTGAAGGGCAAGC
ACCTCTGTCCGTCACCCTTGTTCCCTGGTCCATCCAAGCCATTCTGGGTG
TTGGTCGTAGTGGGTGGAGTCCTCGCTTGTTACTCTCTGCTCGTCACCGT
GGCTTTTATAATCTTCTGGGTTAGATCCAAAAGAAGCCGCCTGCTCCATA
GCGATTACATGAATATGACTCCACGCCGCCCTGGCCCCACAAGGAAACAC
TACCAGCCTTACGCACCACCTAGAGATTTCGCTGCCTATCGGAGCCGCTT
TTCCGTCGTTAAGCGGGGGAGAAAAAAGCTGCTGTACATTTTCAAACAGC
CGTTTATGAGGCCGGTCCAAACGACTCAGGAAGAGGACGGCTGCTCCTGC
CGCTTTCCTGAGGAGGAGGAGGGCGGGTGCGAACTGAGGGTGAAGTTTTC
CAGATCTGCAGATGCACCAGCGTATCAGCAGGGCCAGAACCAACTGTATA
ACGAGCTCAACCTGGGACGCAGGGAAGAGTATGACGTTTTGGACAAGCGC
AGAGGACGGGACCCTGAGATGGGTGGCAAACCAAGACGAAAAAACCCCCA
GGAGGGTCTCTATAATGAGCTGCAGAAGGATAAGATGGCTGAAGCCTATT
CTGAAATAGGCATGAAAGGAGAGCGGAGAAGGGGAAAAGGGCACGACGGT
TTGTACCAGGGACTCAGCACTGCTACGAAGGATACTTATGACGCTCTCCA
CATGCAAGCCCTGCCACCTAGGGCCAAGAGAAGTGGCAGCGGGGAGGGCC
GGGGATCTCTCCTTACATGTGGGGACGTGGAAGAAAATCCGGGGCCTATG
GGTGCCGGCGCCACGGGAAGGGCTATGGATGGCCCGCGACTGCTTCTCCT
GCTGTTGTTGGGCGTGTCTCTCGGAGGCGCTAAGGAGGCCTGTCCAACGG
GCCTCTACACTCACTCCGGTGAATGTTGCAAAGCCTGTAACCTTGGCGAG
GGCGTCGCACAACCTTGTGGTGCTAACCAGACAGTCTGTGAACCATGCCT
GGATTCAGTGACATTCAGCGATGTTGTCTCAGCCACCGAGCCTTGCAAGC
CTTGTACCGAATGTGTGGGCCTTCAGTCCATGTCCGCCCCCTGTGTCGAA
GCCGATGATGCAGTGTGCAGATGTGCCTATGGATATTACCAGGACGAAAC
TACCGGGCGGTGTGAGGCCTGCCGGGTGTGTGAAGCCGGCTCTGGCCTCG
TGTTCAGTTGCCAGGATAAGCAAAACACAGTATGTGAGGAGTGTCCAGAC
GGAACCTACAGCGACGAGGCGAACCACGTCGACCCTTGCTTGCCGTGCAC
CGTCTGCGAGGATACCGAACGCCAGCTGAGAGAGTGTACGCGCTGGGCAG
ACGCTGAGTGCGAGGAGATCCCTGGGAGATGGATCACCCGGAGCACACCT
CCTGAGGGATCAGACAGTACAGCCCCGAGTACCCAAGAACCGGAGGCCCC
TCCAGAGCAGGACCTGATCGCTTCTACAGTTGCTGGCGTGGTGACGACAG
TCATGGGATCCTCACAACCAGTCGTGACGCGGGGCACAACCGACAATCTG
ATTCCTGTCTACTGTAGCATCTTGGCAGCCGTGGTCGTGGGCCTGGTAGC
CTACATCGCCTTTAAGAGATGACCTAGGTAA

Construct S2-1A1-CD28T-CD28-41BB AA (Signal Sequence in Bold; CDRs Underlined)

(SEQ ID NO: 38)
MALPVTALLLPLALLLHAARPQVQLVQSGAEVKKPGASVKVSCKASGYTF
TTYWMHWVRQAPGQGLEWMGEINPSSGRTNYNEKFKTRVTMTRDTSTSTV
YMELSSLRSEDTAVYYCAKLGPGPQYYAMDYVVGQGTMVTVSSGGGGSGG
GGSGGGGSDIQMTQSPSSLSASVGDRVTITCHASQNINVWLSWYQQKPGK
APKLLIYKASKLHTGVPSRFSGSGSGTDFTLTISSLQPEDFATYYCQQGQ
SYPWTFGQGTKLEIKAAALDNEKSNGTIIHVKGKHLCPSPLFPGPSKPFW
VLVVVGGVLACYSLLVTVAFIIFWVRSKRSRLLHSDYMNMTPRRPGPTRK
HYQPYAPPRDFAAYRSRFSVVKRGRKKLLYIFKQPFMRPVQTTQEEDGCS
CRFPEEEEGGCELRVKFSRSADAPAYQQGQNQLYNELNLGRREEYDVLDK
RRGRDPEMGGKPRRKNPQEGLYNELQKDKMAEAYSEIGMKGERRRGKGHD
GLYQGLSTATKDTYDALHMQALPPRAKRSGSGEGRGSLLTCGDVEENPGP
MGAGATGRAMDGPRLLLLLLLGVSLGGAKEACPTGLYTHSGECCKACNLG
EGVAQPCGANQTVCEPCLDSVTFSDVVSATEPCKPCTECVGLQSMSAPCV
EADDAVCRCAYGYYQDETTGRCEACRVCEAGSGLVFSCQDKQNTVCEECP
DGTYSDEANHVDPCLPCTVCEDTERQLRECTRWADAECEEIPGRWITRST
PPEGSDSTAPSTQEPEAPPEQDLIASTVAGVVTTVMGSSQPVVTRGTTDN
LIPVYCSILAAVVVGLVAYIAFKR

Construct S2-1A1-CD28T-CD28 DNA (Signal Sequence in Bold)

(SEQ ID NO: 39)
ATGGCACTCCCCGTAACTGCTCTGCTGCTGCCGTTGGCATTGCTCCTGCA
CGCCGCACGCCCGCAGGTGCAGCTGGTGCAGTCCGGCGCCGAAGTGAAGA
AGCCCGGAGCCAGCGTGAAGGTGAGCTGCAAGGCCTCCGGCTACACCTTC
ACCACCTACTGGATGCACTGGGTCAGACAGGCTCCCGGACAGGGCCTGGA
ATGGATGGGCGAAATCAACCCCTCCTCCGGCAGGACCAACTACAACGAGA
AGTTCAAGACCAGGGTGACCATGACCAGGGACACCAGCACCAGCACCGTG
TACATGGAGCTGTCCAGCCTGAGGAGCGAGGACACCGCCGTGTACTACTG
TGCCAAGCTGGGACCCGGCCCCCAGTACTATGCCATGGACTACTGGGGCC
AAGGCACCATGGTGACCGTGAGCAGCGGAGGCGGAGGATCTGGTGGCGGT
GGTTCTGGCGGCGGAGGCTCCGACATCCAGATGACCCAGAGCCCCTCCAG
CCTGTCCGCTAGCGTGGGCGACAGGGTCACCATTACCTGCCACGCCAGCC
AGAACATCAACGTGTGGCTGAGCTGGTATCAGCAGAAACCCGGCAAGGCT
CCCAAGCTGCTGATCTACAAGGCCAGCAAGCTGCACACCGGCGTGCCCAG
CAGGTTTAGCGGTTCTGGCTCCGGCACCGACTTCACCCTCACCATCAGCA
GCCTGCAGCCCGAAGACTTCGCTACCTACTACTGCCAGCAGGGACAAAGC
TACCCCTGGACCTTCGGCCAGGGAACCAAGCTGGAAATCAAGGCCGCTGC
CCTTGATAATGAAAAGTCAAACGGAACAATCATTCACGTGAAGGGCAAGC
ACCTCTGTCCGTCACCCTTGTTCCCTGGTCCATCCAAGCCATTCTGGGTG
TTGGTCGTAGTGGGTGGAGTCCTCGCTTGTTACTCTCTGCTCGTCACCGT
GGCTTTTATAATCTTCTGGGTTAGATCCAAAAGAAGCCGCCTGCTCCATA
GCGATTACATGAATATGACTCCACGCCGCCCTGGCCCCACAAGGAAACAC
TACCAGCCTTACGCACCACCTAGAGATTTCGCTGCCTATCGGAGCAGGGT
GAAGTTTTCCAGATCTGCAGATGCACCAGCGTATCAGCAGGGCCAGAACC
AACTGTATAACGAGCTCAACCTGGGACGCAGGGAAGAGTATGACGTTTTG
GACAAGCGCAGAGGACGGGACCCTGAGATGGGTGGCAAACCAAGACGAAA
AAACCCCCAGGAGGGTCTCTATAATGAGCTGCAGAAGGATAAGATGGCTG
AAGCCTATTCTGAAATAGGCATGAAAGGAGAGCGGAGAAGGGGAAAAGGG
CACGACGGTTTGTACCAGGGACTCAGCACTGCTACGAAGGATACTTATGA
CGCTCTCCACATGCAAGCCCTGCCACCTAGGGCCAAGAGAAGTGGCAGCG
GGGAGGGCCGGGGATCTCTCCTTACATGTGGGGACGTGGAAGAAAATCCG
GGGCCTATGGGTGCCGGCGCCACGGGAAGGGCTATGGATGGCCCGCGACT
GCTTCTCCTGCTGTTGTTGGGCGTGTCTCTCGGAGGCGCTAAGGAGGCCT
GTCCAACGGGCCTCTACACTCACTCCGGTGAATGTTGCAAAGCCTGTAAC
CTTGGCGAGGGCGTCGCACAACCTTGTGGTGCTAACCAGACAGTCTGTGA
ACCATGCCTGGATTCAGTGACATTCAGCGATGTTGTCTCAGCCACCGAGC
CTTGCAAGCCTTGTACCGAATGTGTGGGCCTTCAGTCCATGTCCGCCCCC
TGTGTCGAAGCCGATGATGCAGTGTGCAGATGTGCCTATGGATATTACCA
GGACGAAACTACCGGGCGGTGTGAGGCCTGCCGGGTGTGTGAAGCCGGCT
CTGGCCTCGTGTTCAGTTGCCAGGATAAGCAAAACACAGTATGTGAGGAG
TGTCCAGACGGAACCTACAGCGACGAGGCGAACCACGTCGACCCTTGCTT
GCCGTGCACCGTCTGCGAGGATACCGAACGCCAGCTGAGAGAGTGTACGC
GCTGGGCAGACGCTGAGTGCGAGGAGATCCCTGGGAGATGGATCACCCGG
AGCACACCTCCTGAGGGATCAGACAGTACAGCCCCGAGTACCCAAGAACC
GGAGGCCCCTCCAGAGCAGGACCTGATCGCTTCTACAGTTGCTGGCGTGG
TGACGACAGTCATGGGATCCTCACAACCAGTCGTGACGCGGGGCACAACC
GACAATCTGATTCCTGTCTACTGTAGCATCTTGGCAGCCGTGGTCGTGGG
CCTGGTAGCCTACATCGCCTTTAAGAGATGACCTAGGTAA

Construct S2-1A1-CD28T-CD28 AA (Signal Sequence in Bold; CDRs Underlined)

(SEQ ID NO: 40)
MALPVTALLLPLALLLHAARPQVQLVQSGAEVKKPGASVKVSCKASGYTF
TTYWMHWVRQAPGQGLEWMGEINPSSGRTNYNEKFKTRVTMTRDTSTSTV
YMELSSLRSEDTAVYYCAKLGPGPQYYAMDYVVGQGTMVTVSSGGGGSGG
GGSGGGGSDIQMTQSPSSLSASVGDRVTITCHASQNINVWLSWYQQKPGK
APKLLIYKASKLHTGVPSRFSGSGSGTDFTLTISSLQPEDFATYYCQQGQ
SYPWTFGQGTKLEIKAAALDNEKSNGTIIHVKGKHLCPSPLFPGPSKPFW
VLVVVGGVLACYSLLVTVAFIIFWVRSKRSRLLHSDYMNMTPRRPGPTRK
HYQPYAPPRDFAAYRSRVKFSRSADAPAYQQGQNQLYNELNLGRREEYDV
LDKRRGRDPEMGGKPRRKNPQEGLYNELQKDKMAEAYSEIGMKGERRRGK
GHDGLYQGLSTATKDTYDALHMQALPPRAKRSGSGEGRGSLLTCGDVEEN
PGPMGAGATGRAMDGPRLLLLLLLGVSLGGAKEACPTGLYTHSGECCKAC
NLGEGVAQPCGANQTVCEPCLDSVTFSDVVSATEPCKPCTECVGLQSMSA
PCVEADDAVCRCAYGYYQDETTGRCEACRVCEAGSGLVFSCQDKQNTVCE
ECPDGTYSDEANHVDPCLPCTVCEDTERQLRECTRWADAECEEIPGRWIT
RSTPPEGSDSTAPSTQEPEAPPEQDLIASTVAGVVTTVMGSSQPVVTRGT
TDNLIPVYCSILAAVVVGLVAYIAFKR

Construct S2-1A1-CD28T-41BB DNA (Signal Sequence in Bold)

(SEQ ID NO: 41)
ATGGCACTCCCCGTAACTGCTCTGCTGCTGCCGTTGGCATTGCTCCTGCA
CGCCGCACGCCCGCAGGTGCAGCTGGTGCAGTCCGGCGCCGAAGTGAAGA
AGCCCGGAGCCAGCGTGAAGGTGAGCTGCAAGGCCTCCGGCTACACCTTC
ACCACCTACTGGATGCACTGGGTCAGACAGGCTCCCGGACAGGGCCTGGA
ATGGATGGGCGAAATCAACCCCTCCTCCGGCAGGACCAACTACAACGAGA
AGTTCAAGACCAGGGTGACCATGACCAGGGACACCAGCACCAGCACCGTG
TACATGGAGCTGTCCAGCCTGAGGAGCGAGGACACCGCCGTGTACTACTG
TGCCAAGCTGGGACCCGGCCCCCAGTACTATGCCATGGACTACTGGGGCC
AAGGCACCATGGTGACCGTGAGCAGCGGAGGCGGAGGATCTGGTGGCGGT
GGTTCTGGCGGCGGAGGCTCCGACATCCAGATGACCCAGAGCCCCTCCAG
CCTGTCCGCTAGCGTGGGCGACAGGGTCACCATTACCTGCCACGCCAGCC
AGAACATCAACGTGTGGCTGAGCTGGTATCAGCAGAAACCCGGCAAGGCT
CCCAAGCTGCTGATCTACAAGGCCAGCAAGCTGCACACCGGCGTGCCCAG
CAGGTTTAGCGGTTCTGGCTCCGGCACCGACTTCACCCTCACCATCAGCA
GCCTGCAGCCCGAAGACTTCGCTACCTACTACTGCCAGCAGGGACAAAGC
TACCCCTGGACCTTCGGCCAGGGAACCAAGCTGGAAATCAAGGCCGCTGC
CCTTGATAATGAAAAGTCAAACGGAACAATCATTCACGTGAAGGGCAAGC
ACCTCTGTCCGTCACCCTTGTTCCCTGGTCCATCCAAGCCATTCTGGGTG
TTGGTCGTAGTGGGTGGAGTCCTCGCTTGTTACTCTCTGCTCGTCACCGT
GGCTTTTATAATCTTCTGGGTTCGCTTTTCCGTCGTTAAGCGGGGGAGAA
AAAAGCTGCTGTACATTTTCAAACAGCCGTTTATGAGGCCGGTCCAAACG
ACTCAGGAAGAGGACGGCTGCTCCTGCCGCTTTCCTGAGGAGGAGGAGGG
CGGGTGCGAACTGAGGGTGAAGTTTTCCAGATCTGCAGATGCACCAGCGT
ATCAGCAGGGCCAGAACCAACTGTATAACGAGCTCAACCTGGGACGCAGG
GAAGAGTATGACGTTTTGGACAAGCGCAGAGGACGGGACCCTGAGATGGG
TGGCAAACCAAGACGAAAAAACCCCCAGGAGGGTCTCTATAATGAGCTGC
AGAAGGATAAGATGGCTGAAGCCTATTCTGAAATAGGCATGAAAGGAGAG
CGGAGAAGGGGAAAAGGGCACGACGGTTTGTACCAGGGACTCAGCACTGC
TACGAAGGATACTTATGACGCTCTCCACATGCAAGCCCTGCCACCTAGGG
CCAAGAGAAGTGGCAGCGGGGAGGGCCGGGGATCTCTCCTTACATGTGGG
GACGTGGAAGAAAATCCGGGGCCTATGGGTGCCGGCGCCACGGGAAGGGC
TATGGATGGCCCGCGACTGCTTCTCCTGCTGTTGTTGGGCGTGTCTCTCG
GAGGCGCTAAGGAGGCCTGTCCAACGGGCCTCTACACTCACTCCGGTGAA
TGTTGCAAAGCCTGTAACCTTGGCGAGGGCGTCGCACAACCTTGTGGTGC
TAACCAGACAGTCTGTGAACCATGCCTGGATTCAGTGACATTCAGCGATG
TTGTCTCAGCCACCGAGCCTTGCAAGCCTTGTACCGAATGTGTGGGCCTT
CAGTCCATGTCCGCCCCCTGTGTCGAAGCCGATGATGCAGTGTGCAGATG
TGCCTATGGATATTACCAGGACGAAACTACCGGGCGGTGTGAGGCCTGCC
GGGTGTGTGAAGCCGGCTCTGGCCTCGTGTTCAGTTGCCAGGATAAGCAA
AACACAGTATGTGAGGAGTGTCCAGACGGAACCTACAGCGACGAGGCGAA
CCACGTCGACCCTTGCTTGCCGTGCACCGTCTGCGAGGATACCGAACGCC
AGCTGAGAGAGTGTACGCGCTGGGCAGACGCTGAGTGCGAGGAGATCCCT
GGGAGATGGATCACCCGGAGCACACCTCCTGAGGGATCAGACAGTACAGC
CCCGAGTACCCAAGAACCGGAGGCCCCTCCAGAGCAGGACCTGATCGCTT
CTACAGTTGCTGGCGTGGTGACGACAGTCATGGGATCCTCACAACCAGTC
GTGACGCGGGGCACAACCGACAATCTGATTCCTGTCTACTGTAGCATCTT
GGCAGCCGTGGTCGTGGGCCTGGTAGCCTACATCGCCTTTAAGAGATGAC
CTAGGTAA

Construct S2-1A1-CD28T-41BB AA (Signal Sequence in Bold; CDRs Underlined)

(SEQ ID NO: 42)
MALPVTALLLPLALLLHAARPQVQLVQSGAEVKKPGASVKVSCKASGYTF
TTYWMHWVRQAPGQGLEWMGEINPSSGRTNYNEKFKTRVTMTRDTSTSTV
YMELSSLRSEDTAVYYCAKLGPGPQYYAMDYVVGQGTMVTVSSGGGGSGG
GGSGGGGSDIQMTQSPSSLSASVGDRVTITCHASQNINVWLSWYQQKPGK
APKLLIYKASKLHTGVPSRFSGSGSGTDFTLTISSLQPEDFATYYCQQGQ
SYPWTFGQGTKLEIKAAALDNEKSNGTIIHVKGKHLCPSPLFPGPSKPFW
VLVVVGGVLACYSLLVTVAFIIFWVRFSVVKRGRKKLLYIFKQPFMRPVQ
TTQEEDGCSCRFPEEEEGGCELRVKFSRSADAPAYQQGQNQLYNELNLGR
REEYDVLDKRRGRDPEMGGKPRRKNPQEGLYNELQKDKMAEAYSEIGMKG
ERRRGKGHDGLYQGLSTATKDTYDALHMQALPPRAKRSGSGEGRGSLLTC
GDVEENPGPMGAGATGRAMDGPRLLLLLLLGVSLGGAKEACPTGLYTHSG
ECCKACNLGEGVAQPCGANQTVCEPCLDSVTFSDVVSATEPCKPCTECVG
LQSMSAPCVEADDAVCRCAYGYYQDETTGRCEACRVCEAGSGLVFSCQDK
QNTVCEECPDGTYSDEANHVDPCLPCTVCEDTERQLRECTRWADAECEEI
PGRWITRSTPPEGSDSTAPSTQEPEAPPEQDLIASTVAGVVTTVMGSSQP
VVTRGTTDNLIPVYCSILAAVVVGLVAYIAFKR

Construct S2-1A1-C8K-CD28 DNA (Signal Sequence in Bold)

(SEQ ID NO: 43)
ATGGCACTCCCCGTAACTGCTCTGCTGCTGCCGTTGGCATTGCTCCTGCA
CGCCGCACGCCCGCAGGTGCAGCTGGTGCAGTCCGGCGCCGAAGTGAAGA
AGCCCGGAGCCAGCGTGAAGGTGAGCTGCAAGGCCTCCGGCTACACCTTC
ACCACCTACTGGATGCACTGGGTCAGACAGGCTCCCGGACAGGGCCTGGA
ATGGATGGGCGAAATCAACCCCTCCTCCGGCAGGACCAACTACAACGAGA
AGTTCAAGACCAGGGTGACCATGACCAGGGACACCAGCACCAGCACCGTG
TACATGGAGCTGTCCAGCCTGAGGAGCGAGGACACCGCCGTGTACTACTG
TGCCAAGCTGGGACCCGGCCCCCAGTACTATGCCATGGACTACTGGGGCC
AAGGCACCATGGTGACCGTGAGCAGCGGAGGCGGAGGATCTGGTGGCGGT
GGTTCTGGCGGCGGAGGCTCCGACATCCAGATGACCCAGAGCCCCTCCAG
CCTGTCCGCTAGCGTGGGCGACAGGGTCACCATTACCTGCCACGCCAGCC
AGAACATCAACGTGTGGCTGAGCTGGTATCAGCAGAAACCCGGCAAGGCT
CCCAAGCTGCTGATCTACAAGGCCAGCAAGCTGCACACCGGCGTGCCCAG
CAGGTTTAGCGGTTCTGGCTCCGGCACCGACTTCACCCTCACCATCAGCA
GCCTGCAGCCCGAAGACTTCGCTACCTACTACTGCCAGCAGGGACAAAGC
TACCCCTGGACCTTCGGCCAGGGAACCAAGCTGGAAATCAAGGCCGCTGC
CTTCGTGCCTGTTTTTCTGCCCGCGAAACCCACAACTACCCCCGCCCCTC
GGCCCCCAACTCCTGCACCAACTATCGCTTCCCAACCCCTGTCTCTGAGA
CCTGAGGCATGCCGCCCCGCGGCAGGCGGCGCCGTGCACACTAGAGGCCT
GGACTTCGCCTGCGATATTTATATCTGGGCCCCCCTTGCCGGGACATGCG
GGGTACTGCTGCTGTCTCTGGTGATTACCCTCTACTGCAACCACAGAAAC
AGATCCAAAAGAAGCCGCCTGCTCCATAGCGATTACATGAATATGACTCC
ACGCCGCCCTGGCCCCACAAGGAAACACTACCAGCCTTACGCACCACCTA
GAGATTTCGCTGCCTATCGGAGCAGGGTGAAGTTTTCCAGATCTGCAGAT
GCACCAGCGTATCAGCAGGGCCAGAACCAACTGTATAACGAGCTCAACCT
GGGACGCAGGGAAGAGTATGACGTTTTGGACAAGCGCAGAGGACGGGACC
CTGAGATGGGTGGCAAACCAAGACGAAAAAACCCCCAGGAGGGTCTCTAT
AATGAGCTGCAGAAGGATAAGATGGCTGAAGCCTATTCTGAAATAGGCAT
GAAAGGAGAGCGGAGAAGGGGAAAAGGGCACGACGGTTTGTACCAGGGAC
TCAGCACTGCTACGAAGGATACTTATGACGCTCTCCACATGCAAGCCCTG
CCACCTAGGGCCAAGAGAAGTGGCAGCGGGGAGGGCCGGGGATCTCTCCT
TACATGTGGGGACGTGGAAGAAAATCCGGGGCCTATGGGTGCCGGCGCCA
CGGGAAGGGCTATGGATGGCCCGCGACTGCTTCTCCTGCTGTTGTTGGGC
GTGTCTCTCGGAGGCGCTAAGGAGGCCTGTCCAACGGGCCTCTACACTCA
CTCCGGTGAATGTTGCAAAGCCTGTAACCTTGGCGAGGGCGTCGCACAAC
CTTGTGGTGCTAACCAGACAGTCTGTGAACCATGCCTGGATTCAGTGACA
TTCAGCGATGTTGTCTCAGCCACCGAGCCTTGCAAGCCTTGTACCGAATG
TGTGGGCCTTCAGTCCATGTCCGCCCCCTGTGTCGAAGCCGATGATGCAG
TGTGCAGATGTGCCTATGGATATTACCAGGACGAAACTACCGGGCGGTGT
GAGGCCTGCCGGGTGTGTGAAGCCGGCTCTGGCCTCGTGTTCAGTTGCCA
GGATAAGCAAAACACAGTATGTGAGGAGTGTCCAGACGGAACCTACAGCG
ACGAGGCGAACCACGTCGACCCTTGCTTGCCGTGCACCGTCTGCGAGGAT
ACCGAACGCCAGCTGAGAGAGTGTACGCGCTGGGCAGACGCTGAGTGCGA
GGAGATCCCTGGGAGATGGATCACCCGGAGCACACCTCCTGAGGGATCAG
ACAGTACAGCCCCGAGTACCCAAGAACCGGAGGCCCCTCCAGAGCAGGAC
CTGATCGCTTCTACAGTTGCTGGCGTGGTGACGACAGTCATGGGATCCTC
ACAACCAGTCGTGACGCGGGGCACAACCGACAATCTGATTCCTGTCTACT
GTAGCATCTTGGCAGCCGTGGTCGTGGGCCTGGTAGCCTACATCGCCTTT
AAGAGATGACCTAGGTAA

Construct S2-1A1-C8K-CD28 AA (Signal Sequence in Bold; CDRs Underlined)

(SEQ ID NO: 44)
MALPVTALLLPLALLLHAARPQVQLVQSGAEVKKPGASVKVSCKASGYTF
TTYWMHWVRQAPGQGLEWMGEINPSSGRTNYNEKFKTRVTMTRDTSTSTV
YMELSSLRSEDTAVYYCAKLGPGPQYYAMDYVVGQGTMVTVSSGGGGSGG
GGSGGGGSDIQMTQSPSSLSASVGDRVTITCHASQNINVWLSWYQQKPGK
APKLLIYKASKLHTGVPSRFSGSGSGTDFTLTISSLQPEDFATYYCQQGQ
SYPWTFGQGTKLEIKAAAFVPVFLPAKPTTTPAPRPPTPAPTIASQPLSL
RPEACRPAAGGAVHTRGLDFACDIYIWAPLAGTCGVLLLSLVITLYCNHR
NRSKRSRLLHSDYMNMTPRRPGPTRKHYQPYAPPRDFAAYRSRVKFSRSA
DAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQA
LPPRAKRSGSGEGRGSLLTCGDVEENPGPMGAGATGRAMDGPRLLLLLLL
GVSLGGAKEACPTGLYTHSGECCKACNLGEGVAQPCGANQTVCEPCLDSV
TFSDVVSATEPCKPCTECVGLQSMSAPCVEADDAVCRCAYGYYQDETTGR
CEACRVCEAGSGLVFSCQDKQNTVCEECPDGTYSDEANHVDPCLPCTVCE
DTERQLRECTRWADAECEEIPGRWITRSTPPEGSDSTAPSTQEPEAPPEQ
DLIASTVAGVVTTVMGSSQPVVTRGTTDNLIPVYCSILAAVVVGLVAYIA
FKR

Construct S2-1A1-C8K-41BB DNA (Signal Sequence in Bold)

(SEQ ID NO: 45)
ATGGCACTCCCCGTAACTGCTCTGCTGCTGCCGTTGGCATTGCTCCTGCA
CGCCGCACGCCCGCAGGTGCAGCTGGTGCAGTCCGGCGCCGAAGTGAAGA
AGCCCGGAGCCAGCGTGAAGGTGAGCTGCAAGGCCTCCGGCTACACCTTC
ACCACCTACTGGATGCACTGGGTCAGACAGGCTCCCGGACAGGGCCTGGA
ATGGATGGGCGAAATCAACCCCTCCTCCGGCAGGACCAACTACAACGAGA
AGTTCAAGACCAGGGTGACCATGACCAGGGACACCAGCACCAGCACCGTG
TACATGGAGCTGTCCAGCCTGAGGAGCGAGGACACCGCCGTGTACTACTG
TGCCAAGCTGGGACCCGGCCCCCAGTACTATGCCATGGACTACTGGGGCC
AAGGCACCATGGTGACCGTGAGCAGCGGAGGCGGAGGATCTGGTGGCGGT
GGTTCTGGCGGCGGAGGCTCCGACATCCAGATGACCCAGAGCCCCTCCAG
CCTGTCCGCTAGCGTGGGCGACAGGGTCACCATTACCTGCCACGCCAGCC
AGAACATCAACGTGTGGCTGAGCTGGTATCAGCAGAAACCCGGCAAGGCT
CCCAAGCTGCTGATCTACAAGGCCAGCAAGCTGCACACCGGCGTGCCCAG
CAGGTTTAGCGGTTCTGGCTCCGGCACCGACTTCACCCTCACCATCAGCA
GCCTGCAGCCCGAAGACTTCGCTACCTACTACTGCCAGCAGGGACAAAGC
TACCCCTGGACCTTCGGCCAGGGAACCAAGCTGGAAATCAAGGCCGCTGC
CTTCGTGCCTGTTTTTCTGCCCGCGAAACCCACAACTACCCCCGCCCCTC
GGCCCCCAACTCCTGCACCAACTATCGCTTCCCAACCCCTGTCTCTGAGA
CCTGAGGCATGCCGCCCCGCGGCAGGCGGCGCCGTGCACACTAGAGGCCT
GGACTTCGCCTGCGATATTTATATCTGGGCCCCCCTTGCCGGGACATGCG
GGGTACTGCTGCTGTCTCTGGTGATTACCCTCTACTGCAACCACAGAAAC
CGCTTTTCCGTCGTTAAGCGGGGGAGAAAAAAGCTGCTGTACATTTTCAA
ACAGCCGTTTATGAGGCCGGTCCAAACGACTCAGGAAGAGGACGGCTGCT
CCTGCCGCTTTCCTGAGGAGGAGGAGGGCGGGTGCGAACTGAGGGTGAAG
TTTTCCAGATCTGCAGATGCACCAGCGTATCAGCAGGGCCAGAACCAACT
GTATAACGAGCTCAACCTGGGACGCAGGGAAGAGTATGACGTTTTGGACA
AGCGCAGAGGACGGGACCCTGAGATGGGTGGCAAACCAAGACGAAAAAAC
CCCCAGGAGGGTCTCTATAATGAGCTGCAGAAGGATAAGATGGCTGAAGC
CTATTCTGAAATAGGCATGAAAGGAGAGCGGAGAAGGGGAAAAGGGCACG
ACGGTTTGTACCAGGGACTCAGCACTGCTACGAAGGATACTTATGACGCT
CTCCACATGCAAGCCCTGCCACCTAGGGCCAAGAGAAGTGGCAGCGGGGA
GGGCCGGGGATCTCTCCTTACATGTGGGGACGTGGAAGAAAATCCGGGGC
CTATGGGTGCCGGCGCCACGGGAAGGGCTATGGATGGCCCGCGACTGCTT
CTCCTGCTGTTGTTGGGCGTGTCTCTCGGAGGCGCTAAGGAGGCCTGTCC
AACGGGCCTCTACACTCACTCCGGTGAATGTTGCAAAGCCTGTAACCTTG
GCGAGGGCGTCGCACAACCTTGTGGTGCTAACCAGACAGTCTGTGAACCA
TGCCTGGATTCAGTGACATTCAGCGATGTTGTCTCAGCCACCGAGCCTTG
CAAGCCTTGTACCGAATGTGTGGGCCTTCAGTCCATGTCCGCCCCCTGTG
TCGAAGCCGATGATGCAGTGTGCAGATGTGCCTATGGATATTACCAGGAC
GAAACTACCGGGCGGTGTGAGGCCTGCCGGGTGTGTGAAGCCGGCTCTGG
CCTCGTGTTCAGTTGCCAGGATAAGCAAAACACAGTATGTGAGGAGTGTC
CAGACGGAACCTACAGCGACGAGGCGAACCACGTCGACCCTTGCTTGCCG
TGCACCGTCTGCGAGGATACCGAACGCCAGCTGAGAGAGTGTACGCGCTG
GGCAGACGCTGAGTGCGAGGAGATCCCTGGGAGATGGATCACCCGGAGCA
CACCTCCTGAGGGATCAGACAGTACAGCCCCGAGTACCCAAGAACCGGAG
GCCCCTCCAGAGCAGGACCTGATCGCTTCTACAGTTGCTGGCGTGGTGAC
GACAGTCATGGGATCCTCACAACCAGTCGTGACGCGGGGCACAACCGACA
ATCTGATTCCTGTCTACTGTAGCATCTTGGCAGCCGTGGTCGTGGGCCTG
GTAGCCTACATCGCCTTTAAGAGATGACCTAGGTAA

Construct S2-1A1-C8K-41BB AA (Signal Sequence in Bold; CDRs Underlined)

(SEQ ID NO: 46)
MALPVTALLLPLALLLHAARPQVQLVQSGAEVKKPGASVKVSCKASGYTF
TTYWMHWVRQAPGQGLEWMGEINPSSGRTNYNEKFKTRVTMTRDTSTSTV
YMELSSLRSEDTAVYYCAKLGPGPQYYAMDYVVGQGTMVTVSSGGGGSGG
GGSGGGGSDIQMTQSPSSLSASVGDRVTITCHASQNINVWLSWYQQKPGK
APKLLIYKASKLHTGVPSRFSGSGSGTDFTLTISSLQPEDFATYYCQQGQ
SYPWTFGQGTKLEIKAAAFVPVFLPAKPTTTPAPRPPTPAPTIASQPLSL
RPEACRPAAGGAVHTRGLDFACDIYIWAPLAGTCGVLLLSLVITLYCNHR
NRFSVVKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYD
ALHMQALPPRAKRSGSGEGRGSLLTCGDVEENPGPMGAGATGRAMDGPRL
LLLLLLGVSLGGAKEACPTGLYTHSGECCKACNLGEGVAQPCGANQTVCE
PCLDSVTFSDVVSATEPCKPCTECVGLQSMSAPCVEADDAVCRCAYGYYQ
DETTGRCEACRVCEAGSGLVFSCQDKQNTVCEECPDGTYSDEANHVDPCL
PCTVCEDTERQLRECTRWADAECEEIPGRWITRSTPPEGSDSTAPSTQEP
EAPPEQDLIASTVAGVVTTVMGSSQPVVTRGTTDNLIPVYCSILAAVVVG
LVAYIAFKR

Construct S2-7A4-CD28T-CD28-41BB DNA (Signal Sequence in Bold)

(SEQ ID NO: 47)
ATGGCACTCCCCGTAACTGCTCTGCTGCTGCCGTTGGCATTGCTCCTGCA
CGCCGCACGCCCGCAGGTGCAGCTGGTGCAGTCTGGGGCTGAGGTGAAGA
AGCCTGGGGCCTCAGTGAAGGTGTCCTGCAAGGCTTCTGGATACAGCTTC
ACCAGTTATGATATCAACTGGGTGCGACAGGCCACTGGACAAGGGCTTGA
GTGGATGGGGTGGATGAACCCGAACAGTGGTAACACAGGCTATGCACAGA
AGTTCCAGGGCAGAGTCACCATGACCAGGGACACCTCCATAAGCACAGCC
TACATGGAACTGAGCAGCCTGAGATCTGAGGACACGGCCGTGTATTACTG
TGGGAGAGCCGGTTACTACTACTACTTCGGTATGGACGTCTGGGGCCAAG
GGACCACGGTCACCGTATCCTCAGGAGGCGGCGGTTCAGGCGGAGGTGGC
TCTGGCGGTGGCGGAAGTGAAATTGTGTTGACGCAGTCTCCAGGCACCCT
GTCTTTGTCTCCAGGGGAAAGAGCCACCCTCTCCTGCAGGGCCGGTCAGA
GTGTTACCAGCAGCTCCTTTGCTTGGTACCAACAGAAACCTGGCCAGGCT
CCCAGGCTCCTCATCTATCAGACATCCACCAGGGCCACTGGCATCCCAGA
CAGGTTCAGTGGCAGTGGGTCTGGGACAGATTTCACTCTCACCATCAGCA
GACTGGAGCCTGAAGATTTTGCAGTGTATTACTGTCAGCAGTATGGTGGC
TCACGGTCGTTCGGCCAAGGGACCAAGGTGGAACTCAAACGAGCCGCTGC
CCTTGATAATGAAAAGTCAAACGGAACAATCATTCACGTGAAGGGCAAGC
ACCTCTGTCCGTCACCCTTGTTCCCTGGTCCATCCAAGCCATTCTGGGTG
TTGGTCGTAGTGGGTGGAGTCCTCGCTTGTTACTCTCTGCTCGTCACCGT
GGCTTTTATAATCTTCTGGGTTAGATCCAAAAGAAGCCGCCTGCTCCATA
GCGATTACATGAATATGACTCCACGCCGCCCTGGCCCCACAAGGAAACAC
TACCAGCCTTACGCACCACCTAGAGATTTCGCTGCCTATCGGAGCCGCTT
TTCCGTCGTTAAGCGGGGGAGAAAAAAGCTGCTGTACATTTTCAAACAGC
CGTTTATGAGGCCGGTCCAAACGACTCAGGAAGAGGACGGCTGCTCCTGC
CGCTTTCCTGAGGAGGAGGAGGGCGGGTGCGAACTGAGGGTGAAGTTTTC
CAGATCTGCAGATGCACCAGCGTATCAGCAGGGCCAGAACCAACTGTATA
ACGAGCTCAACCTGGGACGCAGGGAAGAGTATGACGTTTTGGACAAGCGC
AGAGGACGGGACCCTGAGATGGGTGGCAAACCAAGACGAAAAAACCCCCA
GGAGGGTCTCTATAATGAGCTGCAGAAGGATAAGATGGCTGAAGCCTATT
CTGAAATAGGCATGAAAGGAGAGCGGAGAAGGGGAAAAGGGCACGACGGT
TTGTACCAGGGACTCAGCACTGCTACGAAGGATACTTATGACGCTCTCCA
CATGCAAGCCCTGCCACCTAGGGCCAAGAGAAGTGGCAGCGGGGAGGGCC
GGGGATCTCTCCTTACATGTGGGGACGTGGAAGAAAATCCGGGGCCTATG
GGTGCCGGCGCCACGGGAAGGGCTATGGATGGCCCGCGACTGCTTCTCCT
GCTGTTGTTGGGCGTGTCTCTCGGAGGCGCTAAGGAGGCCTGTCCAACGG
GCCTCTACACTCACTCCGGTGAATGTTGCAAAGCCTGTAACCTTGGCGAG
GGCGTCGCACAACCTTGTGGTGCTAACCAGACAGTCTGTGAACCATGCCT
GGATTCAGTGACATTCAGCGATGTTGTCTCAGCCACCGAGCCTTGCAAGC
CTTGTACCGAATGTGTGGGCCTTCAGTCCATGTCCGCCCCCTGTGTCGAA
GCCGATGATGCAGTGTGCAGATGTGCCTATGGATATTACCAGGACGAAAC
TACCGGGCGGTGTGAGGCCTGCCGGGTGTGTGAAGCCGGCTCTGGCCTCG
TGTTCAGTTGCCAGGATAAGCAAAACACAGTATGTGAGGAGTGTCCAGAC
GGAACCTACAGCGACGAGGCGAACCACGTCGACCCTTGCTTGCCGTGCAC
CGTCTGCGAGGATACCGAACGCCAGCTGAGAGAGTGTACGCGCTGGGCAG
ACGCTGAGTGCGAGGAGATCCCTGGGAGATGGATCACCCGGAGCACACCT
CCTGAGGGATCAGACAGTACAGCCCCGAGTACCCAAGAACCGGAGGCCCC
TCCAGAGCAGGACCTGATCGCTTCTACAGTTGCTGGCGTGGTGACGACAG
TCATGGGATCCTCACAACCAGTCGTGACGCGGGGCACAACCGACAATCTG
ATTCCTGTCTACTGTAGCATCTTGGCAGCCGTGGTCGTGGGCCTGGTAGC
CTACATCGCCTTTAAGAGATGACCTAGGTAA

Construct S2-7A4-CD28T-CD28-41BB AA (Signal Sequence in Bold; CDRs Underlined)

(SEQ ID NO: 48)
MALPVTALLLPLALLLHAARPQVQLVQSGAEVKKPGASVKVSCKASGYSF
TSYDINWVRQATGQGLEWMGWMNPNSGNTGYAQKFQGRVTMTRDTSISTA
YMELSSLRSEDTAVYYCGRAGYYYYFGMDVWGQGTTVTVSSGGGGSGGGG
SGGGGSEIVLTQSPGTLSLSPGERATLSCRAGQSVTSSSFAWYQQKPGQA
PRLLIYQTSTRATGIPDRFSGSGSGTDFTLTISRLEPEDFAVYYCQQYGG
SRSFGQGTKVELKRAAALDNEKSNGTIIHVKGKHLCPSPLFPGPSKPFWV
LVVVGGVLACYSLLVTVAFIIFWVRSKRSRLLHSDYMNMTPRRPGPTRKH
YQPYAPPRDFAAYRSRFSVVKRGRKKLLYIFKQPFMRPVQTTQEEDGCSC
RFPEEEEGGCELRVKFSRSADAPAYQQGQNQLYNELNLGRREEYDVLDKR
RGRDPEMGGKPRRKNPQEGLYNELQKDKMAEAYSEIGMKGERRRGKGHDG
LYQGLSTATKDTYDALHMQALPPRAKRSGSGEGRGSLLTCGDVEENPGPM
GAGATGRAMDGPRLLLLLLLGVSLGGAKEACPTGLYTHSGECCKACNLGE
GVAQPCGANQTVCEPCLDSVTFSDVVSATEPCKPCTECVGLQSMSAPCVE
ADDAVCRCAYGYYQDETTGRCEACRVCEAGSGLVFSCQDKQNTVCEECPD
GTYSDEANHVDPCLPCTVCEDTERQLRECTRWADAECEEIPGRWITRSTP
PEGSDSTAPSTQEPEAPPEQDLIASTVAGVVTTVMGSSQPVVTRGTTDNL
IPVYCSILAAVVVGLVAYIAFKR

Construct S2-7A4-CD28T-CD28 DNA (Signal Sequence in Bold)

(SEQ ID NO: 49)
ATGGCACTCCCCGTAACTGCTCTGCTGCTGCCGTTGGCATTGCTCCTGCA
CGCCGCACGCCCGCAGGTGCAGCTGGTGCAGTCTGGGGCTGAGGTGAAGA
AGCCTGGGGCCTCAGTGAAGGTGTCCTGCAAGGCTTCTGGATACAGCTTC
ACCAGTTATGATATCAACTGGGTGCGACAGGCCACTGGACAAGGGCTTGA
GTGGATGGGGTGGATGAACCCGAACAGTGGTAACACAGGCTATGCACAGA
AGTTCCAGGGCAGAGTCACCATGACCAGGGACACCTCCATAAGCACAGCC
TACATGGAACTGAGCAGCCTGAGATCTGAGGACACGGCCGTGTATTACTG
TGGGAGAGCCGGTTACTACTACTACTTCGGTATGGACGTCTGGGGCCAAG
GGACCACGGTCACCGTATCCTCAGGAGGCGGCGGTTCAGGCGGAGGTGGC
TCTGGCGGTGGCGGAAGTGAAATTGTGTTGACGCAGTCTCCAGGCACCCT
GTCTTTGTCTCCAGGGGAAAGAGCCACCCTCTCCTGCAGGGCCGGTCAGA
GTGTTACCAGCAGCTCCTTTGCTTGGTACCAACAGAAACCTGGCCAGGCT
CCCAGGCTCCTCATCTATCAGACATCCACCAGGGCCACTGGCATCCCAGA
CAGGTTCAGTGGCAGTGGGTCTGGGACAGATTTCACTCTCACCATCAGCA
GACTGGAGCCTGAAGATTTTGCAGTGTATTACTGTCAGCAGTATGGTGGC
TCACGGTCGTTCGGCCAAGGGACCAAGGTGGAACTCAAACGAGCCGCTGC
CCTTGATAATGAAAAGTCAAACGGAACAATCATTCACGTGAAGGGCAAGC
ACCTCTGTCCGTCACCCTTGTTCCCTGGTCCATCCAAGCCATTCTGGGTG
TTGGTCGTAGTGGGTGGAGTCCTCGCTTGTTACTCTCTGCTCGTCACCGT
GGCTTTTATAATCTTCTGGGTTAGATCCAAAAGAAGCCGCCTGCTCCATA
GCGATTACATGAATATGACTCCACGCCGCCCTGGCCCCACAAGGAAACAC
TACCAGCCTTACGCACCACCTAGAGATTTCGCTGCCTATCGGAGCAGGGT
GAAGTTTTCCAGATCTGCAGATGCACCAGCGTATCAGCAGGGCCAGAACC
AACTGTATAACGAGCTCAACCTGGGACGCAGGGAAGAGTATGACGTTTTG
GACAAGCGCAGAGGACGGGACCCTGAGATGGGTGGCAAACCAAGACGAAA
AAACCCCCAGGAGGGTCTCTATAATGAGCTGCAGAAGGATAAGATGGCTG
AAGCCTATTCTGAAATAGGCATGAAAGGAGAGCGGAGAAGGGGAAAAGGG
CACGACGGTTTGTACCAGGGACTCAGCACTGCTACGAAGGATACTTATGA
CGCTCTCCACATGCAAGCCCTGCCACCTAGGGCCAAGAGAAGTGGCAGCG
GGGAGGGCCGGGGATCTCTCCTTACATGTGGGGACGTGGAAGAAAATCCG
GGGCCTATGGGTGCCGGCGCCACGGGAAGGGCTATGGATGGCCCGCGACT
GCTTCTCCTGCTGTTGTTGGGCGTGTCTCTCGGAGGCGCTAAGGAGGCCT
GTCCAACGGGCCTCTACACTCACTCCGGTGAATGTTGCAAAGCCTGTAAC
CTTGGCGAGGGCGTCGCACAACCTTGTGGTGCTAACCAGACAGTCTGTGA
ACCATGCCTGGATTCAGTGACATTCAGCGATGTTGTCTCAGCCACCGAGC
CTTGCAAGCCTTGTACCGAATGTGTGGGCCTTCAGTCCATGTCCGCCCCC
TGTGTCGAAGCCGATGATGCAGTGTGCAGATGTGCCTATGGATATTACCA
GGACGAAACTACCGGGCGGTGTGAGGCCTGCCGGGTGTGTGAAGCCGGCT
CTGGCCTCGTGTTCAGTTGCCAGGATAAGCAAAACACAGTATGTGAGGAG
TGTCCAGACGGAACCTACAGCGACGAGGCGAACCACGTCGACCCTTGCTT
GCCGTGCACCGTCTGCGAGGATACCGAACGCCAGCTGAGAGAGTGTACGC
GCTGGGCAGACGCTGAGTGCGAGGAGATCCCTGGGAGATGGATCACCCGG
AGCACACCTCCTGAGGGATCAGACAGTACAGCCCCGAGTACCCAAGAACC
GGAGGCCCCTCCAGAGCAGGACCTGATCGCTTCTACAGTTGCTGGCGTGG
TGACGACAGTCATGGGATCCTCACAACCAGTCGTGACGCGGGGCACAACC
GACAATCTGATTCCTGTCTACTGTAGCATCTTGGCAGCCGTGGTCGTGGG
CCTGGTAGCCTACATCGCCTTTAAGAGATGACCTAGGTAA

Construct S2-7A4-CD28T-CD28 AA (Signal Sequence in Bold; CDRs Underlined)

(SEQ ID NO: 50)
MALPVTALLLPLALLLHAARPQVQLVQSGAEVKKPGASVKVSCKASGYSF
TSYDINWVRQATGQGLEWMGWMNPNSGNTGYAQKFQGRVTMTRDTSISTA
YMELSSLRSEDTAVYYCGRAGYYYYFGMDVWGQGTTVTVSSGGGGSGGGG
SGGGGSEIVLTQSPGTLSLSPGERATLSCRAGQSVTSSSFAWYQQKPGQA
PRLLIYQTSTRATGIPDRFSGSGSGTDFTLTISRLEPEDFAVYYCQQYGG
SRSFGQGTKVELKRAAALDNEKSNGTIIHVKGKHLCPSPLFPGPSKPFWV
LVVVGGVLACYSLLVTVAFIIFWVRSKRSRLLHSDYMNMTPRRPGPTRKH
YQPYAPPRDFAAYRSRVKFSRSADAPAYQQGQNQLYNELNLGRREEYDVL
DKRRGRDPEMGGKPRRKNPQEGLYNELQKDKMAEAYSEIGMKGERRRGKG
HDGLYQGLSTATKDTYDALHMQALPPRAKRSGSGEGRGSLLTCGDVEENP
GPMGAGATGRAMDGPRLLLLLLLGVSLGGAKEACPTGLYTHSGECCKACN
LGEGVAQPCGANQTVCEPCLDSVTFSDVVSATEPCKPCTECVGLQSMSAP
CVEADDAVCRCAYGYYQDETTGRCEACRVCEAGSGLVFSCQDKQNTVCEE
CPDGTYSDEANHVDPCLPCTVCEDTERQLRECTRWADAECEEIPGRWITR
STPPEGSDSTAPSTQEPEAPPEQDLIASTVAGVVTTVMGSSQPVVTRGTT
DNLIPVYCSILAAVVVGLVAYIAFKR

Construct S2-7A4-CD28T-41BB DNA (Signal Sequence in Bold)

(SEQ ID NO: 51)
ATGGCACTCCCCGTAACTGCTCTGCTGCTGCCGTTGGCATTGCTCCTGCA
CGCCGCACGCCCGCAGGTGCAGCTGGTGCAGTCTGGGGCTGAGGTGAAGA
AGCCTGGGGCCTCAGTGAAGGTGTCCTGCAAGGCTTCTGGATACAGCTTC
ACCAGTTATGATATCAACTGGGTGCGACAGGCCACTGGACAAGGGCTTGA
GTGGATGGGGTGGATGAACCCGAACAGTGGTAACACAGGCTATGCACAGA
AGTTCCAGGGCAGAGTCACCATGACCAGGGACACCTCCATAAGCACAGCC
TACATGGAACTGAGCAGCCTGAGATCTGAGGACACGGCCGTGTATTACTG
TGGGAGAGCCGGTTACTACTACTACTTCGGTATGGACGTCTGGGGCCAAG
GGACCACGGTCACCGTATCCTCAGGAGGCGGCGGTTCAGGCGGAGGTGGC
TCTGGCGGTGGCGGAAGTGAAATTGTGTTGACGCAGTCTCCAGGCACCCT
GTCTTTGTCTCCAGGGGAAAGAGCCACCCTCTCCTGCAGGGCCGGTCAGA
GTGTTACCAGCAGCTCCTTTGCTTGGTACCAACAGAAACCTGGCCAGGCT
CCCAGGCTCCTCATCTATCAGACATCCACCAGGGCCACTGGCATCCCAGA
CAGGTTCAGTGGCAGTGGGTCTGGGACAGATTTCACTCTCACCATCAGCA
GACTGGAGCCTGAAGATTTTGCAGTGTATTACTGTCAGCAGTATGGTGGC
TCACGGTCGTTCGGCCAAGGGACCAAGGTGGAACTCAAACGAGCCGCTGC
CCTTGATAATGAAAAGTCAAACGGAACAATCATTCACGTGAAGGGCAAGC
ACCTCTGTCCGTCACCCTTGTTCCCTGGTCCATCCAAGCCATTCTGGGTG
TTGGTCGTAGTGGGTGGAGTCCTCGCTTGTTACTCTCTGCTCGTCACCGT
GGCTTTTATAATCTTCTGGGTTCGCTTTTCCGTCGTTAAGCGGGGGAGAA
AAAAGCTGCTGTACATTTTCAAACAGCCGTTTATGAGGCCGGTCCAAACG
ACTCAGGAAGAGGACGGCTGCTCCTGCCGCTTTCCTGAGGAGGAGGAGGG
CGGGTGCGAACTGAGGGTGAAGTTTTCCAGATCTGCAGATGCACCAGCGT
ATCAGCAGGGCCAGAACCAACTGTATAACGAGCTCAACCTGGGACGCAGG
GAAGAGTATGACGTTTTGGACAAGCGCAGAGGACGGGACCCTGAGATGGG
TGGCAAACCAAGACGAAAAAACCCCCAGGAGGGTCTCTATAATGAGCTGC
AGAAGGATAAGATGGCTGAAGCCTATTCTGAAATAGGCATGAAAGGAGAG
CGGAGAAGGGGAAAAGGGCACGACGGTTTGTACCAGGGACTCAGCACTGC
TACGAAGGATACTTATGACGCTCTCCACATGCAAGCCCTGCCACCTAGGG
CCAAGAGAAGTGGCAGCGGGGAGGGCCGGGGATCTCTCCTTACATGTGGG
GACGTGGAAGAAAATCCGGGGCCTATGGGTGCCGGCGCCACGGGAAGGGC
TATGGATGGCCCGCGACTGCTTCTCCTGCTGTTGTTGGGCGTGTCTCTCG
GAGGCGCTAAGGAGGCCTGTCCAACGGGCCTCTACACTCACTCCGGTGAA
TGTTGCAAAGCCTGTAACCTTGGCGAGGGCGTCGCACAACCTTGTGGTGC
TAACCAGACAGTCTGTGAACCATGCCTGGATTCAGTGACATTCAGCGATG
TTGTCTCAGCCACCGAGCCTTGCAAGCCTTGTACCGAATGTGTGGGCCTT
CAGTCCATGTCCGCCCCCTGTGTCGAAGCCGATGATGCAGTGTGCAGATG
TGCCTATGGATATTACCAGGACGAAACTACCGGGCGGTGTGAGGCCTGCC
GGGTGTGTGAAGCCGGCTCTGGCCTCGTGTTCAGTTGCCAGGATAAGCAA
AACACAGTATGTGAGGAGTGTCCAGACGGAACCTACAGCGACGAGGCGAA
CCACGTCGACCCTTGCTTGCCGTGCACCGTCTGCGAGGATACCGAACGCC
AGCTGAGAGAGTGTACGCGCTGGGCAGACGCTGAGTGCGAGGAGATCCCT
GGGAGATGGATCACCCGGAGCACACCTCCTGAGGGATCAGACAGTACAGC
CCCGAGTACCCAAGAACCGGAGGCCCCTCCAGAGCAGGACCTGATCGCTT
CTACAGTTGCTGGCGTGGTGACGACAGTCATGGGATCCTCACAACCAGTC
GTGACGCGGGGCACAACCGACAATCTGATTCCTGTCTACTGTAGCATCTT
GGCAGCCGTGGTCGTGGGCCTGGTAGCCTACATCGCCTTTAAGAGATGAC
CTAGGTAA

Construct S2-7A4-CD28T-41BB AA (Signal Sequence in Bold; CDRs Underlined)

(SEQ ID NO: 52)
MALPVTALLLPLALLLHAARPQVQLVQSGAEVKKPGASVKVSCKASGYSF
TSYDINWVRQATGQGLEWMGWMNPNSGNTGYAQKFQGRVTMTRDTSISTA
YMELSSLRSEDTAVYYCGRAGYYYYFGMDVWGQGTTVTVSSGGGGSGGGG
SGGGGSEIVLTQSPGTLSLSPGERATLSCRAGQSVTSSSFAWYQQKPGQA
PRLLIYQTSTRATGIPDRFSGSGSGTDFTLTISRLEPEDFAVYYCQQYGG
SRSFGQGTKVELKRAAALDNEKSNGTIIHVKGKHLCPSPLFPGPSKPFWV
LVVVGGVLACYSLLVTVAFIIFWVRFSVVKRGRKKLLYIFKQPFMRPVQT
TQEEDGCSCRFPEEEEGGCELRVKFSRSADAPAYQQGQNQLYNELNLGRR
EEYDVLDKRRGRDPEMGGKPRRKNPQEGLYNELQKDKMAEAYSEIGMKGE
RRRGKGHDGLYQGLSTATKDTYDALHMQALPPRAKRSGSGEGRGSLLTCG
DVEENPGPMGAGATGRAMDGPRLLLLLLLGVSLGGAKEACPTGLYTHSGE
CCKACNLGEGVAQPCGANQTVCEPCLDSVTFSDVVSATEPCKPCTECVGL
QSMSAPCVEADDAVCRCAYGYYQDETTGRCEACRVCEAGSGLVFSCQDKQ
NTVCEECPDGTYSDEANHVDPCLPCTVCEDTERQLRECTRWADAECEEIP
GRWITRSTPPEGSDSTAPSTQEPEAPPEQDLIASTVAGVVTTVMGSSQPV
VTRGTTDNLIPVYCSILAAVVVGLVAYIAFKR

Construct S2-7A4-C8K-CD28 DNA (Signal Sequence in Bold)

(SEQ ID NO: 53)
ATGGCACTCCCCGTAACTGCTCTGCTGCTGCCGTTGGCATTGCTCCTGCA
CGCCGCACGCCCGCAGGTGCAGCTGGTGCAGTCTGGGGCTGAGGTGAAGA
AGCCTGGGGCCTCAGTGAAGGTGTCCTGCAAGGCTTCTGGATACAGCTTC
ACCAGTTATGATATCAACTGGGTGCGACAGGCCACTGGACAAGGGCTTGA
GTGGATGGGGTGGATGAACCCGAACAGTGGTAACACAGGCTATGCACAGA
AGTTCCAGGGCAGAGTCACCATGACCAGGGACACCTCCATAAGCACAGCC
TACATGGAACTGAGCAGCCTGAGATCTGAGGACACGGCCGTGTATTACTG
TGGGAGAGCCGGTTACTACTACTACTTCGGTATGGACGTCTGGGGCCAAG
GGACCACGGTCACCGTATCCTCAGGAGGCGGCGGTTCAGGCGGAGGTGGC
TCTGGCGGTGGCGGAAGTGAAATTGTGTTGACGCAGTCTCCAGGCACCCT
GTCTTTGTCTCCAGGGGAAAGAGCCACCCTCTCCTGCAGGGCCGGTCAGA
GTGTTACCAGCAGCTCCTTTGCTTGGTACCAACAGAAACCTGGCCAGGCT
CCCAGGCTCCTCATCTATCAGACATCCACCAGGGCCACTGGCATCCCAGA
CAGGTTCAGTGGCAGTGGGTCTGGGACAGATTTCACTCTCACCATCAGCA
GACTGGAGCCTGAAGATTTTGCAGTGTATTACTGTCAGCAGTATGGTGGC
TCACGGTCGTTCGGCCAAGGGACCAAGGTGGAACTCAAACGAGCCGCTGC
CTTCGTGCCTGTTTTTCTGCCCGCGAAACCCACAACTACCCCCGCCCCTC
GGCCCCCAACTCCTGCACCAACTATCGCTTCCCAACCCCTGTCTCTGAGA
CCTGAGGCATGCCGCCCCGCGGCAGGCGGCGCCGTGCACACTAGAGGCCT
GGACTTCGCCTGCGATATTTATATCTGGGCCCCCCTTGCCGGGACATGCG
GGGTACTGCTGCTGTCTCTGGTGATTACCCTCTACTGCAACCACAGAAAC
AGATCCAAAAGAAGCCGCCTGCTCCATAGCGATTACATGAATATGACTCC
ACGCCGCCCTGGCCCCACAAGGAAACACTACCAGCCTTACGCACCACCTA
GAGATTTCGCTGCCTATCGGAGCAGGGTGAAGTTTTCCAGATCTGCAGAT
GCACCAGCGTATCAGCAGGGCCAGAACCAACTGTATAACGAGCTCAACCT
GGGACGCAGGGAAGAGTATGACGTTTTGGACAAGCGCAGAGGACGGGACC
CTGAGATGGGTGGCAAACCAAGACGAAAAAACCCCCAGGAGGGTCTCTAT
AATGAGCTGCAGAAGGATAAGATGGCTGAAGCCTATTCTGAAATAGGCAT
GAAAGGAGAGCGGAGAAGGGGAAAAGGGCACGACGGTTTGTACCAGGGAC
TCAGCACTGCTACGAAGGATACTTATGACGCTCTCCACATGCAAGCCCTG
CCACCTAGGGCCAAGAGAAGTGGCAGCGGGGAGGGCCGGGGATCTCTCCT
TACATGTGGGGACGTGGAAGAAAATCCGGGGCCTATGGGTGCCGGCGCCA
CGGGAAGGGCTATGGATGGCCCGCGACTGCTTCTCCTGCTGTTGTTGGGC
GTGTCTCTCGGAGGCGCTAAGGAGGCCTGTCCAACGGGCCTCTACACTCA
CTCCGGTGAATGTTGCAAAGCCTGTAACCTTGGCGAGGGCGTCGCACAAC
CTTGTGGTGCTAACCAGACAGTCTGTGAACCATGCCTGGATTCAGTGACA
TTCAGCGATGTTGTCTCAGCCACCGAGCCTTGCAAGCCTTGTACCGAATG
TGTGGGCCTTCAGTCCATGTCCGCCCCCTGTGTCGAAGCCGATGATGCAG
TGTGCAGATGTGCCTATGGATATTACCAGGACGAAACTACCGGGCGGTGT
GAGGCCTGCCGGGTGTGTGAAGCCGGCTCTGGCCTCGTGTTCAGTTGCCA
GGATAAGCAAAACACAGTATGTGAGGAGTGTCCAGACGGAACCTACAGCG
ACGAGGCGAACCACGTCGACCCTTGCTTGCCGTGCACCGTCTGCGAGGAT
ACCGAACGCCAGCTGAGAGAGTGTACGCGCTGGGCAGACGCTGAGTGCGA
GGAGATCCCTGGGAGATGGATCACCCGGAGCACACCTCCTGAGGGATCAG
ACAGTACAGCCCCGAGTACCCAAGAACCGGAGGCCCCTCCAGAGCAGGAC
CTGATCGCTTCTACAGTTGCTGGCGTGGTGACGACAGTCATGGGATCCTC
ACAACCAGTCGTGACGCGGGGCACAACCGACAATCTGATTCCTGTCTACT
GTAGCATCTTGGCAGCCGTGGTCGTGGGCCTGGTAGCCTACATCGCCTTT
AAGAGATGACCTAGGTAA

Construct S2-7A4-C8K-CD28 AA (Signal Sequence in Bold; CDRs Underlined)

(SEQ ID NO: 54)
MALPVTALLLPLALLLHAARPQVQLVQSGAEVKKPGASVKVSCKASGYSF
TSYDINWVRQATGQGLEWMGWMNPNSGNTGYAQKFQGRVTMTRDTSISTA
YMELSSLRSEDTAVYYCGRAGYYYYFGMDVWGQGTTVTVSSGGGGSGGGG
SGGGGSEIVLTQSPGTLSLSPGERATLSCRAGQSVTSSSFAWYQQKPGQA
PRLLIYQTSTRATGIPDRFSGSGSGTDFTLTISRLEPEDFAVYYCQQYGG
SRSFGQGTKVELKRAAAFVPVFLPAKPTTTPAPRPPTPAPTIASQPLSLR
PEACRPAAGGAVHTRGLDFACDIYIWAPLAGTCGVLLLSLVITLYCNHRN
RSKRSRLLHSDYMNMTPRRPGPTRKHYQPYAPPRDFAAYRSRVKFSRSAD
APAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQAL
PPRAKRSGSGEGRGSLLTCGDVEENPGPMGAGATGRAMDGPRLLLLLLLG
VSLGGAKEACPTGLYTHSGECCKACNLGEGVAQPCGANQTVCEPCLDSVT
FSDVVSATEPCKPCTECVGLQSMSAPCVEADDAVCRCAYGYYQDETTGRC
EACRVCEAGSGLVFSCQDKQNTVCEECPDGTYSDEANHVDPCLPCTVCED
TERQLRECTRWADAECEEIPGRWITRSTPPEGSDSTAPSTQEPEAPPEQD
LIASTVAGVVTTVMGSSQPVVTRGTTDNLIPVYCSILAAVVVGLVAYIAF
KR

Construct S2-7A4-C8K-41BB DNA (Signal Sequence in Bold)

(SEQ ID NO: 55)
ATGGCACTCCCCGTAACTGCTCTGCTGCTGCCGTTGGCATTGCTCCTGCA
CGCCGCACGCCCGCAGGTGCAGCTGGTGCAGTCTGGGGCTGAGGTGAAGA
AGCCTGGGGCCTCAGTGAAGGTGTCCTGCAAGGCTTCTGGATACAGCTTC
ACCAGTTATGATATCAACTGGGTGCGACAGGCCACTGGACAAGGGCTTGA
GTGGATGGGGTGGATGAACCCGAACAGTGGTAACACAGGCTATGCACAGA
AGTTCCAGGGCAGAGTCACCATGACCAGGGACACCTCCATAAGCACAGCC
TACATGGAACTGAGCAGCCTGAGATCTGAGGACACGGCCGTGTATTACTG
TGGGAGAGCCGGTTACTACTACTACTTCGGTATGGACGTCTGGGGCCAAG
GGACCACGGTCACCGTATCCTCAGGAGGCGGCGGTTCAGGCGGAGGTGGC
TCTGGCGGTGGCGGAAGTGAAATTGTGTTGACGCAGTCTCCAGGCACCCT
GTCTTTGTCTCCAGGGGAAAGAGCCACCCTCTCCTGCAGGGCCGGTCAGA
GTGTTACCAGCAGCTCCTTTGCTTGGTACCAACAGAAACCTGGCCAGGCT
CCCAGGCTCCTCATCTATCAGACATCCACCAGGGCCACTGGCATCCCAGA
CAGGTTCAGTGGCAGTGGGTCTGGGACAGATTTCACTCTCACCATCAGCA
GACTGGAGCCTGAAGATTTTGCAGTGTATTACTGTCAGCAGTATGGTGGC
TCACGGTCGTTCGGCCAAGGGACCAAGGTGGAACTCAAACGAGCCGCTGC
CTTCGTGCCTGTTTTTCTGCCCGCGAAACCCACAACTACCCCCGCCCCTC
GGCCCCCAACTCCTGCACCAACTATCGCTTCCCAACCCCTGTCTCTGAGA
CCTGAGGCATGCCGCCCCGCGGCAGGCGGCGCCGTGCACACTAGAGGCCT
GGACTTCGCCTGCGATATTTATATCTGGGCCCCCCTTGCCGGGACATGCG
GGGTACTGCTGCTGTCTCTGGTGATTACCCTCTACTGCAACCACAGAAAC
CGCTTTTCCGTCGTTAAGCGGGGGAGAAAAAAGCTGCTGTACATTTTCAA
ACAGCCGTTTATGAGGCCGGTCCAAACGACTCAGGAAGAGGACGGCTGCT
CCTGCCGCTTTCCTGAGGAGGAGGAGGGCGGGTGCGAACTGAGGGTGAAG
TTTTCCAGATCTGCAGATGCACCAGCGTATCAGCAGGGCCAGAACCAACT
GTATAACGAGCTCAACCTGGGACGCAGGGAAGAGTATGACGTTTTGGACA
AGCGCAGAGGACGGGACCCTGAGATGGGTGGCAAACCAAGACGAAAAAAC
CCCCAGGAGGGTCTCTATAATGAGCTGCAGAAGGATAAGATGGCTGAAGC
CTATTCTGAAATAGGCATGAAAGGAGAGCGGAGAAGGGGAAAAGGGCACG
ACGGTTTGTACCAGGGACTCAGCACTGCTACGAAGGATACTTATGACGCT
CTCCACATGCAAGCCCTGCCACCTAGGGCCAAGAGAAGTGGCAGCGGGGA
GGGCCGGGGATCTCTCCTTACATGTGGGGACGTGGAAGAAAATCCGGGGC
CTATGGGTGCCGGCGCCACGGGAAGGGCTATGGATGGCCCGCGACTGCTT
CTCCTGCTGTTGTTGGGCGTGTCTCTCGGAGGCGCTAAGGAGGCCTGTCC
AACGGGCCTCTACACTCACTCCGGTGAATGTTGCAAAGCCTGTAACCTTG
GCGAGGGCGTCGCACAACCTTGTGGTGCTAACCAGACAGTCTGTGAACCA
TGCCTGGATTCAGTGACATTCAGCGATGTTGTCTCAGCCACCGAGCCTTG
CAAGCCTTGTACCGAATGTGTGGGCCTTCAGTCCATGTCCGCCCCCTGTG
TCGAAGCCGATGATGCAGTGTGCAGATGTGCCTATGGATATTACCAGGAC
GAAACTACCGGGCGGTGTGAGGCCTGCCGGGTGTGTGAAGCCGGCTCTGG
CCTCGTGTTCAGTTGCCAGGATAAGCAAAACACAGTATGTGAGGAGTGTC
CAGACGGAACCTACAGCGACGAGGCGAACCACGTCGACCCTTGCTTGCCG
TGCACCGTCTGCGAGGATACCGAACGCCAGCTGAGAGAGTGTACGCGCTG
GGCAGACGCTGAGTGCGAGGAGATCCCTGGGAGATGGATCACCCGGAGCA
CACCTCCTGAGGGATCAGACAGTACAGCCCCGAGTACCCAAGAACCGGAG
GCCCCTCCAGAGCAGGACCTGATCGCTTCTACAGTTGCTGGCGTGGTGAC
GACAGTCATGGGATCCTCACAACCAGTCGTGACGCGGGGCACAACCGACA
ATCTGATTCCTGTCTACTGTAGCATCTTGGCAGCCGTGGTCGTGGGCCTG
GTAGCCTACATCGCCTTTAAGAGATGACCTAGGTAA

Construct S2-7A4-C8K-41BB AA (Signal Sequence in Bold; CDRs Underlined)

(SEQ ID NO: 56)
MALPVTALLLPLALLLHAARPQVQLVQSGAEVKKPGASVKVSCKASGYSF
TSYDINWVRQATGQGLEWMGWMNPNSGNTGYAQKFQGRVTMTRDTSISTA
YMELSSLRSEDTAVYYCGRAGYYYYFGMDVWGQGTTVTVSSGGGGSGGGG
SGGGGSEIVLTQSPGTLSLSPGERATLSCRAGQSVTSSSFAWYQQKPGQA
PRLLIYQTSTRATGIPDRFSGSGSGTDFTLTISRLEPEDFAVYYCQQYGG
SRSFGQGTKVELKRAAAFVPVFLPAKPTTTPAPRPPTPAPTIASQPLSLR
PEACRPAAGGAVHTRGLDFACDIYIWAPLAGTCGVLLLSLVITLYCNHRN
RFSVVKRGRKKLLYIFKQPFMRPVQTTQEEDGCSCRFPEEEEGGCELRVK
FSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDA
LHMQALPPRAKRSGSGEGRGSLLTCGDVEENPGPMGAGATGRAMDGPRLL
LLLLLGVSLGGAKEACPTGLYTHSGECCKACNLGEGVAQPCGANQTVCEP
CLDSVTFSDVVSATEPCKPCTECVGLQSMSAPCVEADDAVCRCAYGYYQD
ETTGRCEACRVCEAGSGLVFSCQDKQNTVCEECPDGTYSDEANHVDPCLP
CTVCEDTERQLRECTRWADAECEEIPGRWITRSTPPEGSDSTAPSTQEPE
APPEQDLIASTVAGVVTTVMGSSQPVVTRGTTDNLIPVYCSILAAVVVGL
VAYIAFKR

Construct S2-7A5-CD28T-CD28-41BB DNA (Signal Sequence in Bold)

(SEQ ID NO: 57)
ATGGCACTCCCCGTAACTGCTCTGCTGCTGCCGTTGGCATTGCTCCTGCA
CGCCGCACGCCCGCAGGTGCAGCTGGTGCAGTCTGGGGCTGAGGTGAAGA
AGCCTGGGGCCTCAGTGAAGGTGTCCTGCAAGGCTTCTGGATACAGCTTC
ACCAGTTATGATATCAACTGGGTGCGACAGGCCACTGGACAAGGGCTTGA
GTGGATGGGGTGGATGAACCCGAACAGTGGTAACACAGGCTATGCACAGA
AGTTCCAGGGCAGAGTCACCATGACCAGGGACACCTCCATAAGCACAGCC
TACATGGAACTGAGCAGCCTGAGATCTGAGGACACGGCCGTGTATTACTG
TGGGAGAGCCGGTTACTACTACTACTTCGGTATGGACGTCTGGGGCCAAG
GGACCACGGTCACCGTATCCTCAGGAGGCGGCGGTTCAGGCGGAGGTGGC
TCTGGCGGTGGCGGAAGTGAAATTGTGTTGACGCAGTCTCCAGGCACCCT
GTCTTTGTCTCCAGGGGAAAGAGCCACCCTCTCCTGCAGGGCCGGTCAGA
GTGTTACCAGCAGCTCCTTAGCTTGGTACCAGCAGAAACCTGGCCAGGCT
CCCAGGCTCCTCATCTATCAGACATCCACCAGGGCCACTGGCATCCCAGA
CAGGTTCAGTGGCAGTGGGTCTGGGACAGATTTCACTCTCACCATCAGCA
GACTGGAGCCTGAAGATTTTGCAGTGTATTACTGTCAGCAGTATGGTGGC
TCACGGGCGTTCGGCCAAGGGACCAAGGTGGAACTCAAACGAGCCGCTGC
CCTTGATAATGAAAAGTCAAACGGAACAATCATTCACGTGAAGGGCAAGC
ACCTCTGTCCGTCACCCTTGTTCCCTGGTCCATCCAAGCCATTCTGGGTG
TTGGTCGTAGTGGGTGGAGTCCTCGCTTGTTACTCTCTGCTCGTCACCGT
GGCTTTTATAATCTTCTGGGTTAGATCCAAAAGAAGCCGCCTGCTCCATA
GCGATTACATGAATATGACTCCACGCCGCCCTGGCCCCACAAGGAAACAC
TACCAGCCTTACGCACCACCTAGAGATTTCGCTGCCTATCGGAGCCGCTT
TTCCGTCGTTAAGCGGGGGAGAAAAAAGCTGCTGTACATTTTCAAACAGC
CGTTTATGAGGCCGGTCCAAACGACTCAGGAAGAGGACGGCTGCTCCTGC
CGCTTTCCTGAGGAGGAGGAGGGCGGGTGCGAACTGAGGGTGAAGTTTTC
CAGATCTGCAGATGCACCAGCGTATCAGCAGGGCCAGAACCAACTGTATA
ACGAGCTCAACCTGGGACGCAGGGAAGAGTATGACGTTTTGGACAAGCGC
AGAGGACGGGACCCTGAGATGGGTGGCAAACCAAGACGAAAAAACCCCCA
GGAGGGTCTCTATAATGAGCTGCAGAAGGATAAGATGGCTGAAGCCTATT
CTGAAATAGGCATGAAAGGAGAGCGGAGAAGGGGAAAAGGGCACGACGGT
TTGTACCAGGGACTCAGCACTGCTACGAAGGATACTTATGACGCTCTCCA
CATGCAAGCCCTGCCACCTAGGGCCAAGAGAAGTGGCAGCGGGGAGGGCC
GGGGATCTCTCCTTACATGTGGGGACGTGGAAGAAAATCCGGGGCCTATG
GGTGCCGGCGCCACGGGAAGGGCTATGGATGGCCCGCGACTGCTTCTCCT
GCTGTTGTTGGGCGTGTCTCTCGGAGGCGCTAAGGAGGCCTGTCCAACGG
GCCTCTACACTCACTCCGGTGAATGTTGCAAAGCCTGTAACCTTGGCGAG
GGCGTCGCACAACCTTGTGGTGCTAACCAGACAGTCTGTGAACCATGCCT
GGATTCAGTGACATTCAGCGATGTTGTCTCAGCCACCGAGCCTTGCAAGC
CTTGTACCGAATGTGTGGGCCTTCAGTCCATGTCCGCCCCCTGTGTCGAA
GCCGATGATGCAGTGTGCAGATGTGCCTATGGATATTACCAGGACGAAAC
TACCGGGCGGTGTGAGGCCTGCCGGGTGTGTGAAGCCGGCTCTGGCCTCG
TGTTCAGTTGCCAGGATAAGCAAAACACAGTATGTGAGGAGTGTCCAGAC
GGAACCTACAGCGACGAGGCGAACCACGTCGACCCTTGCTTGCCGTGCAC
CGTCTGCGAGGATACCGAACGCCAGCTGAGAGAGTGTACGCGCTGGGCAG
ACGCTGAGTGCGAGGAGATCCCTGGGAGATGGATCACCCGGAGCACACCT
CCTGAGGGATCAGACAGTACAGCCCCGAGTACCCAAGAACCGGAGGCCCC
TCCAGAGCAGGACCTGATCGCTTCTACAGTTGCTGGCGTGGTGACGACAG
TCATGGGATCCTCACAACCAGTCGTGACGCGGGGCACAACCGACAATCTG
ATTCCTGTCTACTGTAGCATCTTGGCAGCCGTGGTCGTGGGCCTGGTAGC
CTACATCGCCTTTAAGAGATGACCTAGGTAA

Construct S2-7A5-CD28T-CD28-41BB AA (Signal Sequence in Bold; CDRs Underlined)

(SEQ ID NO: 58)
MALPVTALLLPLALLLHAARP
QVQLVQSGAEVKKPGASVKVSCKASGYSF
TSYDINWVRQATGQGLEWMGWMNPNSGNTGYAQKFQGRVTMTRDTSISTA
YMELSSLRSEDTAVYYCGRAGYYYYFGMDVWGQGTTVTVSSGGGGSGGGG
SGGGGSEIVLTQSPGTLSLSPGERATLSCRAGQSVTSSSLAWYQQKPGQA
PRLLIYQTSTRATGIPDRFSGSGSGTDFTLTISRLEPEDFAVYYCQQYGG
SRAFGQGTKVELKRAAALDNEKSNGTIIHVKGKHLCPSPLFPGPSKPFWV
LVVVGGVLACYSLLVTVAFIIFWVRSKRSRLLHSDYMNMTPRRPGPTRKH
YQPYAPPRDFAAYRSRFSVVKRGRKKLLYIFKQPFMRPVQTTQEEDGCSC
RFPEEEEGGCELRVKFSRSADAPAYQQGQNQLYNELNLGRREEYDVLDKR
RGRDPEMGGKPRRKNPQEGLYNELQKDKMAEAYSEIGMKGERRRGKGHDG
LYQGLSTATKDTYDALHMQALPPRAKRSGSGEGRGSLLTCGDVEENPGPM
GAGATGRAMDGPRLLLLLLLGVSLGGAKEACPTGLYTHSGECCKACNLGE
GVAQPCGANQTVCEPCLDSVTFSDVVSATEPCKPCTECVGLQSMSAPCVE
ADDAVCRCAYGYYQDETTGRCEACRVCEAGSGLVFSCQDKQNTVCEECPD
GTYSDEANHVDPCLPCTVCEDTERQLRECTRWADAECEEIPGRWITRSTP
PEGSDSTAPSTQEPEAPPEQDLIASTVAGVVTTVMGSSQPVVTRGTTDNL
IPVYCSILAAVVVGLVAYIAFKR

Construct S2-7A5-CD28T-CD28 DNA (Signal Sequence in Bold)

(SEQ ID NO: 59)
ATGGCACTCCCCGTAACTGCTCTGCTGCTGCCGTTGGCATTGCTCCTGCA
CGCCGCACGCCCGCAGGTGCAGCTGGTGCAGTCTGGGGCTGAGGTGAAGA
AGCCTGGGGCCTCAGTGAAGGTGTCCTGCAAGGCTTCTGGATACAGCTTC
ACCAGTTATGATATCAACTGGGTGCGACAGGCCACTGGACAAGGGCTTGA
GTGGATGGGGTGGATGAACCCGAACAGTGGTAACACAGGCTATGCACAGA
AGTTCCAGGGCAGAGTCACCATGACCAGGGACACCTCCATAAGCACAGCC
TACATGGAACTGAGCAGCCTGAGATCTGAGGACACGGCCGTGTATTACTG
TGGGAGAGCCGGTTACTACTACTACTTCGGTATGGACGTCTGGGGCCAAG
GGACCACGGTCACCGTATCCTCAGGAGGCGGCGGTTCAGGCGGAGGTGGC
TCTGGCGGTGGCGGAAGTGAAATTGTGTTGACGCAGTCTCCAGGCACCCT
GTCTTTGTCTCCAGGGGAAAGAGCCACCCTCTCCTGCAGGGCCGGTCAGA
GTGTTACCAGCAGCTCCTTAGCTTGGTACCAGCAGAAACCTGGCCAGGCT
CCCAGGCTCCTCATCTATCAGACATCCACCAGGGCCACTGGCATCCCAGA
CAGGTTCAGTGGCAGTGGGTCTGGGACAGATTTCACTCTCACCATCAGCA
GACTGGAGCCTGAAGATTTTGCAGTGTATTACTGTCAGCAGTATGGTGGC
TCACGGGCGTTCGGCCAAGGGACCAAGGTGGAACTCAAACGAGCCGCTGC
CCTTGATAATGAAAAGTCAAACGGAACAATCATTCACGTGAAGGGCAAGC
ACCTCTGTCCGTCACCCTTGTTCCCTGGTCCATCCAAGCCATTCTGGGTG
TTGGTCGTAGTGGGTGGAGTCCTCGCTTGTTACTCTCTGCTCGTCACCGT
GGCTTTTATAATCTTCTGGGTTAGATCCAAAAGAAGCCGCCTGCTCCATA
GCGATTACATGAATATGACTCCACGCCGCCCTGGCCCCACAAGGAAACAC
TACCAGCCTTACGCACCACCTAGAGATTTCGCTGCCTATCGGAGCAGGGT
GAAGTTTTCCAGATCTGCAGATGCACCAGCGTATCAGCAGGGCCAGAACC
AACTGTATAACGAGCTCAACCTGGGACGCAGGGAAGAGTATGACGTTTTG
GACAAGCGCAGAGGACGGGACCCTGAGATGGGTGGCAAACCAAGACGAAA
AAACCCCCAGGAGGGTCTCTATAATGAGCTGCAGAAGGATAAGATGGCTG
AAGCCTATTCTGAAATAGGCATGAAAGGAGAGCGGAGAAGGGGAAAAGGG
CACGACGGTTTGTACCAGGGACTCAGCACTGCTACGAAGGATACTTATGA
CGCTCTCCACATGCAAGCCCTGCCACCTAGGGCCAAGAGAAGTGGCAGCG
GGGAGGGCCGGGGATCTCTCCTTACATGTGGGGACGTGGAAGAAAATCCG
GGGCCTATGGGTGCCGGCGCCACGGGAAGGGCTATGGATGGCCCGCGACT
GCTTCTCCTGCTGTTGTTGGGCGTGTCTCTCGGAGGCGCTAAGGAGGCCT
GTCCAACGGGCCTCTACACTCACTCCGGTGAATGTTGCAAAGCCTGTAAC
CTTGGCGAGGGCGTCGCACAACCTTGTGGTGCTAACCAGACAGTCTGTGA
ACCATGCCTGGATTCAGTGACATTCAGCGATGTTGTCTCAGCCACCGAGC
CTTGCAAGCCTTGTACCGAATGTGTGGGCCTTCAGTCCATGTCCGCCCCC
TGTGTCGAAGCCGATGATGCAGTGTGCAGATGTGCCTATGGATATTACCA
GGACGAAACTACCGGGCGGTGTGAGGCCTGCCGGGTGTGTGAAGCCGGCT
CTGGCCTCGTGTTCAGTTGCCAGGATAAGCAAAACACAGTATGTGAGGAG
TGTCCAGACGGAACCTACAGCGACGAGGCGAACCACGTCGACCCTTGCTT
GCCGTGCACCGTCTGCGAGGATACCGAACGCCAGCTGAGAGAGTGTACGC
GCTGGGCAGACGCTGAGTGCGAGGAGATCCCTGGGAGATGGATCACCCGG
AGCACACCTCCTGAGGGATCAGACAGTACAGCCCCGAGTACCCAAGAACC
GGAGGCCCCTCCAGAGCAGGACCTGATCGCTTCTACAGTTGCTGGCGTGG
TGACGACAGTCATGGGATCCTCACAACCAGTCGTGACGCGGGGCACAACC
GACAATCTGATTCCTGTCTACTGTAGCATCTTGGCAGCCGTGGTCGTGGG
CCTGGTAGCCTACATCGCCTTTAAGAGATGACCTAGGTAA

Construct S2-7A5-CD28T-CD28 AA (Signal Sequence in Bold; CDRs Underlined)

(SEQ ID NO: 60)
MALPVTALLLPLALLLHAARP
QVQLVQSGAEVKKPGASVKVSCKASGYSF
TSYDINWVRQATGQGLEWMGWMNPNSGNTGYAQKFQGRVTMTRDTSISTA
YMELSSLRSEDTAVYYCGRAGYYYYFGMDVWGQGTTVTVSSGGGGSGGGG
SGGGGSEIVLTQSPGTLSLSPGERATLSCRAGQSVTSSSLAWYQQKPGQA
PRLLIYQTSTRATGIPDRFSGSGSGTDFTLTISRLEPEDFAVYYCQQYGG
SRAFGQGTKVELKRAAALDNEKSNGTIIHVKGKHLCPSPLFPGPSKPFWV
LVVVGGVLACYSLLVTVAFIIFWVRSKRSRLLHSDYMNMTPRRPGPTRKH
YQPYAPPRDFAAYRSRVKFSRSADAPAYQQGQNQLYNELNLGRREEYDVL
DKRRGRDPEMGGKPRRKNPQEGLYNELQKDKMAEAYSEIGMKGERRRGKG
HDGLYQGLSTATKDTYDALHMQALPPRAKRSGSGEGRGSLLTCGDVEENP
GPMGAGATGRAMDGPRLLLLLLLGVSLGGAKEACPTGLYTHSGECCKACN
LGEGVAQPCGANQTVCEPCLDSVTFSDVVSATEPCKPCTECVGLQSMSAP
CVEADDAVCRCAYGYYQDETTGRCEACRVCEAGSGLVFSCQDKQNTVCEE
CPDGTYSDEANHVDPCLPCTVCEDTERQLRECTRWADAECEEIPGRWITR
STPPEGSDSTAPSTQEPEAPPEQDLIASTVAGVVTTVMGSSQPVVTRGTT
DNLIPVYCSILAAVVVGLVAYIAFKR

Construct S2-7A5-CD28T-41BB DNA (Signal Sequence in Bold)

(SEQ ID NO: 61)
ATGGCACTCCCCGTAACTGCTCTGCTGCTGCCGTTGGCATTGCTCCTGCA
CGCCGCACGCCCGCAGGTGCAGCTGGTGCAGTCTGGGGCTGAGGTGAAGA
AGCCTGGGGCCTCAGTGAAGGTGTCCTGCAAGGCTTCTGGATACAGCTTC
ACCAGTTATGATATCAACTGGGTGCGACAGGCCACTGGACAAGGGCTTGA
GTGGATGGGGTGGATGAACCCGAACAGTGGTAACACAGGCTATGCACAGA
AGTTCCAGGGCAGAGTCACCATGACCAGGGACACCTCCATAAGCACAGCC
TACATGGAACTGAGCAGCCTGAGATCTGAGGACACGGCCGTGTATTACTG
TGGGAGAGCCGGTTACTACTACTACTTCGGTATGGACGTCTGGGGCCAAG
GGACCACGGTCACCGTATCCTCAGGAGGCGGCGGTTCAGGCGGAGGTGGC
TCTGGCGGTGGCGGAAGTGAAATTGTGTTGACGCAGTCTCCAGGCACCCT
GTCTTTGTCTCCAGGGGAAAGAGCCACCCTCTCCTGCAGGGCCGGTCAGA
GTGTTACCAGCAGCTCCTTAGCTTGGTACCAGCAGAAACCTGGCCAGGCT
CCCAGGCTCCTCATCTATCAGACATCCACCAGGGCCACTGGCATCCCAGA
CAGGTTCAGTGGCAGTGGGTCTGGGACAGATTTCACTCTCACCATCAGCA
GACTGGAGCCTGAAGATTTTGCAGTGTATTACTGTCAGCAGTATGGTGGC
TCACGGGCGTTCGGCCAAGGGACCAAGGTGGAACTCAAACGAGCCGCTGC
CCTTGATAATGAAAAGTCAAACGGAACAATCATTCACGTGAAGGGCAAGC
ACCTCTGTCCGTCACCCTTGTTCCCTGGTCCATCCAAGCCATTCTGGGTG
TTGGTCGTAGTGGGTGGAGTCCTCGCTTGTTACTCTCTGCTCGTCACCGT
GGCTTTTATAATCTTCTGGGTTCGCTTTTCCGTCGTTAAGCGGGGGAGAA
AAAAGCTGCTGTACATTTTCAAACAGCCGTTTATGAGGCCGGTCCAAACG
ACTCAGGAAGAGGACGGCTGCTCCTGCCGCTTTCCTGAGGAGGAGGAGGG
CGGGTGCGAACTGAGGGTGAAGTTTTCCAGATCTGCAGATGCACCAGCGT
ATCAGCAGGGCCAGAACCAACTGTATAACGAGCTCAACCTGGGACGCAGG
GAAGAGTATGACGTTTTGGACAAGCGCAGAGGACGGGACCCTGAGATGGG
TGGCAAACCAAGACGAAAAAACCCCCAGGAGGGTCTCTATAATGAGCTGC
AGAAGGATAAGATGGCTGAAGCCTATTCTGAAATAGGCATGAAAGGAGAG
CGGAGAAGGGGAAAAGGGCACGACGGTTTGTACCAGGGACTCAGCACTGC
TACGAAGGATACTTATGACGCTCTCCACATGCAAGCCCTGCCACCTAGGG
CCAAGAGAAGTGGCAGCGGGGAGGGCCGGGGATCTCTCCTTACATGTGGG
GACGTGGAAGAAAATCCGGGGCCTATGGGTGCCGGCGCCACGGGAAGGGC
TATGGATGGCCCGCGACTGCTTCTCCTGCTGTTGTTGGGCGTGTCTCTCG
GAGGCGCTAAGGAGGCCTGTCCAACGGGCCTCTACACTCACTCCGGTGAA
TGTTGCAAAGCCTGTAACCTTGGCGAGGGCGTCGCACAACCTTGTGGTGC
TAACCAGACAGTCTGTGAACCATGCCTGGATTCAGTGACATTCAGCGATG
TTGTCTCAGCCACCGAGCCTTGCAAGCCTTGTACCGAATGTGTGGGCCTT
CAGTCCATGTCCGCCCCCTGTGTCGAAGCCGATGATGCAGTGTGCAGATG
TGCCTATGGATATTACCAGGACGAAACTACCGGGCGGTGTGAGGCCTGCC
GGGTGTGTGAAGCCGGCTCTGGCCTCGTGTTCAGTTGCCAGGATAAGCAA
AACACAGTATGTGAGGAGTGTCCAGACGGAACCTACAGCGACGAGGCGAA
CCACGTCGACCCTTGCTTGCCGTGCACCGTCTGCGAGGATACCGAACGCC
AGCTGAGAGAGTGTACGCGCTGGGCAGACGCTGAGTGCGAGGAGATCCCT
GGGAGATGGATCACCCGGAGCACACCTCCTGAGGGATCAGACAGTACAGC
CCCGAGTACCCAAGAACCGGAGGCCCCTCCAGAGCAGGACCTGATCGCTT
CTACAGTTGCTGGCGTGGTGACGACAGTCATGGGATCCTCACAACCAGTC
GTGACGCGGGGCACAACCGACAATCTGATTCCTGTCTACTGTAGCATCTT
GGCAGCCGTGGTCGTGGGCCTGGTAGCCTACATCGCCTTTAAGAGATGAC
CTAGGTAA

Construct S2-7A5-CD28T-41BB AA (Signal Sequence in Bold; CDRs Underlined)

(SEQ ID NO: 62)
MALPVTALLLPLALLLHAARP
QVQLVQSGAEVKKPGASVKVSCKASGYSF
TSYDINWVRQATGQGLEWMGWMNPNSGNTGYAQKFQGRVTMTRDTSISTA
YMELSSLRSEDTAVYYCGRAGYYYYFGMDVWGQGTTVTVSSGGGGSGGGG
SGGGGSEIVLTQSPGTLSLSPGERATLSCRAGQSVTSSSLAWYQQKPGQA
PRLLIYQTSTRATGIPDRFSGSGSGTDFTLTISRLEPEDFAVYYCQQYGG
SRAFGQGTKVELKRAAALDNEKSNGTIIHVKGKHLCPSPLFPGPSKPFWV
LVVVGGVLACYSLLVTVAFIIFWVRFSVVKRGRKKLLYIFKQPFMRPVQT
TQEEDGCSCRFPEEEEGGCELRVKFSRSADAPAYQQGQNQLYNELNLGRR
EEYDVLDKRRGRDPEMGGKPRRKNPQEGLYNELQKDKMAEAYSEIGMKGE
RRRGKGHDGLYQGLSTATKDTYDALHMQALPPRAKRSGSGEGRGSLLTCG
DVEENPGPMGAGATGRAMDGPRLLLLLLLGVSLGGAKEACPTGLYTHSGE
CCKACNLGEGVAQPCGANQTVCEPCLDSVTFSDVVSATEPCKPCTECVGL
QSMSAPCVEADDAVCRCAYGYYQDETTGRCEACRVCEAGSGLVFSCQDKQ
NTVCEECPDGTYSDEANHVDPCLPCTVCEDTERQLRECTRWADAECEEIP
GRWITRSTPPEGSDSTAPSTQEPEAPPEQDLIASTVAGVVTTVMGSSQPV
VTRGTTDNLIPVYCSILAAVVVGLVAYIAFKR

Construct S2-7A5-C8K-CD28 DNA (Signal Sequence in Bold)

(SEQ ID NO: 63)
ATGGCACTCCCCGTAACTGCTCTGCTGCTGCCGTTGGCATTGCTCCTGCA
CGCCGCACGCCCGCAGGTGCAGCTGGTGCAGTCTGGGGCTGAGGTGAAGA
AGCCTGGGGCCTCAGTGAAGGTGTCCTGCAAGGCTTCTGGATACAGCTTC
ACCAGTTATGATATCAACTGGGTGCGACAGGCCACTGGACAAGGGCTTGA
GTGGATGGGGTGGATGAACCCGAACAGTGGTAACACAGGCTATGCACAGA
AGTTCCAGGGCAGAGTCACCATGACCAGGGACACCTCCATAAGCACAGCC
TACATGGAACTGAGCAGCCTGAGATCTGAGGACACGGCCGTGTATTACTG
TGGGAGAGCCGGTTACTACTACTACTTCGGTATGGACGTCTGGGGCCAAG
GGACCACGGTCACCGTATCCTCAGGAGGCGGCGGTTCAGGCGGAGGTGGC
TCTGGCGGTGGCGGAAGTGAAATTGTGTTGACGCAGTCTCCAGGCACCCT
GTCTTTGTCTCCAGGGGAAAGAGCCACCCTCTCCTGCAGGGCCGGTCAGA
GTGTTACCAGCAGCTCCTTAGCTTGGTACCAGCAGAAACCTGGCCAGGCT
CCCAGGCTCCTCATCTATCAGACATCCACCAGGGCCACTGGCATCCCAGA
CAGGTTCAGTGGCAGTGGGTCTGGGACAGATTTCACTCTCACCATCAGCA
GACTGGAGCCTGAAGATTTTGCAGTGTATTACTGTCAGCAGTATGGTGGC
TCACGGGCGTTCGGCCAAGGGACCAAGGTGGAACTCAAACGAGCCGCTGC
CTTCGTGCCTGTTTTTCTGCCCGCGAAACCCACAACTACCCCCGCCCCTC
GGCCCCCAACTCCTGCACCAACTATCGCTTCCCAACCCCTGTCTCTGAGA
CCTGAGGCATGCCGCCCCGCGGCAGGCGGCGCCGTGCACACTAGAGGCCT
GGACTTCGCCTGCGATATTTATATCTGGGCCCCCCTTGCCGGGACATGCG
GGGTACTGCTGCTGTCTCTGGTGATTACCCTCTACTGCAACCACAGAAAC
AGATCCAAAAGAAGCCGCCTGCTCCATAGCGATTACATGAATATGACTCC
ACGCCGCCCTGGCCCCACAAGGAAACACTACCAGCCTTACGCACCACCTA
GAGATTTCGCTGCCTATCGGAGCAGGGTGAAGTTTTCCAGATCTGCAGAT
GCACCAGCGTATCAGCAGGGCCAGAACCAACTGTATAACGAGCTCAACCT
GGGACGCAGGGAAGAGTATGACGTTTTGGACAAGCGCAGAGGACGGGACC
CTGAGATGGGTGGCAAACCAAGACGAAAAAACCCCCAGGAGGGTCTCTAT
AATGAGCTGCAGAAGGATAAGATGGCTGAAGCCTATTCTGAAATAGGCAT
GAAAGGAGAGCGGAGAAGGGGAAAAGGGCACGACGGTTTGTACCAGGGAC
TCAGCACTGCTACGAAGGATACTTATGACGCTCTCCACATGCAAGCCCTG
CCACCTAGGGCCAAGAGAAGTGGCAGCGGGGAGGGCCGGGGATCTCTCCT
TACATGTGGGGACGTGGAAGAAAATCCGGGGCCTATGGGTGCCGGCGCCA
CGGGAAGGGCTATGGATGGCCCGCGACTGCTTCTCCTGCTGTTGTTGGGC
GTGTCTCTCGGAGGCGCTAAGGAGGCCTGTCCAACGGGCCTCTACACTCA
CTCCGGTGAATGTTGCAAAGCCTGTAACCTTGGCGAGGGCGTCGCACAAC
CTTGTGGTGCTAACCAGACAGTCTGTGAACCATGCCTGGATTCAGTGACA
TTCAGCGATGTTGTCTCAGCCACCGAGCCTTGCAAGCCTTGTACCGAATG
TGTGGGCCTTCAGTCCATGTCCGCCCCCTGTGTCGAAGCCGATGATGCAG
TGTGCAGATGTGCCTATGGATATTACCAGGACGAAACTACCGGGCGGTGT
GAGGCCTGCCGGGTGTGTGAAGCCGGCTCTGGCCTCGTGTTCAGTTGCCA
GGATAAGCAAAACACAGTATGTGAGGAGTGTCCAGACGGAACCTACAGCG
ACGAGGCGAACCACGTCGACCCTTGCTTGCCGTGCACCGTCTGCGAGGAT
ACCGAACGCCAGCTGAGAGAGTGTACGCGCTGGGCAGACGCTGAGTGCGA
GGAGATCCCTGGGAGATGGATCACCCGGAGCACACCTCCTGAGGGATCAG
ACAGTACAGCCCCGAGTACCCAAGAACCGGAGGCCCCTCCAGAGCAGGAC
CTGATCGCTTCTACAGTTGCTGGCGTGGTGACGACAGTCATGGGATCCTC
ACAACCAGTCGTGACGCGGGGCACAACCGACAATCTGATTCCTGTCTACT
GTAGCATCTTGGCAGCCGTGGTCGTGGGCCTGGTAGCCTACATCGCCTTT
AAGAGATGACCTAGGTAA

Construct S2-7A5-C8K-CD28 AA (Signal Sequence in Bold; CDRs Underlined)

(SEQ ID NO: 64)
MALPVTALLLPLALLLHAARP
QVQLVQSGAEVKKPGASVKVSCKASGYSF
TSYDINWVRQATGQGLEWMGWMNPNSGNTGYAQKFQGRVTMTRDTSISTA
YMELSSLRSEDTAVYYCGRAGYYYYFGMDVWGQGTTVTVSSGGGGSGGGG
SGGGGSEIVLTQSPGTLSLSPGERATLSCRAGQSVTSSSLAWYQQKPGQA
PRLLIYQTSTRATGIPDRFSGSGSGTDFTLTISRLEPEDFAVYYCQQYGG
SRAFGQGTKVELKRAAAFVPVFLPAKPTTTPAPRPPTPAPTIASQPLSLR
PEACRPAAGGAVHTRGLDFACDIYIWAPLAGTCGVLLLSLVITLYCNHRN
RSKRSRLLHSDYMNMTPRRPGPTRKHYQPYAPPRDFAAYRSRVKFSRSAD
APAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQAL
PPRAKRSGSGEGRGSLLTCGDVEENPGPMGAGATGRAMDGPRLLLLLLLG
VSLGGAKEACPTGLYTHSGECCKACNLGEGVAQPCGANQTVCEPCLDSVT
FSDVVSATEPCKPCTECVGLQSMSAPCVEADDAVCRCAYGYYQDETTGRC
EACRVCEAGSGLVFSCQDKQNTVCEECPDGTYSDEANHVDPCLPCTVCED
TERQLRECTRWADAECEEIPGRWITRSTPPEGSDSTAPSTQEPEAPPEQD
LIASTVAGVVTTVMGSSQPVVTRGTTDNLIPVYCSILAAVVVGLVAYIAF
KR

Construct S2-7A5-C8K-41BB DNA (Signal Sequence in Bold)

(SEQ ID NO: 65)
ATGGCACTCCCCGTAACTGCTCTGCTGCTGCCGTTGGCATTGCTCCTGCA
CGCCGCACGCCCGCAGGTGCAGCTGGTGCAGTCTGGGGCTGAGGTGAAGA
AGCCTGGGGCCTCAGTGAAGGTGTCCTGCAAGGCTTCTGGATACAGCTTC
ACCAGTTATGATATCAACTGGGTGCGACAGGCCACTGGACAAGGGCTTGA
GTGGATGGGGTGGATGAACCCGAACAGTGGTAACACAGGCTATGCACAGA
AGTTCCAGGGCAGAGTCACCATGACCAGGGACACCTCCATAAGCACAGCC
TACATGGAACTGAGCAGCCTGAGATCTGAGGACACGGCCGTGTATTACTG
TGGGAGAGCCGGTTACTACTACTACTTCGGTATGGACGTCTGGGGCCAAG
GGACCACGGTCACCGTATCCTCAGGAGGCGGCGGTTCAGGCGGAGGTGGC
TCTGGCGGTGGCGGAAGTGAAATTGTGTTGACGCAGTCTCCAGGCACCCT
GTCTTTGTCTCCAGGGGAAAGAGCCACCCTCTCCTGCAGGGCCGGTCAGA
GTGTTACCAGCAGCTCCTTAGCTTGGTACCAGCAGAAACCTGGCCAGGCT
CCCAGGCTCCTCATCTATCAGACATCCACCAGGGCCACTGGCATCCCAGA
CAGGTTCAGTGGCAGTGGGTCTGGGACAGATTTCACTCTCACCATCAGCA
GACTGGAGCCTGAAGATTTTGCAGTGTATTACTGTCAGCAGTATGGTGGC
TCACGGGCGTTCGGCCAAGGGACCAAGGTGGAACTCAAACGAGCCGCTGC
CTTCGTGCCTGTTTTTCTGCCCGCGAAACCCACAACTACCCCCGCCCCTC
GGCCCCCAACTCCTGCACCAACTATCGCTTCCCAACCCCTGTCTCTGAGA
CCTGAGGCATGCCGCCCCGCGGCAGGCGGCGCCGTGCACACTAGAGGCCT
GGACTTCGCCTGCGATATTTATATCTGGGCCCCCCTTGCCGGGACATGCG
GGGTACTGCTGCTGTCTCTGGTGATTACCCTCTACTGCAACCACAGAAAC
CGCTTTTCCGTCGTTAAGCGGGGGAGAAAAAAGCTGCTGTACATTTTCAA
ACAGCCGTTTATGAGGCCGGTCCAAACGACTCAGGAAGAGGACGGCTGCT
CCTGCCGCTTTCCTGAGGAGGAGGAGGGCGGGTGCGAACTGAGGGTGAAG
TTTTCCAGATCTGCAGATGCACCAGCGTATCAGCAGGGCCAGAACCAACT
GTATAACGAGCTCAACCTGGGACGCAGGGAAGAGTATGACGTTTTGGACA
AGCGCAGAGGACGGGACCCTGAGATGGGTGGCAAACCAAGACGAAAAAAC
CCCCAGGAGGGTCTCTATAATGAGCTGCAGAAGGATAAGATGGCTGAAGC
CTATTCTGAAATAGGCATGAAAGGAGAGCGGAGAAGGGGAAAAGGGCACG
ACGGTTTGTACCAGGGACTCAGCACTGCTACGAAGGATACTTATGACGCT
CTCCACATGCAAGCCCTGCCACCTAGGGCCAAGAGAAGTGGCAGCGGGGA
GGGCCGGGGATCTCTCCTTACATGTGGGGACGTGGAAGAAAATCCGGGGC
CTATGGGTGCCGGCGCCACGGGAAGGGCTATGGATGGCCCGCGACTGCTT
CTCCTGCTGTTGTTGGGCGTGTCTCTCGGAGGCGCTAAGGAGGCCTGTCC
AACGGGCCTCTACACTCACTCCGGTGAATGTTGCAAAGCCTGTAACCTTG
GCGAGGGCGTCGCACAACCTTGTGGTGCTAACCAGACAGTCTGTGAACCA
TGCCTGGATTCAGTGACATTCAGCGATGTTGTCTCAGCCACCGAGCCTTG
CAAGCCTTGTACCGAATGTGTGGGCCTTCAGTCCATGTCCGCCCCCTGTG
TCGAAGCCGATGATGCAGTGTGCAGATGTGCCTATGGATATTACCAGGAC
GAAACTACCGGGCGGTGTGAGGCCTGCCGGGTGTGTGAAGCCGGCTCTGG
CCTCGTGTTCAGTTGCCAGGATAAGCAAAACACAGTATGTGAGGAGTGTC
CAGACGGAACCTACAGCGACGAGGCGAACCACGTCGACCCTTGCTTGCCG
TGCACCGTCTGCGAGGATACCGAACGCCAGCTGAGAGAGTGTACGCGCTG
GGCAGACGCTGAGTGCGAGGAGATCCCTGGGAGATGGATCACCCGGAGCA
CACCTCCTGAGGGATCAGACAGTACAGCCCCGAGTACCCAAGAACCGGAG
GCCCCTCCAGAGCAGGACCTGATCGCTTCTACAGTTGCTGGCGTGGTGAC
GACAGTCATGGGATCCTCACAACCAGTCGTGACGCGGGGCACAACCGACA
ATCTGATTCCTGTCTACTGTAGCATCTTGGCAGCCGTGGTCGTGGGCCTG
GTAGCCTACATCGCCTTTAAGAGATGACCTAGGTAA

Construct S2-7A5-C8K-41BB AA (Signal Sequence in Bold; CDRs Underlined)

(SEQ ID NO: 66)
MALPVTALLLPLALLLHAARP
QVQLVQSGAEVKKPGASVKVSCKASGYSF
TSYDINWVRQATGQGLEWMGWMNPNSGNTGYAQKFQGRVTMTRDTSISTA
YMELSSLRSEDTAVYYCGRAGYYYYFGMDVWGQGTTVTVSSGGGGSGGGG
SGGGGSEIVLTQSPGTLSLSPGERATLSCRAGQSVTSSSLAWYQQKPGQA
PRLLIYQTSTRATGIPDRFSGSGSGTDFTLTISRLEPEDFAVYYCQQYGG
SRAFGQGTKVELKRAAAFVPVFLPAKPTTTPAPRPPTPAPTIASQPLSLR
PEACRPAAGGAVHTRGLDFACDIYIWAPLAGTCGVLLLSLVITLYCNHRN
RFSVVKRGRKKLLYIFKQPFMRPVQTTQEEDGCSCRFPEEEEGGCELRVK
FSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDA
LHMQALPPRAKRSGSGEGRGSLLTCGDVEENPGPMGAGATGRAMDGPRLL
LLLLLGVSLGGAKEACPTGLYTHSGECCKACNLGEGVAQPCGANQTVCEP
CLDSVTFSDVVSATEPCKPCTECVGLQSMSAPCVEADDAVCRCAYGYYQD
ETTGRCEACRVCEAGSGLVFSCQDKQNTVCEECPDGTYSDEANHVDPCLP
CTVCEDTERQLRECTRWADAECEEIPGRWITRSTPPEGSDSTAPSTQEPE
APPEQDLIASTVAGVVTTVMGSSQPVVTRGTTDNLIPVYCSILAAVVVGL
VAYIAFKR

Construct S2-14C1-CD28T-CD28-41BB DNA (Signal Sequence in Bold)

(SEQ ID NO: 67)
ATGGCACTCCCCGTAACTGCTCTGCTGCTGCCGTTGGCATTGCTCCTGCA
CGCCGCACGCCCGCAGGTGCAGCTGGTGCAGTCTGGGGCTGAGGTGAAGA
AGCCTGGGGCCTCAGTGAAGGTGTCCTGCAAGGCTTCTGGATACACCTTC
ACCGGCTACTATATGCACTGGGTGCGACAGGCCCCTGGACAAGGGCTTGA
GTGGATGGGATGGATCAACCCTAATAGTGGTGGCACAAACTCTGCACAGA
AGTTTCAGGGCAGGGTCACCATGACCAGGGACACGTCCATCAGTACAGCC
TACATGGAGCTGAACAGGCTGAGATCTGACGACACGGCCGTTTATTACTG
TGCGAGAGGATGGCTACAGACGTACTACTTTGACAACTGGGGCCAGGGAA
CCCTGGTCACCGTATCCTCAGGAGGCGGCGGTTCAGGCGGAGGTGGCTCT
GGCGGTGGCGGAAGTGACATCGTGATGACCCAGTCTCCAGACTCCCTGGC
TGTGTCTCTGGGCGAGAGGGCCACCATCTACTGCAAGTCCAGCCAGACTG
TTTTGACCAGCTCCAACAATAAGAACTTCTTAGCTTGGTACCAACAGAAA
CTAGGACAGCCTCCTAAGCTGCTCATTTCCTGGGCCTCTACCCGGGAATC
CGGGGTCCCTGACCGATTCAGTGGCAGCGGGTCTGGGACAGATTTCACTC
TCACCATCAGCAGCCTGCAGGCTGAAGATGTGGCAATTTATTACTGTCAG
CACTATTATACTAGTCCACTCACTTTCGGCGGCGGGACCAAGGTGGAGAT
CAAACGAGCCGCTGCCCTTGATAATGAAAAGTCAAACGGAACAATCATTC
ACGTGAAGGGCAAGCACCTCTGTCCGTCACCCTTGTTCCCTGGTCCATCC
AAGCCATTCTGGGTGTTGGTCGTAGTGGGTGGAGTCCTCGCTTGTTACTC
TCTGCTCGTCACCGTGGCTTTTATAATCTTCTGGGTTAGATCCAAAAGAA
GCCGCCTGCTCCATAGCGATTACATGAATATGACTCCACGCCGCCCTGGC
CCCACAAGGAAACACTACCAGCCTTACGCACCACCTAGAGATTTCGCTGC
CTATCGGAGCCGCTTTTCCGTCGTTAAGCGGGGGAGAAAAAAGCTGCTGT
ACATTTTCAAACAGCCGTTTATGAGGCCGGTCCAAACGACTCAGGAAGAG
GACGGCTGCTCCTGCCGCTTTCCTGAGGAGGAGGAGGGCGGGTGCGAACT
GAGGGTGAAGTTTTCCAGATCTGCAGATGCACCAGCGTATCAGCAGGGCC
AGAACCAACTGTATAACGAGCTCAACCTGGGACGCAGGGAAGAGTATGAC
GTTTTGGACAAGCGCAGAGGACGGGACCCTGAGATGGGTGGCAAACCAAG
ACGAAAAAACCCCCAGGAGGGTCTCTATAATGAGCTGCAGAAGGATAAGA
TGGCTGAAGCCTATTCTGAAATAGGCATGAAAGGAGAGCGGAGAAGGGGA
AAAGGGCACGACGGTTTGTACCAGGGACTCAGCACTGCTACGAAGGATAC
TTATGACGCTCTCCACATGCAAGCCCTGCCACCTAGGGCCAAGAGAAGTG
GCAGCGGGGAGGGCCGGGGATCTCTCCTTACATGTGGGGACGTGGAAGAA
AATCCGGGGCCTATGGGTGCCGGCGCCACGGGAAGGGCTATGGATGGCCC
GCGACTGCTTCTCCTGCTGTTGTTGGGCGTGTCTCTCGGAGGCGCTAAGG
AGGCCTGTCCAACGGGCCTCTACACTCACTCCGGTGAATGTTGCAAAGCC
TGTAACCTTGGCGAGGGCGTCGCACAACCTTGTGGTGCTAACCAGACAGT
CTGTGAACCATGCCTGGATTCAGTGACATTCAGCGATGTTGTCTCAGCCA
CCGAGCCTTGCAAGCCTTGTACCGAATGTGTGGGCCTTCAGTCCATGTCC
GCCCCCTGTGTCGAAGCCGATGATGCAGTGTGCAGATGTGCCTATGGATA
TTACCAGGACGAAACTACCGGGCGGTGTGAGGCCTGCCGGGTGTGTGAAG
CCGGCTCTGGCCTCGTGTTCAGTTGCCAGGATAAGCAAAACACAGTATGT
GAGGAGTGTCCAGACGGAACCTACAGCGACGAGGCGAACCACGTCGACCC
TTGCTTGCCGTGCACCGTCTGCGAGGATACCGAACGCCAGCTGAGAGAGT
GTACGCGCTGGGCAGACGCTGAGTGCGAGGAGATCCCTGGGAGATGGATC
ACCCGGAGCACACCTCCTGAGGGATCAGACAGTACAGCCCCGAGTACCCA
AGAACCGGAGGCCCCTCCAGAGCAGGACCTGATCGCTTCTACAGTTGCTG
GCGTGGTGACGACAGTCATGGGATCCTCACAACCAGTCGTGACGCGGGGC
ACAACCGACAATCTGATTCCTGTCTACTGTAGCATCTTGGCAGCCGTGGT
CGTGGGCCTGGTAGCCTACATCGCCTTTAAGAGATGACCTAGGTAA

Construct S2-14C1-CD28T-CD28-41BB AA (Signal Sequence in Bold; CDRs Underlined)

(SEQ ID NO: 68)
MALPVTALLLPLALLLHAARP
QVQLVQSGAEVKKPGASVKVSCKASGYTF
TGYYMHWVRQAPGQGLEWMGWINPNSGGTNSAQKFQGRVTMTRDTSISTA
YMELNRLRSDDTAVYYCARGWLQTYYFDNWGQGTLVTVSSGGGGSGGGGS
GGGGSDIVMTQSPDSLAVSLGERATIYCKSSQTVLTSSNNKNFLAWYQQK
LGQPPKLLISWASTRESGVPDRFSGSGSGTDFTLTISSLQAEDVAIYYCQ
HYYTSPLTFGGGTKVEIKRAAALDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRSRFSVVKRGRKKLLYIFKQPFMRPVQTTQEE
DGCSCRFPEEEEGGCELRVKFSRSADAPAYQQGQNQLYNELNLGRREEYD
VLDKRRGRDPEMGGKPRRKNPQEGLYNELQKDKMAEAYSEIGMKGERRRG
KGHDGLYQGLSTATKDTYDALHMQALPPRAKRSGSGEGRGSLLTCGDVEE
NPGPMGAGATGRAMDGPRLLLLLLLGVSLGGAKEACPTGLYTHSGECCKA
CNLGEGVAQPCGANQTVCEPCLDSVTFSDVVSATEPCKPCTECVGLQSMS
APCVEADDAVCRCAYGYYQDETTGRCEACRVCEAGSGLVFSCQDKQNTVC
EECPDGTYSDEANHVDPCLPCTVCEDTERQLRECTRWADAECEEIPGRWI
TRSTPPEGSDSTAPSTQEPEAPPEQDLIASTVAGVVTTVMGSSQPVVTRG
TTDNLIPVYCSILAAVVVGLVAYIAFKR

Construct S2-14C1-CD28T-CD28 DNA (Signal Sequence in Bold)

(SEQ ID NO: 69)
ATGGCACTCCCCGTAACTGCTCTGCTGCTGCCGTTGGCATTGCTCCTGCA
CGCCGCACGCCCGCAGGTGCAGCTGGTGCAGTCTGGGGCTGAGGTGAAGA
AGCCTGGGGCCTCAGTGAAGGTGTCCTGCAAGGCTTCTGGATACACCTTC
ACCGGCTACTATATGCACTGGGTGCGACAGGCCCCTGGACAAGGGCTTGA
GTGGATGGGATGGATCAACCCTAATAGTGGTGGCACAAACTCTGCACAGA
AGTTTCAGGGCAGGGTCACCATGACCAGGGACACGTCCATCAGTACAGCC
TACATGGAGCTGAACAGGCTGAGATCTGACGACACGGCCGTTTATTACTG
TGCGAGAGGATGGCTACAGACGTACTACTTTGACAACTGGGGCCAGGGAA
CCCTGGTCACCGTATCCTCAGGAGGCGGCGGTTCAGGCGGAGGTGGCTCT
GGCGGTGGCGGAAGTGACATCGTGATGACCCAGTCTCCAGACTCCCTGGC
TGTGTCTCTGGGCGAGAGGGCCACCATCTACTGCAAGTCCAGCCAGACTG
TTTTGACCAGCTCCAACAATAAGAACTTCTTAGCTTGGTACCAACAGAAA
CTAGGACAGCCTCCTAAGCTGCTCATTTCCTGGGCCTCTACCCGGGAATC
CGGGGTCCCTGACCGATTCAGTGGCAGCGGGTCTGGGACAGATTTCACTC
TCACCATCAGCAGCCTGCAGGCTGAAGATGTGGCAATTTATTACTGTCAG
CACTATTATACTAGTCCACTCACTTTCGGCGGCGGGACCAAGGTGGAGAT
CAAACGAGCCGCTGCCCTTGATAATGAAAAGTCAAACGGAACAATCATTC
ACGTGAAGGGCAAGCACCTCTGTCCGTCACCCTTGTTCCCTGGTCCATCC
AAGCCATTCTGGGTGTTGGTCGTAGTGGGTGGAGTCCTCGCTTGTTACTC
TCTGCTCGTCACCGTGGCTTTTATAATCTTCTGGGTTAGATCCAAAAGAA
GCCGCCTGCTCCATAGCGATTACATGAATATGACTCCACGCCGCCCTGGC
CCCACAAGGAAACACTACCAGCCTTACGCACCACCTAGAGATTTCGCTGC
CTATCGGAGCAGGGTGAAGTTTTCCAGATCTGCAGATGCACCAGCGTATC
AGCAGGGCCAGAACCAACTGTATAACGAGCTCAACCTGGGACGCAGGGAA
GAGTATGACGTTTTGGACAAGCGCAGAGGACGGGACCCTGAGATGGGTGG
CAAACCAAGACGAAAAAACCCCCAGGAGGGTCTCTATAATGAGCTGCAGA
AGGATAAGATGGCTGAAGCCTATTCTGAAATAGGCATGAAAGGAGAGCGG
AGAAGGGGAAAAGGGCACGACGGTTTGTACCAGGGACTCAGCACTGCTAC
GAAGGATACTTATGACGCTCTCCACATGCAAGCCCTGCCACCTAGGGCCA
AGAGAAGTGGCAGCGGGGAGGGCCGGGGATCTCTCCTTACATGTGGGGAC
GTGGAAGAAAATCCGGGGCCTATGGGTGCCGGCGCCACGGGAAGGGCTAT
GGATGGCCCGCGACTGCTTCTCCTGCTGTTGTTGGGCGTGTCTCTCGGAG
GCGCTAAGGAGGCCTGTCCAACGGGCCTCTACACTCACTCCGGTGAATGT
TGCAAAGCCTGTAACCTTGGCGAGGGCGTCGCACAACCTTGTGGTGCTAA
CCAGACAGTCTGTGAACCATGCCTGGATTCAGTGACATTCAGCGATGTTG
TCTCAGCCACCGAGCCTTGCAAGCCTTGTACCGAATGTGTGGGCCTTCAG
TCCATGTCCGCCCCCTGTGTCGAAGCCGATGATGCAGTGTGCAGATGTGC
CTATGGATATTACCAGGACGAAACTACCGGGCGGTGTGAGGCCTGCCGGG
TGTGTGAAGCCGGCTCTGGCCTCGTGTTCAGTTGCCAGGATAAGCAAAAC
ACAGTATGTGAGGAGTGTCCAGACGGAACCTACAGCGACGAGGCGAACCA
CGTCGACCCTTGCTTGCCGTGCACCGTCTGCGAGGATACCGAACGCCAGC
TGAGAGAGTGTACGCGCTGGGCAGACGCTGAGTGCGAGGAGATCCCTGGG
AGATGGATCACCCGGAGCACACCTCCTGAGGGATCAGACAGTACAGCCCC
GAGTACCCAAGAACCGGAGGCCCCTCCAGAGCAGGACCTGATCGCTTCTA
CAGTTGCTGGCGTGGTGACGACAGTCATGGGATCCTCACAACCAGTCGTG
ACGCGGGGCACAACCGACAATCTGATTCCTGTCTACTGTAGCATCTTGGC
AGCCGTGGTCGTGGGCCTGGTAGCCTACATCGCCTTTAAGAGATGACCTA
GGTAA

Construct S2-14C1-CD28T-CD28 AA (Signal Sequence in Bold; CDRs Underlined)

(SEQ ID NO: 70)
MALPVTALLLPLALLLHAARP
QVQLVQSGAEVKKPGASVKVSCKASGYTF
TGYYMHWVRQAPGQGLEWMGWINPNSGGTNSAQKFQGRVTMTRDTSISTA
YMELNRLRSDDTAVYYCARGWLQTYYFDNWGQGTLVTVSSGGGGSGGGGS
GGGGSDIVMTQSPDSLAVSLGERATIYCKSSQTVLTSSNNKNFLAWYQQK
LGQPPKLLISWASTRESGVPDRFSGSGSGTDFTLTISSLQAEDVAIYYCQ
HYYTSPLTFGGGTKVEIKRAAALDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRSRVKFSRSADAPAYQQGQNQLYNELNLGRRE
EYDVLDKRRGRDPEMGGKPRRKNPQEGLYNELQKDKMAEAYSEIGMKGER
RRGKGHDGLYQGLSTATKDTYDALHMQALPPRAKRSGSGEGRGSLLTCGD
VEENPGPMGAGATGRAMDGPRLLLLLLLGVSLGGAKEACPTGLYTHSGEC
CKACNLGEGVAQPCGANQTVCEPCLDSVTFSDVVSATEPCKPCTECVGLQ
SMSAPCVEADDAVCRCAYGYYQDETTGRCEACRVCEAGSGLVFSCQDKQN
TVCEECPDGTYSDEANHVDPCLPCTVCEDTERQLRECTRWADAECEEIPG
RWITRSTPPEGSDSTAPSTQEPEAPPEQDLIASTVAGVVTTVMGSSQPVV
TRGTTDNLIPVYCSILAAVVVGLVAYIAFKR

Construct S2-14C1-CD28T-41BB DNA (Signal Sequence in Bold)

(SEQ ID NO: 71)
ATGGCACTCCCCGTAACTGCTCTGCTGCTGCCGTTGGCATTGCTCCTGCA
CGCCGCACGCCCGCAGGTGCAGCTGGTGCAGTCTGGGGCTGAGGTGAAGA
AGCCTGGGGCCTCAGTGAAGGTGTCCTGCAAGGCTTCTGGATACACCTTC
ACCGGCTACTATATGCACTGGGTGCGACAGGCCCCTGGACAAGGGCTTGA
GTGGATGGGATGGATCAACCCTAATAGTGGTGGCACAAACTCTGCACAGA
AGTTTCAGGGCAGGGTCACCATGACCAGGGACACGTCCATCAGTACAGCC
TACATGGAGCTGAACAGGCTGAGATCTGACGACACGGCCGTTTATTACTG
TGCGAGAGGATGGCTACAGACGTACTACTTTGACAACTGGGGCCAGGGAA
CCCTGGTCACCGTATCCTCAGGAGGCGGCGGTTCAGGCGGAGGTGGCTCT
GGCGGTGGCGGAAGTGACATCGTGATGACCCAGTCTCCAGACTCCCTGGC
TGTGTCTCTGGGCGAGAGGGCCACCATCTACTGCAAGTCCAGCCAGACTG
TTTTGACCAGCTCCAACAATAAGAACTTCTTAGCTTGGTACCAACAGAAA
CTAGGACAGCCTCCTAAGCTGCTCATTTCCTGGGCCTCTACCCGGGAATC
CGGGGTCCCTGACCGATTCAGTGGCAGCGGGTCTGGGACAGATTTCACTC
TCACCATCAGCAGCCTGCAGGCTGAAGATGTGGCAATTTATTACTGTCAG
CACTATTATACTAGTCCACTCACTTTCGGCGGCGGGACCAAGGTGGAGAT
CAAACGAGCCGCTGCCCTTGATAATGAAAAGTCAAACGGAACAATCATTC
ACGTGAAGGGCAAGCACCTCTGTCCGTCACCCTTGTTCCCTGGTCCATCC
AAGCCATTCTGGGTGTTGGTCGTAGTGGGTGGAGTCCTCGCTTGTTACTC
TCTGCTCGTCACCGTGGCTTTTATAATCTTCTGGGTTCGCTTTTCCGTCG
TTAAGCGGGGGAGAAAAAAGCTGCTGTACATTTTCAAACAGCCGTTTATG
AGGCCGGTCCAAACGACTCAGGAAGAGGACGGCTGCTCCTGCCGCTTTCC
TGAGGAGGAGGAGGGCGGGTGCGAACTGAGGGTGAAGTTTTCCAGATCTG
CAGATGCACCAGCGTATCAGCAGGGCCAGAACCAACTGTATAACGAGCTC
AACCTGGGACGCAGGGAAGAGTATGACGTTTTGGACAAGCGCAGAGGACG
GGACCCTGAGATGGGTGGCAAACCAAGACGAAAAAACCCCCAGGAGGGTC
TCTATAATGAGCTGCAGAAGGATAAGATGGCTGAAGCCTATTCTGAAATA
GGCATGAAAGGAGAGCGGAGAAGGGGAAAAGGGCACGACGGTTTGTACCA
GGGACTCAGCACTGCTACGAAGGATACTTATGACGCTCTCCACATGCAAG
CCCTGCCACCTAGGGCCAAGAGAAGTGGCAGCGGGGAGGGCCGGGGATCT
CTCCTTACATGTGGGGACGTGGAAGAAAATCCGGGGCCTATGGGTGCCGG
CGCCACGGGAAGGGCTATGGATGGCCCGCGACTGCTTCTCCTGCTGTTGT
TGGGCGTGTCTCTCGGAGGCGCTAAGGAGGCCTGTCCAACGGGCCTCTAC
ACTCACTCCGGTGAATGTTGCAAAGCCTGTAACCTTGGCGAGGGCGTCGC
ACAACCTTGTGGTGCTAACCAGACAGTCTGTGAACCATGCCTGGATTCAG
TGACATTCAGCGATGTTGTCTCAGCCACCGAGCCTTGCAAGCCTTGTACC
GAATGTGTGGGCCTTCAGTCCATGTCCGCCCCCTGTGTCGAAGCCGATGA
TGCAGTGTGCAGATGTGCCTATGGATATTACCAGGACGAAACTACCGGGC
GGTGTGAGGCCTGCCGGGTGTGTGAAGCCGGCTCTGGCCTCGTGTTCAGT
TGCCAGGATAAGCAAAACACAGTATGTGAGGAGTGTCCAGACGGAACCTA
CAGCGACGAGGCGAACCACGTCGACCCTTGCTTGCCGTGCACCGTCTGCG
AGGATACCGAACGCCAGCTGAGAGAGTGTACGCGCTGGGCAGACGCTGAG
TGCGAGGAGATCCCTGGGAGATGGATCACCCGGAGCACACCTCCTGAGGG
ATCAGACAGTACAGCCCCGAGTACCCAAGAACCGGAGGCCCCTCCAGAGC
AGGACCTGATCGCTTCTACAGTTGCTGGCGTGGTGACGACAGTCATGGGA
TCCTCACAACCAGTCGTGACGCGGGGCACAACCGACAATCTGATTCCTGT
CTACTGTAGCATCTTGGCAGCCGTGGTCGTGGGCCTGGTAGCCTACATCG
CCTTTAAGAGATGACCTAGGTAA

Construct S2-14C1-CD28T-41BB AA (Signal Sequence in Bold; CDRs Underlined)

(SEQ ID NO: 72)
MALPVTALLLPLALLLHAARPQVQLVQSGAEVKKPGASVKVSCKASGYTF
TGYYMHWVRQAPGQGLEWMGWINPNSGGTNSAQKFQGRVTMTRDTSISTA
YMELNRLRSDDTAVYYCARGWLQTYYFDNWGQGTLVTVSSGGGGSGGGGS
GGGGSDIVMTQSPDSLAVSLGERATIYCKSSQTVLTSSNNKNFLAWYQQK
LGQPPKLLISWASTRESGVPDRFSGSGSGTDFTLTISSLQAEDVAIYYCQ
HYYTSPLTFGGGTKVEIKRAAALDNEKSNGTIIHVKGKHLCPSPLFPGPS
KPFWVLVVVGGVLACYSLLVTVAFIIFWVRFSVVKRGRKKLLYIFKQPFM
RPVQTTQEEDGCSCRFPEEEEGGCELRVKFSRSADAPAYQQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPRAKRSGSGEGRGS
LLTCGDVEENPGPMGAGATGRAMDGPRLLLLLLLGVSLGGAKEACPTGLY
THSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFS
CQDKQNTVCEECPDGTYSDEANHVDPCLPCTVCEDTERQLRECTRWADAE
CEEIPGRWITRSTPPEGSDSTAPSTQEPEAPPEQDLIASTVAGVVTTVMG
SSQPVVTRGTTDNLIPVYCSILAAVVVGLVAYIAFKR

Construct S2-14C1-C8K-CD28 DNA (Signal Sequence in Bold)

(SEQ ID NO: 73)
ATGGCACTCCCCGTAACTGCTCTGCTGCTGCCGTTGGCATTGCTCCTGCA
CGCCGCACGCCCGCAGGTGCAGCTGGTGCAGTCTGGGGCTGAGGTGAAGA
AGCCTGGGGCCTCAGTGAAGGTGTCCTGCAAGGCTTCTGGATACACCTTC
ACCGGCTACTATATGCACTGGGTGCGACAGGCCCCTGGACAAGGGCTTGA
GTGGATGGGATGGATCAACCCTAATAGTGGTGGCACAAACTCTGCACAGA
AGTTTCAGGGCAGGGTCACCATGACCAGGGACACGTCCATCAGTACAGCC
TACATGGAGCTGAACAGGCTGAGATCTGACGACACGGCCGTTTATTACTG
TGCGAGAGGATGGCTACAGACGTACTACTTTGACAACTGGGGCCAGGGAA
CCCTGGTCACCGTATCCTCAGGAGGCGGCGGTTCAGGCGGAGGTGGCTCT
GGCGGTGGCGGAAGTGACATCGTGATGACCCAGTCTCCAGACTCCCTGGC
TGTGTCTCTGGGCGAGAGGGCCACCATCTACTGCAAGTCCAGCCAGACTG
TTTTGACCAGCTCCAACAATAAGAACTTCTTAGCTTGGTACCAACAGAAA
CTAGGACAGCCTCCTAAGCTGCTCATTTCCTGGGCCTCTACCCGGGAATC
CGGGGTCCCTGACCGATTCAGTGGCAGCGGGTCTGGGACAGATTTCACTC
TCACCATCAGCAGCCTGCAGGCTGAAGATGTGGCAATTTATTACTGTCAG
CACTATTATACTAGTCCACTCACTTTCGGCGGCGGGACCAAGGTGGAGAT
CAAACGAGCCGCTGCCTTCGTGCCTGTTTTTCTGCCCGCGAAACCCACAA
CTACCCCCGCCCCTCGGCCCCCAACTCCTGCACCAACTATCGCTTCCCAA
CCCCTGTCTCTGAGACCTGAGGCATGCCGCCCCGCGGCAGGCGGCGCCGT
GCACACTAGAGGCCTGGACTTCGCCTGCGATATTTATATCTGGGCCCCCC
TTGCCGGGACATGCGGGGTACTGCTGCTGTCTCTGGTGATTACCCTCTAC
TGCAACCACAGAAACAGATCCAAAAGAAGCCGCCTGCTCCATAGCGATTA
CATGAATATGACTCCACGCCGCCCTGGCCCCACAAGGAAACACTACCAGC
CTTACGCACCACCTAGAGATTTCGCTGCCTATCGGAGCAGGGTGAAGTTT
TCCAGATCTGCAGATGCACCAGCGTATCAGCAGGGCCAGAACCAACTGTA
TAACGAGCTCAACCTGGGACGCAGGGAAGAGTATGACGTTTTGGACAAGC
GCAGAGGACGGGACCCTGAGATGGGTGGCAAACCAAGACGAAAAAACCCC
CAGGAGGGTCTCTATAATGAGCTGCAGAAGGATAAGATGGCTGAAGCCTA
TTCTGAAATAGGCATGAAAGGAGAGCGGAGAAGGGGAAAAGGGCACGACG
GTTTGTACCAGGGACTCAGCACTGCTACGAAGGATACTTATGACGCTCTC
CACATGCAAGCCCTGCCACCTAGGGCCAAGAGAAGTGGCAGCGGGGAGGG
CCGGGGATCTCTCCTTACATGTGGGGACGTGGAAGAAAATCCGGGGCCTA
TGGGTGCCGGCGCCACGGGAAGGGCTATGGATGGCCCGCGACTGCTTCTC
CTGCTGTTGTTGGGCGTGTCTCTCGGAGGCGCTAAGGAGGCCTGTCCAAC
GGGCCTCTACACTCACTCCGGTGAATGTTGCAAAGCCTGTAACCTTGGCG
AGGGCGTCGCACAACCTTGTGGTGCTAACCAGACAGTCTGTGAACCATGC
CTGGATTCAGTGACATTCAGCGATGTTGTCTCAGCCACCGAGCCTTGCAA
GCCTTGTACCGAATGTGTGGGCCTTCAGTCCATGTCCGCCCCCTGTGTCG
AAGCCGATGATGCAGTGTGCAGATGTGCCTATGGATATTACCAGGACGAA
ACTACCGGGCGGTGTGAGGCCTGCCGGGTGTGTGAAGCCGGCTCTGGCCT
CGTGTTCAGTTGCCAGGATAAGCAAAACACAGTATGTGAGGAGTGTCCAG
ACGGAACCTACAGCGACGAGGCGAACCACGTCGACCCTTGCTTGCCGTGC
ACCGTCTGCGAGGATACCGAACGCCAGCTGAGAGAGTGTACGCGCTGGGC
AGACGCTGAGTGCGAGGAGATCCCTGGGAGATGGATCACCCGGAGCACAC
CTCCTGAGGGATCAGACAGTACAGCCCCGAGTACCCAAGAACCGGAGGCC
CCTCCAGAGCAGGACCTGATCGCTTCTACAGTTGCTGGCGTGGTGACGAC
AGTCATGGGATCCTCACAACCAGTCGTGACGCGGGGCACAACCGACAATC
TGATTCCTGTCTACTGTAGCATCTTGGCAGCCGTGGTCGTGGGCCTGGTA
GCCTACATCGCCTTTAAGAGATGACCTAGGTAA

Construct S2-14C1-C8K-CD28 AA (Signal Sequence in Bold; CDRs Underlined)

(SEQ ID NO: 74)
MALPVTALLLPLALLLHAARPQVQLVQSGAEVKKPGASVKVSCKASGYTF
TGYYMHWVRQAPGQGLEWMGWINPNSGGTNSAQKFQGRVTMTRDTSISTA
YMELNRLRSDDTAVYYCARGWLQTYYFDNWGQGTLVTVSSGGGGSGGGGS
GGGGSDIVMTQSPDSLAVSLGERATIYCKSSQTVLTSSNNKNFLAWYQQK
LGQPPKLLISWASTRESGVPDRFSGSGSGTDFTLTISSLQAEDVAIYYCQ
HYYTSPLTFGGGTKVEIKRAAAFVPVFLPAKPTTTPAPRPPTPAPTIASQ
PLSLRPEACRPAAGGAVHTRGLDFACDIYIWAPLAGTCGVLLLSLVITLY
CNHRNRSKRSRLLHSDYMNMTPRRPGPTRKHYQPYAPPRDFAAYRSRVKF
SRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDAL
HMQALPPRAKRSGSGEGRGSLLTCGDVEENPGPMGAGATGRAMDGPRLLL
LLLLGVSLGGAKEACPTGLYTHSGECCKACNLGEGVAQPCGANQTVCEPC
LDSVTFSDVVSATEPCKPCTECVGLQSMSAPCVEADDAVCRCAYGYYQDE
TTGRCEACRVCEAGSGLVFSCQDKQNTVCEECPDGTYSDEANHVDPCLPC
TVCEDTERQLRECTRWADAECEEIPGRWITRSTPPEGSDSTAPSTQEPEA
PPEQDLIASTVAGVVTTVMGSSQPVVTRGTTDNLIPVYCSILAAVVVGLV
AYIAFKR

Construct S2-14C1-C8K-41BB DNA (Signal Sequence in Bold)

(SEQ ID NO: 75)
ATGGCACTCCCCGTAACTGCTCTGCTGCTGCCGTTGGCATTGCTCCTGCA
CGCCGCACGCCCGCAGGTGCAGCTGGTGCAGTCTGGGGCTGAGGTGAAGA
AGCCTGGGGCCTCAGTGAAGGTGTCCTGCAAGGCTTCTGGATACACCTTC
ACCGGCTACTATATGCACTGGGTGCGACAGGCCCCTGGACAAGGGCTTGA
GTGGATGGGATGGATCAACCCTAATAGTGGTGGCACAAACTCTGCACAGA
AGTTTCAGGGCAGGGTCACCATGACCAGGGACACGTCCATCAGTACAGCC
TACATGGAGCTGAACAGGCTGAGATCTGACGACACGGCCGTTTATTACTG
TGCGAGAGGATGGCTACAGACGTACTACTTTGACAACTGGGGCCAGGGAA
CCCTGGTCACCGTATCCTCAGGAGGCGGCGGTTCAGGCGGAGGTGGCTCT
GGCGGTGGCGGAAGTGACATCGTGATGACCCAGTCTCCAGACTCCCTGGC
TGTGTCTCTGGGCGAGAGGGCCACCATCTACTGCAAGTCCAGCCAGACTG
TTTTGACCAGCTCCAACAATAAGAACTTCTTAGCTTGGTACCAACAGAAA
CTAGGACAGCCTCCTAAGCTGCTCATTTCCTGGGCCTCTACCCGGGAATC
CGGGGTCCCTGACCGATTCAGTGGCAGCGGGTCTGGGACAGATTTCACTC
TCACCATCAGCAGCCTGCAGGCTGAAGATGTGGCAATTTATTACTGTCAG
CACTATTATACTAGTCCACTCACTTTCGGCGGCGGGACCAAGGTGGAGAT
CAAACGAGCCGCTGCCTTCGTGCCTGTTTTTCTGCCCGCGAAACCCACAA
CTACCCCCGCCCCTCGGCCCCCAACTCCTGCACCAACTATCGCTTCCCAA
CCCCTGTCTCTGAGACCTGAGGCATGCCGCCCCGCGGCAGGCGGCGCCGT
GCACACTAGAGGCCTGGACTTCGCCTGCGATATTTATATCTGGGCCCCCC
TTGCCGGGACATGCGGGGTACTGCTGCTGTCTCTGGTGATTACCCTCTAC
TGCAACCACAGAAACCGCTTTTCCGTCGTTAAGCGGGGGAGAAAAAAGCT
GCTGTACATTTTCAAACAGCCGTTTATGAGGCCGGTCCAAACGACTCAGG
AAGAGGACGGCTGCTCCTGCCGCTTTCCTGAGGAGGAGGAGGGCGGGTGC
GAACTGAGGGTGAAGTTTTCCAGATCTGCAGATGCACCAGCGTATCAGCA
GGGCCAGAACCAACTGTATAACGAGCTCAACCTGGGACGCAGGGAAGAGT
ATGACGTTTTGGACAAGCGCAGAGGACGGGACCCTGAGATGGGTGGCAAA
CCAAGACGAAAAAACCCCCAGGAGGGTCTCTATAATGAGCTGCAGAAGGA
TAAGATGGCTGAAGCCTATTCTGAAATAGGCATGAAAGGAGAGCGGAGAA
GGGGAAAAGGGCACGACGGTTTGTACCAGGGACTCAGCACTGCTACGAAG
GATACTTATGACGCTCTCCACATGCAAGCCCTGCCACCTAGGGCCAAGAG
AAGTGGCAGCGGGGAGGGCCGGGGATCTCTCCTTACATGTGGGGACGTGG
AAGAAAATCCGGGGCCTATGGGTGCCGGCGCCACGGGAAGGGCTATGGAT
GGCCCGCGACTGCTTCTCCTGCTGTTGTTGGGCGTGTCTCTCGGAGGCGC
TAAGGAGGCCTGTCCAACGGGCCTCTACACTCACTCCGGTGAATGTTGCA
AAGCCTGTAACCTTGGCGAGGGCGTCGCACAACCTTGTGGTGCTAACCAG
ACAGTCTGTGAACCATGCCTGGATTCAGTGACATTCAGCGATGTTGTCTC
AGCCACCGAGCCTTGCAAGCCTTGTACCGAATGTGTGGGCCTTCAGTCCA
TGTCCGCCCCCTGTGTCGAAGCCGATGATGCAGTGTGCAGATGTGCCTAT
GGATATTACCAGGACGAAACTACCGGGCGGTGTGAGGCCTGCCGGGTGTG
TGAAGCCGGCTCTGGCCTCGTGTTCAGTTGCCAGGATAAGCAAAACACAG
TATGTGAGGAGTGTCCAGACGGAACCTACAGCGACGAGGCGAACCACGTC
GACCCTTGCTTGCCGTGCACCGTCTGCGAGGATACCGAACGCCAGCTGAG
AGAGTGTACGCGCTGGGCAGACGCTGAGTGCGAGGAGATCCCTGGGAGAT
GGATCACCCGGAGCACACCTCCTGAGGGATCAGACAGTACAGCCCCGAGT
ACCCAAGAACCGGAGGCCCCTCCAGAGCAGGACCTGATCGCTTCTACAGT
TGCTGGCGTGGTGACGACAGTCATGGGATCCTCACAACCAGTCGTGACGC
GGGGCACAACCGACAATCTGATTCCTGTCTACTGTAGCATCTTGGCAGCC
GTGGTCGTGGGCCTGGTAGCCTACATCGCCTTTAAGAGATGACCTAGGTA
A

Construct S2-14C1-C8K-41BB AA (Signal Sequence in Bold; CDRs Underlined)

(SEQ ID NO: 76)
MALPVTALLLPLALLLHAARPQVQLVQSGAEVKKPGASVKVSCKASGYTF
TGYYMHWVRQAPGQGLEWMGWINPNSGGTNSAQKFQGRVTMTRDTSISTA
YMELNRLRSDDTAVYYCARGWLQTYYFDNWGQGTLVTVSSGGGGSGGGGS
GGGGSDIVMTQSPDSLAVSLGERATIYCKSSQTVLTSSNNKNFLAWYQQK
LGQPPKLLISWASTRESGVPDRFSGSGSGTDFTLTISSLQAEDVAIYYCQ
HYYTSPLTFGGGTKVEIKRAAAFVPVFLPAKPTTTPAPRPPTPAPTIASQ
PLSLRPEACRPAAGGAVHTRGLDFACDIYIWAPLAGTCGVLLLSLVITLY
CNHRNRFSVVKRGRKKLLYIFKQPFMRPVQTTQEEDGCSCRFPEEEEGGC
ELRVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTATK
DTYDALHMQALPPRAKRSGSGEGRGSLLTCGDVEENPGPMGAGATGRAMD
GPRLLLLLLLGVSLGGAKEACPTGLYTHSGECCKACNLGEGVAQPCGANQ
TVCEPCLDSVTFSDVVSATEPCKPCTECVGLQSMSAPCVEADDAVCRCAY
GYYQDETTGRCEACRVCEAGSGLVFSCQDKQNTVCEECPDGTYSDEANHV
DPCLPCTVCEDTERQLRECTRWADAECEEIPGRWITRSTPPEGSDSTAPS
TQEPEAPPEQDLIASTVAGVVTTVMGSSQPVVTRGTTDNLIPVYCSILAA
VVVGLVAYIAFKR

Human STEAP1 NM_012449 (NP_036581) AA

(SEQ ID NO: 77)
MESRKDITNQEELWKMKPRRNLEEDDYLHKDTGETSMLKRPVLLHLHQTA
HADEFDCPSELQHTQELFPQWHLPIKIAAIIASLTFLYTLLREVIHPLAT
SHQQYFYKIPILVINKVLPMVSITLLALVYLPGVIAAIVQLHNGTKYKKF
PHWLDKWMLTRKQFGLLSFFFAVLHAIYSLSYPMRRSYRYKLLNWAYQQV
QQNKEDAWIEHDVWRMEIYVSLGIVGLAILALLAVTSIPSVSDSLTWREF
HYIQSKLGIVSLLLGTIHALIFAWNKWIDIKQFVWYTPPTFMIAVFLPIV
VLIFKSILFLPCLRKKILKIRHGWEDVTKINKTEICSQL

CAR Signal Peptide DNA

(SEQ ID NO: 78)
ATGGCACTCCCCGTAACTGCTCTGCTGCTGCCGTTGGCATTGCTCCTGCA
CGCCGCACGCCCG

CAR Signal Peptide:

(SEQ ID NO: 79)
MALPVTALLLPLALLLHAARP

scFv G4S linker DNA

(SEQ ID NO: 80)
GGCGGTGGAGGCTCCGGAGGGGGGGGCTCTGGCGGAGGGGGCTCC

scFv G4s Linker:

(SEQ ID NO: 81)
GGGGSGGGGSGGGGS

scFv Whitlow Linker DNA

(SEQ ID NO: 82)
GGGTCTACATCCGGCTCCGGGAAGCCCGGAAGTGGCGAAGGTAGTACAAA
GGGG

scFv Whitlow Linker:

(SEQ ID NO: 83)
GSTSGSGKPGSGEGSTKG

4-1BB Nucleic Acid Sequence (Intracellular Domain)

(SEQ ID NO: 84)
AAGCGCGGCAGGAAGAAGCTCCTCTACATTTTTAAGCAGCCTTTTATGAG
GCCCGTACAGACAACACAGGAGGAAGATGGCTGTAGCTGCAGATTTCCCG
AGGAGGAGGAAGGTGGGTGCGAGCTG

4-1BB AA (Intracellular Domain)

(SEQ ID NO: 85)
KRGRKKLLYIFKQPFMRPVQTTQEEDGCSCRFPEEEEGGCEL

OX40 AA

(SEQ ID NO: 86)
RRDQRLPPDAHKPPGGGSFRTPIQEEQADAHSTLAKI

### INCORPORATION BY REFERENCE

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention. To the extent that any of the definitions or terms provided in the references incorporated by reference differ from the terms and discussion provided herein, the present terms and definitions control.

### EQUIVALENTS

The foregoing written specification is considered to be sufficient to enable one skilled in the art to practice the invention. The foregoing description and examples detail certain preferred embodiments of the invention and describe the best mode contemplated by the inventors. It will be appreciated, however, that no matter how detailed the foregoing may appear in text, the invention may be practiced in many ways and the invention should be construed in accordance with the appended claims and any equivalents thereof.

The following examples, including the experiments conducted and results achieved, are provided for illustrative purposes only and are not to be construed as limiting the present invention.

### Example 1

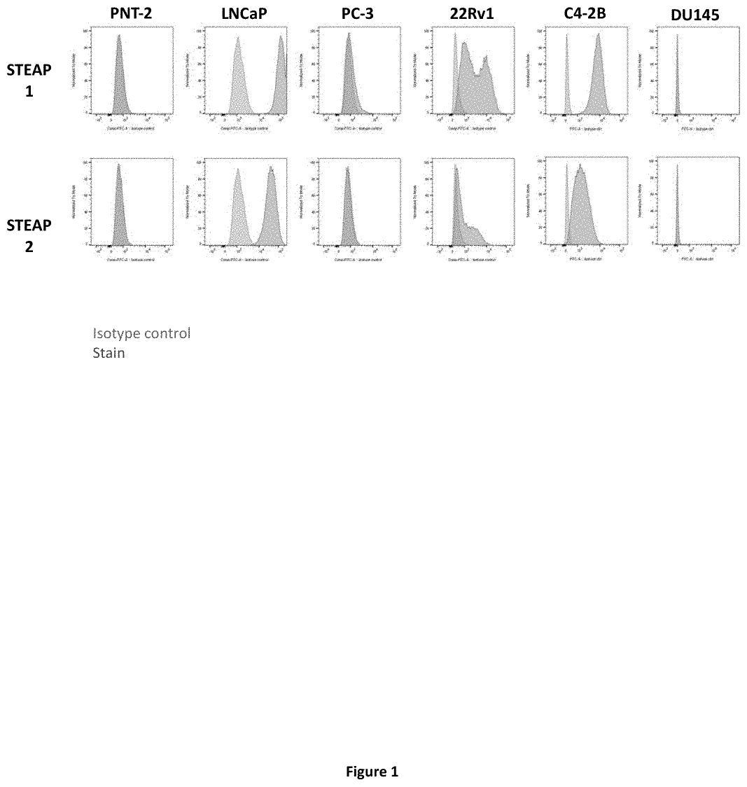

PNT-2, LNCaP, PC-3, 22Rv1, C4-2B, and DU145 cell lines were cultured in RPMI1640 (Lonza)+10% FBS (Corning)+1× Penicillin Streptomycin L-Glutamine (Corning) (R10) medium and maintained at a cell density between 0.5-2.0×10⁶ cells/ml. PNT-2 and DU145 are negative control cell lines. To examine cell surface STEAP1 expression, cells were incubated with an anti-STEAP1 antibody (2F3) or an IgG1 isotype control antibody (BD Pharmingen) in stain buffer (BD Pharmingen) for 30 minutes at 4° C. Cells were then washed and resuspended in stain buffer with propidium iodide (BD Pharmingen) prior to data acquisition. STEAP1 expression on target cells is shown in FIG. 1.

### Example 2

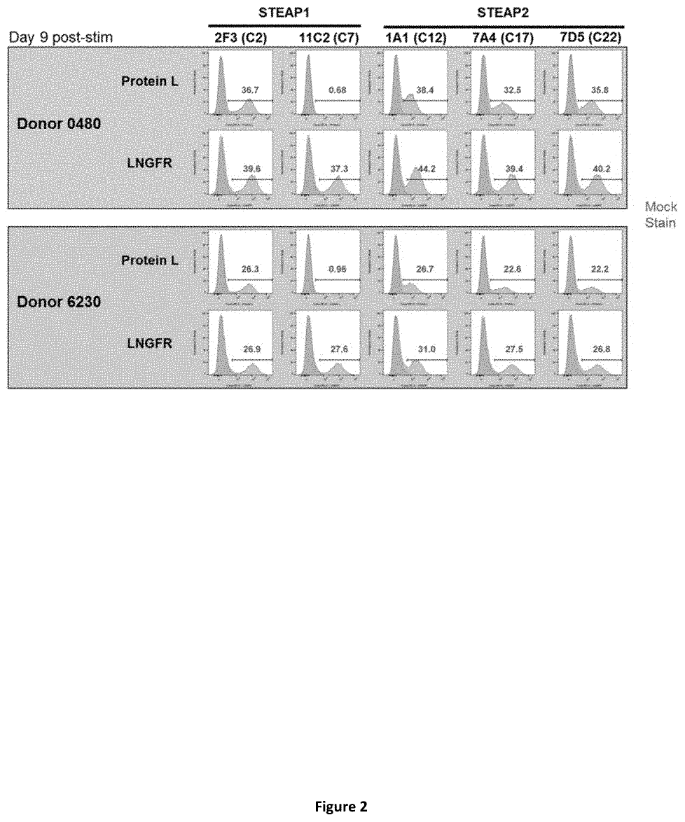

Plasmids encoding a T7 promoter, CAR construct and a beta globin stabilizing sequence were linearized by overnight digestion of 10 μgDNA with EcoRI and BamHI (NEB). DNA was then digested for 2 hours at 50° C. with proteinase K (Thermo Fisher, 600 U/ml) purified with phenol/chloroform and precipitated by adding sodium acetate and two volumes of ethanol. Pellets were then dried, resuspended in RNAse/DNAse-free water and quantified using NanoDrop. One μg of the linear DNA was then used for in vitro transcription using the mMESSAGE mMACHINE T7 Ultra (Thermo Fisher) following the manufacturer's instructions. RNA was further purified using the MEGAClear Kit (Thermo Fisher) following the manufacturer's instructions and quantified using NanoDrop. mRNA integrity was assessed using mobility on an agarose gel. PBMCs were isolated from healthy donor leukopaks (Hemacare) using ficoll-paque density centrifugation per manufacturer's instructions. PBMCs were stimulated using OKT3 (50 ng/ml, Miltenyi Biotec) in R10 medium+IL-2 (300 IU/ml, Proleukin®, Prometheus® Therapeutics and Diagnostics). Seven days post-stimulation, T cells were washed twice in Opti-MEM medium (Thermo Fisher Scientific) and resuspended at a final concentration of 2.5×10⁷ cells/ml in Opti-MEM medium. Ten μg of mRNA was used per electroporation. Electroporation of cells was performed using a Gemini X2 system (Harvard Apparatus BTX) to deliver a single 400 V pulse for 0.5 ms in 2 mm cuvettes (Harvard Apparatus BTX). Cells were immediately transferred to R10+IL-2 medium and allowed to recover for 6 hours. To examine CAR expression, T cells were stained with LNGFR or biotinylated Protein L (Thermo Scientific) in stain buffer (BD Pharmingen) for 30 minutes at 4° C. Cells were then washed and stained with anti-LNGFR-PE or PE Streptavidin (BD Pharmingen) in stain buffer for 30 minutes at 4° C. Cells were then washed and resuspended in stain buffer with propidium iodide (BD Pharmingen) prior to data acquisition. Expression of STEAP1 CARs in electroporated T cells is shown in FIG. 2.

### Example 3

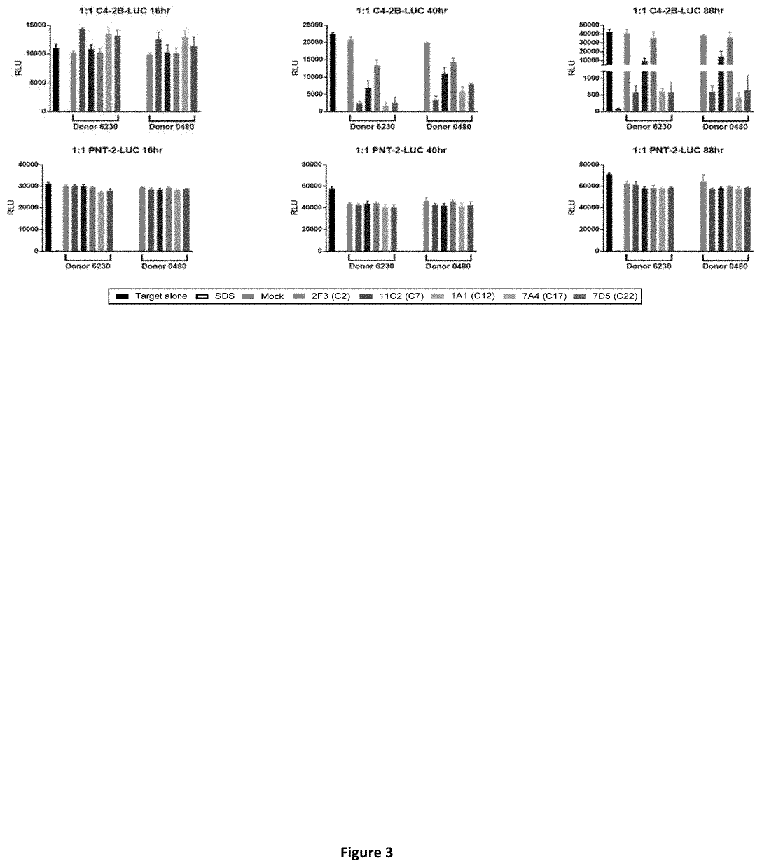

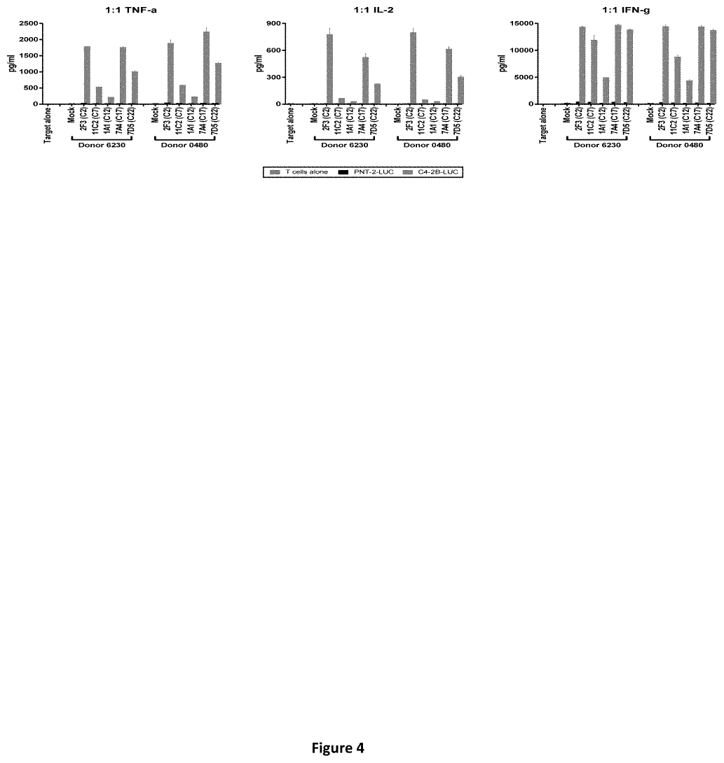

To examine cytolytic activity in electroporated STEAP1 CART cells, effector cells were cultured with target cells at a 1:1 E:T ratio in R10 medium. Sixteen hours post-coculture, supernatants were analyzed by Luminex (EMD Millipore) and target cell viability was assessed by flow cytometric analysis of propidium iodide (PI) uptake by CD3-negative cells. Cytolytic activity of electroporated CART cells is shown in FIG. 3 and cytokine production is shown in FIG. 4.

### Example 4

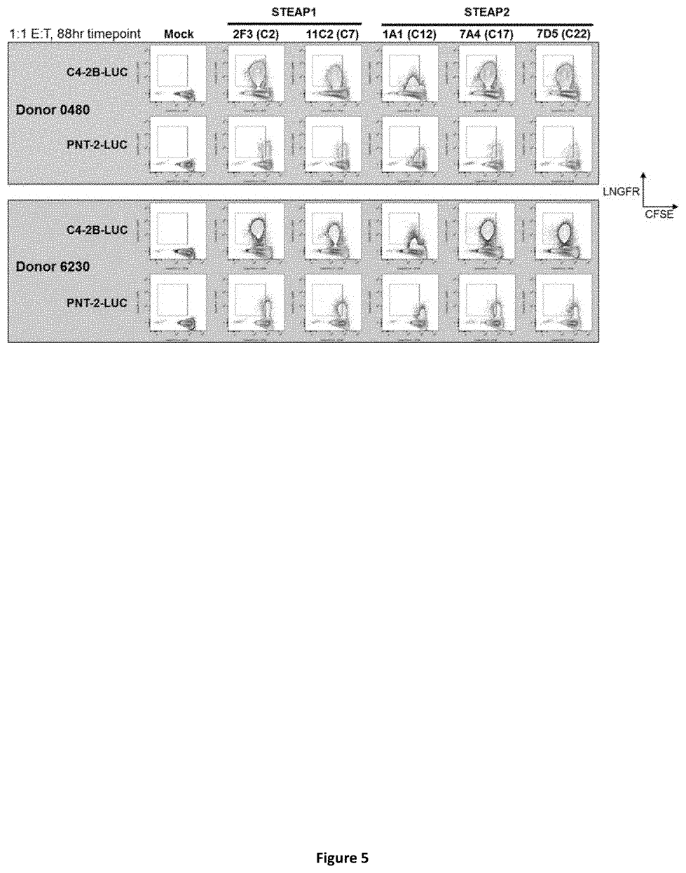

To assess CAR T cell proliferation in response to STEAP1 expressing target cells, T cells were labeled with CFSE prior to co-culture with target cells at a 1:1 E:T ratio in R10 medium. Five days later, T cell proliferation was assessed by flow cytometric analysis of CFSE dilution. Proliferation of STEAP1 CAR T cells is shown in FIG. 5.

1. A chimeric antigen receptor comprising:
(i) an antigen binding molecule that specifically binds to STEAP1, wherein the antigen binding molecule comprises:
a) a variable heavy chain CDR1 comprising an amino acid sequence differing by not more than 3, 2, 1, or 0 amino acid residues from that of SEQ ID Nos:89, 99, 109, 119, 129, or 139; or
b) a variable heavy chain CDR2 comprising an amino acid sequence differing by not more than 3, 2, 1, or 0 amino acid residues from that of SEQ ID Nos:90, 100, 110, 120, 130, or 140; or
c) a variable heavy chain CDR3 comprising an amino acid sequence differing by not more than 3, 2, 1, or 0 amino acid residues from that of SEQ ID Nos:91, 101, 111, 121, 131, or 141; or
d) a variable light chain CDR1 comprising an amino acid sequence differing by not more than 3, 2, 1, or 0 amino acid residues from that of SEQ ID Nos:94, 104, 114, 124, 134, or 144; or
e) a variable light chain CDR2 comprising an amino acid sequence differing by not more than 3, 2, 1, or 0 amino acid residues from that of SEQ ID Nos:95, 105, 115, 125, 135, or 145; or
f) a variable light chain CDR3 comprising an amino acid sequence differing by not more than 3, 2, 1, or 0 amino acid residues from that of SEQ IDs:96, 106, 116, 126, 136, or 146; or
g) a variable heavy chain CDR1 comprising an amino acid sequence of a variable heavy chain CDR1 sequence of clone 2F3, clone 11C2, clone 1A1, clone 7A4, or clone 7A5, or clone 14C1; or
h) a variable heavy chain CDR2 comprising an amino acid sequence of a variable heavy chain CDR2 sequence of clone 2F3, clone 11C2, clone 1A1, clone 7A4, or clone 7A5, or clone 14C1; or
i) a variable heavy chain CDR3 comprising an amino acid sequence of a variable heavy chain CDR3 sequence of clone 2F3, clone 11C2, clone 1A1, clone 7A4, or clone 7A5, or clone 14C1; or
j) a variable light chain CDR1 comprising an amino acid sequence of a variable light chain CDR1 sequence of clone 2F3, clone 11C2, clone 1A1, clone 7A4, or clone 7A5, or clone 14C1; or
k) a variable light chain CDR2 comprising an amino acid sequence of a variable light chain CDR2 sequence of clone 2F3, clone 11C2, clone 1A1, clone 7A4, or clone 7A5, or clone 14C1; or
l) a variable light chain CDR3 comprising an amino acid sequence of a variable light chain CDR3 sequence of clone 2F3, clone 11C2, clone 1A1, clone 7A4, or clone 7A5, or clone 14C1; or
m) a variable heavy chain sequence differing by not more than 10, 9, 8, 7, 6, 5, 4, 3, 2, 1, or 0 residues from the variable heavy chain sequence of clone 2F3, clone 11C2, clone 1A1, clone 7A4, or clone 7A5, or clone 14C1; or
n) a variable light chain sequence differing by not more than 10, 9, 8, 7, 6, 5, 4, 3, 2, 1, or 0 residues from the variable light chain sequence of clone 2F3, clone 11C2, clone 1A1, clone 7A4, or clone 7A5, or clone 14C1: or
(ii) at least one of:
(a) a VH region of clone 2F3 and a VL region of clone 2F3;
(b) a VH region of clone 11C2 and a VL region of clone 11C2;
€ a VH region of clone 1A1 and a VL region of clone 1A1;
(d) a VH region of clone 7A4 and a VL region of clone 7A4;
€ a VH region of clone 7A5 and a VL region of clone 7A5; or
(f) a VH region of clone 14C1 and a VL region of clone 14C1,
wherein the VH and VL region is linked by at least one linker.

2. The chimeric antigen receptor according to claim 1 further comprising at least one costimulatory domain, wherein the costimulatory domain is a signaling region of CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1 (CD1 1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class I molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

3. The chimeric antigen receptor according to claim 1 further comprising at least one activating domain.

4. (canceled)

5. (canceled)

6. The chimeric antigen receptor according to claim 2 wherein the CD28 costimulatory domain comprises a sequence that differs at no more than 10, 9, 8, 7, 6, 5, 4, 3, 2, 1, or 0 amino acid residues from the sequence of SEQ ID NO:2, SEQ ID NO:4, SEQ ID NO:6, or SEQ ID NO:8, or wherein the CD8 costimulatory domain comprises a sequence that differs at no more than 10, 9, 8, 7, 6, 5, 4, 3, 2, 1, or 0 amino acid residues from the sequence of SEQ ID NO:14.

7. (canceled)

8. The chimeric antigen receptor according to claim 3 wherein the activating domain comprises CD3, wherein the CD3 comprises CD3 zeta that comprises a sequence that differs at no more than 10, 9, 8, 7, 6, 5, 4, 3, 2, 1, or 0 amino acid residues from the sequence of SEQ ID NO:10.

9. (canceled)

10. (canceled)

11. The chimeric antigen receptor according to claim 1 wherein the costimulatory domain comprises a sequence that differs at no more than 10, 9, 8, 7, 6, 5, 4, 3, 2, 1, or 0 amino acid residues from the sequence of SEQ ID NO:2 and the activating domain comprises a sequence that differs at no more than 10, 9, 8, 7, 6, 5, 4, 3, 2, 1, or 0 amino acid residues from the sequence of SEQ ID NO:10.

12. A polynucleotide encoding the chimeric antigen receptor of claim 1.

13. A vector comprising the polynucleotide of claim 12, wherein the vector is a retroviral vector, a DNA vector, a plasmid, a RNA vector, an adenoviral vector, an adenovirus associated vector, a lentiviral vector, or any combination thereof.

14. (canceled)

15. An immune cell comprising the vector of claim 13, wherein the immune cell is a T cell, tumor infiltrating lymphocyte (TIL), NK cell, TCR-expressing cell, dendritic cell, or NK-T cell.

16-20. (canceled)

21. A pharmaceutical composition comprising an immune cell of claim 15.

22. (canceled)

23. The chimeric antigen receptor according to claim 1, wherein the linker comprises the scFv G45 linker or the scFv Whitlow linker.

24-37. (canceled)

38. An isolated polypeptide comprising the amino acid sequence of construct 2F3-CD28T-CD28-41BB, construct 2F3-CD28T-CD28, construct 2F3-CD28T-41BB, construct 2F3-C8K-CD28, construct 2F3-C8K-41BB, construct 11C2-CD28T-CD28-41BB, construct 11C2-CD28T-CD28, construct 11C2-CD28T-41BB, construct 11C2-C8K-CD28, construct 11C2-C8K-41BB, construct 1A1-CD28T-CD28-41BB, construct 1A1-CD28T-CD28, construct 1A1-CD28T-41BB, construct 1A1-C8K-CD28, construct 1A1-C8K-41BB, construct 7A4-CD28T-CD28-41BB, construct 7A4-CD28T-CD28, construct 7A4-CD28T-41BB, construct 7A4-C8K-CD28, construct 7A4-C8K-41BB, construct 7A5-CD28T-CD28-41BB, construct 7A5-CD28T-CD28, construct 7A5-CD28T-41BB, construct 7A5-C8K-CD28, construct 7A5-C8K-41BB, construct 14C1-CD28T-CD28-41BB, construct 14C1-CD28T-CD28, construct 14C1-CD28T-41BB, construct 14C1-C8K-CD28, or construct 14C1-C8K-41BB2F3 CD28.

39-43. (canceled)

44. An isolated polynucleotide encoding:
(i) a chimeric antigen receptor (CAR) or T cell receptor (TCR) comprising an antigen binding molecule that specifically binds to STEAP1, wherein the antigen binding molecule comprises a variable heavy chain CDR3 comprising the amino acid sequence of a variable heavy chain CDR3 of clone 2F3, clone 11C2, clone 1A1, clone 7A4, clone 7A5, or clone 14C1: or
(ii) a chimeric antigen receptor (CAR) or T cell receptor (TCR), said CAR or TCR comprising an antigen binding molecule that specifically binds to STEAP1, wherein the antigen binding molecule comprises:
a. a variable heavy chain sequence differing by not more than 10, 9, 8, 7, 6, 5, 4, 3, 2, 1, or 0 residues from the variable heavy chain sequence of clone 2F3, clone 11C2, clone 1A1, clone 7A4, or clone 7A5, or clone 14C1, and/or
a variable light chain sequence differing by not more than 10, 9, 8, 7, 6, 5, 4, 3, 2, 1, or 0 residues from the variable light chain sequence of clone 2F3, clone 11C2, clone 1A1, clone 7A4, or clone 7A5, or clone 14C1.

45. The polynucleotide according to claim 44 further comprising an activating domain, wherein the activating domain is CD3, wherein the CD3 is CD3 zeta.

46-48. (canceled)

49. The polynucleotide according to claim 44 further comprising a costimulatory domain, wherein the costimulatory domain is a signaling region of CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1 (CD1 1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class I molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

50-71. (canceled)

72. A method of treating a disease or disorder in a subject in need thereof comprising administering to the subject the chimeric antigen receptor according to claim 1.

73-76. (canceled)

77. The method according to any of claim 72, wherein the disease or disorder is cancer.

78. (canceled)

79. (canceled)


SEQUENCE LISTING (147 sequences)

SEQ ID NO: 1 - DNA, 294 bp, Unknown
  source: /note=“Description of Unknown CD28T sequence”
cttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagc

SEQ ID NO: 2 - protein, 98 aa, Unknown
  source: /note=“Description of Unknown CD28T sequence”
LDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - DNA, 90 bp, Unknown
  source: /note=“Description of Unknown CD28T sequence”
cttgataatgaaaagtcaaacggaacaatcattcacgtgaagggcaagcacctctgtccgtcacccttgttccctggtccatccaagcca

SEQ ID NO: 4 - protein, 30 aa, Unknown
  source: /note=“Description of Unknown CD28T sequence”
LDNEKSNGTIIHVKGKHLCPSPLFPGPSKP

SEQ ID NO: 5 - DNA, 81 bp, Unknown
  source: /note=“Description of Unknown CD28 sequence”
ttctgggtgttggtcgtagtgggtggagtcctcgcttgttactctctgctcgtcaccgtggcttttataatcttctgggtt

SEQ ID NO: 6 - protein, 27 aa, Unknown
  source: /note=“Description of Unknown CD28 sequence”
FWVLVVVGGVLACYSLLVTVAFIIFWV

SEQ ID NO: 7 - DNA, 123 bp, Unknown
  source: /note=“Description of Unknown CD28 sequence”
agatccaaaagaagccgcctgctccatagcgattacatgaatatgactccacgccgccctggccccacaaggaaacactaccagccttacgcaccacctagagatttcgctgcctatcggagc

SEQ ID NO: 8 - protein, 41 aa, Unknown
  source: /note=“Description of Unknown CD28 sequence”
RSKRSRLLHSDYMNMTPRRPGPTRKHYQPYAPPRDFAAYRS

SEQ ID NO: 9 - DNA, 336 bp, Unknown
  source: /note=“Description of Unknown CD3 zeta sequence”
agggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 10 - protein, 112 aa, Unknown
  source: /note=“Description of Unknown CD3 zeta sequence”
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 117 bp, Unknown
  source: /note=“Description of Unknown CD28 sequence”
attgaggtgatgtatccaccgccttacctggataacgaaaagagtaacggtaccatcattcacgtgaaaggtaaacacctgtgtccttctcccctcttccccgggccatcaaagccc

SEQ ID NO: 12 - protein, 39 aa, Unknown
  source: /note=“Description of Unknown CD28 sequence”
IEVMYPPPYLDNEKSNGTIIHVKGKHLCPSPLFPGPSKP

SEQ ID NO: 13 - DNA, 288 bp, Unknown
  source: /note=“Description of Unknown CD8 sequence”
gctgcagcattgagcaactcaataatgtattttagtcactttgtaccagtgttcttgccggctaagcctactaccacacccgctccacggccacctaccccagctcctaccatcgcttcacagcctctgtccctgcgcccagaggcttgccgaccggccgcagggggcgctgttcataccagaggactggatttcgcctgcgatatctatatctgggcacccctggccggaacctgcggcgtactcctgctgtccctggtcatcacgctctattgtaatcacaggaac

SEQ ID NO: 14 - protein, 96 aa, Unknown
  source: /note=“Description of Unknown CD8 sequence”
AAALSNSIMYFSHFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 15 - DNA, 141 bp, Unknown
  source: /note=“Description of Unknown 4-1BB sequence”
cgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactg

SEQ ID NO: 16 - protein, 47 aa, Unknown
  source: /note=“Description of Unknown 4-1BB sequence”
RFSVVKRGRKKLLYIFKQPFMRPVQTTQEEDGCSCRFPEEEEGGCEL

SEQ ID NO: 17 - DNA, 2477 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagagcggagccgaggtgaagaagcccggagccagcgtgaaggtgagctgcaaagccagcggctacaccttctccacctactggatcgagtgggtgagacaggccccggacagaggctggaatggatgggagagatcctgcccggcagcggcaacaccgacttcaacgagaagttccagggcagagtgaccttcaccgccgataccagcagcgacaccgcctacatggaactgagcagcctgagaagcgaggataccgccgtctactactgcaccagatggggctactacggcaccaggggctatttcaacgtgtggggccagggaaccctcgtgaccgtgagcagcggaggcggaggatctggtggcggtggttctggcggcggaggctccgagattgtgctgacccagagccctgctacactgagcctgagccccggcgagagagccacactgagctgcagagccagcagcagcgtgagctacatgcactggttccagcaaaagcccggccaggcccctaggctgctgatctacagcacatccaacctggccagcggcatccctgccagattcagcggttctggctccggcaccgactacaccctgaccatctccagcctggagcccgaggactttgccgtgtattactgccagcagaggaggagcttcccctacacattcggccagggcaccaaactggagatcaaggccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagccgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 18 - protein, 822 aa, Artificial Sequence
  source: /note=“Description of Artificial Sequence Synthetic polypeptide”
MALPVTALLLPLALLLHAARPQVQLVQSGAEVKKPGASVKVSCKASGYTFSTYWIEWVRQAPGQRLEWMGEILPGSGNTDFNEKFQGRVTFTADTSSDTAYMELSSLRSEDTAVYYCTRWGYYGTRGYFNVWGQGTLVTVSSGGGGSGGGGSGGGGSEIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIKAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 19 - DNA, 2337 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagagcggagccgaggtgaagaagcccggagccagcgtgaaggtgagctgcaaagccagcggctacaccttctccacctactggatcgagtgggtgagacaggcccccggacagaggctggaatggatgggagagatcctgcccggcagcggcaacaccgacttcaacgagaagttccagggcagagtgaccttcaccgccgataccagcagcgacaccgcctacatggaactgagcagcctgagaagcgaggataccgccgtctactactgcaccagatggggctactacggcaccaggggctatttcaacgtgtggggccagggaaccctcgtgaccgtgagcagcggaggcggaggatctggtggcggtggttctggcggcggaggctccgagattgtgctgacccagagccctgctacactgagcctgagccccggcgagagagccacactgagctgcagagccagcagcagcgtgagctacatgcactggttccagcaaaagcccggccaggcccctaggctgctgatctacagcacatccaacctggccagcggcatccctgccagattcagcggttctggctccggcaccgactacaccctgaccatctccagcctggagcccgaggactttgccgtgtattactgccagcagaggaggagcttcccctacacattcggccagggcaccaaactggagatcaaggccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 20 - protein, 775 aa, Artificial Sequence
  source: /note=“Description of Artificial Sequence Synthetic polypeptide”
MALPVTALLLPLALLLHAARPQVQLVQSGAEVKKPGASVKVSCKASGYTFSTYWIEWVRQAPGQRLEWMGEILPGSGNTDFNEKFQGRVTFTADTSSDTAYMELSSLRSEDTAVYYCTRWGYYGTRGYFNVWGQGTLVTVSSGGGGSGGGGSGGGGSEIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIKAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 21 - DNA, 2355 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagagcggagccgaggtgaagaagcccggagccagcgtgaaggtgagctgcaaagccagcggctacaccttctccacctactggatcgagtgggtgagacaggcccccggacagaggctggaatggatgggagagatcctgcccggcagcggcaacaccgacttcaacgagaagttccagggcagagtgaccttcaccgccgataccagcagcgacaccgcctacatggaactgagcagcctgagaagcgaggataccgccgtctactactgcaccagatggggctactacggcaccaggggctatttcaacgtgtggggccagggaaccctcgtgaccgtgagcagcggaggcggaggatctggtggcggtggttctggcggcggaggctccgagattgtgctgacccagagccctgctacactgagcctgagccccggcgagagagccacactgagctgcagagccagcagcagcgtgagctacatgcactggttccagcaaaagcccggccaggcccctaggctgctgatctacagcacatccaacctggccagcggcatccctgccagattcagcggttctggctccggcaccgactacaccctgaccatctccagcctggagcccgaggactttgccgtgtattactgccagcagaggaggagcttcccctacacattcggccagggcaccaaactggagatcaaggccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttcgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 22 - protein, 781 aa, Artificial Sequence
  source: /note=“Description of Artificial Sequence Synthetic polypeptide”
MALPVTALLLPLALLLHAARPQVQLVQSGAEVKKPGASVKVSCKASGYTFSTYWIEWVRQAPGQRLEWMGEILPGSGNTDFNEKFQGRVTFTADTSSDTAYMELSSLRSEDTAVYYCTRWGYYGTRGYFNVWGQGTLVTVSSGGGGSGGGGSGGGGSEIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIKAAALDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 23 - DNA, 2415 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagagcggagccgaggtgaagaagcccggagccagcgtgaaggtgagctgcaaagccagcggctacaccttctccacctactggatcgagtgggtgagacaggcccccggacagaggctggaatggatgggagagatcctgcccggcagcggcaacaccgacttcaacgagaagttccagggcagagtgaccttcaccgccgataccagcagcgacaccgcctacatggaactgagcagcctgagaagcgaggataccgccgtctactactgcaccagatggggctactacggcaccaggggctatttcaacgtgtggggccagggaaccctcgtgaccgtgagcagcggaggcggaggatctggtggcggtggttctggcggcggaggctccgagattgtgctgacccagagccctgctacactgagcctgagccccggcgagagagccacactgagctgcagagccagcagcagcgtgagctacatgcactggttccagcaaaagcccggccaggcccctaggctgctgatctacagcacatccaacctggccagcggcatccctgccagattcagcggttctggctccggcaccgactacaccctgaccatctccagcctggagcccgaggactttgccgtgtattactgccagcagaggaggagcttcccctacacattcggccagggcaccaaactggagatcaaggccgctgccttcgtgcctgtttttctgcccgcgaaacccacaactacccccgcccctcggcccccaactcctgcaccaactatcgcttcccaacccctgtctctgagacctgaggcatgccgccccgcggcaggcggcgccgtgcacactagaggcctggacttcgcctgcgatatttatatctgggccccccttgccgggacatgcggggtactgctgctgtctctggtgattaccctctactgcaaccacagaaacagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 24 - protein, 801 aa, Artificial Sequence
  source: /note=“Description of Artificial Sequence Synthetic polypeptide”
MALPVTALLLPLALLLHAARPQVQLVQSGAEVKKPGASVKVSCKASGYTFSTYWIEWVRQAPGQRLEWMGEILPGSGNTDFNEKFQGRVTFTADTSSDTAYMELSSLRSEDTAVYYCTRWGYYGTRGYFNVWGQGTLVTVSSGGGGSGGGGSGGGGSEIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIKAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 25 - DNA, 2433 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagagcggagccgaggtgaagaagcccggagccagcgtgaaggtgagctgcaaagccagcggctacaccttctccacctactggatcgagtgggtgagacaggcccccggacagaggctggaatggatgggagagatcctgcccggcagcggcaacaccgacttcaacgagaagttccagggcagagtgaccttcaccgccgataccagcagcgacaccgcctacatggaactgagcagcctgagaagcgaggataccgccgtctactactgcaccagatggggctactacggcaccaggggctatttcaacgtgtggggccagggaaccctcgtgaccgtgagcagcggaggcggaggatctggtggcggtggttctggcggcggaggctccgagattgtgctgacccagagccctgctacactgagcctgagccccggcgagagagccacactgagctgcagagccagcagcagcgtgagctacatgcactggttccagcaaaagcccggccaggcccctaggctgctgatctacagcacatccaacctggccagcggcatccctgccagattcagcggttctggctccggcaccgactacaccctgaccatctccagcctggagcccgaggactttgccgtgtattactgccagcagaggaggagcttcccctacacattcggccagggcaccaaactggagatcaaggccgctgccttcgtgcctgtttttctgcccgcgaaacccacaactacccccgcccctcggcccccaactcctgcaccaactatcgcttcccaacccctgtctctgagacctgaggcatgccgccccgcggcaggcggcgccgtgcacactagaggcctggacttcgcctgcgatatttatatctgggccccccttgccgggacatgcggggtactgctgctgtctctggtgattaccctctactgcaaccacagaaaccgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 26 - protein, 807 aa, Artificial Sequence
  source: /note=“Description of Artificial Sequence Synthetic polypeptide”
MALPVTALLLPLALLLHAARPQVQLVQSGAEVKKPGASVKVSCKASGYTFSTYWIEWVRQAPGQRLEWMGEILPGSGNTDFNEKFQGRVTFTADTSSDTAYMELSSLRSEDTAVYYCTRWGYYGTRGYFNVWGQGTLVTVSSGGGGSGGGGSGGGGSEIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIKAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 27 - DNA, 2493 bp, Artificial Sequence
  source: /note=“Description of Artificial Sequence Synthetic polynucleotide”
atggcactccccgtaactgctctgctgctgccgttggcattgctcctgcacgccgcacgcccggagattcagctggtgcagagcggcgccgaggtgaaaaagcccggagccaccgtgaagatcagctgcaaggccagcggctacacattcaccaactacggcatgaactgggtgcaacaggcccccggccagggactggagtggatgggctggatgaacacatacaccggcgagcccacctacgccgacaagttccagggcagggtgacattcacactcgataccagcgccagaaccgtgtatatggaactgagcagcctgaggagcgaggacaccgctgtgtacttctgcgcaagggctggaggccagctgagacctggcgctatggactactggggccagggcaccatggtgaccgtgagctccggaggcggaggatctggtggcggtggttctggcggcggaggctccgacatcgtgctgacccagacacctctctccctgtccgtgacccccggacagcctgcctccatttcctgtaaggcctcccagagcgtggactatgacggcgacagcttcatgaactggtacctgcagaagcccggacaacccccccagctgctgatctacgtggccagcaacctggagtccggcgtgcctgacaggttttccggctccggcagcggcaccgacttcaccctgaagatcagcagggtggaagccgaggacgtgggcgtgtactactgccagcagagcaacgaggagccccctaccttcggacagggcaccaagctggagatcaaggccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagccgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 28 - protein, 827 aa, Artificial Sequence
  source: /note=“Description of Artificial Sequence Synthetic polypeptide”
MALPVTALLLPLALLLHAARPEIQLVQSGAEVKKPGATVKISCKASGYTFTNYGMNWVQQAPGQGLEWMGWMNTYTGEPTYADKFQGRVTFTLDTSARTVYMELSSLRSEDTAVYFCARAGGQLRPGAMDYWGQGTMVTVSSGGGGSGGGGSGGGGSDIVLTQTPLSLSVTPGQPASISCKASQSVDYDGDSFMNWYLQKPGQPPQLLIYVASNLESGVPDRFSGSGSGTDFTLKISRVEAEDVGVYYCQQSNEEPPTFGQGTKLEIKAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 29 - DNA, 2352 bp, Artificial Sequence
  source: /note=“Description of Artificial Sequence Synthetic polynucleotide”
atggcactccccgtaactgctctgctgctgccgttggcattgctcctgcacgccgcacgcccggagattcagctggtgcagagcggcgccgaggtgaaaaagcccggagccaccgtgaagatcagctgcaaggccagcggctacacattcaccaactacggcatgaactgggtgcaacaggcccccggccagggactggagtggatgggctggatgaacacatacaccggcgagcccacctacgccgacaagttccagggcagggtgacattcacactcgataccagcgccagaaccgtgtatatggaactgagcagcctgaggagcgaggacaccgctgtgtacttctgcgcaagggctggaggccagctgagacctggcgctatggactactggggccagggcaccatggtgaccgtgagctccggaggcggaggatctggtggcggtggttctggcggcggaggctccgacatcgtgctgacccagacacctctctccctgtccgtgacccccggacagcctgcctccatttcctgtaaggcctcccagagcgtggactatgacggcgacagcttcatgaactggtacctgcagaagcccggacaacccccccagctgctgatctacgtggccagcaacctggagtccggcgtgcctgacaggttttccggctccggcagcggcaccgacttcaccctgaagatcagcagggtggaagccgaggacgtgggcgtgtactactgccagcagagcaacgaggagccccctaccttcggacagggcaccaagctggagatcaaggccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 30 - protein, 780 aa, Artificial Sequence
  source: /note=“Description of Artificial Sequence Synthetic polypeptide”
MALPVTALLLPLALLLHAARPEIQLVQSGAEVKKPGATVKISCKASGYTFTNYGMNWVQQAPGQGLEWMGWMNTYTGEPTYADKFQGRVTFTLDTSARTVYMELSSLRSEDTAVYFCARAGGQLRPGAMDYWGQGTMVTVSSGGGGSGGGGSGGGGSDIVLTQTPLSLSVTPGQPASISCKASQSVDYDGDSFMNWYLQKPGQPPQLLIYVASNLESGVPDRFSGSGSGTDFTLKISRVEAEDVGVYYCQQSNEEPPTFGQGTKLEIKAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 31 - DNA, 2370 bp, Artificial Sequence
  source: /note=“Description of Artificial Sequence Synthetic polynucleotide”
atggcactccccgtaactgctctgctgctgccgttggcattgctcctgcacgccgcacgcccggagattcagctggtgcagagcggcgccgaggtgaaaaagcccggagccaccgtgaagatcagctgcaaggccagcggctacacattcaccaactacggcatgaactgggtgcaacaggcccccggccagggactggagtggatgggctggatgaacacatacaccggcgagcccacctacgccgacaagttccagggcagggtgacattcacactcgataccagcgccagaaccgtgtatatggaactgagcagcctgaggagcgaggacaccgctgtgtacttctgcgcaagggctggaggccagctgagacctggcgctatggactactggggccagggcaccatggtgaccgtgagctccggaggcggaggatctggtggcggtggttctggcggcggaggctccgacatcgtgctgacccagacacctctctccctgtccgtgacccccggacagcctgcctccatttcctgtaaggcctcccagagcgtggactatgacggcgacagcttcatgaactggtacctgcagaagcccggacaacccccccagctgctgatctacgtggccagcaacctggagtccggcgtgcctgacaggttttccggctccggcagcggcaccgacttcaccctgaagatcagcagggtggaagccgaggacgtgggcgtgtactactgccagcagagcaacgaggagccccctaccttcggacagggcaccaagctggagatcaaggccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttcgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 32 - protein, 786 aa, Artificial Sequence
  source: /note=“Description of Artificial Sequence Synthetic polypeptide”
MALPVTALLLPLALLLHAARPEIQLVQSGAEVKKPGATVKISCKASGYTFTNYGMNWVQQAPGQGLEWMGWMNTYTGEPTYADKFQGRVTFTLDTSARTVYMELSSLRSEDTAVYFCARAGGQLRPGAMDYWGQGTMVTVSSGGGGSGGGGSGGGGSDIVLTQTPLSLSVTPGQPASISCKASQSVDYDGDSFMNWYLQKPGQPPQLLIYVASNLESGVPDRFSGSGSGTDFTLKISRVEAEDVGVYYCQQSNEEPPTFGQGTKLEIKAAALDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 33 - DNA, 2430 bp, Artificial Sequence
  source: /note=“Description of Artificial Sequence Synthetic polynucleotide”
atggcactccccgtaactgctctgctgctgccgttggcattgctcctgcacgccgcacgcccggagattcagctggtgcagagcggcgccgaggtgaaaaagcccggagccaccgtgaagatcagctgcaaggccagcggctacacattcaccaactacggcatgaactgggtgcaacaggcccccggccagggactggagtggatgggctggatgaacacatacaccggcgagcccacctacgccgacaagttccagggcagggtgacattcacactcgataccagcgccagaaccgtgtatatggaactgagcagcctgaggagcgaggacaccgctgtgtacttctgcgcaagggctggaggccagctgagacctggcgctatggactactggggccagggcaccatggtgaccgtgagctccggaggcggaggatctggtggcggtggttctggcggcggaggctccgacatcgtgctgacccagacacctctctccctgtccgtgacccccggacagcctgcctccatttcctgtaaggcctcccagagcgtggactatgacggcgacagcttcatgaactggtacctgcagaagcccggacaacccccccagctgctgatctacgtggccagcaacctggagtccggcgtgcctgacaggttttccggctccggcagcggcaccgacttcaccctgaagatcagcagggtggaagccgaggacgtgggcgtgtactactgccagcagagcaacgaggagccccctaccttcggacagggcaccaagctggagatcaaggccgctgccttcgtgcctgtttttctgcccgcgaaacccacaactacccccgcccctcggcccccaactcctgcaccaactatcgcttcccaacccctgtctctgagacctgaggcatgccgccccgcggcaggcggcgccgtgcacactagaggcctggacttcgcctgcgatatttatatctgggccccccttgccgggacatgcggggtactgctgctgtctctggtgattaccctctactgcaaccacagaaacagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 34 - protein, 806 aa, Artificial Sequence
  source: /note=“Description of Artificial Sequence Synthetic polypeptide”
MALPVTALLLPLALLLHAARPEIQLVQSGAEVKKPGATVKISCKASGYTFTNYGMNWVQQAPGQGLEWMGWMNTYTGEPTYADKFQGRVTFTLDTSARTVYMELSSLRSEDTAVYFCARAGGQLRPGAMDYWGQGTMVTVSSGGGGSGGGGSGGGGSDIVLTQTPLSLSVTPGQPASISCKASQSVDYDGDSFMNWYLQKPGQPPQLLIYVASNLESGVPDRFSGSGSGTDFTLKISRVEAEDVGVYYCQQSNEEPPTFGQGTKLEIKAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 35 - DNA, 2448 bp, Artificial Sequence
  source: /note=“Description of Artificial Sequence Synthetic polynucleotide”
atggcactccccgtaactgctctgctgctgccgttggcattgctcctgcacgccgcacgcccggagattcagctggtgcagagcggcgccgaggtgaaaaagcccggagccaccgtgaagatcagctgcaaggccagcggctacacattcaccaactacggcatgaactgggtgcaacaggcccccggccagggactggagtggatgggctggatgaacacatacaccggcgagcccacctacgccgacaagttccagggcagggtgacattcacactcgataccagcgccagaaccgtgtatatggaactgagcagcctgaggagcgaggacaccgctgtgtacttctgcgcaagggctggaggccagctgagacctggcgctatggactactggggccagggcaccatggtgaccgtgagctccggaggcggaggatctggtggcggtggttctggcggcggaggctccgacatcgtgctgacccagacacctctctccctgtccgtgacccccggacagcctgcctccatttcctgtaaggcctcccagagcgtggactatgacggcgacagcttcatgaactggtacctgcagaagcccggacaacccccccagctgctgatctacgtggccagcaacctggagtccggcgtgcctgacaggttttccggctccggcagcggcaccgacttcaccctgaagatcagcagggtggaagccgaggacgtgggcgtgtactactgccagcagagcaacgaggagccccctaccttcggacagggcaccaagctggagatcaaggccgctgccttcgtgcctgtttttctgcccgcgaaacccacaactacccccgcccctcggcccccaactcctgcaccaactatcgcttcccaacccctgtctctgagacctgaggcatgccgccccgcggcaggcggcgccgtgcacactagaggcctggacttcgcctgcgatatttatatctgggccccccttgccgggacatgcggggtactgctgctgtctctggtgattaccctctactgcaaccacagaaaccgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 36 - protein, 812 aa, Artificial Sequence
  source: /note=“Description of Artificial Sequence Synthetic polypeptide”
MALPVTALLLPLALLLHAARPEIQLVQSGAEVKKPGATVKISCKASGYTFTNYGMNWVQQAPGQGLEWMGWMNTYTGEPTYADKFQGRVTFTLDTSARTVYMELSSLRSEDTAVYFCARAGGQLRPGAMDYWGQGTMVTVSSGGGGSGGGGSGGGGSDIVLTQTPLSLSVTPGQPASISCKASQSVDYDGDSFMNWYLQKPGQPPQLLIYVASNLESGVPDRFSGSGSGTDFTLKISRVEAEDVGVYYCQQSNEEPPTFGQGTKLEIKAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 37 - DNA, 2481 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtccggcgccgaagtgaagaagcccggagccagcgtgaaggtgagctgcaaggcctccggctacaccttcaccacctactggatgcactgggtcagacaggctcccggacagggcctggaatggatgggcgaaatcaacccctcctccggcaggaccaactacaacgagaagttcaagaccagggtgaccatgaccagggacaccagcaccagcaccgtgtacatggagctgtccagcctgaggagcgaggacaccgccgtgtactactgtgccaagctgggacccggcccccagtactatgccatggactactggggccaaggcaccatggtgaccgtgagcagcggaggcggaggatctggtggcggtggttctggcggcggaggctccgacatccagatgacccagagcccctccagcctgtccgctagcgtgggcgacagggtcaccattacctgccacgccagccagaacatcaacgtgtggctgagctggtatcagcagaaacccggcaaggctcccaagctgctgatctacaaggccagcaagctgcacaccggcgtgcccagcaggtttagcggttctggctccggcaccgacttcaccctcaccatcagcagcctgcagcccgaagacttcgctacctactactgccagcagggacaaagctacccctggaccttcggccagggaaccaagctggaaatcaaggccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagccgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 38 - protein, 823 aa, Artificial Sequence
  source: /note=“Description of Artificial Sequence Synthetic polypeptide”
MALPVTALLLPLALLLHAARPQVQLVQSGAEVKKPGASVKVSCKASGYTFTTYWMHWVRQAPGQGLEWMGEINPSSGRTNYNEKFKTRVTMTRDTSTSTVYMELSSLRSEDTAVYYCAKLGPGPQYYAMDYWGQGTMVTVSSGGGGSGGGGSGGGGSDIQMTQSPSSLSASVGDRVTITCHASQNINVWLSWYQQKPGKAPKLLIYKASKLHTGVPSRFSGSGSGTDFTLTISSLQPEDFATYYCQQGQSYPWTFGQGTKLEIKAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 39 - DNA, 2340 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtccggcgccgaagtgaagaagcccggagccagcgtgaaggtgagctgcaaggcctccggctacaccttcaccacctactggatgcactgggtcagacaggctcccggacagggcctggaatggatgggcgaaatcaacccctcctccggcaggaccaactacaacgagaagttcaagaccagggtgaccatgaccagggacaccagcaccagcaccgtgtacatggagctgtccagcctgaggagcgaggacaccgccgtgtactactgtgccaagctgggacccggcccccagtactatgccatggactactggggccaaggcaccatggtgaccgtgagcagcggaggcggaggatctggtggcggtggttctggcggcggaggctccgacatccagatgacccagagcccctccagcctgtccgctagcgtgggcgacagggtcaccattacctgccacgccagccagaacatcaacgtgtggctgagctggtatcagcagaaacccggcaaggctcccaagctgctgatctacaaggccagcaagctgcacaccggcgtgcccagcaggtttagcggttctggctccggcaccgacttcaccctcaccatcagcagcctgcagcccgaagacttcgctacctactactgccagcagggacaaagctacccctggaccttcggccagggaaccaagctggaaatcaaggccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 40 - protein, 776 aa, Artificial Sequence
  source: /note=“Description of Artificial Sequence Synthetic polypeptide”
MALPVTALLLPLALLLHAARPQVQLVQSGAEVKKPGASVKVSCKASGYTFTTYWMHWVRQAPGQGLEWMGEINPSSGRTNYNEKFKTRVTMTRDTSTSTVYMELSSLRSEDTAVYYCAKLGPGPQYYAMDYWGQGTMVTVSSGGGGSGGGGSGGGGSDIQMTQSPSSLSASVGDRVTITCHASQNINVWLSWYQQKPGKAPKLLIYKASKLHTGVPSRFSGSGSGTDFTLTISSLQPEDFATYYCQQGQSYPWTFGQGTKLEIKAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 41 - DNA, 2358 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtccggcgccgaagtgaagaagcccggagccagcgtgaaggtgagctgcaaggcctccggctacaccttcaccacctactggatgcactgggtcagacaggctcccggacagggcctggaatggatgggcgaaatcaacccctcctccggcaggaccaactacaacgagaagttcaagaccagggtgaccatgaccagggacaccagcaccagcaccgtgtacatggagctgtccagcctgaggagcgaggacaccgccgtgtactactgtgccaagctgggacccggcccccagtactatgccatggactactggggccaaggcaccatggtgaccgtgagcagcggaggcggaggatctggtggcggtggttctggcggcggaggctccgacatccagatgacccagagcccctccagcctgtccgctagcgtgggcgacagggtcaccattacctgccacgccagccagaacatcaacgtgtggctgagctggtatcagcagaaacccggcaaggctcccaagctgctgatctacaaggccagcaagctgcacaccggcgtgcccagcaggtttagcggttctggctccggcaccgacttcaccctcaccatcagcagcctgcagcccgaagacttcgctacctactactgccagcagggacaaagctacccctggaccttcggccagggaaccaagctggaaatcaaggccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttcgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 42 - protein, 782 aa, Artificial Sequence
  source: /note=“Description of Artificial Sequence Synthetic polypeptide”
MALPVTALLLPLALLLHAARPQVQLVQSGAEVKKPGASVKVSCKASGYTFTTYWMHWVRQAPGQGLEWMGEINPSSGRTNYNEKFKTRVTMTRDTSTSTVYMELSSLRSEDTAVYYCAKLGPGPQYYAMDYWGQGTMVTVSSGGGGSGGGGSGGGGSDIQMTQSPSSLSASVGDRVTITCHASQNINVWLSWYQQKPGKAPKLLIYKASKLHTGVPSRFSGSGSGTDFTLTISSLQPEDFATYYCQQGQSYPWTFGQGTKLEIKAAALDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 43 - DNA, 2418 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtccggcgccgaagtgaagaagcccggagccagcgtgaaggtgagctgcaaggcctccggctacaccttcaccacctactggatgcactgggtcagacaggctcccggacagggcctggaatggatgggcgaaatcaacccctcctccggcaggaccaactacaacgagaagttcaagaccagggtgaccatgaccagggacaccagcaccagcaccgtgtacatggagctgtccagcctgaggagcgaggacaccgccgtgtactactgtgccaagctgggacccggcccccagtactatgccatggactactggggccaaggcaccatggtgaccgtgagcagcggaggcggaggatctggtggcggtggttctggcggcggaggctccgacatccagatgacccagagcccctccagcctgtccgctagcgtgggcgacagggtcaccattacctgccacgccagccagaacatcaacgtgtggctgagctggtatcagcagaaacccggcaaggctcccaagctgctgatctacaaggccagcaagctgcacaccggcgtgcccagcaggtttagcggttctggctccggcaccgacttcaccctcaccatcagcagcctgcagcccgaagacttcgctacctactactgccagcagggacaaagctacccctggaccttcggccagggaaccaagctggaaatcaaggccgctgccttcgtgcctgtttttctgcccgcgaaacccacaactacccccgcccctcggcccccaactcctgcaccaactatcgcttcccaacccctgtctctgagacctgaggcatgccgccccgcggcaggcggcgccgtgcacactagaggcctggacttcgcctgcgatatttatatctgggccccccttgccgggacatgcggggtactgctgctgtctctggtgattaccctctactgcaaccacagaaacagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 44 - protein, 802 aa, Artificial Sequence
  source: /note=“Description of Artificial Sequence Synthetic polypeptide”
MALPVTALLLPLALLLHAARPQVQLVQSGAEVKKPGASVKVSCKASGYTFTTYWMHWVRQAPGQGLEWMGEINPSSGRTNYNEKFKTRVTMTRDTSTSTVYMELSSLRSEDTAVYYCAKLGPGPQYYAMDYWGQGTMVTVSSGGGGSGGGGSGGGGSDIQMTQSPSSLSASVGDRVTITCHASQNINVWLSWYQQKPGKAPKLLIYKASKLHTGVPSRFSGSGSGTDFTLTISSLQPEDFATYYCQQGQSYPWTFGQGTKLEIKAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 45 - DNA, 2436 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtccggcgccgaagtgaagaagcccggagccagcgtgaaggtgagctgcaaggcctccggctacaccttcaccacctactggatgcactgggtcagacaggctcccggacagggcctggaatggatgggcgaaatcaacccctcctccggcaggaccaactacaacgagaagttcaagaccagggtgaccatgaccagggacaccagcaccagcaccgtgtacatggagctgtccagcctgaggagcgaggacaccgccgtgtactactgtgccaagctgggacccggcccccagtactatgccatggactactggggccaaggcaccatggtgaccgtgagcagcggaggcggaggatctggtggcggtggttctggcggcggaggctccgacatccagatgacccagagcccctccagcctgtccgctagcgtgggcgacagggtcaccattacctgccacgccagccagaacatcaacgtgtggctgagctggtatcagcagaaacccggcaaggctcccaagctgctgatctacaaggccagcaagctgcacaccggcgtgcccagcaggtttagcggttctggctccggcaccgacttcaccctcaccatcagcagcctgcagcccgaagacttcgctacctactactgccagcagggacaaagctacccctggaccttcggccagggaaccaagctggaaatcaaggccgctgccttcgtgcctgtttttctgcccgcgaaacccacaactacccccgcccctcggcccccaactcctgcaccaactatcgcttcccaacccctgtctctgagacctgaggcatgccgccccgcggcaggcggcgccgtgcacactagaggcctggacttcgcctgcgatatttatatctgggccccccttgccgggacatgcggggtactgctgctgtctctggtgattaccctctactgcaaccacagaaaccgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 46 - protein, 808 aa, Artificial Sequence
  source: /note=“Description of Artificial Sequence Synthetic polypeptide”
MALPVTALLLPLALLLHAARPQVQLVQSGAEVKKPGASVKVSCKASGYTFTTYWMHWVRQAPGQGLEWMGEINPSSGRTNYNEKFKTRVTMTRDTSTSTVYMELSSLRSEDTAVYYCAKLGPGPQYYAMDYWGQGTMVTVSSGGGGSGGGGSGGGGSDIQMTQSPSSLSASVGDRVTITCHASQNINVWLSWYQQKPGKAPKLLIYKASKLHTGVPSRFSGSGSGTDFTLTISSLQPEDFATYYCQQGQSYPWTFGQGTKLEIKAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 47 - DNA, 2481 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtctggggctgaggtgaagaagcctggggcctcagtgaaggtgtcctgcaaggcttctggatacagcttcaccagttatgatatcaactgggtgcgacaggccactggacaagggcttgagtggatggggtggatgaacccgaacagtggtaacacaggctatgcacagaagttccagggcagagtcaccatgaccagggacacctccataagcacagcctacatggaactgagcagcctgagatctgaggacacggccgtgtattactgtgggagagccggttactactactacttcggtatggacgtctggggccaagggaccacggtcaccgtatcctcaggaggcggcggttcaggcggaggtggctctggcggtggcggaagtgaaattgtgttgacgcagtctccaggcaccctgtctttgtctccaggggaaagagccaccctctcctgcagggccggtcagagtgttaccagcagctcctttgcttggtaccaacagaaacctggccaggctcccaggctcctcatctatcagacatccaccagggccactggcatcccagacaggttcagtggcagtgggtctgggacagatttcactctcaccatcagcagactggagcctgaagattttgcagtgtattactgtcagcagtatggtggctcacggtcgttcggccaagggaccaaggtggaactcaaacgagccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagccgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 48 - protein, 823 aa, Artificial Sequence
  source: /note=“Description of Artificial Sequence Synthetic polypeptide”
MALPVTALLLPLALLLHAARPQVQLVQSGAEVKKPGASVKVSCKASGYSFTSYDINWVRQATGQGLEWMGWMNPNSGNTGYAQKFQGRVTMTRDTSISTAYMELSSLRSEDTAVYYCGRAGYYYYFGMDVWGQGTTVTVSSGGGGSGGGGSGGGGSEIVLTQSPGTLSLSPGERATLSCRAGQSVTSSSFAWYQQKPGQAPRLLIYQTSTRATGIPDRFSGSGSGTDFTLTISRLEPEDFAVYYCQQYGGSRSFGQGTKVELKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 49 - DNA, 2340 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtctggggctgaggtgaagaagcctggggcctcagtgaaggtgtcctgcaaggcttctggatacagcttcaccagttatgatatcaactgggtgcgacaggccactggacaagggcttgagtggatggggtggatgaacccgaacagtggtaacacaggctatgcacagaagttccagggcagagtcaccatgaccagggacacctccataagcacagcctacatggaactgagcagcctgagatctgaggacacggccgtgtattactgtgggagagccggttactactactacttcggtatggacgtctggggccaagggaccacggtcaccgtatcctcaggaggcggcggttcaggcggaggtggctctggcggtggcggaagtgaaattgtgttgacgcagtctccaggcaccctgtctttgtctccaggggaaagagccaccctctcctgcagggccggtcagagtgttaccagcagctcctttgcttggtaccaacagaaacctggccaggctcccaggctcctcatctatcagacatccaccagggccactggcatcccagacaggttcagtggcagtgggtctgggacagatttcactctcaccatcagcagactggagcctgaagattttgcagtgtattactgtcagcagtatggtggctcacggtcgttcggccaagggaccaaggtggaactcaaacgagccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 50 - protein, 776 aa, Artificial Sequence
  source: /note=“Description of Artificial Sequence Synthetic polypeptide”
MALPVTALLLPLALLLHAARPQVQLVQSGAEVKKPGASVKVSCKASGYSFTSYDINWVRQATGQGLEWMGWMNPNSGNTGYAQKFQGRVTMTRDTSISTAYMELSSLRSEDTAVYYCGRAGYYYYFGMDVWGQGTTVTVSSGGGGSGGGGSGGGGSEIVLTQSPGTLSLSPGERATLSCRAGQSVTSSSFAWYQQKPGQAPRLLIYQTSTRATGIPDRFSGSGSGTDFTLTISRLEPEDFAVYYCQQYGGSRSFGQGTKVELKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 51 - DNA, 2358 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtctggggctgaggtgaagaagcctggggcctcagtgaaggtgtcctgcaaggcttctggatacagcttcaccagttatgatatcaactgggtgcgacaggccactggacaagggcttgagtggatggggtggatgaacccgaacagtggtaacacaggctatgcacagaagttccagggcagagtcaccatgaccagggacacctccataagcacagcctacatggaactgagcagcctgagatctgaggacacggccgtgtattactgtgggagagccggttactactactacttcggtatggacgtctggggccaagggaccacggtcaccgtatcctcaggaggcggcggttcaggcggaggtggctctggcggtggcggaagtgaaattgtgttgacgcagtctccaggcaccctgtctttgtctccaggggaaagagccaccctctcctgcagggccggtcagagtgttaccagcagctcctttgcttggtaccaacagaaacctggccaggctcccaggctcctcatctatcagacatccaccagggccactggcatcccagacaggttcagtggcagtgggtctgggacagatttcactctcaccatcagcagactggagcctgaagattttgcagtgtattactgtcagcagtatggtggctcacggtcgttcggccaagggaccaaggtggaactcaaacgagccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttcgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 52 - protein, 782 aa, Artificial Sequence
  source: /note=“Description of Artificial Sequence Synthetic polypeptide”
MALPVTALLLPLALLLHAARPQVQLVQSGAEVKKPGASVKVSCKASGYSFTSYDINWVRQATGQGLEWMGWMNPNSGNTGYAQKFQGRVTMTRDTSISTAYMELSSLRSEDTAVYYCGRAGYYYYFGMDVWGQGTTVTVSSGGGGSGGGGSGGGGSEIVLTQSPGTLSLSPGERATLSCRAGQSVTSSSFAWYQQKPGQAPRLLIYQTSTRATGIPDRFSGSGSGTDFTLTISRLEPEDFAVYYCQQYGGSRSFGQGTKVELKRAAALDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 53 - DNA, 2418 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtctggggctgaggtgaagaagcctggggcctcagtgaaggtgtcctgcaaggcttctggatacagcttcaccagttatgatatcaactgggtgcgacaggccactggacaagggcttgagtggatggggtggatgaacccgaacagtggtaacacaggctatgcacagaagttccagggcagagtcaccatgaccagggacacctccataagcacagcctacatggaactgagcagcctgagatctgaggacacggccgtgtattactgtgggagagccggttactactactacttcggtatggacgtctggggccaagggaccacggtcaccgtatcctcaggaggcggcggttcaggcggaggtggctctggcggtggcggaagtgaaattgtgttgacgcagtctccaggcaccctgtctttgtctccaggggaaagagccaccctctcctgcagggccggtcagagtgttaccagcagctcctttgcttggtaccaacagaaacctggccaggctcccaggctcctcatctatcagacatccaccagggccactggcatcccagacaggttcagtggcagtgggtctgggacagatttcactctcaccatcagcagactggagcctgaagattttgcagtgtattactgtcagcagtatggtggctcacggtcgttcggccaagggaccaaggtggaactcaaacgagccgctgccttcgtgcctgtttttctgcccgcgaaacccacaactacccccgcccctcggcccccaactcctgcaccaactatcgcttcccaacccctgtctctgagacctgaggcatgccgccccgcggcaggcggcgccgtgcacactagaggcctggacttcgcctgcgatatttatatctgggccccccttgccgggacatgcggggtactgctgctgtctctggtgattaccctctactgcaaccacagaaacagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 54 - protein, 802 aa, Artificial Sequence
  source: /note=“Description of Artificial Sequence Synthetic polypeptide”
MALPVTALLLPLALLLHAARPQVQLVQSGAEVKKPGASVKVSCKASGYSFTSYDINWVRQATGQGLEWMGWMNPNSGNTGYAQKFQGRVTMTRDTSISTAYMELSSLRSEDTAVYYCGRAGYYYYFGMDVWGQGTTVTVSSGGGGSGGGGSGGGGSEIVLTQSPGTLSLSPGERATLSCRAGQSVTSSSFAWYQQKPGQAPRLLIYQTSTRATGIPDRFSGSGSGTDFTLTISRLEPEDFAVYYCQQYGGSRSFGQGTKVELKRAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 55 - DNA, 2436 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtctggggctgaggtgaagaagcctggggcctcagtgaaggtgtcctgcaaggcttctggatacagcttcaccagttatgatatcaactgggtgcgacaggccactggacaagggcttgagtggatggggtggatgaacccgaacagtggtaacacaggctatgcacagaagttccagggcagagtcaccatgaccagggacacctccataagcacagcctacatggaactgagcagcctgagatctgaggacacggccgtgtattactgtgggagagccggttactactactacttcggtatggacgtctggggccaagggaccacggtcaccgtatcctcaggaggcggcggttcaggcggaggtggctctggcggtggcggaagtgaaattgtgttgacgcagtctccaggcaccctgtctttgtctccaggggaaagagccaccctctcctgcagggccggtcagagtgttaccagcagctcctttgcttggtaccaacagaaacctggccaggctcccaggctcctcatctatcagacatccaccagggccactggcatcccagacaggttcagtggcagtgggtctgggacagatttcactctcaccatcagcagactggagcctgaagattttgcagtgtattactgtcagcagtatggtggctcacggtcgttcggccaagggaccaaggtggaactcaaacgagccgctgccttcgtgcctgtttttctgcccgcgaaacccacaactacccccgcccctcggcccccaactcctgcaccaactatcgcttcccaacccctgtctctgagacctgaggcatgccgccccgcggcaggcggcgccgtgcacactagaggcctggacttcgcctgcgatatttatatctgggccccccttgccgggacatgcggggtactgctgctgtctctggtgattaccctctactgcaaccacagaaaccgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 56 - protein, 808 aa, Artificial Sequence
  source: /note=“Description of Artificial Sequence Synthetic polypeptide”
MALPVTALLLPLALLLHAARPQVQLVQSGAEVKKPGASVKVSCKASGYSFTSYDINWVRQATGQGLEWMGWMNPNSGNTGYAQKFQGRVTMTRDTSISTAYMELSSLRSEDTAVYYCGRAGYYYYFGMDVWGQGTTVTVSSGGGGSGGGGSGGGGSEIVLTQSPGTLSLSPGERATLSCRAGQSVTSSSFAWYQQKPGQAPRLLIYQTSTRATGIPDRFSGSGSGTDFTLTISRLEPEDFAVYYCQQYGGSRSFGQGTKVELKRAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 57 - DNA, 2481 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtctggggctgaggtgaagaagcctggggcctcagtgaaggtgtcctgcaaggcttctggatacagcttcaccagttatgatatcaactgggtgcgacaggccactggacaagggcttgagtggatggggtggatgaacccgaacagtggtaacacaggctatgcacagaagttccagggcagagtcaccatgaccagggacacctccataagcacagcctacatggaactgagcagcctgagatctgaggacacggccgtgtattactgtgggagagccggttactactactacttcggtatggacgtctggggccaagggaccacggtcaccgtatcctcaggaggcggcggttcaggcggaggtggctctggcggtggcggaagtgaaattgtgttgacgcagtctccaggcaccctgtctttgtctccaggggaaagagccaccctctcctgcagggccggtcagagtgttaccagcagctccttagcttggtaccagcagaaacctggccaggctcccaggctcctcatctatcagacatccaccagggccactggcatcccagacaggttcagtggcagtgggtctgggacagatttcactctcaccatcagcagactggagcctgaagattttgcagtgtattactgtcagcagtatggtggctcacgggcgttcggccaagggaccaaggtggaactcaaacgagccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagccgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 58 - protein, 823 aa, Artificial Sequence
  source: /note=“Description of Artificial Sequence Synthetic polypeptide”
MALPVTALLLPLALLLHAARPQVQLVQSGAEVKKPGASVKVSCKASGYSFTSYDINWVRQATGQGLEWMGWMNPNSGNTGYAQKFQGRVTMTRDTSISTAYMELSSLRSEDTAVYYCGRAGYYYYFGMDVWGQGTTVTVSSGGGGSGGGGSGGGGSEIVLTQSPGTLSLSPGERATLSCRAGQSVTSSSLAWYQQKPGQAPRLLIYQTSTRATGIPDRFSGSGSGTDFTLTISRLEPEDFAVYYCQQYGGSRAFGQGTKVELKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 59 - DNA, 2340 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtctggggctgaggtgaagaagcctggggcctcagtgaaggtgtcctgcaaggcttctggatacagcttcaccagttatgatatcaactgggtgcgacaggccactggacaagggcttgagtggatggggtggatgaacccgaacagtggtaacacaggctatgcacagaagttccagggcagagtcaccatgaccagggacacctccataagcacagcctacatggaactgagcagcctgagatctgaggacacggccgtgtattactgtgggagagccggttactactactacttcggtatggacgtctggggccaagggaccacggtcaccgtatcctcaggaggcggcggttcaggcggaggtggctctggcggtggcggaagtgaaattgtgttgacgcagtctccaggcaccctgtctttgtctccaggggaaagagccaccctctcctgcagggccggtcagagtgttaccagcagctccttagcttggtaccagcagaaacctggccaggctcccaggctcctcatctatcagacatccaccagggccactggcatcccagacaggttcagtggcagtgggtctgggacagatttcactctcaccatcagcagactggagcctgaagattttgcagtgtattactgtcagcagtatggtggctcacgggcgttcggccaagggaccaaggtggaactcaaacgagccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 60 - protein, 776 aa, Artificial Sequence
  source: /note=“Description of Artificial Sequence Synthetic polypeptide”
MALPVTALLLPLALLLHAARPQVQLVQSGAEVKKPGASVKVSCKASGYSFTSYDINWVRQATGQGLEWMGWMNPNSGNTGYAQKFQGRVTMTRDTSISTAYMELSSLRSEDTAVYYCGRAGYYYYFGMDVWGQGTTVTVSSGGGGSGGGGSGGGGSEIVLTQSPGTLSLSPGERATLSCRAGQSVTSSSLAWYQQKPGQAPRLLIYQTSTRATGIPDRFSGSGSGTDFTLTISRLEPEDFAVYYCQQYGGSRAFGQGTKVELKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 61 - DNA, 2358 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtctggggctgaggtgaagaagcctggggcctcagtgaaggtgtcctgcaaggcttctggatacagcttcaccagttatgatatcaactgggtgcgacaggccactggacaagggcttgagtggatggggtggatgaacccgaacagtggtaacacaggctatgcacagaagttccagggcagagtcaccatgaccagggacacctccataagcacagcctacatggaactgagcagcctgagatctgaggacacggccgtgtattactgtgggagagccggttactactactacttcggtatggacgtctggggccaagggaccacggtcaccgtatcctcaggaggcggcggttcaggcggaggtggctctggcggtggcggaagtgaaattgtgttgacgcagtctccaggcaccctgtctttgtctccaggggaaagagccaccctctcctgcagggccggtcagagtgttaccagcagctccttagcttggtaccagcagaaacctggccaggctcccaggctcctcatctatcagacatccaccagggccactggcatcccagacaggttcagtggcagtgggtctgggacagatttcactctcaccatcagcagactggagcctgaagattttgcagtgtattactgtcagcagtatggtggctcacgggcgttcggccaagggaccaaggtggaactcaaacgagccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttcgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 62 - protein, 782 aa, Artificial Sequence
  source: /note=“Description of Artificial Sequence Synthetic polypeptide”
MALPVTALLLPLALLLHAARPQVQLVQSGAEVKKPGASVKVSCKASGYSFTSYDINWVRQATGQGLEWMGWMNPNSGNTGYAQKFQGRVTMTRDTSISTAYMELSSLRSEDTAVYYCGRAGYYYYFGMDVWGQGTTVTVSSGGGGSGGGGSGGGGSEIVLTQSPGTLSLSPGERATLSCRAGQSVTSSSLAWYQQKPGQAPRLLIYQTSTRATGIPDRFSGSGSGTDFTLTISRLEPEDFAVYYCQQYGGSRAFGQGTKVELKRAAALDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 63 - DNA, 2418 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtctggggctgaggtgaagaagcctggggcctcagtgaaggtgtcctgcaaggcttctggatacagcttcaccagttatgatatcaactgggtgcgacaggccactggacaagggcttgagtggatggggtggatgaacccgaacagtggtaacacaggctatgcacagaagttccagggcagagtcaccatgaccagggacacctccataagcacagcctacatggaactgagcagcctgagatctgaggacacggccgtgtattactgtgggagagccggttactactactacttcggtatggacgtctggggccaagggaccacggtcaccgtatcctcaggaggcggcggttcaggcggaggtggctctggcggtggcggaagtgaaattgtgttgacgcagtctccaggcaccctgtctttgtctccaggggaaagagccaccctctcctgcagggccggtcagagtgttaccagcagctccttagcttggtaccagcagaaacctggccaggctcccaggctcctcatctatcagacatccaccagggccactggcatcccagacaggttcagtggcagtgggtctgggacagatttcactctcaccatcagcagactggagcctgaagattttgcagtgtattactgtcagcagtatggtggctcacgggcgttcggccaagggaccaaggtggaactcaaacgagccgctgccttcgtgcctgtttttctgcccgcgaaacccacaactacccccgcccctcggcccccaactcctgcaccaactatcgcttcccaacccctgtctctgagacctgaggcatgccgccccgcggcaggcggcgccgtgcacactagaggcctggacttcgcctgcgatatttatatctgggccccccttgccgggacatgcggggtactgctgctgtctctggtgattaccctctactgcaaccacagaaacagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 64 - protein, 802 aa, Artificial Sequence
  source: /note=“Description of Artificial Sequence Synthetic polypeptide”
MALPVTALLLPLALLLHAARPQVQLVQSGAEVKKPGASVKVSCKASGYSFTSYDINWVRQATGQGLEWMGWMNPNSGNTGYAQKFQGRVTMTRDTSISTAYMELSSLRSEDTAVYYCGRAGYYYYFGMDVWGQGTTVTVSSGGGGSGGGGSGGGGSEIVLTQSPGTLSLSPGERATLSCRAGQSVTSSSLAWYQQKPGQAPRLLIYQTSTRATGIPDRFSGSGSGTDFTLTISRLEPEDFAVYYCQQYGGSRAFGQGTKVELKRAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 65 - DNA, 2436 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtctggggctgaggtgaagaagcctggggcctcagtgaaggtgtcctgcaaggcttctggatacagcttcaccagttatgatatcaactgggtgcgacaggccactggacaagggcttgagtggatggggtggatgaacccgaacagtggtaacacaggctatgcacagaagttccagggcagagtcaccatgaccagggacacctccataagcacagcctacatggaactgagcagcctgagatctgaggacacggccgtgtattactgtgggagagccggttactactactacttcggtatggacgtctggggccaagggaccacggtcaccgtatcctcaggaggcggcggttcaggcggaggtggctctggcggtggcggaagtgaaattgtgttgacgcagtctccaggcaccctgtctttgtctccaggggaaagagccaccctctcctgcagggccggtcagagtgttaccagcagctccttagcttggtaccagcagaaacctggccaggctcccaggctcctcatctatcagacatccaccagggccactggcatcccagacaggttcagtggcagtgggtctgggacagatttcactctcaccatcagcagactggagcctgaagattttgcagtgtattactgtcagcagtatggtggctcacgggcgttcggccaagggaccaaggtggaactcaaacgagccgctgccttcgtgcctgtttttctgcccgcgaaacccacaactacccccgcccctcggcccccaactcctgcaccaactatcgcttcccaacccctgtctctgagacctgaggcatgccgccccgcggcaggcggcgccgtgcacactagaggcctggacttcgcctgcgatatttatatctgggccccccttgccgggacatgcggggtactgctgctgtctctggtgattaccctctactgcaaccacagaaaccgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 66 - protein, 808 aa, Artificial Sequence
  source: /note=“Description of Artificial Sequence Synthetic polypeptide”
MALPVTALLLPLALLLHAARPQVQLVQSGAEVKKPGASVKVSCKASGYSFTSYDINWVRQATGQGLEWMGWMNPNSGNTGYAQKFQGRVTMTRDTSISTAYMELSSLRSEDTAVYYCGRAGYYYYFGMDVWGQGTTVTVSSGGGGSGGGGSGGGGSEIVLTQSPGTLSLSPGERATLSCRAGQSVTSSSLAWYQQKPGQAPRLLIYQTSTRATGIPDRFSGSGSGTDFTLTISRLEPEDFAVYYCQQYGGSRAFGQGTKVELKRAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 67 - DNA, 2496 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtctggggctgaggtgaagaagcctggggcctcagtgaaggtgtcctgcaaggcttctggatacaccttcaccggctactatatgcactgggtgcgacaggcccctggacaagggcttgagtggatgggatggatcaaccctaatagtggtggcacaaactctgcacagaagtttcagggcagggtcaccatgaccagggacacgtccatcagtacagcctacatggagctgaacaggctgagatctgacgacacggccgtttattactgtgcgagaggatggctacagacgtactactttgacaactggggccagggaaccctggtcaccgtatcctcaggaggcggcggttcaggcggaggtggctctggcggtggcggaagtgacatcgtgatgacccagtctccagactccctggctgtgtctctgggcgagagggccaccatctactgcaagtccagccagactgttttgaccagctccaacaataagaacttcttagcttggtaccaacagaaactaggacagcctcctaagctgctcatttcctgggcctctacccgggaatccggggtccctgaccgattcagtggcagcgggtctgggacagatttcactctcaccatcagcagcctgcaggctgaagatgtggcaatttattactgtcagcactattatactagtccactcactttcggcggcgggaccaaggtggagatcaaacgagccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagccgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 68 - protein, 828 aa, Artificial Sequence
  source: /note=“Description of Artificial Sequence Synthetic polypeptide”
MALPVTALLLPLALLLHAARPQVQLVQSGAEVKKPGASVKVSCKASGYTFTGYYMHWVRQAPGQGLEWMGWINPNSGGTNSAQKFQGRVTMTRDTSISTAYMELNRLRSDDTAVYYCARGWLQTYYFDNWGQGTLVTVSSGGGGSGGGGSGGGGSDIVMTQSPDSLAVSLGERATIYCKSSQTVLTSSNNKNFLAWYQQKLGQPPKLLISWASTRESGVPDRFSGSGSGTDFTLTISSLQAEDVAIYYCQHYYTSPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 69 - DNA, 2355 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtctggggctgaggtgaagaagcctggggcctcagtgaaggtgtcctgcaaggcttctggatacaccttcaccggctactatatgcactgggtgcgacaggcccctggacaagggcttgagtggatgggatggatcaaccctaatagtggtggcacaaactctgcacagaagtttcagggcagggtcaccatgaccagggacacgtccatcagtacagcctacatggagctgaacaggctgagatctgacgacacggccgtttattactgtgcgagaggatggctacagacgtactactttgacaactggggccagggaaccctggtcaccgtatcctcaggaggcggcggttcaggcggaggtggctctggcggtggcggaagtgacatcgtgatgacccagtctccagactccctggctgtgtctctgggcgagagggccaccatctactgcaagtccagccagactgttttgaccagctccaacaataagaacttcttagcttggtaccaacagaaactaggacagcctcctaagctgctcatttcctgggcctctacccgggaatccggggtccctgaccgattcagtggcagcgggtctgggacagatttcactctcaccatcagcagcctgcaggctgaagatgtggcaatttattactgtcagcactattatactagtccactcactttcggcggcgggaccaaggtggagatcaaacgagccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 70 - protein, 781 aa, Artificial Sequence
  source: /note=“Description of Artificial Sequence Synthetic polypeptide”
MALPVTALLLPLALLLHAARPQVQLVQSGAEVKKPGASVKVSCKASGYTFTGYYMHWVRQAPGQGLEWMGWINPNSGGTNSAQKFQGRVTMTRDTSISTAYMELNRLRSDDTAVYYCARGWLQTYYFDNWGQGTLVTVSSGGGGSGGGGSGGGGSDIVMTQSPDSLAVSLGERATIYCKSSQTVLTSSNNKNFLAWYQQKLGQPPKLLISWASTRESGVPDRFSGSGSGTDFTLTISSLQAEDVAIYYCQHYYTSPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 71 - DNA, 2373 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtctggggctgaggtgaagaagcctggggcctcagtgaaggtgtcctgcaaggcttctggatacaccttcaccggctactatatgcactgggtgcgacaggcccctggacaagggcttgagtggatgggatggatcaaccctaatagtggtggcacaaactctgcacagaagtttcagggcagggtcaccatgaccagggacacgtccatcagtacagcctacatggagctgaacaggctgagatctgacgacacggccgtttattactgtgcgagaggatggctacagacgtactactttgacaactggggccagggaaccctggtcaccgtatcctcaggaggcggcggttcaggcggaggtggctctggcggtggcggaagtgacatcgtgatgacccagtctccagactccctggctgtgtctctgggcgagagggccaccatctactgcaagtccagccagactgttttgaccagctccaacaataagaacttcttagcttggtaccaacagaaactaggacagcctcctaagctgctcatttcctgggcctctacccgggaatccggggtccctgaccgattcagtggcagcgggtctgggacagatttcactctcaccatcagcagcctgcaggctgaagatgtggcaatttattactgtcagcactattatactagtccactcactttcggcggcgggaccaaggtggagatcaaacgagccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttcgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 72 - protein, 787 aa, Artificial Sequence
  source: /note=“Description of Artificial Sequence Synthetic polypeptide”
MALPVTALLLPLALLLHAARPQVQLVQSGAEVKKPGASVKVSCKASGYTFTGYYMHWVRQAPGQGLEWMGWINPNSGGTNSAQKFQGRVTMTRDTSISTAYMELNRLRSDDTAVYYCARGWLQTYYFDNWGQGTLVTVSSGGGGSGGGGSGGGGSDIVMTQSPDSLAVSLGERATIYCKSSQTVLTSSNNKNFLAWYQQKLGQPPKLLISWASTRESGVPDRFSGSGSGTDFTLTISSLQAEDVAIYYCQHYYTSPLTFGGGTKVEIKRAAALDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 73 - DNA, 2433 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtctggggctgaggtgaagaagcctggggcctcagtgaaggtgtcctgcaaggcttctggatacaccttcaccggctactatatgcactgggtgcgacaggcccctggacaagggcttgagtggatgggatggatcaaccctaatagtggtggcacaaactctgcacagaagtttcagggcagggtcaccatgaccagggacacgtccatcagtacagcctacatggagctgaacaggctgagatctgacgacacggccgtttattactgtgcgagaggatggctacagacgtactactttgacaactggggccagggaaccctggtcaccgtatcctcaggaggcggcggttcaggcggaggtggctctggcggtggcggaagtgacatcgtgatgacccagtctccagactccctggctgtgtctctgggcgagagggccaccatctactgcaagtccagccagactgttttgaccagctccaacaataagaacttcttagcttggtaccaacagaaactaggacagcctcctaagctgctcatttcctgggcctctacccgggaatccggggtccctgaccgattcagtggcagcgggtctgggacagatttcactctcaccatcagcagcctgcaggctgaagatgtggcaatttattactgtcagcactattatactagtccactcactttcggcggcgggaccaaggtggagatcaaacgagccgctgccttcgtgcctgtttttctgcccgcgaaacccacaactacccccgcccctcggcccccaactcctgcaccaactatcgcttcccaacccctgtctctgagacctgaggcatgccgccccgcggcaggcggcgccgtgcacactagaggcctggacttcgcctgcgatatttatatctgggccccccttgccgggacatgcggggtactgctgctgtctctggtgattaccctctactgcaaccacagaaacagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 74 - protein, 807 aa, Artificial Sequence
  source: /note=“Description of Artificial Sequence Synthetic polypeptide”
MALPVTALLLPLALLLHAARPQVQLVQSGAEVKKPGASVKVSCKASGYTFTGYYMHWVRQAPGQGLEWMGWINPNSGGTNSAQKFQGRVTMTRDTSISTAYMELNRLRSDDTAVYYCARGWLQTYYFDNWGQGTLVTVSSGGGGSGGGGSGGGGSDIVMTQSPDSLAVSLGERATIYCKSSQTVLTSSNNKNFLAWYQQKLGQPPKLLISWASTRESGVPDRFSGSGSGTDFTLTISSLQAEDVAIYYCQHYYTSPLTFGGGTKVEIKRAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 75 - DNA, 2451 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtgcagtctggggctgaggtgaagaagcctggggcctcagtgaaggtgtcctgcaaggcttctggatacaccttcaccggctactatatgcactgggtgcgacaggcccctggacaagggcttgagtggatgggatggatcaaccctaatagtggtggcacaaactctgcacagaagtttcagggcagggtcaccatgaccagggacacgtccatcagtacagcctacatggagctgaacaggctgagatctgacgacacggccgtttattactgtgcgagaggatggctacagacgtactactttgacaactggggccagggaaccctggtcaccgtatcctcaggaggcggcggttcaggcggaggtggctctggcggtggcggaagtgacatcgtgatgacccagtctccagactccctggctgtgtctctgggcgagagggccaccatctactgcaagtccagccagactgttttgaccagctccaacaataagaacttcttagcttggtaccaacagaaactaggacagcctcctaagctgctcatttcctgggcctctacccgggaatccggggtccctgaccgattcagtggcagcgggtctgggacagatttcactctcaccatcagcagcctgcaggctgaagatgtggcaatttattactgtcagcactattatactagtccactcactttcggcggcgggaccaaggtggagatcaaacgagccgctgccttcgtgcctgtttttctgcccgcgaaacccacaactacccccgcccctcggcccccaactcctgcaccaactatcgcttcccaacccctgtctctgagacctgaggcatgccgccccgcggcaggcggcgccgtgcacactagaggcctggacttcgcctgcgatatttatatctgggccccccttgccgggacatgcggggtactgctgctgtctctggtgattaccctctactgcaaccacagaaaccgcttttccgtcgttaagcgggggagaaaaaagctgctgtacattttcaaacagccgtttatgaggccggtccaaacgactcaggaagaggacggctgctcctgccgctttcctgaggaggaggagggcgggtgcgaactgagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagggccaagagaagtggcagcggggagggccggggatctctccttacatgtggggacgtggaagaaaatccggggcctatgggtgccggcgccacgggaagggctatggatggcccgcgactgcttctcctgctgttgttgggcgtgtctctcggaggcgctaaggaggcctgtccaacgggcctctacactcactccggtgaatgttgcaaagcctgtaaccttggcgagggcgtcgcacaaccttgtggtgctaaccagacagtctgtgaaccatgcctggattcagtgacattcagcgatgttgtctcagccaccgagccttgcaagccttgtaccgaatgtgtgggccttcagtccatgtccgccccctgtgtcgaagccgatgatgcagtgtgcagatgtgcctatggatattaccaggacgaaactaccgggcggtgtgaggcctgccgggtgtgtgaagccggctctggcctcgtgttcagttgccaggataagcaaaacacagtatgtgaggagtgtccagacggaacctacagcgacgaggcgaaccacgtcgacccttgcttgccgtgcaccgtctgcgaggataccgaacgccagctgagagagtgtacgcgctgggcagacgctgagtgcgaggagatccctgggagatggatcacccggagcacacctcctgagggatcagacagtacagccccgagtacccaagaaccggaggcccctccagagcaggacctgatcgcttctacagttgctggcgtggtgacgacagtcatgggatcctcacaaccagtcgtgacgcggggcacaaccgacaatctgattcctgtctactgtagcatcttggcagccgtggtcgtgggcctggtagcctacatcgcctttaagagatgacctaggtaa

SEQ ID NO: 76 - protein, 813 aa, Artificial Sequence
  source: /note=“Description of Artificial Sequence Synthetic polypeptide”
MALPVTALLLPLALLLHAARPQVQLVQSGAEVKKPGASVKVSCKASGYTFTGYYMHWVRQAPGQGLEWMGWINPNSGGTNSAQKFQGRVTMTRDTSISTAYMELNRLRSDDTAVYYCARGWLQTYYFDNWGQGTLVTVSSGGGGSGGGGSGGGGSDIVMTQSPDSLAVSLGERATIYCKSSQTVLTSSNNKNFLAWYQQKLGQPPKLLISWASTRESGVPDRFSGSGSGTDFTLTISSLQAEDVAIYYCQHYYTSPLTFGGGTKVEIKRAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAKRSGSGEGRGSLLTCGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 77 - protein, 339 aa, Homo sapiens
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQL

SEQ ID NO: 78 - DNA, 63 bp, Artificial Sequence
  source: /note=“Description of Artificial Sequence Synthetic oligonucleotide”
atggcactccccgtaactgctctgctgctgccgttggcattgctcctgcacgccgcacgcccg

SEQ ID NO: 79 - protein, 21 aa, Artificial Sequence
  source: /note=“Description of Artificial Sequence Synthetic peptide”
MALPVTALLLPLALLLHAARP

SEQ ID NO: 80 - DNA, 45 bp, Artificial Sequence
  source: /note=“Description of Artificial Sequence Synthetic oligonucleotide”
ggcggtggaggctccggaggggggggctctggcggagggggctcc

SEQ ID NO: 81 - protein, 15 aa, Artificial Sequence
  source: /note=“Description of Artificial Sequence Synthetic peptide”
GGGGSGGGGSGGGGS

SEQ ID NO: 82 - DNA, 54 bp, Artificial Sequence
  source: /note=“Description of Artificial Sequence Synthetic oligonucleotide”
gggtctacatccggctccgggaagcccggaagtggcgaaggtagtacaaagggg

SEQ ID NO: 83 - protein, 18 aa, Artificial Sequence
  source: /note=“Description of Artificial Sequence Synthetic peptide”
GSTSGSGKPGSGEGSTKG

SEQ ID NO: 84 - DNA, 126 bp, Unknown
  source: /note=“Description of Unknown 4-1BB sequence”
aagcgcggcaggaagaagctcctctacatttttaagcagccttttatgaggcccgtacagacaacacaggaggaagatggctgtagctgcagatttcccgaggaggaggaaggtgggtgcgagctg

SEQ ID NO: 85 - protein, 42 aa, Unknown
  source: /note=“Description of Unknown 4-1BB sequence”
KRGRKKLLYIFKQPFMRPVQTTQEEDGCSCRFPEEEEGGCEL

SEQ ID NO: 86 - protein, 37 aa, Unknown
  source: /note=“Description of Unknown OX40 sequence”
RRDQRLPPDAHKPPGGGSFRTPIQEEQADAHSTLAKI

SEQ ID NO: 87 - DNA, 363 bp, Artificial Sequence
  source: /note=“Description of Artificial Sequence Synthetic polynucleotide”
caggttcagctgcagcagtctggagctgagatgatgaagcctggggcctcagtgaagatatcctgcaaggctactggctacacattcagtacctactggatagagtgggtaaagcagaggcctggacatggccttgagtggattggagagattttacctggaagtggtaatactgacttcaatgagaagttcaagggcaaggccacattcactgcagatacatcctccgacacagcctacatgcatctcagcagcctgacatctgaggactctgccgtctattactgtacaagatgggggtactacggtactagggggtacttcaatgtctggggcgcagggtccacggtcaccgtctcctca

SEQ ID NO: 88 - protein, 121 aa, Artificial Sequence
  source: /note=“Description of Artificial Sequence Synthetic polypeptide”
QVQLVQSGAEVKKPGASVKVSCKASGYTFSTYWIEWVRQAPGQRLEWMGEILPGSGNTDFNEKFQGRVTFTADTSSDTAYMELSSLRSEDTAVYYCTRWGYYGTRGYFNVWGQGTLVTVSS

SEQ ID NO: 89 - protein, 5 aa, Artificial Sequence
  source: /note=“Description of Artificial Sequence Synthetic peptide”
TYWIE

SEQ ID NO: 90 - protein, 17 aa, Artificial Sequence
  source: /note=“Description of Artificial Sequence Synthetic peptide”
EILPGSGNTDFNEKFQG

SEQ ID NO: 91 - protein, 12 aa, Artificial Sequence
  source: /note=“Description of Artificial Sequence Synthetic peptide”
WGYYGTRGYFNV

SEQ ID NO: 92 - DNA, 318 bp, Artificial Sequence
  source: /note=“Description of Artificial Sequence Synthetic polynucleotide”
caaattgttctcacccagtctccagcaatcatgtctgcatctccaggggagaaggtcaccataacgtgcagtgccagctcaagtgtaagttacatgcactggttccagcagaagccaggcacttctcccaaactctggatttatagcacctccaacctggcttctggagtccctgctcgcttcagtggcagtggatctgggacctcttactctctcacaatcagccgaatggaggctgaagatgctgccacttattactgccagcaaaggcgtagtttcccgtacacgttcggaggggggaccaagctggaaattaaa

SEQ ID NO: 93 - protein, 106 aa, Artificial Sequence [sequence omitted]

SEQ ID NO: 94 - protein, 10 aa, Artificial Sequence
  source: /note=“Description of Artificial Sequence Synthetic peptide”
RASSSVSYMH

SEQ ID NO: 95 - protein, 7 aa, Artificial Sequence
  source: /note=“Description of Artificial Sequence Synthetic peptide”
STSNLAS

SEQ ID NO: 96 - protein, 9 aa, Artificial Sequence
  source: /note=“Description of Artificial Sequence Synthetic peptide”
QQRRSFPYT

SEQ ID NO: 97 - DNA, 363 bp, Artificial Sequence
  source: /note=“Description of Artificial Sequence Synthetic polynucleotide”
cagatccagttggtgcagtctggacctgaactgaagaagcctggagagacagtcaagatctcctgcaaggcttctggatataccttcacaaactatggaatgaactgggtgaagcaggctccaggaaagggtttacagtggatgggctggatgaacacttatactggagagccaacatatgctgatgacttcaagggacggtttgccttctctttggaaacctctgccagaactgtctctttggacatcaacgacctcaaaaatgaggacacggctacatatttctgtacaagagcagggggacaactcaggcccggggctatggactactggggtcaaggaacctcagtcaccgtctcctca

SEQ ID NO: 98 - protein, 119 aa, Artificial Sequence
  source: /note=“Description of Artificial Sequence Synthetic polypeptide”
QLVQSGAEVKKPGATVKISCKASGYTFTNYGMNWVQQAPGQGLEWMGWMNTYTGEPTYADKFQGRVTFTLDTSARTVYMELSSLRSEDTAVYFCARAGGQLRPGAMDYWGQGTMVTVSS

SEQ ID NO: 99 - protein, 5 aa, Artificial Sequence
  source: /note=“Description of Artificial Sequence Synthetic peptide”
NYGMN

SEQ ID NO: 100 - protein, 17 aa, Artificial Sequence
  source: /note=“Description of Artificial Sequence Synthetic peptide”
WMNTYTGEPTYADKFQG

SEQ ID NO: 101 - protein, 12 aa, Artificial Sequence
  source: /note=“Description of Artificial Sequence Synthetic peptide”
AGGQLRPGAMDY

SEQ ID NO: 102 - DNA, 333 bp, Artificial Sequence
  source: /note=“Description of Artificial Sequence Synthetic polynucleotide”
gacattgtgctgacccaatctccagcttctttggctgtgtctctagggcagagggccaccatctcctgcaaggccagccaaagtgttgattatgatggtgatagttttatgaactggtaccaacagaaaccaggacagccacccaaactcctcatctatgttgcatccaatctagaatctgggatcccagacaggtttagtggcagtgggtctgggacagacttcaccctcaacatccatcctgtggaggaggaggatgctgcaacctattattgtcagcaaagtaatgaggaacctccgacgttcggtggaggcaccaagctggaaatcaaa

SEQ ID NO: 103 - protein, 111 aa, Artificial Sequence
  source: /note=“Description of Artificial Sequence Synthetic polypeptide”
DIVLTQTPLSLSVTPGQPASISCKASQSVDYDGDSFMNWYLQKPGQPPQLLIYVASNLESGVPDRFSGSGSGTDFTLKISRVEAEDVGVYYCQQSNEEPPTFGQGTKLEIK

SEQ ID NO: 104 - protein, 15 aa, Artificial Sequence
  source: /note=“Description of Artificial Sequence Synthetic peptide”
KASQSVDYDGDSFMN

SEQ ID NO: 105 - protein, 7 aa, Artificial Sequence
  source: /note=“Description of Artificial Sequence Synthetic peptide”
VASNLES

SEQ ID NO: 106 - protein, 9 aa, Artificial Sequence
  source: /note=“Description of Artificial Sequence Synthetic peptide”
QQSNEEPPT

SEQ ID NO: 107 - DNA, 360 bp, Artificial Sequence
  source: /note=“Description of Artificial Sequence Synthetic polynucleotide”
cagatacaactggtggagtctgggggaggcgtggtccagcctgggaggtccctgagactctcctgtgtagcgtctggattcaccttcaagaactatggcatgcactgggtccgccaggctccaggcaaggggctggagtgggtggcagttatttggtatgatggaagtaatgaatactatggagaccccgtgaagggccgattcaccatctccagagacaactccaagaacatgttgtatctgcaaatgaacagcctgagagccgatgacacggctgtgtattactgtgcgaggtcgggaatagcagtggctggggcctttgactactggggccagggaaccctggtcaccgtctcctca

SEQ ID NO: 108 - protein, 121 aa, Artificial Sequence
  source: /note=“Description of Artificial Sequence Synthetic polypeptide”
QVQLVQSGAEVKKPGASVKVSCKASGYTFTTYWMHWVRQAPGQGLEWMGEINPSSGRTNYNEKFKTRVTMTRDTSTSTVYMELSSLRSEDTAVYYCAKLGPGPQYYAMDYWGQGTMVTVSS

SEQ ID NO: 109 - protein, 5 aa, Artificial Sequence
  source: /note=“Description of Artificial Sequence Synthetic peptide”
TYWMH

SEQ ID NO: 110 - protein, 17 aa, Artificial Sequence
  source: /note=“Description of Artificial Sequence Synthetic peptide”
EINPSSGRTNYNEKFKT

SEQ ID NO: 111 - protein, 12 aa, Artificial Sequence
  source: /note=“Description of Artificial Sequence Synthetic peptide”
LGPGPQYYAMDY

SEQ ID NO: 112 - DNA, 327 bp, Artificial Sequence
  source: /note=“Description of Artificial Sequence Synthetic polynucleotide”
gaaattgtgttgacgcagtctccagacaccctgtctttgtctccaggggaaaaagccaccctctcctgcagggccagtcagagtgttagcagcagcttcttggcctggtaccagcagaaacctggacaggctcccagtctcctcatctatgttgcatccagaagggccgctggcatccctgacaggttcagtggcagtgggtctgggacagacttcactctcaccatcagcagactggagcctgaagattttggaatgttttactgtcaacactatggtaggacaccattcactttcggccctgggaccaaagtggatatcaaacga

SEQ ID NO: 113 - protein, 107 aa, Artificial Sequence
  source: /note=“Description of Artificial Sequence Synthetic polypeptide”
DIQMTQSPSSLSASVGDRVTITCHASQNINVWLSWYQQKPGKAPKLLIYKASKLHTGVPSRFSGSGSGTDFTLTISSLQPEDFATYYCQQGQSYPWTFGQGTKLEIK

SEQ ID NO: 114 - protein, 11 aa, Artificial Sequence
  source: /note=“Description of Artificial Sequence Synthetic peptide”
HASQNINVWLS

SEQ ID NO: 115 - protein, 7 aa, Artificial Sequence
  source: /note=“Description of Artificial Sequence Synthetic peptide”
KASKLHT

SEQ ID NO: 116 - protein, 9 aa, Artificial Sequence
  source: /note=“Description of Artificial Sequence Synthetic peptide”
QQGQSYPWT

SEQ ID NO: 117 - DNA, 357 bp, Artificial Sequence
  source: /note=“Description of Artificial Sequence Synthetic polynucleotide”
caggtgcagctggtgcagtctggggctgaggtgaagaagcctggggcctcagtgaaggtctcctgcaaggcttctggatacaccttcaccggctactatatacactgggtgcgacaggcccctgaacaagggcttgagtggatgggatggatcaaccctaacagtggtggcacaaactatgcacagaagtttcagggcagggtcaccatggccagggacacgtccatcagcacagtttacatggacctgagcaggctgagatctgacgacacggccgtgtattactgtgcgagaatacgcggtggtaactcggtctttgactactggggccagggaaccctggtcaccgtctcctca

SEQ ID NO: 118 - protein, 120 aa, Artificial Sequence
  source: /note=“Description of Artificial Sequence Synthetic polypeptide”
QVQLVQSGAEVKKPGASVKVSCKASGYSFTSYDINWVRQATGQGLEWMGWMNPNSGNTGYAQKFQGRVTMTRDTSISTAYMELSSLRSEDTAVYYCGRAGYYYYFGMDVWGQGTTVTVSS

SEQ ID NO: 119 - protein, 5 aa, Artificial Sequence
  source: /note=“Description of Artificial Sequence Synthetic peptide”
SYDIN

SEQ ID NO: 120 - protein, 17 aa, Artificial Sequence
  source: /note=“Description of Artificial Sequence Synthetic peptide”
WMNPNSGNTGYAQKFQG

SEQ ID NO: 121 - protein, 11 aa, Artificial Sequence
  source: /note=“Description of Artificial Sequence Synthetic peptide”
AGYYYYFGMDV

SEQ ID NO: 122 - DNA, 342 bp, Artificial Sequence
  source: /note=“Description of Artificial Sequence Synthetic polynucleotide”
gacatcgtgatgacccagtctccagactccctggctgtgtctctgggcgagagggccaccatcaactgcaagtccacccagagtattttatacacctccaacaataagaacttcttagcttggtaccagcagaaaccagggcagcctcctaaactgctcatttcctgggcatctatccgggaatccggggtccctgaccgattcagtggcagcgggtctgggacagatttcgctctcaccatcagcagcctgcaggctgaagatgtggcagtttattactgtcaacaatattttagtactatgttcagttttggccaggggaccaagctggagatcaaacga

SEQ ID NO: 123 - protein, 108 aa, Artificial Sequence
  source: /note=“Description of Artificial Sequence Synthetic polypeptide”
EIVLTQSPGTLSLSPGERATLSCRAGQSVTSSSFAWYQQKPGQAPRLLIYQTSTRATGIPDRFSGSGSGTDFTLTISRLEPEDFAVYYCQQYGGSRSFGQGTKVELKR

SEQ ID NO: 124 - protein, 12 aa, Artificial Sequence
  source: /note=“Description of Artificial Sequence Synthetic peptide”
RAGQSVTSSSFA

SEQ ID NO: 125 - protein, 7 aa, Artificial Sequence
  source: /note=“Description of Artificial Sequence Synthetic peptide”
QTSTRAT

SEQ ID NO: 126 - protein, 8 aa, Artificial Sequence
  source: /note=“Description of Artificial Sequence Synthetic peptide”
QQYGGSRS

SEQ ID NO: 127 - DNA, 360 bp, Artificial Sequence
  source: /note=“Description of Artificial Sequence Synthetic polynucleotide”
caggtgcagctgcaggagtcgggcccaggactggtgaagccttcacagaccctgtccctcacctgcactgtctctggtggctccatcagtagtggtgcatactactggacttggatccgccagcacccagggaagggcctggagtggattgggtacatccattacagtgggagcacctactccaacccgtccctcaagagtcgaattaccatatcgttagacacgtctaagaaccagttctccctgaagctgaactctgtgactgccgcggacacggccgtgtattactgtgcgagacaagaggactacggtggtttgtttgactactggggccagggaaccctggtcaccgtttcctca

SEQ ID NO: 128 - protein, 120 aa, Artificial Sequence; identical to SEQ ID NO: 118
  source: /note=“Description of Artificial Sequence Synthetic polypeptide”

SEQ ID NO: 129 - protein, 5 aa, Artificial Sequence
  source: /note=“Description of Artificial Sequence Synthetic peptide”
SYDIN

SEQ ID NO: 130 - protein, 17 aa, Artificial Sequence
  source: /note=“Description of Artificial Sequence Synthetic peptide”
WMNPNSGNTGYAQKFQG

SEQ ID NO: 131 - protein, 11 aa, Artificial Sequence
  source: /note=“Description of Artificial Sequence Synthetic peptide”
AGYYYYFGMDV

SEQ ID NO: 132 - DNA, 324 bp, Artificial Sequence
  source: /note=“Description of Artificial Sequence Synthetic polynucleotide”
gaaatagtgatgacgcagtctccagccaccctgtctgtgtctccaggggaaagaatcaccctctcctgcagggccagtcagagtgttaccaccgacttagcctggtaccagcagatgcctggacaggctccccggctcctcatctatgatgcttccaccagggccactggtttcccagccagattcagtggcagtgggtctgggacagacttcacgctcaccatcagcagcctgcaggctgaagattttgcagtttattactgtcaacattataaaacctggcctctcactttcggcggagggactaaggtggagatcaaacga

SEQ ID NO: 133 - protein, 108 aa, Artificial Sequence
  source: /note=“Description of Artificial Sequence Synthetic polypeptide”
EIVLTQSPGTLSLSPGERATLSCRAGQSVTSSSLAWYQQKPGQAPRLLIYQTSTRATGIPDRFSGSGSGTDFTLTISRLEPEDFAVYYCQQYGGSRAFGQGTKVELKR

SEQ ID NO: 134 - protein, 12 aa, Artificial Sequence
  source: /note=“Description of Artificial Sequence Synthetic peptide”
RAGQSVTSSSLA

SEQ ID NO: 135 - protein, 7 aa, Artificial Sequence
  source: /note=“Description of Artificial Sequence Synthetic peptide”
QTSTRAT

SEQ ID NO: 136 - protein, 8 aa, Artificial Sequence
  source: /note=“Description of Artificial Sequence Synthetic peptide”
QQYGGSRA

SEQ ID NO: 137 - DNA, 360 bp, Artificial Sequence; identical to SEQ ID NO: 127
  source: /note=“Description of Artificial Sequence Synthetic polynucleotide”

SEQ ID NO: 138 - protein, 119 aa, Artificial Sequence
  source: /note=“Description of Artificial Sequence Synthetic polypeptide”
QVQLVQSGAEVKKPGASVKVSCKASGYTFTGYYMHWVRQAPGQGLEWMGWINPNSGGTNSAQKFQGRVTMTRDTSISTAYMELNRLRSDDTAVYYCARGWLQTYYFDNWGQGTLVTVSS

SEQ ID NO: 139 - protein, 5 aa, Artificial Sequence
  source: /note=“Description of Artificial Sequence Synthetic peptide”
GYYMH

SEQ ID NO: 140 - protein, 17 aa, Artificial Sequence
  source: /note=“Description of Artificial Sequence Synthetic peptide”
WINPNSGGTNSAQKFQG

SEQ ID NO: 141 - protein, 10 aa, Artificial Sequence
  source: /note=“Description of Artificial Sequence Synthetic peptide”
GWLQTYYFDN

SEQ ID NO: 142 - DNA, 324 bp, Artificial Sequence; identical to SEQ ID NO: 132
  source: /note=“Description of Artificial Sequence Synthetic polynucleotide”

SEQ ID NO: 143 - protein, 114 aa, Artificial Sequence
  source: /note=“Description of Artificial Sequence Synthetic polypeptide”
DIVMTQSPDSLAVSLGERATIYCKSSQTVLTSSNNKNFLAWYQQKLGQPPKLLISWASTRESGVPDRFSGSGSGTDFTLTISSLQAEDVAIYYCQHYYTSPLTFGGGTKVEIKR

SEQ ID NO: 144 - protein, 13 aa, Artificial Sequence
  source: /note=“Description of Artificial Sequence Synthetic peptide”
TVLTSSNNKNFLA

SEQ ID NO: 145 - protein, 7 aa, Artificial Sequence
  source: /note=“Description of Artificial Sequence Synthetic peptide”
WASTRES

SEQ ID NO: 146 - protein, 9 aa, Artificial Sequence
  source: /note=“Description of Artificial Sequence Synthetic peptide”
QHYYTSPLT

SEQ ID NO: 147 - DNA, 6762 bp, Artificial Sequence
  source: /note=“Description of Artificial Sequence Synthetic polynucleotide”
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgacccggggatggcgcgccagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagccgccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgcgatcgcggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtccttcgaagtagatctttgtcgatcctaccatccactcgacacacccgccagcggccgctgccaagcttccgagctctcgaattaattcacggtacccaccatggcctagggagactagtcgaatcgatatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtccttttcatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggttaattaaagtacctttaagaccaatgacttacaaggcagctgtagatcttagccactttttaaaagaaaaggggggactggaagggcgaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcaggcatgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttggcgcgccatcgtcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac